SEQUENCE LISTING (125 sequences)

SEQ ID NO: 1 - RNA, 21 nt, Arabidopsis thaliana
  source 1..21: mol_type = other RNA
tcggaccaggcttcattcccc

SEQ ID NO: 2 - RNA, 21 nt, Arabidopsis thaliana
  source 1..21: mol_type = other RNA
tcggaccaggcttcattcccc

SEQ ID NO: 3 - RNA, 21 nt, Arabidopsis thaliana
  source 1..21: mol_type = other RNA
tcggaccaggcttcattcccc

SEQ ID NO: 4 - RNA, 21 nt, Arabidopsis thaliana
  source 1..21: mol_type = other RNA
ttccacagctttcttgaactg

SEQ ID NO: 5 - RNA, 21 nt, Arabidopsis thaliana
  source 1..21: mol_type = other RNA
ttccacagctttcttgaactt

SEQ ID NO: 6 - RNA, 20 nt, Arabidopsis thaliana
  source 1..20: mol_type = other RNA
tgacagaagagagtgagcac

SEQ ID NO: 7 - RNA, 21 nt, Arabidopsis thaliana
  source 1..21: mol_type = other RNA
tcgcttggtgcaggtcgggaa

SEQ ID NO: 8 - RNA, 20 nt, Arabidopsis thaliana
  source 1..20: mol_type = other RNA
tgacagaagagagtgagcac

SEQ ID NO: 9 - RNA, 21 nt, Arabidopsis thaliana
  source 1..21: mol_type = other RNA
tcgataaacctctgcatccag

SEQ ID NO: 10 - RNA, 21 nt, Arabidopsis thaliana
  source 1..21: mol_type = other RNA
tcgataaacctctgcatccag

SEQ ID NO: 11 - RNA, 21 nt, Arabidopsis thaliana
  source 1..21: mol_type = other RNA
gttcaataaagctgtgggaag

SEQ ID NO: 12 - RNA, 21 nt, Arabidopsis thaliana
  source 1..21: mol_type = other RNA
tcgcttggtgcaggtcgggaa

SEQ ID NO: 13 - RNA, 20 nt, Arabidopsis thaliana
  source 1..20: mol_type = other RNA
tgacagaagagagtgagcac

SEQ ID NO: 14 - RNA, 20 nt, Arabidopsis thaliana
  source 1..20: mol_type = other RNA
tgacagaagagagtgagcac

SEQ ID NO: 15 - RNA, 20 nt, Arabidopsis thaliana
  source 1..20: mol_type = other RNA
tgacagaagagagtgagcac

SEQ ID NO: 16 - RNA, 21 nt, Arabidopsis thaliana
  source 1..21: mol_type = other RNA
tggagaagcagggcacgtgcg

SEQ ID NO: 17 - RNA, 21 nt, Arabidopsis thaliana
  source 1..21: mol_type = other RNA
atgcactgcctcttccctggc

SEQ ID NO: 18 - RNA, 21 nt, Arabidopsis thaliana
  source 1..21: mol_type = other RNA
tcggaccaggcttcatccccc

SEQ ID NO: 19 - RNA, 21 nt, Arabidopsis thaliana
  source 1..21: mol_type = other RNA
tgcctggctccctgtatgcca

SEQ ID NO: 20 - RNA, 21 nt, Arabidopsis thaliana
  source 1..21: mol_type = other RNA
ttgacagaagatagagagcac

SEQ ID NO: 21 - RNA, 21 nt, Arabidopsis thaliana
  source 1..21: mol_type = other RNA
ttgacagaagatagagagcac

SEQ ID NO: 22 - RNA, 21 nt, Arabidopsis thaliana
  source 1..21: mol_type = other RNA
tggagaagcagggcacgtgca

SEQ ID NO: 23 - RNA, 21 nt, Arabidopsis thaliana
  source 1..21: mol_type = other RNA
ttcttgtggattccttggaaa

SEQ ID NO: 24 - RNA, 21 nt, Arabidopsis thaliana
  source 1..21: mol_type = other RNA
acagtttgtgttttgttttgt

SEQ ID NO: 25 - RNA, 21 nt, Arabidopsis thaliana
  source 1..21: mol_type = other RNA
tggaagaaggtgagacttgca

SEQ ID NO: 26 - RNA, 24 nt, Arabidopsis thaliana
  source 1..24: mol_type = other RNA
tcctgtgtttcctttgatgcgtgg

SEQ ID NO: 27 - RNA, 21 nt, Arabidopsis thaliana
  source 1..21: mol_type = other RNA
ctttgtctacaattttggaaa

SEQ ID NO: 28 - RNA, 21 nt, Arabidopsis thaliana
  source 1..21: mol_type = other RNA
ctgaagtgtttgggggaactc

SEQ ID NO: 29 - RNA, 22 nt, Arabidopsis thaliana
  source 1..22: mol_type = other RNA
ttcgaggcctattaaacctctg

SEQ ID NO: 30 - RNA, 20 nt, Arabidopsis thaliana
  source 1..20: mol_type = other RNA
agaggtgaccattggagatg

SEQ ID NO: 31 - RNA, 21 nt, Arabidopsis thaliana
  source 1..21: mol_type = other RNA
gctaagagcggttctgatgga

SEQ ID NO: 32 - RNA, 21 nt, Arabidopsis thaliana
  source 1..21: mol_type = other RNA
gctaagagcggttctgatgga

SEQ ID NO: 33 - RNA, 21 nt, Arabidopsis thaliana
  source 1..21: mol_type = other RNA
agaagcaaaatgacgactcgg

SEQ ID NO: 34 - RNA, 21 nt, Arabidopsis thaliana
  source 1..21: mol_type = other RNA
acagtttgtgttttgttttgt

SEQ ID NO: 35 - RNA, 21 nt, Arabidopsis thaliana
  source 1..21: mol_type = other RNA
ggaatcttgatgatgctgcat

SEQ ID NO: 36 - RNA, 21 nt, Arabidopsis thaliana
  source 1..21: mol_type = other RNA
ccgtatcttggccttgtcatt

SEQ ID NO: 37 - RNA, 22 nt, Arabidopsis thaliana
  source 1..22: mol_type = other RNA
ttggggacgagatgttttgttg

SEQ ID NO: 38 - RNA, 21 nt, Arabidopsis thaliana
  source 1..21: mol_type = other RNA
tcatcttcatcatcatcgtca

SEQ ID NO: 39 - RNA, 21 nt, Arabidopsis thaliana
  source 1..21: mol_type = other RNA
gatcatgttcgcagtttcacc

SEQ ID NO: 40 - RNA, 20 nt, Arabidopsis thaliana
  source 1..20: mol_type = other RNA
gcagcaccattaagattcac

SEQ ID NO: 41 - RNA, 21 nt, Arabidopsis thaliana
  source 1..21: mol_type = other RNA
cgtagttgcagagcttgacgg

SEQ ID NO: 42 - RNA, 21 nt, Arabidopsis thaliana
  source 1..21: mol_type = other RNA
ccttctcatcgatggtctaga

SEQ ID NO: 43 - RNA, 20 nt, Arabidopsis thaliana
  source 1..20: mol_type = other RNA
gcagcaccattaagattcac

SEQ ID NO: 44 - RNA, 24 nt, Arabidopsis thaliana
  source 1..24: mol_type = other RNA
attaacgctggcggttgcggcagc

SEQ ID NO: 45 - RNA, 22 nt, Arabidopsis thaliana
  source 1..22: mol_type = other RNA
ttggggacgagatgttttgttg

SEQ ID NO: 46 - RNA, 21 nt, Arabidopsis thaliana
  source 1..21: mol_type = other RNA
tggttttggacacgtgaaaat

SEQ ID NO: 47 - RNA, 21 nt, Arabidopsis thaliana
  source 1..21: mol_type = other RNA
tgagccaaggatgacttgccg

SEQ ID NO: 48 - RNA, 21 nt, Arabidopsis thaliana
  source 1..21: mol_type = other RNA
cttcttaagtgctgataatgc

SEQ ID NO: 49 - RNA, 21 nt, Arabidopsis thaliana
  source 1..21: mol_type = other RNA
taactattttgagaagaagtg

SEQ ID NO: 50 - RNA, 21 nt, Arabidopsis thaliana
  source 1..21: mol_type = other RNA
tgagccaaggatgacttgccg

SEQ ID NO: 51 - RNA, 22 nt, Arabidopsis thaliana
  source 1..22: mol_type = other RNA
tcttgcttaaatgagtattcca

SEQ ID NO: 52 - RNA, 22 nt, Arabidopsis thaliana
  source 1..22: mol_type = other RNA
ttgtgttgcgtttctgttgatt

SEQ ID NO: 53 - RNA, 21 nt, Arabidopsis thaliana
  source 1..21: mol_type = other RNA
gcgtatgaggagccatgcata

SEQ ID NO: 54 - RNA, 21 nt, Arabidopsis thaliana
  source 1..21: mol_type = other RNA
tgttaaggagtgttaacggtg

SEQ ID NO: 55 - RNA, 21 nt, Arabidopsis thaliana
  source 1..21: mol_type = other RNA
tgccaaaggagagttgccctg

SEQ ID NO: 56 - RNA, 21 nt, Arabidopsis thaliana
  source 1..21: mol_type = other RNA
tgttaaggagtgttaacggtg

SEQ ID NO: 57 - RNA, 21 nt, Arabidopsis thaliana
  source 1..21: mol_type = other RNA
tgtgttctcaggtcacccctt

SEQ ID NO: 58 - RNA, 21 nt, Arabidopsis thaliana
  source 1..21: mol_type = other RNA
ttcgcaggagagatagcgcca

SEQ ID NO: 59 - RNA, 21 nt, Arabidopsis thaliana
  source 1..21: mol_type = other RNA
ttagagttttctggatactta

SEQ ID NO: 60 - RNA, 21 nt, Arabidopsis thaliana
  source 1..21: mol_type = other RNA
gctctctatacttctgtcacc

SEQ ID NO: 61 - RNA, 21 nt, Arabidopsis thaliana
  source 1..21: mol_type = other RNA
tgccaaaggagagttgccctg

SEQ ID NO: 62 - RNA, 21 nt, Arabidopsis thaliana
  source 1..21: mol_type = other RNA
atttgtacacctagatctgta

SEQ ID NO: 63 - RNA, 21 nt, Arabidopsis thaliana
  source 1..21: mol_type = other RNA
tgttaaggagtgttaacggtg

SEQ ID NO: 64 - RNA, 21 nt, Arabidopsis thaliana
  source 1..21: mol_type = other RNA
tgattggaaatttcgttgact

SEQ ID NO: 65 - RNA, 21 nt, Arabidopsis thaliana
  source 1..21: mol_type = other RNA
aagctcaggagggatagcgcc

SEQ ID NO: 66 - RNA, 22 nt, Arabidopsis thaliana
  source 1..22: mol_type = other RNA
tagaccgatgtcaacaaacaag

SEQ ID NO: 67 - RNA, 21 nt, Arabidopsis thaliana
  source 1..21: mol_type = other RNA
taactaaacattggtgtagta

SEQ ID NO: 68 - RNA, 21 nt, Arabidopsis thaliana
  source 1..21: mol_type = other RNA
tgttaaggagtgttaacggtg

SEQ ID NO: 69 - RNA, 21 nt, Arabidopsis thaliana
  source 1..21: mol_type = other RNA
caatttctagtgggtcgtatt

SEQ ID NO: 70 - RNA, 21 nt, Arabidopsis thaliana
  source 1..21: mol_type = other RNA
aagctcaggagggatagcgcc

SEQ ID NO: 71 - RNA, 22 nt, Arabidopsis thaliana
  source 1..22: mol_type = other RNA
ttggggacgacatcttttgttg

SEQ ID NO: 72 - RNA, 21 nt, Arabidopsis thaliana
  source 1..21: mol_type = other RNA
tggagaagatacgcaagaaag

SEQ ID NO: 73 - RNA, 21 nt, Arabidopsis thaliana
  source 1..21: mol_type = other RNA
acagtggtcatctggtgggct

SEQ ID NO: 74 - RNA, 21 nt, Arabidopsis thaliana
  source 1..21: mol_type = other RNA
ctttatatccgcatttgcgca

SEQ ID NO: 75 - RNA, 24 nt, Arabidopsis thaliana
  source 1..24: mol_type = other RNA
tgggttgagttgagttgagttggc

SEQ ID NO: 76 - RNA, 21 nt, Arabidopsis thaliana
  source 1..21: mol_type = other RNA
ggactgttgtctggctcgagg

SEQ ID NO: 77 - RNA, 21 nt, Arabidopsis thaliana
  source 1..21: mol_type = other RNA
gagctccttgaagttcaatgg

SEQ ID NO: 78 - RNA, 21 nt, Arabidopsis thaliana
  source 1..21: mol_type = other RNA
ggactgttgtctggctcgagg

SEQ ID NO: 79 - RNA, 21 nt, Arabidopsis thaliana
  source 1..21: mol_type = other RNA
tttaggtcgagcttcattgga

SEQ ID NO: 80 - RNA, 21 nt, Arabidopsis thaliana
  source 1..21: mol_type = other RNA
ttggtgttatgtgtagtcttc

SEQ ID NO: 81 - RNA, 21 nt, Arabidopsis thaliana
  source 1..21: mol_type = other RNA
ttagagttttctggatactta

SEQ ID NO: 82 - RNA, 21 nt, Arabidopsis thaliana
  source 1..21: mol_type = other RNA
acaaagttttatactgacaat

SEQ ID NO: 83 - RNA, 21 nt, Arabidopsis thaliana
  source 1..21: mol_type = other RNA
cagccaaggatgacttgccgg

SEQ ID NO: 84 - RNA, 21 nt, Arabidopsis thaliana
  source 1..21: mol_type = other RNA
tttgtgacatctaggtgcttt

SEQ ID NO: 85 - RNA, 21 nt, Arabidopsis thaliana
  source 1..21: mol_type = other RNA
tcaggtatgattgacttcaaa

SEQ ID NO: 86 - RNA, 21 nt, Arabidopsis thaliana
  source 1..21: mol_type = other RNA
tcaaggaacggattttgttaa

SEQ ID NO: 87 - RNA, 21 nt, Arabidopsis thaliana
  source 1..21: mol_type = other RNA
acatatgatctgcatctttgc

SEQ ID NO: 88 - RNA, 21 nt, Arabidopsis thaliana
  source 1..21: mol_type = other RNA
ttagattcacgcacaaactcg

SEQ ID NO: 89 - RNA, 21 nt, Arabidopsis thaliana
  source 1..21: mol_type = other RNA
cacgtgttctactactccaac

SEQ ID NO: 90 - RNA, 21 nt, Arabidopsis thaliana
  source 1..21: mol_type = other RNA
ttcttgcatatgttctttatc

SEQ ID NO: 91 - RNA, 21 nt, Arabidopsis thaliana
  source 1..21: mol_type = other RNA
tcggaccaggcttcatccccc

SEQ ID NO: 92 - RNA, 20 nt, Arabidopsis thaliana
  source 1..20: mol_type = other RNA
acttggctgattctattatt

SEQ ID NO: 93 - RNA, 21 nt, Arabidopsis thaliana
  source 1..21: mol_type = other RNA
ggccggtggtcgcgagaggga

SEQ ID NO: 94 - RNA, 21 nt, Arabidopsis thaliana
  source 1..21: mol_type = other RNA
tggcaggaaagacataatttt

SEQ ID NO: 95 - RNA, 21 nt, Arabidopsis thaliana
  source 1..21: mol_type = other RNA
tgccaaaggagatttgccctg

SEQ ID NO: 96 - RNA, 21 nt, Arabidopsis thaliana
  source 1..21: mol_type = other RNA
tcactggtaccaatcattcca

SEQ ID NO: 97 - RNA, 21 nt, Arabidopsis thaliana
  source 1..21: mol_type = other RNA
atgggacatcgagcatttaat

SEQ ID NO: 98 - RNA, 21 nt, Arabidopsis thaliana
  source 1..21: mol_type = other RNA
tggcttggtttatgtacaccg

SEQ ID NO: 99 - RNA, 21 nt, Arabidopsis thaliana
  source 1..21: mol_type = other RNA
tgggtggcaaacaaagacgac

SEQ ID NO: 100 - RNA, 21 nt, Arabidopsis thaliana
  source 1..21: mol_type = other RNA
tgagaatgcaaatccttagct

SEQ ID NO: 101 - RNA, 21 nt, Arabidopsis thaliana
  source 1..21: mol_type = other RNA
ttgattcccaatccaagcaag

SEQ ID NO: 102 - RNA, 20 nt, Arabidopsis thaliana
  source 1..20: mol_type = other RNA
gttcgaggcacgttgggagg

SEQ ID NO: 103 - RNA, 22 nt, Arabidopsis thaliana
  source 1..22: mol_type = other RNA
taatcctaccaataacttcagc

SEQ ID NO: 104 - RNA, 21 nt, Arabidopsis thaliana
  source 1..21: mol_type = other RNA
atcagtttcttgttcgtttca

SEQ ID NO: 105 - RNA, 21 nt, Arabidopsis thaliana
  source 1..21: mol_type = other RNA
ttgaattgaagtgcttgaatt

SEQ ID NO: 106 - RNA, 21 nt, Arabidopsis thaliana
  source 1..21: mol_type = other RNA
ttagatgaccatcaacaaact

SEQ ID NO: 107 - RNA, 21 nt, Arabidopsis thaliana
  source 1..21: mol_type = other RNA
tatgatcatcagaaaacagtg

SEQ ID NO: 108 - RNA, 21 nt, Arabidopsis thaliana
  source 1..21: mol_type = other RNA
atagtttctcttgttctgcac

SEQ ID NO: 109 - RNA, 21 nt, Arabidopsis thaliana
  source 1..21: mol_type = other RNA
ttttcttctacttcttgcaca

SEQ ID NO: 110 - RNA, 20 nt, Arabidopsis thaliana
  source 1..20: mol_type = other RNA
ataaatcccaacatcttcca

SEQ ID NO: 111 - RNA, 21 nt, Arabidopsis thaliana
  source 1..21: mol_type = other RNA
gcaacatcttcaagattcaga

SEQ ID NO: 112 - RNA, 20 nt, Arabidopsis thaliana
  source 1..20: mol_type = other RNA
tctcgcgcttgtacggcttt

SEQ ID NO: 113 - RNA, 21 nt, Arabidopsis thaliana
  source 1..21: mol_type = other RNA
taaactaatcacggaaatgca

SEQ ID NO: 114 - RNA, 22 nt, Arabidopsis thaliana
  source 1..22: mol_type = other RNA
tgatctcttcgtactcttcttg

SEQ ID NO: 115 - RNA, 23 nt, Arabidopsis thaliana
  source 1..23: mol_type = other RNA
gctcactctctttttgtcataac

SEQ ID NO: 116 - RNA, 24 nt, Arabidopsis thaliana
  source 1..24: mol_type = other RNA
ttaaagctccaccatgagtccaat

SEQ ID NO: 117 - RNA, 21 nt, Arabidopsis thaliana
  source 1..21: mol_type = other RNA
aggtgctgagtgtgctagtgc

SEQ ID NO: 118 - RNA, 20 nt, Arabidopsis thaliana
  source 1..20: mol_type = other RNA
acaaaatccgtctttgaaga

SEQ ID NO: 119 - RNA, 21 nt, Arabidopsis thaliana
  source 1..21: mol_type = other RNA
ttgcttaaagattttctatgt

SEQ ID NO: 120 - RNA, 21 nt, Arabidopsis thaliana
  source 1..21: mol_type = other RNA
ctgaagtgtttggggggactc

SEQ ID NO: 121 - RNA, 21 nt, Arabidopsis thaliana
  source 1..21: mol_type = other RNA
ctgaagtgtttggggggactc

SEQ ID NO: 122 - RNA, 21 nt, Arabidopsis thaliana
  source 1..21: mol_type = other RNA
ttcttcgtgaatatctggcat

SEQ ID NO: 123 - RNA, 21 nt, Arabidopsis thaliana
  source 1..21: mol_type = other RNA
taagctgccagcatgatcttg

SEQ ID NO: 124 - RNA, 21 nt, Arabidopsis thaliana
  source 1..21: mol_type = other RNA
atcatgctatctctttggatt

SEQ ID NO: 125 - RNA, 21 nt, Arabidopsis thaliana
  source 1..21: mol_type = other RNA
ctgaagtgtttggggggactc

(19) United States
(12) Patent Application Publication
(10) Pub. No.: US 2026/0021157 A1
(43) Pub. Date: Jan. 22, 2026

(54) COMPOSITIONS CONTAINING ABIOTICALLY-STRESSED PLANT-DERIVED EXOSOME-LIKE NANOPARTICLES

(71) Applicant: Exotropin LLC, New York, NY (US)

(72) Inventors: Jodi Gurney, Park Ridge, IL (US); Robin Smith, New York, NY (US)

(73) Assignee: Exotropin LLC, New York, NY (US)

(21) Appl. No.: 19/270,704

(22) Filed: Jul. 16, 2025

Related U.S. Application Data
(60) Provisional application No. 63/672,020, filed on Jul. 16, 2024.

(51) Int. Cl. A61K 36/886; A61K 9/00; A61K 9/51; A61K 31/713

(52) U.S. Cl. CPC A61K 36/886; A61K 9/0014; A61K 9/5176; A61K 31/713; A61K 2236/11

(57) ABSTRACT

The present disclosure provides a composition containing a purified population of plant-derived exosome-like nanoparticles isolated from tissue of a vascular plant, wherein the exosome-like nanoparticles comprise a tuned cargo comprising a protein signature and an miRNA signature, wherein the tuned cargo of the plant-derived exosome-like nanoparticles is a result of exposure of the plant to combinations of abiotic stress conditions that cause the plant to modulate its signaling pathways and metabolism to ensure its survival in a challenging environment. The tuned cargo of the plant-derived exosome-like nanoparticles can modulate bioactivities of mammalian cells directly or indirectly. The present disclosure also provides a method for improving appearance of human skin and human hair health, including eyelashes and eyebrows, comprising applying a composition comprising the abiotically stressed plant-derived exosome-like nanoparticles containing the tuned cargo and a carrier; and applying the composition topically.

### CROSS-REFERENCE TO RELATED APPLICATIONS

This application claims the benefit of priority to U.S. Provisional Application No. 63/672,020 (filed Jul. 16, 2024), which is incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 16, 2025, is named 2751_00101US_SL.xml and is 111,353 bytes in size. The sequence listing contained in this .XML file is part of the specification and is hereby incorporated by reference herein in its entirety.

### BACKGROUND OF THE INVENTION

### Mammalian Exosomes: General Principles

Exosomes are released by most if not all mammalian cell types, including platelets, blood cells, dendritic cells, mast cells, T cells, B cells, epithelial cells, endothelial cells, mesenchymal stem cells, smooth muscle cells, neuronal cells and many tumor cells. [Zhang, J. et al. Genomics Proteomics Bioinformatics (2015) 13: 17-24, citing Liao J., et al. Int J Mol Sci. 2014; 15:15530-15551; Kopers-Lalic, D. et al. Adv. Drug delivery rev. (2012) doi: 10.1016/j.addr.2012.07.006, citing Thery V., et al. Nat. Rev. Immunol. (2002) 2: 569-579].

Mammalian exosomes, which consist of lipid membranes, are spherical nanovesicles with a diameter of 40-150 nm (approximately 100 nm on average) [Kim, J. et al. Asian J. of Pharmaceutical Sci. (2022) 17: 53-69, citing Sinha, D. et al. Cancers (2021) 13 (2): 326; Villa, F. et al. Pharmaceutics (2019) 11 (11): 557; Zhang, Y. et al. Cell Biosi. (2019) 9 (1): 19; Doyle, L M and Wang, MZ. Cells (2019) 8 (7): 727]. They are constitutively generated by the inward budding of the plasma membrane to form early endosomes. The partial early endosomes integrate the surrounding lamina to generate intraluminal vesicles (ILVs), which encapsulate exosomes within large intracellular multivesicular bodies (MVBs). The subsequent fusion of MVBs with the plasma membrane leads to the secretion of exosomes from most ILVs into the extracellular space [Id., citing Zhang, Y. et al. Cell Biosci. (2019) 9 (1): 19; Hessvik, N P and Llorente, A. Cell Mol. Life Sci. (2018) 75 (2): 193-208; Farooqi, A A et al. Biotechnol. Adv. (2018) 36 (1): 328-334; Joshi, B S et al. ACS Nano (2020) 14 (4): 4444-4455]. Mammalian exosomes are typically defined by their size, composition and specific exosome marker proteins, such as CD9, CD81, CD63, flotillin and TSG101 [Id., citing Zhang, Y. et al. Cell Biosci. (2019) (1): 19; Yue, B. et al. Cell Prolif. (2020) 53 (7): e12857; Kalluri, R. and LeBleu, VS. Science (2020) 367 (6378): eaau6977]. Mammalian exosome architecture, components, and molecular processing reflect the processes taking place in their cells of origin [Id., citing Hu, Q. et al. Precis. Clin. Med. (2020) 3 (1): 54-66; Gluszko, A. et al. Biomed. Res. Int. (2019) 2019: 1628029].

Most mammalian EVs comprising exosomes share a core set of proteins and lipids. Mammalian-derived exosomes are characterized by the presence of specific lipids, such as phosphatidylserine, cholesterol, sphingomyelins, and ceramides. There seems to be a conserved protein repertoire in exosomes across cell-types and species [Kopers-Lalic, D. et al. Adv. Drug delivery rev. (2012) doi: 10.1016/j.addr.2012.07.006, citing Simpson, R J, et al. Proteomics (2008) 8: 4083-4099]. For example, the endosomal proteins such as Alix and TSG101, which are components of the mammalian ESCRT system, have been identified in the majority of the exosomes studied for their protein content thus far. In addition, heat shock proteins, which are involved in protein trafficking, are frequently found in exosomes [Id., citing van Dommelen, S M et al. J. Control. Release (2011) 161: 635-644]. Mammalian exosomes are further enriched in tetraspanins, like CD9, CD63, CD81 and CD82, which are important molecules for protein-protein interactions in cellular membranes. Tetraspanins bind many proteins, including integrins and MHC molecules [Id., citing Thery, C., et al. Nat. Rev. Immunol. (2009) 9: 581-593; Escola, J M. J. Biol. Chem. (1998) 273: 20121-20127; Keller, S. et al. Immunol. Lett (2006) 107: 102-108; Stoorvogel, W. et al. Traffic (2002) 3: 321-330]. Specific Rab proteins, a highly conserved family of small GTPases that function as molecular switches and coordinate membrane traffic [Id., citing Stenmark, H. Nat. Rev. Mol. Cell Biol. (2009) 10: 513-525], are often observed in exosomes by mass-spectrometry. Exosomes are also rich in annexins, membrane trafficking proteins that are involved in fusion events. Furthermore, cytoskeletal proteins like myosin, actin, and tubulin are present in exosomes. Finally, metabolic enzymes, antigen presentation molecules, ribosomal proteins and signal transduction molecules have been shown to be present in exosomes [Id., citing Mathivanan, S., et al. Proteomics (2008) 8: 4083-4099]

Mammalian exosomes may also carry (functional) genetic material, most notably small RNA molecules [Kopers-Lalic, D. et al. Adv. Drug delivery rev. (2012) doi: 10.1016/j.addr.2012.07.006, citing Zomer, A. et al. Commun. Integr. Biol. (2010) 3: 447-450; Gibbings, D, Voinnet, O. Trends Cell Biol. (2010) 20: 491-501]. Of all RNA molecules detected in exosomes, the class of 22 nt long, non-coding miRNAs has received attention since the discovery that miRNAs can be functionally transferred to recipient cells [Id., citing Pegtel, D M et al. Proc. Nat. Acad. Sci. USA (2010) 107: 6328-6333, Valadi, H. et al. Nat. Cell Biol. (2007) 9: 654-659]. MiRNAs regulate gene expression by binding imperfectly to the 3′ untranslated region of the target mRNA that results in translational repression of the mRNA into protein [Id., citing Bartel, DP. Cell (2004) 116: 281-297; Brennecke, J. et al. PLoS Biol. (20: e85 e85]. The term “non-coding RNAs (ncRNAs”) as used herein refers to functional RNA molecules that are transcribed from DNA but not translated into proteins. High-throughput sequencing technology confirmed that over 98% of the human genome is transcribed into ncRNAs, which are divided into two main groups: the small non-coding RNAs (<200 nucleotides) and the long non-coding RNAs (lncRNAs) (>200 nucleotides). In general, ncRNAs play a role in heterochromatin formation, histone modification and DNA methylation, leading to regulate gene expression at the transcriptional and post-transcriptional level. Epigenetic related ncRNAs include miRNA, siRNA, piRNA and lncRNA. Non-coding RNA types are summarized in Table 1.

TABLE 1
Non-coding RNA types.

| | | | Typical Size | |
|---|---|---|---|---|
| RNA | Functions | Coding | (nt = nucleotides) | |
| microRNA | Post-transcriptional | No | 17-24 | nt |
| (miRNA) | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA (SRP | ribonucleoprotein | | | |
| RNA) | complex that directs | | | |
| | protein trafficking | | | |
| Transfer RNA | Adapter for | No | 76-90 | nt |
| (tRNA) | matching amino | | | |
| | acid to mRNA | | | |
| Ribosomal RNA | RNA component of | No | 185 (1.9 kb) 28S | |
| (rRNA) | ribosomes | | (5.0 kb) | |
| Small | RNA processing | No | ≈150 | nt |
| nuclear RNA | such as mRNA | | | |
| (snRNA) | splicing | | | |
| Small | Guiding chemical | No | 20-24 | nt |
| nucleolar RNA | modifications of | | | |
| (snoRNA) | other RNAs | | | |
| Long noncoding | Many, including in- | No | >100 | nt |

| | |
|---|---|
| RNA (IncRNA) | transcription and |
| | post-transcription |
| | regulation |

mRNA

mRNAs are a large family of coding RNA molecules that specify protein sequence information. Studies have reported that mammalian EVs contain a substantial proportion of their parent cells' mRNA pool, many of which are cell type-specific mRNA. [Shao, H. et al. Chem Rev. (2018) 118 (4): 1917-1950, citing Wei, Z. et al. Nat. Commun. (2017) 8: 11:45; Batagov, AO, Kurochkin, IF. Biol. Direct (2013) 8: 12] These mRNA molecules, often in fragmented form, reside within EVs and are protected from RNase degradation. Furthermore, the fraction of polyadenylated mRNA molecules in EVs suggest that some of them (<2 kb) are capable of encoding polypeptides in support of protein synthesis (i.e., functionality in protein translation). This has been confirmed in multiple studies through different translation assays in recipient cells [Id., citing Valadi, H. et al. Nat. Cell Biol. (2007) 9: 654-659; Skog, J. et al. Nat. Cell Biol. (200: 1470-1476; Lai, C P et al. Nat. Commun. (2015) 6: 7029].

miRNA

miRNAs are a class of small, noncoding RNAs (typically 17-24 nucleotides) which mediate post-transcriptional gene silencing usually by targeting the 3′ untranslated region of mRNAs. By suppressing protein translation, EV miRNAs are powerful regulators for a wide range of biological processes [Id., citing Mittelbrunn, M. et al. Nat. Commun. (2011) 2: 282; Redzic, J S et al. Semin. Cancer Biol. (2014) 28: 14-23]. miRNAs can also exist in multiple stable forms when circulating in bodily fluids. For example, in addition to being packaged into EVs, circulating miRNAs can also be loaded onto high-density lipoprotein [Id., citing Vickers, K C et al. Nat. Cell Biol. (2011) 13: 423-433; Wagner, J. et al. Arterioscler. Thromb. Vasc. Biol. (2013) 33: 1392-1400] or bound to argonaute 2 (AGO2) protein, a family of proteins that play a role in RNA interference, outside the vesicles [Id.: citing Arroyo, et al. Proc. Natl Acad. Sci. USA (2011) 108: 5003-5008; Turchinovich, A. et al. Methods Mol. Biol. (2013) 1024: 97-107]. The distribution of miRNAs within EVs remains unclear [Id., citing Min, PK & Chan, SY. Eur. J. Clin. Invest. (2015) 45: 860-874; Turchinovich, A. et al. Methods Mol. Biol. (2013): 97: 97-107; Chevillet, J R et al. Proc. Natl. Acad. Sci. USA (2014) 111: 14888-14893]. As in the case of mRNA, miRNA profiles in EVs reflect their cell of origin but differs somewhat from their parental cells. Some miRNAs have been found preferentially sorted into EVs and remaining functional in recipient cells to regulate protein translation. [Id., citing Villarroya-Beltri, C. et al. nat. Commun. (2013) 4: 2980; Koppers-Lalic, D. et al. Cell Rep. (2014) 8 (6): 1649-1658; Santangelo, L. et al. Cell Rep. (2016) 17: 799-808; Teng, Y. et al. Nat. Commu. (2017) 8: 14448]

### Other RNA Types

In addition to mRNA and miRNA, many noncoding RNA types have been identified in EVs through next generation sequencing [Id., citing Huang, X. et al. BMC Genomics (2013) 14: 319; Conley, A. et al. RNA Biol. (2017) 14: 305-316]. These RNAs include transfer RNA (tRNA), ribosomal RNA (rRNA), small nuclear RNA (snRNA), small nucleolar RNA (snoRNA), as well as long noncoding RNA (lncRNA) [Id., citing Wei, Z. et al. Nat. Commun. (2017) 8: 1145; Huang, X. et al. BMC Genomics (2013) 14: 319; Crescitelli, R. et al. J. Extracell. Vesicles (2013) 2: 20677].

Small (about 20-30 nucleotides (nt)) noncoding RNAs regulate eukaryotic genes and genomes (Carthew, R W and Sontheimer, EJ. Cell (2009) 136: 642-55). This regulation can occur at multiple levels of genome function, including chromatin structure, chromosome segregation, transcription, RNA processing, RNA stability, and translation (Id.). The effects of small RNAs on gene expression and control are generally inhibitory, and the corresponding regulatory mechanisms are therefore collectively subsumed under the heading of RNA silencing (Id.). The central theme that runs throughout is that the small RNAs serve as specificity factors that direct bound effector proteins to target nucleic acid molecules via base-pairing interactions (Id.). Invariably, the core component of the effector machinery is a member of the Argonaute protein superfamily (Id.).

There are three main categories of small RNAs: short interfering RNAs (siRNAs), microRNAs (miRNAs), and piwi-interacting RNAs (piRNAs) (Id.). siRNAs and miRNAs are the most broadly distributed in both phylogenetic and physiological terms and are characterized by the double-stranded nature of their precursors (Id.). In contrast, piRNAs are primarily found in animals, exert their functions most clearly in the germline, and derive from precursors that are poorly understood, but appear to be single stranded (Id.). Where siRNAs and miRNAs bind to members of the Ago clade of Argonaute proteins, piRNAs bind to members of the Piwi clade (Id.).

The signature components of RNA silencing are Dicers, Agos, and ˜21-23 nt duplex-derived RNAs (Id.). Both siRNA and miRNA small RNAs depend on Dicer enzymes to excise them from their precursors, and Ago proteins to support their silencing effector functions (Id.).

RNase III enzymes, which are dsRNA-specific nucleases, are the source of miRNA/siRNA biogenesis (Id.). One class of large RNase III enzymes has several domains in a specific order from the amino to carboxy terminus: a DEXD/H ATPase domain, a DUF283 domain, a PAZ domain, two tandem RNase III domains, and a dsRNA-binding domain (Id.). Some members of this family differ slightly from this arrangement (Id.).

The PAZ and RNase III domains play central roles in excising siRNAs preferentially from ends of dsRNA molecules. PAZ domains are shared with Argonaute proteins and are specialized to bind RNA ends, especially duplex ends with short (˜2 nt) 3′ overhangs. An end engages the Dicer PAZ domain, and the substrate dsRNA then extends approximately two helical turns along the surface of the protein before it reaches a single processing center that resides in a cleft of an intramolecular dimer involving the RNase III domains. Each of the two RNase IIII active sites cleaves one of the two strands, leading to staggered duplex scission to generate new ends with ˜2-3′ nt overhangs. The reaction leaves a 5′ monophosphate on the product ends, consistent with a requirement for this group during later stages of silencing. This general model pertains equally to pre-miRNA stem-loop substrates and to long, perfectly base-paired dsRNAs. In some species, different functional categories of small RNAs exhibit slightly different lengths; this appears to be dictated by the distance between the PAZ domain and the processing center in the relevant Dicer enzyme (Id.).

The roles of the ATPase domain probably vary among different forms of Dicer (Id.). ATP promotes dsRNA processing by Drosophila Dicer 2 and C. elegans Dcr-1, and mutations predicted to cripple ATPase activity in Drosophila Dicer-2 specifically abolish dsRNA processing. In contrast, ATP is dispensable for dsRNA processing by human Dcr (hDcr), and an ATPase defective mutant exhibits no processing defect (Id.).

Dicers isolated from their natural sources generally are found in a heterodimeric complex with a protein that contains two or three double stranded Ras binding domains (dsRBDs); the Ras-binding domain (RBD) is an independent domain of about 75 residues, which is sufficient for GTP-dependent binding of Ras and other G alpha GTPases. Both hDcr and Drosophila Dcr-2 process dsRNAs effectively in the absence of the heterodimeric partner (TRBP and R2D2, respectively). In at least some cases, the role of Dicer in silencing extends beyond dsRNA processing and into the pathway of RISC assembly; this activity is much more dependent on the dsRBD partner protein (Id.).

### Argonautes

The Argonaute superfamily can be divided into three separate subgroups: the Piwi clade that binds piRNAs, the Ago clade that associates with miRNAs and siRNAs, and a third clade described in nematodes. All gene regulatory phenomena involving ˜20-30 nt RNAs are thought to require one or more Argonaute proteins, which are the central, defining components of an RNA-induced silencing complex (RISC). The double-stranded products of Dicer enter into an RISC assembly pathway that involves duplex unwinding, culminating in the stable association of only one of the two strands with the Ago effector protein. This guide strand directs target recognition by Watson-Crick base pairing; the other strand of the original small RNA duplex (the passenger strand) is discarded (Id.).

Argonaute proteins are defined by the presence of four domains: the PAZ domain (shared with Dicer enzymes), the PIWI domain that is unique to the Argonaute superfamily, and the N and Mid domains. The overall protein structure is bi-lobed, with one lobe consisting of the PAZ domain and the other lobe consisting of the PIWI domain flanked by N-terminal (N) and middle (Mid) domains. The Argonaute PAZ domain has RNA 3′ terminus binding activity, and the co-crystal structures reveal that this function is used in guide strand binding. The other end of the guide strand engages a 5′ phosphate binding pocket in the Mid domain, and the remainder of the guide tracks along a positively charged surface to which each of the domains contributes. The protein-DNA contacts are dominated by sugar-phosphate backbone interactions. Guide strand nucleotides 2-6, which are especially important for target recognition, are stacked with their Watson-Crick faces exposed and available for base pairing (Id.).

The PIWI domain adopts an RNase H-like fold that in some cases can catalyze guide strand-dependent endonucleolytic cleavage of a base pair target. This initial cut represents the critical first step in a subset of small RNA silencing events that proceed through RNA destabilization. Not all Argonaute proteins have endonucleolytic activity, and those that lack it usually also lack critical active-site residues that coordinate a presumptive catalytic metal ion (Id.).

In humans, four of the eight Argonaute proteins are from the Ago clade and associate with both siRNAs and miRNAs (Id.).

### MicroRNA Biogenesis

MicroRNAs are found in plant and animal branches of Eukaryotes and are encoded by a bewildering array of genes. Transcription of miRNAs is typically performed by RNA polymerase II, and transcripts are capped and polyadenylated. Although some animal miRNAs are individually produced from separate transcription units, many more are produced from transcription units that make more than one product. A transcript may encode clusters of distinct miRNAs, or it may encode miRNA and protein. The latter type of transcript is organized such that the miRNA sequence is located within an intron. Many new animal miRNAs are thought to arise from accumulation of nucleotide sequence changes and not from gene duplication (Carthew, R W and Sontheimer, E J. Cell (2009) 136: 642-55).

The resulting primary or pri-miRNA transcript extends both 5′ and 3′ from the miRNA sequence, and two sequential processing reactions trim the transcript into the mature miRNA. Processing depends on the miRNA sequence folding into a step-loop structure. A typical animal pri-miRNA consists of an imperfectly paired stem of ˜33 bp, with a terminal loop and flanking segments. The first processing step, which occurs in the nucleus, excises the stem-loop from the remainder of the transcript to create a pre-miRNA product. For most pri-miRNAs, a nuclear member of the RNase III family (Drosha in animals) carries out this cleavage reaction. Although Drosha catalyzes pri-miRNA processing, it depends on a protein cofactor, which contains two dsRBD domains and stably associates with the ribonuclease to form the microprocessor complex (Id.).

An alternative pathway uses splicing of pri-miRNA transcripts to liberate introns that precisely mimic the structural features of pre-miRNAs. These introns then enter the miRNA processing pathway without the aid of the Microprocessor (Id.).

The second processing step excises the terminal loop from the pre-miRNA stem to create a mature miRNA duplex of approximately 22 bp length. In animals, the pre-miRNA is exported from the nucleus, and the canonical Dicer enzyme carries out the cleavage reaction in the cytoplasm (Id.).

MicroRNAs behave like traditional polymeric products of gene activity, such that most species of a miRNA have highly exact ends, although there is a little variation. This feature of miRNAs may allow them to interact with greater specificity on substrate mRNAs without a need for stringent complementarity or large overlap (Id.).

Consequently, the processing machinery is constructed to produce miRNA duplexes with highly exact ends. The first cut, carried out by Drosha with the aid of its dsRBD domain binding partner protein (called DGCR8), is most critical. DGCR8 directly interacts with the pri-miRNA stem and flanking single-stranded segments. The cleavage site is determined by the distance from the stem-flank junction, which is precisely one turn of a dsRNA helix (11 bp) and is the minimal processing length for an RNase III enzyme. Although Drosha carries out the cleavage reaction, it relies upon DGCR8 to serve as a molecular anchor that properly positions Drosha's catalytic site the correct distance from the stem-flank junction. Thus, the endpoint of the stem is a critical determinant for one end of the mature miRNA (Id.).

The second cut performed by Dicer defines the other end of the mature miRNA. Dicer will cleave anywhere along a dsRNA molecule but has a strong preference for the terminus. The PAZ domain of Dicer interacts with the 3′ overhang at the terminus and determines the cleavage site in a ruler-like fashion. The RNase III catalytic sites are positioned two helical turns or 22 bp away from the terminus/PAZ portion of the Dicer-RNA complex (Id.).

While regulation of miRNA biogenesis has not been extensively studied, a surprising number of miRNA genes are formed under the control of the very targets that they regulate. A rationale behind these double-negative regulatory relationships is that tight regulation of miRNA biogenesis is crucial. Mis-expression of miRNAs frequently mimics loss of function phenotypes for their targets. This would be prevented if biogenesis of a miRNA is strictly controlled by its targets. The restriction would also explain how off-targeting effects by wayward miRNAs are carefully limited (Id.).

### MicroRNA Associations

The mature miRNA duplex is a short-lived entity; it is rapidly unwound when it associates with an Ago protein. Unwinding occurs so rapidly after duplex formation, because the two processes are physically coupled due to Ago2's presence in a complex with Dicer and TRBP, the double-stranded RNA binding protein that loads siRNA into the RISC (Id.).

miRNA unwinding is accompanied by differential strand retention, i.e., one strand is retained while the other strand is lost. Strand retention is based on the relative thermodynamic stability of the duplex's ends. Although the rule is that the 5′ terminus of the retained strand is at the less stably base-paired end of the duplex, this rule is not absolute. The other strand is appreciably detected in Ago complexes, lending ambiguity to the notion of strand asymmetry. Although either strand can become stably associated with Ago proteins, the more commonly associated strand is termed the miRNA strand; the other strand is called the miRNA* strand. miRNA unwinding is not accompanied by cleavage of the ejected strand by the associated Ago (Id.).

The mammalian Dicer/Ag/miRNA complex is associated with other proteins, e.g., Gemin3, Gemin4, Mov10, and Imp8, as well as the mammalian protein GW182, associates with Ago2. GW182 is both necessary and sufficient for miRNA-bound Ago to silence gene expression. Thus miRNA-bound Ago in association with GW182 can be thought of as the miRISC complex (Id.).

Post-Transcriptional Repression by miRNAs

A miRNA acts as an adaptor for miRISC to specifically recognize and regulate particular mRNAs. If miRISC is tethered to a heterologous RNA recognition factor, the factor enables miRISC to recognize and repress mRNAs that lack miRNA-binding sites. With few exceptions, miRNA-binding sites in animal mRNAs lie in the 3′ untranslated region (UTR) and are usually present in multiple copies. Most animal miRNAs bind with mismatches and bulges, although a key feature of recognition involves Watson-Crick base pairing of miRNA nucleotides 2-8, representing the seed region (Id.).

While it was thought that perfect complementarity allows Ago-catalyzed cleavage of the mRNA strand, whereas central mismatches exclude cleavage and promote repression of mRNA translation, it appears that translational repression is the default mechanism by which miRNAs repress gene expression, both in animals and plants. Perfectly complementary miRNAs may additionally engage in mRNA cleavage such that their effects are the result of both mechanisms (Id.).

The mechanisms by which miRISC regulates translation have been subject to ongoing debate. The fundamental issue of whether repression occurs at translation initiation or post-initiation has not yet been resolved. There are three competing models for how miRISC represses initiation. One proposes that there is competition between miRISC and eIF4E for binding to the mRNA 5′ cap structure. A second model has proposed that miRISC stimulates de-adenylation of the mRNA tail; translation is repressed because the cap and PABP1-free tail of the deadenylated mRNA are unable to circularize. A third model has proposed that miRISC blocks association of the 60S ribosomal subunit with the 40S preinitiation complex, i.e., the recruitment of eIF6 by miRISC may repress translation by preventing the assembly of translationally competent ribosomes at the start codon (Id.).

It is unclear why some targets are degraded and others are not (Id.).

Without being limited by any particular theory, it appears that the mode of regulation of any miRNA (repression vs. activation) in the context of the whole cell and the myriad activities that affect posttranscriptional gene regulation may be context dependent (Id.).

The cell's position in the cell cycle is one such context. For example, mammalian miRNA let-7 and an artificial miRNA (CXCR-4) repress translation in proliferating human cells, but change into translational activators when the cell cycle is arrested at the G1 checkpoint by serum starvation. Aphidicollin-induced arrest at G1 also generates translational activation, whereas nocodazole-induced arrest at G2/M generates translational repression. Lymphocyte growth arrest induces TNFα expression that is required for macrophage maturation; miR-369-3p switches from a repressor to an activator of TNFα translation when cells in culture are growth arrested (Id., citing Vasudevan, S. et al. Science (2007) 318: 1931-1934).

Binding site position is another context. Interaction of miR-10a with the 5′UTR of certain ribosomal subunit mRNAs leads to their activated translation, whereas interaction with the 3′UTR leads to repression (Id., citing Orom, U A et al. (2008) Mol. Cell 30: 460-471).

Another context is how small RNA regulation is organized and modulated within the cell. Ago proteins are frequently associated with membrane trafficking compartments, such as the Golgi and ER (Id., citing Cikaluk, D. E. et al. Mol. Biol. Cell (1999) 10: 3357-3372). It has been hypothesized that miRISC factors might become anchored in certain subcellular compartments, e.g., P bodies or GW bodies, two separate pools of sequestered non-translating RNAs (Patel, P H, et al. PLos One (2016) 11(3): e015029). Subunits of miRISC (miRNAs, Ago and GW1821) and their repressed targets also are enriched in GW bodies. While GW bodies are not essential for miRNA repression, GW body formation requires an intact miRNA pathway (Carthew, R W and Sontheimer, E J. Cell (2009) 136: 642-655).

miRNA Expression

MicroRNAs regulate gene expression at the post-transcriptional level. The exact functional outcome of an miRNA may be determined by multiple features, including the cell type affected, the inducing signal, and the transcriptomic profile of the cell, which ultimately affect the availability and ability to engage different target mRNAs and bring about its unique responses. Indeed, data suggest that miRNAs may play different roles in diverse biological contexts. [Lee, H-M et al. BMB Rep. (2016) 49 (6): 311-318].

### ESCRT in Mammalian Cells

The endosomal sorting complex required for transport (ESCRT) machinery responsible for sorting the cargo proteins of ILVs is divided into four complexes, ESCRT-0, -I, -II, -III, which work cooperatively to generate MVBs with associated proteins VPS4, VTA1, and ALG-2 interacting protein (ALIX).

The ESCRT-0 complex is composed of two subunits, hepatocyte growth factor-regulated tyrosine kinase (HRS) and STAM, both of which can engage ubiquitinylated substrates destined for lysosomal degradation. EXCRIT-0 recruits TSG101 of the ESCRT-I complex and isolates the ubiquitinated proteins in the endosomal membrane. The ESCRT-I complex is essential and responsible for cargo sorting in the MVBs, as it induces budding by deforming the plasma membrane. Subsequently, ESCRT-I activates ESCRT-III, which is responsible for the concentration of MVBs' cargo molecules using the ESCRT-II complex or ALIX, and the ESCRT-III/VPS4 complex triggers the constriction and induces the cleavage of the vesicle buds during abscission (meaning the act of cutting off). Afterwards, MVBs interact with a specific combination of soluble N-ethylmaleimide-sensitive factor attachment protein receptors (SNAREs) within the plasma membrane, and mammalian-derived exosomes are secreted to the extracellular milieu of the cell [Id., citing Kalluri, R. and LeBleu, V S, Science (2020) 367 (6478): eaau6977; Vietri, M. et al. Nat. Rev. Mol. Cell Biol. (2019) 21 (1): 25-42].

Depending on the particular composition of their exosomal proteins, the fate and function of mammalian-derived exosomes in their biogenesis, cargo selection, targeting ability, and endocytosis under both physiological and pathological conditions can vary. Among the protein cargoes of mammalian-derived exosomes, those proteins participating in cell adhesion (e.g., integrin, lactadherin, ICAM), intracellular trafficking (e.g., RAB GTPases, annexin), signal transduction (e.g., protein kinases, β-catenin, 14-3-3, G proteins), biogenesis factors (e.g., ALIX, TSG101, syntenin, ubiquitin, clathrin, VPS32, VPS4), and as well as chaperones (e.g., HSP70, HSP90), have been evaluated [Id., citing van Niel, G. et al. Nat. Rev. Mol. Cell Biol. (2018) 19: 213-228]. Generally, mammalian-derived exosomes are characterized by the presence of specific lipids such as phosphatidylserine, cholesterol, sphingomyelins, and ceramides, which are responsible for intercellular signaling as well as being essential in structural stability. In addition, they may also carry genetic material such as messenger RNA (mRNA), microRNAs (miRNAs), and non-coding RNAs. Overall, mammalian-derived exosomes are not only considered to be effective intercellular transporters of proteins, lipids, and nucleic acids, but also novel regulators that can alter the physiological and pathological functions of both recipient and parent cells via their various components.

Although, the application of mammalian derived exosomes is promising, several major issues limit the clinical use of these exosomes, including (1) their low production yield, (2) the time-consuming and laborious production processes, and (3) the difficulties involved with achieving high-quality and uniform exosomes [Id., citing [Li, P. et al. Theranostics (2017) 7(3): 789-804]; van den Boom, J G et al. Nat. Biotechnol. (2011) 29 (4): 325-326; Lobb, R J et al. J. Extracell. Vesicles (2015) 4 (1): 27031; Vader, P. et al. Adv. Drug Deliv. Rev. (2016) 106 (Part A): 148-156; van Deun, J. et al. J. Extracell. Vesicles (2014) 3 (1): 24858].

### Plant-Derived Exosome-Like Nanovesicles (PELNVs).

Evidence of the existence of plant extracellular vesicles dates back to 1967. [Halperin, W. and Jensen, W A. J. Ultrastruct. Res. (1967) 18: 428-443]. Notwithstanding that many aspects of plant-derived exosome-like nanovesicles are not fully understood, they may provide therapeutic advantages compared with mammalian-derived exosomes or artificial nanoparticles, including facile large-scale production [Subha, D. et al. Discover Nano 92023) 18: 146, citing Li, Z. et al. Sci. Rep. (2018) 8(1): 14644], low toxicity, reduced immunogenicity [Id., citing Deng, Z. et al. Mol. Ther. (2017) 25 (7): 1641-1654], efficient cellular uptake [Id., citing Wang, Q. et al. Nat. Commun. (2013) 4: 1867] and high biocompatibility and stability [Id., citing Zhang, M. et al. Biomaterials (2016) 101: 321-340].

WO2020/180311 entitled “Plant-based exosome compositions and use thereof for rejuvenating skin” describes isolation of exosomes from leaf flesh of a plant from the Asphodelaceae family, more particularly of the Aloe genus, including Aloe vera, Aloe barbadensis Miller, Aloe aborenscens, or Aloe vera L. conditioned by growing the plant under conditions that included a heat shock of the plant at a temperature of from about 33° C. to about 45° C. for about 1 hour to about 3 hours. The level of heat shock stress response molecule HSP70 in the heat-shocked plant exosome was from about 10 times to about 20 times higher than the level of heat shock stress response molecule in the exosomes from non-heat-shocked plants.

Plant-derived exosome-like nanovesicle (PELNV) preparation has been attempted using many common edible plants, such as grapefruit, grape, lemon, broccoli, carrot, apple, coconut, and ginger. [Kim, J. et al. Asian J. of Pharmaceutical Sci. (2022) 17: 53-69, citing Ju, S. et al. Mol. Ther. (2013) 21 (7): 1345-1357; Wang, B. et al. Mol. Ther. (2014) 22 (30: 522-534; Wang, Q. et al. Cancer Res. (2015) 75(12): 2520-2529; Brahmbhatt, M. et al. Nutr. Cancer (2013) 65 (2): 263-272; Raimondo, S. et al. Oncotarget (2015) 6 (23): 19514-19527; Deng, Z. et al. Mol. Ther. (2017) 25 (7): 1641-1654; Mu, J. et al. Mol. Nutr. Food Res. (2014) 58 (7): 1561-1573; Zhao, Z. et al. J. Agric. Food Chem. (2018) 66 (11): 2749-2757; Yu, S. et al. Food Chem. (2019) 272: 372-378; Fujita, D. et al. Mol. Pharm. (2018) 15 (12): 5772-5780; Zhang, M. et al. Mol. Ther. (2016) 24 (10) 1783-1796]. PENLV are known to be similar to mammalian-derived exosomes in terms of such properties as size distribution, surface electric charge, morphology, density, and certain components [Id., citing Ju, S. et al. Mol. Ther. (2013) 21 (7): 1345-1357; Raimondo, S. et al. Oncotarget (2015): 19514 19514-27; Deng, Z. et al. Mol. Ther. (2017) 25 (7): 1641-1654]. Like mammalian-derived exosomes, PELNVs also comprise biomolecules, such as RNAs, proteins, and lipids, that regulate physiological processes [Id., citing Teng, Y. et al. Cell Host Microbe (2018) 24 (5): 637-652].

Despite many similarities between PELNVs and mammalian-derived exosomes, the two groups of vesicles show some differences. First, the lipid bilayer of mammalian-derived exosomes are mainly composed of cholesterol, glycoshingolipids, ceramides and phosphatidylserine, which provide stability and a unique rigidity [Id., citing Stemersch, S. et al. J. Control Release (2016) 244: 167-83; Nishio, M. et al. Bioscens. Bioelectron (2020) 150: 111918; Mashouri, L. et al. Mol Cancer (2019) 18: 75; Waldenstrom, A. and Ronquist, G. Circ. Res. (2014) 114 (2): 315-324]. In contrast, the exosomal membranes of PELNVs are enriched with phosphatidic acid (PA), phosphatidylcholines (PC), digalactosyldiacylglycerol (DGDG), and monogalactosyldiacylglycerol (MGDG) [Id., citing Teng, Y. et al. Cell Host Microbe (2018) 24 (5): 637-652], which provide inherent mammalian-cell-regulating activities in the intestinal microenvironment.

Second, there is no canonical ESCRT-0 complex in higher plants; instead, TOM1-like (TOL) proteins with conserved VHS (VPS27, HRS, STAM) domains, which are considered to have a general membrane targeting/cargo recognition role in vesicular trafficking [see Lohi, O. et al. FEBS Lett. (2002) 513 (1): 19-23] act as substitutions for ESCRT-0 as ubiquitin binding proteins and play a role in the vacuolar sorting of the auxin efflux facilitator PIN-FORMED 2 (PIN2) in the early endosome [Kim, J. et al. Asian J. of Pharmaceutical Sci. (2022) 17: 53-69, citing Gao, C. et al. Trends Plant Sci. (2017) 22 (11): 986-998]. The cargo is subsequently transported to the ESCRT-1 and ESCRT-II complexes via the ubiquitin-binding proteins, the ESCRT-II complex then stimulates and recruits ESCRT-III through an interaction between VPS25 and VSP20. The ESCRT-III complex constricts the plasma membrane and cleaves the necks of the buds that form on the cytosolic face to release ILVs containing cargo into the endosome [Id., citing Yanez-Mo, M. et al. J. Extracell. Vesicles (2015) 4: 27066; Cui, Y. et al. Mol. Plant (2016) 9 (6): 774-786].

Third, surface markers to differentiate between types of extracellular vesicles in plants are yet to be identified, and the mechanism underlying trafficking of the EVs is unknown. [Subha, D. et al. Discover Nano (2023) 18: 146].

Fourth, each PELNV has different characteristics and components depending on the cell of origin. For example, Zhuang et al. reported that a 6-shogaol rich in ginger-derived exosome-like nanoparticles (GDENs) activates Nrf2 by regulating TLR4/TRIF pathway, protecting against alcohol-induced liver damage through the anti-inflammatory actions of this pathway [Id., citing Zhang, X. et al. J. Extracell. Vesicles (2015) 4 (1): 28713]. Ju et al. reported that grape-exosome like nanoparticles induced the recovery of intestinal stem cells through the Wnt/β-catenin signaling pathway, which regulates genes including AXIN-2, Cycline D1, c-MYC, and EGF [Id., citing Ju, S. et al. Mol Ther. (2013) 21 (7): 1345-1357]. In terms of the cellular uptake of grape-derived exosome-like nanoparticles (GELNs), GELNs showed a high selectivity to intestinal stem cells and were significantly inhibited by a cytochalasin-D inhibitor, known as micropinocytosis inhibitor. In contrast, a clathrin-mediated endocytosis inhibitor did not affect their uptake [Id.].

Fifth, beneficially, unlike mammals, plants do not harbor zoonotic or human pathogens.

Plants produce EVs in response to numerous biotic and abiotic environmental stresses, including pathogen infection and attack. [Kim, J. et al. Asian J. of Pharmaceutical Sci. (2022) 17: 53-69, citing An, Q. et al. New Phytol. (2006) 172 (3): 563-576; An, Q. et al. Cell Microbiol. (2006) 8 (6): 1009-1019). Qianli et al., who observed the proliferation of intravacuolar MVBs in the cytoplasm and the structural perturbation of the organelle and its membrane by trafficking in barley leaf cells, provided evidence of the generation and secretion of plant-derived exosome-like nanovesicles [Id., citing Qianli, AJVB, and Huckelhoven, R. Plant Signal Behav. (2007) 2 (1): 4-7]. Additionally, it has been reported that enhancement of fungal infections induces the proliferation of MVBs at the site of infection by stimulating plant innate immune responses in plant defense [Id., An, Q. et al. New Phytol. (2006) 172 (3): 563-576; Wang, F. et al. PLoS Pathog. (2014) 10 (7): e1004243].

Evidence indicates that plant extracellular vesicles play a multifaceted role in plant defense [Subha, D. et al. Discover Nano 92023 18: 146]. They function as mobile pockets highly enriched with antimicrobials and plant-defense proteins which initiate the plant immune response at the site of pathogen entry. For example, defense-related proteins involved in the myrosinase-glycosinolate system such as penetration resistance protein 3 (PEN3), which provides cell wall defense against pathogens, the nitrate sensor NRT1, the myrosinase epithiospecific modifier 1, and reactive oxygen species (ROS) signaling proteins were identified in the EV proteome [Id. citing Rutter, B D and Innes, R W. Plant Physiol. (2017) 173: 728-41; Rutter, B D, and Innes, R W. Curr. Opin. Plant Biol. (2018) 44: 16-22]. Plant EVs also can hold cell wall remodeling enzymes. On sensing an infection, the plant tries to strengthen its defense by improving cell wall integrity, while the pathogen takes advantage of the cell wall metabolism in the host to establish the infection [Id., citing Vincent, D. et al. Front. Plant Sci. (2020) 10: 1626]. Cell wall remodeling proteins are transported through EVs in several cases. Proteomic studies on EVs from sunflower seedlings revealed that 47% of the identified proteins are cell wall-associated including the enzymes in-charge of polysaccharide reorganization [Id., citing Regente, M. et al. Exp. Bot. (2017) 68: 5485-5495].

When the proteome of the plant EVs from Arabidopsis was analyzed, a total of 93 RNA binding proteins RBPs) were recognized. Those with ssRNA binding capacity are AGO1, DEAD-box RNA helicase RH11, RH37, RH52, Annexin 1 (ANN1) and annexin 2 (ANN2). These proteins were also easily detected by western blotting in isolated EVs thereby confirming the proteome results. These RBPs co-localize with the TET8 marker and the EV associated small RNAs previously identified (such as TAS1c-SiR483, TAS2-SiR 453 and miR166) were also found associated with this subgroup of exosome like EVs. EV localized RBPs specifically bind with the small RNAs and aid in their selective loading into the EVs. The Annexins (ANN1 and ANN2), which were also found in the EVs do not bind with ssRNAs specifically; it has been suggested that they might contribute to the stabilization of the RNA inside the vesicles [Id.].

### Plant Heat Shock Proteins Counter Plant Biotic and Abiotic Stresses

Plants continuously confront harsh environments like high/low temperatures, salinity, drought, light stresses, flooding, physical wounding and chemical pollutants (e.g., heavy metals), which produce secondary stresses, such as osmotic and oxidative stresses, [al Haq, S. et al. Intl J. Molec. Sci. (2019) 20: 5321, citing Swindell, W R et al. BMC Genom. (2007) 8: 125; Al-Whaibi, MH. J. King Saud Univ. Sci. (2011) 23: 139-150; Guo, M. et al. Front. Plant Sci. (2016) 7: 114; Xu, Y. et al. Int. J. Proteom. (2011) 2011: 529648]. Biotic stresses like pathogens (e.g., viruses, bacteria, and fungi) and pests (e.g., nematodes, insects, and rodents) also restrict plant productivity [Id., citing Dodds, P. and Rathjen, J. Nat. Rev. Genet. (2010) 11: 539; Li, R. et al. PLoS One (2015) 10: e0143261; Jones, J T et al. Mol. Plant Pathol. (2013) 14: 946-561; Rybicki, EP. Arch. Virol. (2015) 160: 17-20; Gorovits, R. et al. Cell Stress Chaperones (2017) 22: 345-355]. Negative effects of these stresses on plant germination [Id., citing Cheng, L. et al. J. Integr. Plant Biol. (2009) 51: 489-499], include stunted growth [Id., citing Srivastava, S. et al. J. Environ. Biol. (2012) 33: 657; Wahid, A. et al. Environ. Exp. Bot. (2007) 61: 199-223], sunburn and scorching of leaves [Id., citing Rodruguez, M. et al. Plants Biotecnologia (2005) 22: 1-10], loss of photosynthetic pigment, decreased production of photo-assimilates, and depletion of carbohydrate reserves which results in starvation [Id., citing Tan, W. et al. J. Plant Physiol. (2011) 168: 2063-2071; Jiang, C. et al. Plant Cell Environ. (2009) 32: 1046-1059; Demirevska-Kepova, K. et al. Biol. Plant (2005) 49: 521-525; Djanaguiraman, M. et al. J. Agron. Crop Sci. (2009) 195: 213-224]. Abiotic stresses also negatively affect the reproductive characteristics of plants by enhancing male sterility [Id., citing Young, L W et al. J. Exp. Biol. (2004) 55: 485-495] and increasing premature flower and fruit drop [Id., citing Tubiello, F N et al. Proc. Natl Acad. Sci. USA (2007) 104: 19686-19690] which results in significant low yield and quality. It has been reported that an increase in temperature by 1° C. results in a 4-10% yield decrease [Id., citing Wang, X. et al. J. Cereal Sci. (2012) 55: 331-6]. As a consequence of these stresses, reactive oxygen species (ROS) are produced which lead to oxidative stress and, ultimately, results in cell death. ROS could be singlet oxygen (¹O₂), superoxide radical (O₂·—), hydrogen peroxide (H₂O₂) and hydroxyl radical (OH—), which are produced in cell organelles such as mitochondria, peroxisomes and chloroplasts in oxidative stress situations and react with all types of macromolecules like pigments, proteins, lipids and DNA [Id., citing Karuppanapandian, T. et al. Plant Physiol. Biochem. (2011) 49: 168-177; Moller, I M et al. Ann. Rev. Plant Biol. (2007) 58: 459-481].

Plants respond morphologically to elevated temperature and light stress by changing their leaf orientation [Id., citing Wahid, A. et al. Environ. Exp. Biol. (2007) 61: 199-223], anatomically by altering stomatal conductance and increased leaf pubescence [Id., citing Zhang, J. et al. Field Crop Res. (2006) 97: 111-119; Banon, S. et al. Sci. Hortic. (Amst.) 2004 101: 333-342], and phenologically by shifting and improvising the developmental stages to escape the abiotic stress condition [Id., citing Sato, S. et al. Ann. Bot. (2006) 97: 731-8]. Plants also change their metabolic processes and physiology to retain root hydraulic conductance [Id., citing Morales, D. et al. Biol. Plant (2003) 47: 203], accumulation of compatible osmolytes, such as sugars, sugar alcohols, proline and phenolic compounds under saline and water-logged conditions, as well as high temperature and water deficit conditions [Id., citing Wahid, A. and Close, TJ. Biol. Plant (2007) 51: 104-109]. Moreover, plants manage to maintain photosynthetic machinery [Id., citing Salvucci, M E et al. Physiol. Plant (2004) 120: 179-186] by changing their assimilate partitioning (e.g., a shift occurs from symplastic, where the symplast pathway involves protoplasts that are found within the living cytoplasm cells, to apoplastic (a non-living route since the water solution moves in the spaces between cells and along the cell wall) [Id., citing Wahid, A. and Ghazanfar, A. J. Plant Physiol. (2006) 163: 723-730]. During the onset of the stress situations, plants also improvise the hormonal balance of abscisic acid (ABA), ethylene, and salicylic acid (SA) as a signaling molecule in the systemic acquired resistance. Similarly, jasmonic acid (JA) and other steroids enhance stress tolerance and resistance [Id., citing Wang, LJ and Li, SH. Plant Sci. (2006) 170: 685-694]. Furthermore, secondary metabolites, such as isopropanioid, carotenoid, flavonoid, anthocyanin, lignin, and isoprenoids [Id., citing Wahid, A. and Ghazanfar, A. J. Plant Physiol. (2006) 163: 723-730; Sharkey, TD. Plant Cell Environ. (2005) 28: 269-277], also are produced and accumulated. Most studies indicate that plant responses to two or more factors are unique and differ from the response to one factor only. [Al-Whabi, MH, J. King Saud University-Science, J. King Saud Univ.—Science (2011) 23: 139-150].

Besides these adaptations, plants also have sophisticated adaptive systems at the cellular and molecular levels. During the onset of stress, plants reduce the synthesis of normal protein production and transcribe and translate heat shock proteins (HSPs). Added to transcriptional regulations, plants also have some sophisticated post-transcriptional modifications which help the plant to cope with these stresses, such as alternative splicing and micro RNA (miRNA). Alternative splicing, which generates multiple copies from a single gene, helps the plants to mitigate abiotic stresses [al Haq, S. et al. Intl J. Molec. Sci. (2019) 20: 5321, citing Laloum, T. et al. Trends Plant Sci. (2018) 23: 140-150]. An important plant post-transcriptional modification strategy is miRNA, which binds to the mRNA at any point to repress translation or direct cleavage of the mRNA. Some plant miRNAs also are involved with abiotic stress tolerance [Id., citing Zhang, B. et al. Dev. Biol. (2006) 289: 3-16].

HSPs are proteins characterized by the presence of a carboxylic terminal called heat-shock domain [Al-Whaibi, MH., J. King Saud Univ.—Science (2011) 23: 139-150, citing Helm, K W et al. Mol. Cell Biol. (1993) 13: 238-247]. The concentration of HSPs dramatically increases when cells are grown at higher temperatures where HSPs help newly synthesized proteins to fold or protect proteins that might misfold and thereby lose their potential functional conformation during a stress event, such as biotic and/or abiotic stress [al Haq, S. et al. Intl J. Molec. Sci. (2019) 20: 5321, citing Weinmann, H. and Ottow, E. In Comprehensive Medicinal Chemistry II, Taylor, J B and Triggle, D J Eds. Elsevier: Oxford UK (2007); pp. 221-251]. These stress-responsive biomolecules act as molecular chaperones which perform under stress situations [Id., citing Ahuja, I. et al. Trends Plant Sci. (2010) 15: 664-674]. The main functions of HSPs are proper protein folding, unfolding and transport, in conjunction with their localization in the cell, and, subsequently, disposal and degradation of the non-native proteins, [Id., citing Balchin, D. et al. Science (2016) 353: acc4353; Hartl, F U et al. Nature (2011) 475: 324-332; Benesova, M. et al. PLoS ONE (2012) 7: e38017].

HSPs are important in the plant life cycle as their role extends beyond the protection from biotic and abiotic stresses. Although HSPs (with the exception of ubiquitin) were first characterized due to their response to high temperatures, now many HSPs are found in normal, non-stressed cells, and are produced at particular stages of the cell cycle, or during development in the absence of stress [Id., citing Vierling, E. Annu Rev. Plant Biol. (1991) 42: 579-620]. For example, beside stress-responsive biomolecules, HSPs also are involved in plant growth and development under normal conditions, like the flowers, seeds and fruits set, development [Id., citing Eck, ERHB et al. J. Biosci. (2007) 32: 501-510], tuberization [Id., citing Lehesranta, S J et al. Proteomics (2006) 6: 6042-6052; Agrawal, L. et al. J. Proteome Res. (2013) 12: 4904-4930; Agrawal, L. et al. J. Proteome Rs. (2008) 7: 3803-17; Ahn, Y J and Zimmerman, J L. Plant Cell Environ. (2006) 29: 95-104] and nutrient uptake [Id., citing Shekhar, S. et al. J. Proteom. (2016) 143: 306-17]. HSPs are found in different compartments of the cells, such as cytoplasm, nucleus, and cell organelles, e.g., mitochondria, chloroplasts and endoplasmic reticulum [Id., citing Water, E R et al. J. Exp. Biol. (1996) 325-338; Boston, R S et al. In Post-Transcriptional Control of Gene Expression in Plants, Springer: Berlin, Germany (1996), pp. 191-222].

HSP expression is controlled by transcription factors known as the heat shock factors (HSF). Among the HSF classes, HSFA positively regulates plant tolerance to anoxia, heat, osmotic and oxidative stresses [Id., citing Zhuang, L. et al. Intl J. Mol. Sci. (2018) 19: 2702]. HSFA1, found in tomato plants, is considered a master regulator of signal perception, transduction and controlling the expression of stress-responsive genes, including HSPs [Id., citing Guo, M. et al. Front Plant Sci. (2016) 7: 114; Mishra, S K et al. Genes Dev. (2002) 16: 1555-1567], thus, increased expression of HSPs and other stress responsive genes. HSFs play an important and significant role in modifying physiological and biochemical processes, which leads to the development of tolerance to stresses [Id., citing Kotak, S. et al. Curr. Opin. Plant Biol. (2007) 10: 310-316; Scharf, K D et al. Biophys. Acta Gene Regul. Mech. (2012) 1819: 104-119]. HSPs show a response to biotic and abiotic stress situations by up- or down-regulation, but, sensing signals and transduction, particularly in biotic stress, remains to be explored [Id., citing Singh, R K et al. Sci. Rep. (2016) 6: 32641].

### Classification and Nomenclature of HSPs

Heat shock proteins are conserved in almost all organisms from bacteria, to fungi, plants and animals, including human beings. HSPs are classified and named based on the molecular weight in kilo Dalton (kDa), which ranges from 8-200 kDa [Id]. Based on molecular weight, HSPs generally are classified into the following sub-families: HSP100, HSP90, HSP70, HSP60, and small HSPs (sHsps), which are characterized by a conserved sequence of 80-100 amino acid residues, with a molecular weight ranging from 13 to 43 kDa [Reddy, V S et al. Cell Stress and Chaperones (2018) 23: 441-454; al Haq, S. et al. Intl J. Molec. Sci. (2019) 20: 5321, citing Schoffi, F. et al. Sci. Research (1999) 81-98; Schlesinger, MJ. J. Biol. Chem. (1990) 265: 12111-4; Kotak, S. et al. Plant Cell (2007) 19: 182-195; Shamovsky, I. and Nudler, E. Cell Mol. Life Sci. (2008) 65: 855-861; Guo, M. et al. Frong. Plant Sci. (2015) 6: 806]. These chaperone families are involved in maintaining cell homeostasis, transportation of newly synthesized proteins across cell organelles, and folding-preventing misfolded, denatured and aggregated proteins caused by stress conditions [Id., citing Balchin, D. et al. Science (2016) 353: acc4353; Ratajczak, E. et al. J. Mol. Biol. (2009) 386: 178-89; Tyedmers, J. et al. Nat. Rev. Mol. Cell Biol. (2010) 11: 777-788].

The genes which encode different HSPs are found in different cell compartments, such as the nucleus, mitochondria, chloroplast, endoplasmic reticulum and cytosol [Id., citing Liu, D. et al. Plant Physiol. Biochem. (2006) 44: 380-386]. Similarly, the accumulation of these HSPs in different parts of the cell also depends on the intensity of the stress. Nuclear HSPs, for instance, are accumulating in the cytosol at the lower and higher temperatures of 27° C. and 43° C., respectively, while the same aggregate in chloroplast is at 37° C. [Id., citing Waters, E R et al., J. Exp. Bot. (1996) 47: 325-338].

Different HSPs are found and differentially expressed in different species and in different genotypes but in the same species, as investigated by Korotaeva et al., (2001) and Nieto-Sotelo et al., (2002) [Korotaeva, N E et al. Russ. J. Plant Physiol. (2001) 48: 798-803; Nieto-Sotelo, J. Plant Cell (2002) 14: 1621-33] in small HSPs where five sHSPs showed a response to a higher temperature (42° C.) in maize but only one is expressed in wheat and rice. Likewise, HSP68 is expressed in mitochondria under stress situations in potatoes, tomatoes, and soybeans [Id., citing Neumann, D. et al. Planta (1993) 190: 32-43].

The most studied species of plant is Arabidopsis thaliana, where the response to heat-shock treatment occurs through the participation of a number of Hsps: 13 HSPS20; 8 Hsp70; 7 Hsp90, 8 Hsp100, and 21 transcription factors (Hsfs) [Al-Whaibi, MH, J. King Saud University—Science (2011) 23: 139-150, citing Swindell, W R et al, BMC Genomics (2007) 8: 125], but in tomato there are at least 15 Hsfs (Id., citing von Koskull-Doring, P. et al Trends Plant Sci. (2007) 12: 452-457).

Higher plants are characterized by the presence of at least 20 types of sHsps. sHsps are usually undetectable in plant cells under physiological conditions, but are induced upon stress and plant tolerance to stress, including drought, salinity, oxidized species, and low temperatures [Id., citing Low, D. et al Planta (2000) 211: 575-582; Hamilton, E V and Heckathorn, SA Plant Physiol. (2001) 126: 1266-1274; Scharf, K D et al Cell Stress Chaperones (2001) 6: 225-237; Zhang, J-H et al. Scientia Horticulturae (2008) 117: 231-240].

Furthermore, the sHsps of A. thaliana and Lycopersicon esculenium are divided into three subclasses [Id., citing Scharf, K D et al Cell Stress Chaperones (2001) 5: 225-237; Siddique, M et al, Cell Stress Chaperones (2003) 8: 381-394]. These included: subclass C1, represented by 6 proteins in A. thaliana and five proteins in L. esculenium; subclass C11 represented by two genes in both plants; and subclass CIII represented by one gene in both plants. A study reported the presence of other groups in the cytoplasm of A. thaliana cells, and could be categorized into subclasses: CIV, CV, CVI, and CII. Each subclass has its own distinct characteristics and role.

There are six groups of genes that encode for the sHsps. The grouping is based on the sequence similarity and the location of these proteins in the cell. There are two classes of proteins (Class I and Class II) in the cytoplasm encoded by two groups of genes. Other locations are chloroplasts, endoplasmic reticulum, mitochondria and membranes [Id., citing Vierling, E. Annu. Rev. Plant Phys. (1991) 42: 579-620; Waters, E R et al. H. Exp. Bot. (1996) 47: 325-338]. The expression of genes for these sHsps is limited in the absence of environmental stress and occurs in some stages of growth and development of plants, such as embryogenesis, germination, development of pollen grains, and fruit ripening [Id., citing Sun, W. et al. Biochim Biophys. Acta (2002) 1577: 1-9].

Some HSPs showed a tissue-specific response to stress situations; HSP101 was expressed more in reproductive parts like tassels, ear, and endosperm, than in vegetative parts like leaves and roots in maize [al Haq, S. et al. Intl J. Molec. Sci. (2019) 20: 5321, citing Young, T E et al. Plant Physiol. (2001) 127: 777-791]. Some HSPs responded differently to the varying length of stresses. As reported by Heckathorn et al. [Id., citing Heckathorn, S A et al. Int. J. Plant Sci. (1998) 159: 39-45], HSP45, a nuclear protein, accumulated in the chloroplast after a 3 h exposure to heat stress, which returned to its native state after removal of stress. Similarly, HSPs are triggered differently at different development stages. HSP45, for example, showed a response in the whole plant to a stress situation, while HSP64 and HSP72 only showed expression in the reproductive parts i.e., pollens [Id., citing Frova, C. et al. Dev. Genet. (1989) 10: 324-332; Ristic, Z. et al. J. Plant Physiol. (1996) 149: 424-432].

### Regulation of HSPs

When plants are exposed to stress, the synthesis of normal proteins is decreased while the expression of special genes are up-regulated, and, as a result, synthesis of HSPs is triggered. The transcriptional regulation of HSPs to respond to stresses is called the heat shock response (HSR) [Id., citing Shamovsky, I., Nudler, E. Cell Mol. Life Sci. (2008) 65: 855-861].

The transcription of HSPs is controlled by regulatory proteins called heat stress transcription factors (HSFs), which are located in the cytoplasm in an inactive state. HSFs are considered as transcriptional activators for heat shock [Al-Whaibi, M. J. King Saud Univ.—Science (2011) 23: 139-150, citing Clos, J. et al Cell (1990) 63: 1085-1097; Baniwal, S K et al. J. Biosci. (2004) 29: 471-487; Hu, W. et al Plant Sci. (2009) 176: 583-590]. Each factor has one carboxylic terminal (C-terminal) and three amino terminals (N-terminal) and has the amino acid leucine [Id., citing Schuetz, T J et al. Proc. Natl Acad Sci. USA (1991) 88: 6911-15].

Plants are characterized by a large number of transcriptional factors, at least 21 [Id., citing Nover L. and Baniwal, SK. In Intl Symposium on Environmental Factors, Cellular Stress and Evolution, Varanasi, India, October 13-15 (2006), p. 15]. The factors have been classified into three classes (HsfA, HsfB, and HsfC) according to the structural differences in their aggregation in triples (oligomerization domains). While each factor has its role in the regulatory network, all cooperate in regulating many functions and different stages of response to periodic heat stress (triggering, maintenance, and recovery.

The HSR is regulated by the heat stress transcription factors (HSFs) in the promoter region, which bind to cis-acting elements known as HSE (Heat Shock Elements) [al Haq, S. et al. Intl J. Molec. Sci. (2019) 20: 5321, citing Akerfelt, M. et al. Nat. Rev. Mol. Cell Biol. (2010) 11: 545-552; Pirkkala, L. et al. FAEB J. (2001) 15: 1118-1131]. HSFs are classified as three types: HSFA, HSFB, and HSFC; the functions of these classes vary from each other. Among the HSFs, HSFA, which is found in the cytosol in a monomeric state, regulates the HSPs cycle. The activity of the HSFA, under normal conditions, is regulated negatively by HSP90s, which are checked in the form of phosphoproteins [Id., citing Ali, A. et al. Mol. Cell Biol. (1998) 18: 4949-4960]. During the onset of stress, this repression is reversed and HSP90 dissociates and changes into a functional trimer state. The HSFA homo-trimer then binds to the Heat Shock Element (HSE) in the promoter region [Sou, J. et al. Cell (1998) 94: 471-480], transcription occurs and HSPs are synthesized [Id., citing Calderwood, S K et al. Sign. Transduct. Insights (2010) 2: 13-24]. Among the HSFAs, HSFA1 acts as the master regulator in tomatoes [Id., citing Mishra, S K et al. Genes Dev. (2002) 16: 1555-67]. HSFA2 is structurally and functionally the same as HSFA1 but is expressed only in stressed plants. Under stress situations, HSFA2 forms a super activator hetero-oligomer structure with HSFA1, which is more efficient than the individual HSFs, which not only regulate the down-stream stress related HSP genes, but also the protective enzyme genes such as GST, GR, POX and APX [Id., citing Zhuang, L. et al. Int. J. Mol. Sci. (2018) 19: 2702; Scharf, K D et al. Biochim. Biophys. Acta Gene Regul. Mech. (2012) 1819: 104-119]. Some studies also report that HSP gene expression positively regulates protective enzyme activities. In Arabidopsis, for example, overexpression of HSP17.8 enhanced superoxide dismutase (SOD) activity and, in tobacco, HSP16.9 increased the activities of ROS scavenging by ROS scavenger gene expression, e.g., peroxidase (POD), catalase (CAT) and SOD [Id., citing Driedonks, N. et al. Front. Plant Sci. (2015) 6: 999].

Post-transcriptional modification, such as alternative splicing, also regulate the HSFs. HSFA2 under heat stress, for instance, binds to its own promoter region and activates its own transcription in a positive auto-regulatory loop. Similarly, HSFA is regulated by DREB2 under stress, which in turn regulates the stress related genes in many plants [Id., citing Laloum, T. et al. Trends Plant Sci. (2018) 23: 140-150] Similarly, miRNAs also play a vital role in the stress response by down-regulation of stress-related genes. For example, some miRNAs are reported to exert positive regulation in drought, cold, salinity, hormones and nutrient starvation stresses, such as miR159, miR319, miR395, miR402 [Id., citing Zhang, B. et al. Dev. Biol. (2006) 289: 3-16]. Conversely, in Arabidopsis short term heat stress, miR398 negatively regulates the expression of cold shock domain CSD1, CSD2 and capsanthin-capsorubin synthase (CCS), which yield SOD [Id., citing Driedonks, N. et al. Front. Plant Sci. (2015) 6: 999]

### Biotic Stress Tolerance

Plant growth, development, yield, and quality are affected adversely by several biotic factors such as pathogenic bacteria, fungi, viruses, and nematodes. Biotic factors directly deprive their host plants of their nutrients, which result in reduced plant vigor, growth, productivity and sometimes leads to death of the host plants. Biotic stresses are a major cause of pre- and post-harvest losses. Animals have an immune system, which helps them to adapt to biotic stresses such as new diseases and memorized the past infections. Although plants lack this adaptive immune system, they have evolved several sophisticated strategies to counteract these biotic stresses. These defense mechanisms are stored in the plant's genome at the genetic level, which encode thousands of stress resistance genes. HSP response to biotic stresses depend on the nature of the causal organisms and plant genotypes, either susceptible or resistant, and the developmental stage [Id., citing Dodds, P. and Rathjen, J. Nat. Rev. Genet. (2010) 11: 539].

### Abiotic Stress Tolerance

Abiotic stresses are extreme environmental conditions like extreme temperatures, water deficit, and ion imbalance due to heavy metals and salinity, which pose a serious threat to plants survival, yield and quality.

### High Temperature Stress

Several studies have indicated that many high molecular weight HSPs showed a response under high-temperature stress, such as HSP118, HSP114, HSP110, HSP108, HSP104, HSP103, HSP101, HSP100 and HSP97, respectively, [Id., citing Young, T E et al. Plant Physiol. (2001) 127: 777-91, Singla, S L et al. J. Biosci. (1998) 23: 337-45]. Among the HSP100 class, a significant high temperature response (HTR) was shown by HSP101 [Id., citing Lee, U. et al. Plant J. (2007) 49: 115-127] and also was involved in thermo-tolerance in Arabidopsis [Id., citing Queitsch, C. et al. (2000) 12: 479-492]. It was confirmed further in maize that HSP101 was involved in thermo-tolerance [Id., citing Nieto-Sotelo, J. et al. Plant Cell (2002) 14: 1621-33]. Merret et al., (2017) and Mcloughlin et al., (2016) [Id., citing Merret, R. et al. Plant Physiol. (2017) 174: 1216-1225; McLoughlin, F. et al. Plant Physiol. (2016) 172: 1221-36] confirmed the role of HSP101 in thermotolerance in Arabidopsis but also established that this played a role in recovery after heat shock. Besides the role of HSP101 as a chaperone, HSP100 also was involved in development [Id., citing Pyatrikas, D V et al. Russ. J. Plant Physiol. (2014) 61: 80-80]. Low molecular weight HSPs i.e., HSP18.1 and HSP17.9, accumulated in the pea while it was treated for four hours at 42° C. The response and expression of HSPs also were development stage and different tissue specific. In maize subjected to 40° C., HSPs (−101, −70 and −17.6) were induced. Above 36° C., fertilization was reduced, although HSPs were induced in female reproductive parts but, when studied, mature pollens were more sensitive to heat stress [Id., citing Dupuis, I., and Dumas, C. Plant Physiol. (1990) 94: 665-670]. In contrast, HSP70s were expressed more in tomato pollens [Id., citing Duck, N B and Folk, W R. Plant Mol. Biol. (1994) 26]: 1031-1039]. In Arabidopsis HSP70 was expressed more in mitochondria under high temperature stress [Id., citing Sung, D Y et al. Plant Physiol. (2001) 126: 789-800]. Chloroplast HSP70.1, 70.2 and mitochondrial HSP22 also were involved in seed development besides its role as a chaperone [Id., citing Su, PH and Li, H. Plant Physiol. (2008) 146: 1231-1241; Avelange-Macherel, M H. Plant Cell Environ. (2015) 38: 1299-1311].

HSP90 also showed increased expression under heat stress situations. HSP90 has been reported in rice and Arabidopsis [Id., citing Hu, W. et al. Plant Sci. (2009) 176: 583-590; Prasad, B D et al. PLoS ONE (2010) 5: e12761] and all classes of HSP90 (A, B, and C) in soybeans [Id., citing Xu, J. et al. PLoS ONE (2013) 8: e69810]. Under normal conditions, HSP90 negatively regulated HSFs and kept the regulation of all HSPs checked [Id., citing Yamada, K. et al. J. Biol. Chem. (2007) 282: 37794-37804].

HSP70s and HSP60s chaperonin families are the most studied of the chaperones under heat stress, which maintained protein proper folding using ATPs [Id., citing Hartl, F U et al. Nature (2011) 475: 324-332]. Cytosolic HSP70 was involved in heat stress tolerance in Arabidopsis [Id., citing Jungkunz, I. et al. Plant J. (2011) 66: 983-995]. HSP70s have been studied under high temperature stress in a variety of plant crops, such as witch-grass and alfalfa [Id., citing Song, G. et al. Plant Cell Resp. (2018) 37: 1485-1497; Li, Z. et al. J. Plant Res. (107) 130: 387-96]; vegetables like pepper, tomato, cabbage, potato; ornamental plants like chrysanthemum [Id., citing Guo, M. et al. Plant Si. (2016) 252: 246-256; Huang, L. et al. Protoplasma (2019) 256: 39-51; Usman, M G, et al. Cell Stress Chaperones (2018) 23: 223-234; Zhang, S. et al. Plant Cell Physiol. (2017) 59: 58-71; Lee, SS. Et al. Curr. Genom. (2010) 19: 12-20; Liu, J. et al. Sci. Rep. (2018) 8: 16628; Zhang, Y. et al. Plant Omics (2014) 7: 229]; grain such as wheat [Id., citing Id, H W et al. J. Mol Sci. (2018) 19: 1594]; and tea [Id., citing Chen, J. et al. Int. J. Mol. Sci. (2018) 19: 2633]. Xu et al., (2010) [Id., citing Xu, C. and Huang, B. Crop Sci. (2010) 50: 2543-2552] observed the expression pattern of chloroplast HSP60, not only in normal conditions but also under high-temperature and drought situations. It was responsible for ribulose biphosphate carboxylase/oxygenase (Rubisco, a key enzyme in photosynthesis) assembly, protection and also chloroplast development. Low molecular weight HSPs such as the HSP10, HSP20 and HSP40 families were up-regulated under high-temperature stress situations in various plant crops [Id., citing Xu, Y. et al. Intl J. Prteom. (2011) 2011: 529648; Huang, L. et al. Protoplasma (2019) 256: 39-51; Kumar, N. et al. 3 Biotech (2017) 7: 205; Liao, J L et al. J. Exp. Bot. (2013) 65: 655-671; Lin, C J et al. J. Agric. Food Chem. (2010) 58: 10545-10552; Majoul, T. et al. Proteomics (2004) 4: 505-13; Wang, X. et al. Genes Genom. (2018) 11: 1-8]. Some small HSPs were also genotype-specific and were up-regulated in resistant cultured varieties bred by humans (“cultivars”), such as foxtail millet, while some small HSPs were down-regulated in sensitive genotypes [Id., citing Singh, R K et al. Sci. Rep. (1016) 6: 32641]. Some co-chaperones were involved in thermo-tolerance as HSP40. Correlation of small HSPs with HSP100, HSP70 and HSP60 suggested their role as holders in disaggregation and protein folding [Zhang, Y. et al. Plant Omics (2014) 7: 229].

### Low-Temperature Stress

Cold stress affects plant enzymes, membrane plasticity, changes physiology and metabolism, sometimes causes water starvation and desiccation, which creates a stress condition for the plant that adversely affects plant growth, development and yield. Low temperature also is associated with protein disfunction and denaturing, which induce the accumulation of HSPs [Id. citing Hashimoto, M. and Komatsu, S. Proteomics (2007) 7: 1293-1202; Hlavackova, I. et al. Int. J. Mol. Sci. (2013) 14: 8000-8024; Kosova, K. et al. J. Proteom. (2011) 74: 1301-1322]. Many HSPs responded to cold stress and were up-regulated in Arabidopsis, tobacco, maize, rapeseed, chicory, poplar, wheat and barley [Id., citing Hlavackova, I. wt al. Intl J. Mol Sci. (2013) 14: 8000-8024; Bae, M S et al. Plant J. (2003) 36: 652-63; Jin, Y. et al. Afr. J. Biotechnol. (2011) 10: 18991-19004; Kollipara, K P et al. Plant Physiol. (2002) 129: 974-992; Reddy, R K et al. lant Sci. (1998) 131: 131-37; Degand, H. et al. Proteomics (2009) 9: 2903-2907; Ghosh, D. and Xu, J. Front. Plant Sci. (2014) 5: 6; Vitamvas, P. et al. Proteomics (2012) 12: 68-85; Ono, K. et al. Plant Sci. (2001) 160: 455-61]. Under low-temperature stress situations, HSPs were induced and translocated into various cell organelles to protect them from cold stress [Id., citing Bae, M S et al. Plant J. (2003) 36: 652-663]. Bae et al., (2003) investigated this in Arabidopsis treated with cold stress at 40° C. for 6 h. HSP70s were up-regulated and their traffic from the cytoplasm to the nucleus was observed. A similar event was observed in the pea mitochondria when treated at 4° C. for 36 h [Id., citing Taylor, N L et al. Mol. Cell Proteom. (2005) 4: 1122-1133]. Some HSPs accumulated tissue specifically upon low-temperature exposure, as in poplar, where HSPs were accumulated in leaves [Id., citing Renaut, J. et al. Plant Biol. (2004) 6: 81-90]. Regarding rice, low-temperature stress and a gradual decrease in the temperature from 15° C. to 0° C., with an interval of 5° C., up-regulated HSP95 and HSP75, and HSP70 accumulated in the chloroplast, as this was the part of the plant vulnerable to low temperature [Id., citing Hahn, M. and Walbot, V. Plant Physiol. (1989) 91: 930-938; cui, S. et al. Proteomics (2005) 5: 3162-3172]. Some of the HSPs, like HSP90 in wheat and HSP60 and HSP21 in sunflowers, are down-regulated to cold stress [Id., citing Vitamvas, P. et al. Proteomix (2012) 12: 68-65; Balbuena, T S et al. J. Proteome Res. (2011) 10: 2330-2346]. A similar trend also was reported by Hlavackova et al., (2013) and Rinalducci et al., (2011) [Id., citing Hlavackova, I. et al. Intl J. Mol. Sci. (2013) 14: 8000-8024; Rinalducci, S. et al. J. Proteom. (2011) 74: 643-659] where Rubisco stability was associated with down-regulation of HSP60 and HSP21 in winter wheat.

### Drought Stress

Drought stress, in combination with other abiotic stresses such as high light and temperature stress, negatively affects plant morphological, physiological and molecular characteristics, which leads to lowered photosynthesis, hormonal imbalance, mineral nutrient starvation and an ultimate oxidative stress [Id., citing Komatsu, S. et al. J. Proteome Res. (2011) 10: 3993-4004]. Removal of water disrupts the normal structure of the lipid bilayer plasma membrane. This results in the displacement of membrane proteins, denaturation of membrane-based enzymes and, as a result, membrane permeability, physiology and metabolism are adversely affected [Id., citing Salehi-Lisar, SY and Bakhshayeshan-Agdam, H. Drought Stress in Plants: Causes, Consequences, and Tolerance, Physiology and Biochemistry; Springer International Publishing: Cham, Switzerland; Berlin, Germany, 2016; pp. 1-16; Chaves, M M et al. Funct. Plant Biol. (2003) 30: 239-264]. Dehydration stress also affects the quantity and quality of normal plant proteins and, as a result, stress related proteins including HSPs are induced. For example, HSP70 was up-regulated in drought stress in the seedling of upland rice [Id., citing Reddy, P S et al. PLoS ONE (2014) 9: e89125]. Similarly, transgenic Arabidopsis and sugarcane also showed HSP up-regulation and demonstrated drought tolerance [Id, citing Yer, E N et al. Gene (2018) 678: 324-36; Subba, P. et al. J. Proteome Res. (2013) 12: 5025-5047]. The expression pattern of HSPs is also genotype-specific; Burke J J et al., [Id., citin Burke, J J et al. Plant Physiol. (1985) 78: 394-398] studied combined drought and heat stress in irrigated and non-irrigated cotton, where more HSPs accumulated in non-irrigated cotton. Maize heat tolerant and sensitive cultivars were studied under high temperature and dehydration situations, where HSP accumulation was more in drought stress conditions [Id., citing Ramanjulu, S. and Bartels, D. Plant Cell Environ. (2002) 25: 141-151]. The same was demonstrated by Benesova et al., [Id., citing Bensevova, M. et al. PLoS One (2012) 7: e38017], where HSP70 and HSP26 were induced in drought-stressed maize. A study on chickpea HSP70 reported that HSPs were first down-regulated in the early stage of growth in drought-tolerant cultivars (meaning a plant that has been produced in cultivation by selective breeding). In contrast, HSPs were abundant in drought-sensitive cultivars, which indicated that HSPs responded to drought not only in the specific genotypes but, also, during the developmental stage. Similarly, small HSPs expressed highly in drought-tolerant cultivars as compared to those that were sensitive in chickpea [Id., citing Subba, P. et al. J. Proteome Res. (2013) 12: 5025-5047]. The same trend also was observed in poplar and Kentucky bluegrass [Id., citing Xu, C. and Huang, B. Crop Sci. (2010) 50: 2543-2552; Burke, J J et al. Plant Physiol. (195) 78: 78: 394-398]. HSP17.7 showed drought tolerance in transgenic rice, and other HSPs also were involved in the acclimation of bryophytes to drought stress [Id., citing Agrawal, L. et al. Front. Plant Sci. (2016) 7: 1466; Ristic, Z. et al. Plant Physiol. (1991) 97: 1430-1434]. Proteomics studies revealed that nuclear and HSPs in the extracellular matrix were both up-regulated to drought stress [Id., citing Cruz de Carvalho, R. et al. Plant Cell Environ. (2014) 37: 1499-1515; Pandey, A. et al. Mol. Cell Proteom. (2008) 7: 88-107; Pandey, A. et al. J. Proteom Res. (2010) 3443-64; Bhushan, D. et al. J. Proteome Res. (2011) 10: 2027-2046].

### Salinity Stress

Studies show that many HSPs are induced and up-regulated in saline stress situations like HSP70 in rice seedlings [Id., citing Ngara, R. et al. Proteomics (2014) 14: 611-621], wheat [Sobhanian, H. et al. J. Proteo. (2011) 74: 1323-37], and poplar HSP70-9, -12 and -33 [Id., citing Manaa, A. et al. J. Exp. Bot. (2011) 62: 2797-2813]. Furthermore, HSP40 in rice [Id., citing Wang, X. et al. Genes Genom. (2018) 11: 1-8] and poplar, HSP100-21 and -75), HSP90-9 and -12), HSP60-31, -33, -38 and -49), HSP40-113 and -117, and HSP21 were also up-regulated under salt stress [Id., citing Manaa, A. et al. J. Exp. Bot. (2011) 62: 2797-2813]. In wheat hybrid Jinan 177 and its salt-resistant hybrid, protein profiling showed HSPs and chaperones were induced highly under salt stress [Id., citing Wang, M. et al. Proteomics (2008) 8: 1470-89]. HSPs were studied in relation to programmed cell death (PCD) in a rice root at higher salt, where mitochondrial HSP70 were the up-regulated proteins that possibly were involved in PCD regulation [Id., citing Han, F. et al. Biochim. Biophys. Acta Proteins Proteom. (2009) 1794: 1625-1634]. Soybean proteomic studies showed a differential HSPs expression of HSP90, chloroplast HSP70, HSP60 and HSP20 under salt stress [Id., citing Song, H. et al. Plant Mol. Biol. Report (2009) 27: 342-349]. Different HSPs in Arabidopsis like HSP 90 [Id., citing Xu, J. et al. PLoS ONE (2013) 8: e69810; Choudhary, M K, et al. Mol Cell Proteom. (2009) 8: 1579-98], HSP100, Clp (B1, B2), Clp (D1, D2) and small HSPs in rice [Id., citing Muthusamy, S K et al. Front. Plant Sci. (2016) 7: 929; Song, H. et al. Planta (2009) 229: 955-64] showed tolerance to high salinity stress. The role of HSPs in response to salinity stress is also genotype-specific, as recorded in soybean, where HSPs were induced more in salt resistant cultivars [Id., citing Pi, E. et al. Mol. Cell Proteom. (2016) 15: 266-288].

### Light Stress

As autotrophs (meaning an organism that is able to form nutritional organic substances from simple inorganic substances such as carbon dioxide), plants require light for photosynthesis. Excess light damages the photosynthetic apparatus and plants undergo a phenomenon known as photorespiration. During this process, toxic chemicals, rather than sugars, along with ROS, are produced. These toxic chemicals in the chloroplast can damage the photosystem II permanently by excessive absorption of light [Id., citing Timperio, A M et al. J. Proteom. (2008) 71: 391-411; Kumar, M. et al. Front. Plant Sci. (2017) 7: 2023]. Rossel et al., [Id. citing Rossel, J B et al. Plant Physiol. (2002) 130: 1109-1120] reported that many HSPs were up-regulated upon high light stress (HLS) in Arabidopsis. A similar over-accumulation of nuclear HSP70 was observed in Chlamydomonas. The thylakoid proteome analysis of Arabidopsis was studied with respect to high light saturation involving isoforms of chloroplast HSP70 along with the accumulation of other osmolytes like anthocyanins and ascorbates. In the marine ecosystem where low light created a stress, HSP70, ClpB1, Sti, and HSP60 were up-regulated [Id., citing Kumar, M. et al. Front. Plant Sci. (2017) 7: 2023]. Under high light saturation, small HSP23 was seen to be involved in the post-transcriptional regulation in a Chenopodium rubrum cell suspension [Id., citing Debel, K. et al. Planta 91997](201(3): 326-33).

### Chemical Pollutant Stress

Plant productivity is restricted by chemical pollutants in the soil media, such as heavy metals. These pollutants affect plant growth either by displacement of essential cations from specific binding sites or by generation of oxidative stress by the generation of ROS [Al HaQ. S. et al. Intl J. Molec. Sci. (201 20: 5321, citing Sharma, SS. And Detz, KJ. Trends Plant Sci. (2009) 14: 43-50)

HSPs can be induced by heavy metal stress. For example, HSP70s were differentially expressed and accumulated in the roots of tomatoes [Id., citing Rodriguez-Celma, J. et al. J. Proteom. (2010) 73: 1694-1706]. Similarly, the HSP70 sub-family, DnaK (Bip), was up-regulated in rice seedlings [Id., citing Ahsan, N. et al. C. R. Biol. (2007) 330: 735-468]. Arabidopsis exposure to cadmium stress induced many HSPs [Id., citing Sarry, J. et al. Proteomics (2006) 6: 2180-2198]. Similarly, increased expression was reported for HSP80 and HSP17.9 in rice [Id., citing Ahsan, N. et al. C R Biol. (2007) 330: 735-746], HSP 90s in Lotus corniculatus [Id., citing Nacascues, J. et al. New Phytol. (2012) 193: 625-636], HSP17.7 in carrots and HSP26 in soybeans [Id., citing Czmecka, E et al. Mol. Cell Biol. (1988) 8: 1113-1133] under cadmium, lead and arsenic stresses. Using a comparative proteomic analysis of poplar under cadmium stress, a differential expression pattern of HSP was noted. Similarly, in soybeans, two-folds higher accumulation of HSP was recorded in Cd-accumulating genotypes, while there was less HSP70 expression in lower Cd-accumulating varieties, which showed that HSP expression was also genotype-specific [Id., citing Hossain, Z. et al. Amino Acids (2012) 43: 2393-2416]. When flax was cultured on heavy metal treated media, many heavy metal binding proteins, including HSP70 accumulation, were enhanced, while HSP83 showed down-regulation. HSP90.3 enhanced cadmium stress tolerance by lowering germination potential in Arabidopsis, mediating the antioxidant enzymes [Id., citing Song, H M et al. Biol. Plant (2012) 56: 197-199].

### Flooding Stress

Waterlogging/flooding is also an environmentally limiting factor that hinders plant growth and development. A gradual decrease of redox potential and oxygen in the soil are the ill effects of flooding [Id., citing Hossain, Z. et al. J. Plant Physiol. (2009) 166: 1391-1404]. Studies show that HSPs are involved in plant resistance against flooding stress by up-regulation and higher gene expression, which is organ-specific. As noticed by Chen et al., [Id., citing Chen, Y. et al. Proteome Sci. (2014) 12: 33] in the soybean plasma membrane where HSP70 accumulated more than 10 fold, this occurrence occurred more in cotyledon than the roots of the soybean. Discussed in another proteomic study by the same group of researchers, HSP60 was differentially regulated in soybeans [Id., citing Komatsu, S. et al. PLoS ONE (2013) 8: e65301]. In contrast, HSPs were induced in flooding stress, but were not mandatory for resistance in flooding stress and were genotype specific. As for resistant and susceptible cultivars of rice to anoxia and hypoxia conditions, HSPs were more up-regulated in the sensitive cultivars than resistant genotypes [Id., citing Komatsu, S. et al. J. Proteome Res. (2011) 10: 3993-4004]. Proteomics study of flooding stress in relation to PCD in maize revealed that HSP70s were up-regulated [Salehi-Lisar, S. Y.; Bakhshayeshan-Agdam, H. Drought Stress in Plants: Causes, Consequences, and Tolerance, Physiology and Biochemistry; Springer International Publishing: Cham, Switzerland; Berlin, Germany, 2016; pp. 1-16]. The same pattern of flooding tolerance was studied in rice protoplast where ectopic mtHSP70 expression protected H₂O₂ induced PCD [Id., citing Komatsu, S. et al. J. Proteome Res. (2011) 10: 3993-4004]. Similarly, in Arabidopsis anoxia tolerance was enhanced via HSFA2-mediated production of HSP70 and HSP101.

### Oxidative/Combined Stress

Since plants are exposed to many stresses simultaneously, such as light, this creates high temperature stress that leads to dehydration. Such situations lead to oxidative or secondary stress and plants have to adjust their signaling pathways and metabolism to ensure their growth and development [Id., citing Mittler, R. et al. Trends Biochem. Sci. (2012) 37: 118-125; Ngara, R. et al. Proteomics (2014) 14: 611-621; Kilian, J. et al. Plant J. (2007) 50: 347-363; Scarpeci, T E et al. Plant Signal Behav. (2008) 3: 856-857]. Oxidative stress generates ROS which, in high concentrations, are harmful to cellular structures. HSPs respond to multiple stress situations and enable the plants to cope with the challenging environment. For example, HSP70 expression was higher in tobacco to heat stress but was even higher to the combined stress of heat and drought [Id., citing Rizhsky, L. et al. Plant Physiol. (2002) 130: 1143-1151]. Ectopic expression of genes from soybeans in Arabidopsis GmHSP90 showed tolerance to heat, salinity and osmotic stresses, although response in salinity was not as high as to combined stresses [Id., citing Xu, J. et al. PLoS ONE (2013) 8: e69810]. A similar pattern was observed with small HSPs in rice to multiple stresses [Id., citing Zou, J. et al. J. Plant Physiol. (2012) 169: 628-635; Want, A. et al. Plant Breed (2015) 134: 384-393]. Overexpression of HSP17.6 in Arabidopsis enhanced tolerance to salinity combined with dehydration, but no response was noted to high temperature stress only [Id., citing Sun, W. et al. Plant J. 2001] 27: 407-15]. Single or combined stresses led to the production of ROS and oxidative stress which, if not checked timely, are very detrimental to plants [Id., citing Hossain, Z. et al. J. Plant Physiol. (2009) 166: 1391-1404]. Under oxidative stress, overexpression of organelle and cytosolic HSP90 enhanced tolerance in Arabidopsis. Similar results were reported by Nishizawa-Yokoi et al. [Nishizawa-Yokoi, A. et al. Plant Cell Physiol. (2010) 51: 486-496], where HSP90 regulated HSFA2, which enhanced tolerance to oxidative stress. Queitsch et al. [Id., citing Queitsch, C. et al. Plant Cell (2000) 12: 479-492] reported oxidative stress accumulated HSP100/Clp B, ClpC2 and ClpD1 in rice. HSPs protected vital cellular parts under oxidative stress, as demonstrated by Downs, C A et al. [Downs, C A et al. J. Plant Physiol. (1999) 155: 488-496], where small HSPs protected the photosystem II from oxidative stress and photo-inhibition. Different organelle HSPs also responded to oxidative stress. mtHSP22 accumulation was enhanced in tomatoes under oxidative stress [Id., citing Banzet, N. et al. Plant J. (1998) 13: 519-577]. Small HSPs responded to oxidative stress, as HSP16.4 and HSP17 accumulated in multiple stress situations in Arabidopsis and carrots, respectively [Id., citing Jiang, C. et al. Plant Cell Environ. (2009) 32: 1046-1059; Sarry, J. et al. Proteomics (2006) 6: 2180-98].

### Genetic Engineering/Induction Studies

Al-Whaibi et al [J. King Saud Univ—Science (2011) 23: 39-50] describe a series of field tests to modulate induction of HSPs in plants. In general, the expression of HSPs and their factors Hsfs was induced largely by heat, cold, salinity and osmotic stresses; they emphasized the importance of studying stress combinations to end up with tolerant plants. The response to other stress factors depends on protein class and tissues. For example, under all types of stresses, high expression response for class HSP20 was recorded. Wounding of the roots of the plant stimulated after 12 h the expression of several genes from classes HSP20; HSP70 and HSP100. High response of expression of genes for HSPs and Hsfs occurred under UV-B stress in aerial tissues (shoot), but there was no expression in non-aerial tissues [Id., citing Sindell, W R et al. BMC Genomics (2007) 8: 125].

The response of plants to heat shock results in changes in the level of enzymes, cellular membrane structure, photosynthesis activity and protein metabolism [Id., citing Singla, S L et al. In: Prasad, M N V (Ed), Plant Ecophysiology, John Wiley, New York, (1997) pp. 101-127]. It has been reported that high temperature changed the properties of membranes of the nucleus, ER, mitochondria and chloroplasts of the rice plant O. saiiva [Id., citing Pareek, A. et al. J. Biosci. (1998) 23: 361-367].

Scientists have modified plant cells to show an increase in cold stress tolerance by increasing gene expression of glycerol 3-phosphate acyltransferase from Cucurbita maxima and A. thaliana in tobacco plant cells, resulting in an increase in the degree of unsaturation of the lipids in the thylakoid membranes of the chloroplast. Therefore, increasing the degree of unsaturation of fatty acids leads to an increase in cold tolerance.

In an attempt to increase salinity tolerance of the wheat plant, one report disclosed that transgenic plants that contained a gene (CtHSR1) from the yeast Candida tropicalis were subjected to water stress, high salinity and heat stresses under operating greenhouse conditions and in the field. Stress conditions were withholding watering the plant for two weeks (water stress), watering in the presence of 400 mM NaCl (salinity stress), and subjecting the plants to 46° C. for 2 h followed by a 3 day period recovery at 28° C. (heat stress). The results showed improvement of growth under both drought and heat stresses and lesser but still significant to salinity stress [Id., citing Blumwald, E. and Arif, A.].

miRNAs in Plants: Their Role in Abiotic Stresses

A growing body of research has demonstrated that miRNAs act on target genes and are involved in various biological functions of plants. Small RNA high-throughput sequencing has been widely used to identify and functionally analyze miRNAs in plants (Zhang, F. et al. Front. Plant Sci. (2022) 13: 919243, citing Sunkar, R. et al. Plant Cell (2005) 17: 1397-1411; Fahlgren, N. et al. PLoS One (2007) 2: e219 et al.; Creighton, C J et al., Brief Bioinform. (2009) 10: 490-497). According to the records registered in miRBase (mirbase.org), 38,589 hairpin precursors and 48,860 mature microRNAs have been identified through experimental or computational approaches from 271 organisms, including more than 70 plants, such as Arabidopsis thaliana (326 precursors, 428 mature), Oryza sativa (604 precursors, 738 mature), Zea mays (174 precursors, 325 mature), Triticum aestivum (122 precursors, 125 mature), Glycine max (684 precursors, 756 mature), Solanum tuberosum (224 precursors, 343 mature), Nicotiana tabacum (162 precursors, 164 mature), Solanum lycopersicum (112 precursors, 147 mature), Gossypium raimondii (296 precursors, 296 mature), Medicago truncatula (672 precursors, 756 mature), Populus trichocarpa (352 precursors, 401 mature), Sorghum bicolor (205 precursors, 241 mature), Brassica napus (90 precursors, 92 mature), Vitis vinifera (163 precursors, 186 mature), and so on [Id.].

SQUAMOSA Promoter-Binding Protein-Like (SPL) genes encode plant-specific transcription factors that play important roles in plant phase transition, flower and fruit development, plant architecture, gibberellins signaling, sporogenesis, and response to copper and fungal toxins. [Chen, X. et al. J. Integr. Plant Biol. (2010) 52 (11): 946-951]. The SQUAMOSA promoter binding protein (SBP)-box proteins are plant-specific transcriptional factors in plants. [Abdulllah, M. et al. Front. Genet. (2018) 9: 64]. MYB proteins are key factors in regulatory networks controlling development, metabolism and responses to biotic and abiotic stresses. [Dubos, C. et al. Trends in PlantSci. (2010) 15 (10): P573-P581].

A preliminary statistical analysis showed that several miRNAs (miR156, miR159, miR160, miR164, and miR172) are the most studied miRNA families that showed diverse roles in diverse stresses and diverse plant species. MiR156-targeted SQUAMOSA promoter binding protein like (SPL) transcription factors (TFs) modulate plant architecture, including grain size, panicle branching, and higher grain productivity in rice [Id., citing Jiao, Y. et al. Nat. Genet. (2010) 42: 541-544; Miura, K. et al. Nat. Genet. (2010) 42: 545-549; Wang S. et al., Nat. Genet. (2012) 44: 950-954] and modulate plant architecture and tuberization in potato and temporal regulation of shoot development in Arabidopsis thaliana [Id., citing Gruber, A J and Zavolan, M. Epigenomics (2013) 5: 671-83; Yao, Q. et al. Curr. Opin. Chem. Biol. (2019) 51: 11-17]. The miRNA156-targeted SPL/SBP (SQUAMOSA promoter binding protein)-box protein transcription factors (TFs) regulate tomato ovary and fruit development [Id., citing Ferreira e Silva, G F et al., Plant J. (2014) 78: 604-618]. The miRNA159 regulated MYB TFs in the regulation of programmed cell death in Arabidopsis [Id., citing Alonso-Peral, M M et al., Plant Physiol. (2010) 154: 757-771], floral development and stem elongation in rice [Tsuji, H. et al. Plant J. (2006) 47: 427-444], anther development and heat response in wheat [Id., citing Wang Y. et al., PLoS ONE (2012) 7: e48445], leaf and floral development in tomato [Id., citing Buxdorf, K. et al. Planta (2010) 232: 1009-1022], and targeting of isotrichodermin C-15 hydroxylase and its involvement in immune response of cotton [Id., citing Zhang, T. et al. Nat. Plants (2016) 2: 16153]. The miR160 regulates a group of repressor auxin response factors (ARFs), which are mainly involved in auxin hypersensitivity and regulation of floral organ development, seed germination, and post-germination stages in Arabidopsis thaliana [Id., citing Liu, PP. et al. Plant J. (2007) 52: 133-146; Liu, X. et al. Plant J. (2010) 62: 416-428], ovary patterning, floral organ abscission and lamina outgrowth in tomato [Id., citing Turner, M. et al. Plant Physiol. (2013) 162: 2042-2055], growth and developmental defects of rice [Id., citing Huang, J. et al. Sci. Repts (2016) 6: 29938], and inhibition of symbiotic nodule development in soybean [Id., citing Turner, M. et al. Plant Physiol. (2013) 162: 2042-55]. The miR164-directed cleavage of NACl mRNA affects lateral root development in Arabidopsis [Id., citing Guo, H S et al., Plant Cell (2005) 17: 1376-1386], maize [Id., citing Li, J. et al., BMC Plant Biol. (2012) 12: 220], drought resistance in rice [Id., citing Fang, Y. et al. J. Exp. Bot. (2014) 65: 2119-2135], and boundary specification in tomato [Id., citing Berger, Y. et al. Development (2009) 136: 823-832] and negatively regulates the resistance of wheat to stripe rust [Id., citing Turner, M. et al. Plant Physiol. (2013) 162: 2042-2055]. miR172 suppressing AP2 genes induce flowering, spikelet determinacy, and floral organ abnormalities in rice (Id., citing Zhu, H. et al., BMC Genomics (2009) 20: 33; Lee, Y S et al. Rice (2014) 7: 31), promote vegetative phase change in maize [Id., citing Lauter, N. et al. Proc. Natl Acad. Sci. USA (2005) 102: 9412-9417], regulate soybean nodulation [Id., citing Yan, Z. et al. Mol. Plant Microbe Interact. (2013) 26: 1371-1377], and affect cleistogamous flowering in barley [Id., citing Nair, S K et al. Proc. Natl Acad. Sci. USA (2010) 107: 490-495] and graft-transmissible induction of potato tuberization [Id., citing Martin, A. et al. Development (2009) 136: 2873-2881]. The expression patterns of miR159, miR160, miR166, miR396, miR393, etc. have significant alterations in drought and salt stress, which suggests that these miRNAs play a vital role in abiotic stress alleviation in chickpea [Id., citing Jatan, R. et al., Genomics (2019) 111: 509-519; Jatan, R. et al. Environ. Exp. Bot. (2019) 157: 217-227]. Moreover, miR164a-CUC1, miR167-NRAMP1, miR393a-5p-TIR1, and miR396a-5p-GRF1 modules might be involved in the regulation of root, leaf, and flower development of Arabidopsis during Pseudomonas putida-inoculation [Id., citing Jatan, R. et al., Int. J. Mol. Sci. (2020) 21: 5468].

Plant miRNAs also are implicated in abiotic stress response mechanisms with regard to oxidative stress and effects on DNA in different plant species [Id., citing Pagano, L. et al., Environ. Exp. Bot. (2021) 184: 104369]. miRNAs play a key role in responding to unfavorable conditions, such as low temperature stress [Id., citing Aslam, M. et al. Int. J. Mol. Sci. (2020) 21: 8441], high temperature stress [Id., citing Zhang, M. et al. Intl J. Mol Sci. (2019) 20: 1754]; drought stress [Id., citing Ni, Z. et al. Biochem. Biophys. Res. Commun. (2012) 427: 330-35], salt stress [Id., citing Nguyen, D Q et al. Intl J. Mol. Sci. (2020) 21: 7879]; and heavy metal stress [Id., citing Ding, Y. et al. J. Agric. Food Chem. (2020) 68: 1958-1965] through regulating expression of related target genes in plants.

### Anatomy and Physiology of the Skin

The skin is the largest organ in the body, consists of several layers and plays an important role in biologic homeostasis and is comprised of the epidermis and the dermis. The epidermis, which is composed of several layers beginning with the stratum corneum, is the outermost layer of the skin, and the innermost skin layer is the deep dermis. The skin has multiple functions, including thermal regulation, metabolic function (vitamin D metabolism), and immune functions. FIG. 1 presents a schematic diagram of the anatomy of the skin.

In humans, the usual thickness of the skin is from 1-2 mm, although there is considerable variation in different parts of the body. The relative proportions of the epidermis and dermis also vary, and a thick skin is found in regions where there is a thickening of either or both layers. For example, on the interscapular (between the shoulder blades) region of the back, where the dermis is particularly thick, the skin may be more than 5 mm thick, whereas on the eyelids it may be less than 0.5 mm. Generally, the skin is thicker on the dorsal or extensor surfaces of the body than on the ventral or flexor surfaces; however, this is not the case for the hands and feet. The skin of the palms and soles is thicker than on any dorsal surface except the intrascapular region. The palms and soles have a characteristically thickened epidermis, in addition to a thick dermis

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy. On the thick skin of the palms and soles, the fields form long, narrow ridges separated by parallel coursing furrows, and in the fingertips these ridges are arranged in the complicated loops, whorls (verticil) and spirals that give the fingerprints characteristic for each individual. These ridges are more prominent in those regions where the epidermis is thickest.

Where there is an epidermal ridge externally there is a corresponding narrower projection, called a “rete peg,” on the dermal surface. Dermal papillae on either side of each rete peg project irregularly into the epidermis. In the palms and soles, and other sensitive parts of the skin, the dermal papillae are numerous, tall and often branched, and vary in height (from 0.05 mm to 0.2 mm). Where mechanical demands are slight and the epidermis is thinner, such as on the abdomen and face, the papillae are low and fewer in number.

### Epidermis

The epidermis provides the body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically [See FIG. 2]. The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

### Stratum Corneum and the Acid Mantle

Stratum corneum is an avascular, multilayer structure that functions as a barrier to the environment and prevents transepidermal water loss. Recent studies have shown that enzymatic activity is involved in the formation of an acid mantle in the stratum corneum. Together, the acid mantle and stratum corneum make the skin less permeable to water and other polar compounds and indirectly protect the skin from invasion by microorganisms. Normal surface skin pH is between 4 and 6.5 in healthy people; it varies according to area of skin on the body. This low pH forms an acid mantle that enhances the skin barrier function.

### Other Layers of the Epidermis

Other layers of the epidermis below the stratum corneum include the stratum lucidum, stratum granulosum, stratum germinativum, and stratum basale. Each contains living cells with specialized functions (FIG. 2). For example, melanin, which is produced by melanocytes in the epidermis, is responsible for the color of the skin. Langerhans cells are involved in immune processing.

### Dermal Appendages

Dermal appendages, which include hair follicles, sebaceous and sweat glands, fingernails, and toenails, originate in the epidermis and protrude into the dermis hair follicles and sebaceous and sweat glands contribute epithelial cells for rapid re-epithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet. Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

### Dermis

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis and the deep dermis.

The superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts. The structural integrity of the dermis plays a role in the normal function and youthful appearance of the skin.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly.

### Hair

Hair is a filamentous outgrowth of protein found only on mammals. The hair of non-human mammal species is commonly referred to as fur. In some species, hair is absent at certain stages of life.

Hair grows from hair follicles deep in the dermis and projects from the epidermis of the skin.

Human skin has two types of hair: vellus hair and terminal hair. Much of human hair is short, under-pigmented vellus hair rather than terminal hair. The most noticeable part of human hair is the hair on the head, which can grow longer than on most mammals and is denser than most hair found elsewhere on the body. The term “scalp” refers to the integument of the upper part of the head, usually including the associated subcutaneous structures. The scalp is the anatomical area bordered by the face anteriorly and the neck to the sides and posteriorly. A healthy scalp is characterized by clean, hydrated skin and good blood circulation, balanced oil production, and the absence of inflammation, itching and flaking.

Vellus hair is short, fine, “peach fuzz” body hair. It is a very soft, generally pale, and short hair that grows in most places on the human body in both sexes. It is usually less than two cm long and the follicles are not connected to sebaceous glands. It is most easily observed in women and children, as they have less terminal hair to obscure it. It is also found in pre-adolescents and in male pattern baldness.

Terminal hair is developed hair, which is generally longer, coarser, thicker and darker than the shorter and finer vellus hair. Phases of growth in terminal hair are more apparent than in vellus hair; it generally has a longer anagen phase. It has associated sebaceous glands, whereas a vellus hair may not. Under certain conditions, such as puberty, some vellus hair may become androgenic hair. Under other conditions, such as male pattern baldness, it may revert to a vellus-like state.

Each hair comprises two structures: the follicle in the skin and the shaft we see. The follicle contains several layers. At the base of the follicle is a projection called a papilla, which contains capillaries, or tiny blood vessels, that feed the cells. The dermal papilla consists of an egg-shaped accumulation of mesenchymal stem cells (MSCs) surrounded by ground substance that is rich in acid mucopolysaccharides (AMPs). The living part of the hair, the area surrounding the papilla called the bulb, is the only part fed by the capillaries. The bulb encompasses the dermal papilla and the hair matrix. The hair matrix surrounds the top and sides of the dermal papilla. It is the actively growing portion of the follicle, which consists of a collection of epidermal cells that rapidly divide, move upward, and give rise to the hair shaft and the internal root sheath. The cells in the bulb divide every 23 to 72 hours, faster than any other cells in the body.

The hair follicle contains stem cells from different developmental origins, such as epithelial stem cells, melanocyte stem cells, and mesenchymal stem cells (MSCs) [Wang, B. et al. World J. Stem Cells (2020) 12(6): 462-470, citing Kiani, M T et al. ACS Biomater. Sci. Eng. (2018) 4: 1193-1207]. These stem cells continuously self-renew, differentiate, regulate hair follicle development, and contribute to hair follicle cycles which consist of the growth phase (anagen), regression phase (catagen), and rest phase (telogen) throughout adult life [Id., citing Agabalyan, N A et al. Exp. Dermatol. (2017) 26: 505-509]. During catagen and telogen, follicles prepare their stem cells for the next anagen. During anagen, bulge stem cells are activated by induction signals from the dermal papilla and migrate downward to the bulb region, where they proliferate and differentiate to regenerate the inner and outer root sheath, matrix, and hair shaft. Human hair follicle-derived MSCs are dermal papilla or sheath cells from human hair follicles that express the MSC immunophenotype and possess multi-lineage differentiation potential [Id., citing Liu, J Y et al. Tissue Eng. Part A (2010) 16: 2553-2564].

The follicle is surrounded by two sheaths—an inner and outer sheath. These sheaths protect and mold the growing hair shaft. The inner sheath follows the hair shaft and ends below the opening of a sebaceous (oil) gland, which produces sebum, a natural conditioner and sometimes an apocrine (scent) gland. The outer sheath continues all the way up to the gland. An erector pili muscle attaches below the gland to a fibrous layer around the outer sheath. When this muscle contracts, it causes the hair to stand up.

The primary component of the hair fiber is keratin. Keratins are proteins, long chains (polymers) of amino acids. The hair shaft contains three layers of keratin. The inner layer, which is called the medulla, may not be present. The next layer is the cortex, which makes up the majority of the hair shaft. The outer layer is the cuticle, which is formed by tightly packed scales in an overlapping structure similar to roof shingles. Most hair conditioning products attempt to affect the cuticle. Pigment cells are distributed throughout the cortex and medulla giving the hair its characteristic color.

### Eyelashes and Eyebrows

The term “eyebrow” refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris such as dandruff and other small objects from falling into the eyes, as well as providing a more sensitive sense for detecting objects being near the eye, like small insects. Eyebrows also have an important facilitative function in communication, strengthening facial expressions such as surprise, confusion, or anger.

The terms “eyelash” and “lash” are used interchangeably to refer to one of the hairs that grow at the edge of the eyelid. The eyelashes consist of curved sensory hairs originating from the eyelid margins. Compared to scalp skin, the skin of the eyelids contains a thinner epidermis and no hypodermis [Nguyen, B. et al. Am. J. Clin. Dermatol. (2023) 24: 55-67, citing Thibaut, S. et al. Br. J. Dermatol. (2010) 162 (2): 304-310]. Eyelashes are rooted approximately 2 mm deep into the dermis and lack the arrector pili muscles associated with most other hair follicles [Id., citing Thibaut, S. et al. Br. J. Dermatol. (2010) 162 (2): 304-310; Aumond, S. and Bitton, E. J. Optom. (2018) 11(4): 211-22]. Humans typically have about 90-160 eyelashes on the lower lids, spread across 5-6 rows, and 75-80 eyelashes on the lower lids, dispersed between 3-4 rows [Id., citing Aumond, S. and Bitton, E. J. Optom. (2018) 11(4): 211-222]. Eyelashes protect the eye from debris and provide a warning that an object (such as an insect or dust mite) is near the eye (which then is closed reflexively).

The inside of the nose contains small hairs called cilia. These cilia and nasal mucus clean the air drawn into the nose of the microscopic particles we inhale, including dust, pollen, and pollutants, for ultimate passage to the lungs.

### Hair Growth

There are three stages of hair growth: catagen, telogen, and anagen.

Anagen is the active growth phase of the hair during which the cells in the root of the hair are dividing rapidly. Anagen hairs are anchored deeply into the subcutaneous fat and cannot be pulled out easily. When a new hair is formed, it pushes the club hair up the follicle and eventually out. During this phase, the hair grows about 1 cm every 28 days. Scalp hair stays in this active phase of growth for 2-6 years. Human subjects that have difficulty growing their hair beyond a certain length have a short active phase of growth. Human subjects that have very long hair have a long active phase of growth. The hair on the arms, legs, eyelashes, and eyebrows have a very short active growth phase of about 30-45 days, which is why they are so much shorter than scalp hair.

The anagen phase is followed by a catagen phase. The catagen phase is a transitional stage that lasts for about 2-3 weeks. About 3% of all hairs are in this phase at any time. During this time growth stops and the outer root sheath shrinks and attaches to the root of the hair. This is the formation of what is known as a club hair.

After catagen, the hair goes into a telogen phase. Telogen is the resting phase, which accounts for 10-15% of all hairs. It lasts for about 100 days for hairs on the scalp and much longer for hairs on the eyebrow, eyelash, arm and leg. During this phase, the hair follicle is completely at rest and the club hair is completely formed. As compared with anagen hair, telogen hair is located higher in the skin and can be pulled out relatively easily. Pulling out a hair in this phase will reveal a solid, hard, dry, white material at the root. Normally, about 25-100 telogen hairs are shed each day.

In the normal scalp, approximately 80 to 90 percent of follicles are growing (anagen), about 5 to 10 percent are resting (telogen), and 1 to 3 percent are undergoing involution (catagen) Each day up to 75 hairs in telogen are shed from the scalp and about the same number of follicles enter anagen.

### Hair Loss

The term “alopecia” is a medical term for the absence or loss of hair including eyelashes, eyebrows, and scalp hair, as a result of illness, functional disorder, or hereditary disposition. For example, the term “Alopecia adnata” refers to underdevelopment of the eyelashes. Alopecia frequently occurs in patients undergoing treatment for cancer or suffering from other diseases, such as AIDS, where cytotoxic drugs are used.

Hair loss typically is categorized as scarring or nonscarring. Scarring alopecia, also known as “cicatricial alopecia”, refers to a collection of hair loss disorders that may be diagnosed in up to 3% of hair loss patients. It occurs worldwide in otherwise healthy men and women of all ages. While there are many forms of scarring alopecia, the common theme is a potentially permanent and irreversible destruction of hair follicles and their replacement with scar tissue. Examples include bullous diseases, chemical alopecia, discoid lupus erythematosus, folliculitis (severe), lichen planopilaris, dissecting cellulitis, and tumors.

The term “nonscarring alopecia” refers to hair loss without permanent destruction of the hair follicle. Examples include anagen effluvium, androgenetic alopecia, chemical alopecia, folliculitis (mild), inherited disorders of the hair shaft, telogen effluvium, alopecia areata, and traumatic alopecia.

The term “anagen effluvium” refers to the hair loss associated with chemotherapeutic agents that cause immediate destruction and release of anagen hair.

The term “androgenic alopecia” refers to a gradual decrease of scalp hair density in adults with transformation of terminal to vellus hairs, which become lost as a result of familial increased susceptibility of hair follicles to androgen secretion following puberty. The most common form of androgenic alopecia is male pattern baldness. The most common form of androgenic alopecia in women is female pattern alopecia, a diffuse partial hair loss in the centroparietal area of the scalp, with preservation of the frontal and temporal hairlines. When it occurs in females, it is associated with other evidence of excessive androgen activity, such as hirsuitism.

The term “telogen effluvium” refers to a condition resulting from an abrupt shift of large numbers of anagen hairs to telogen hairs on the scalp, with a corresponding change in the ratio of anagen hair to telogen hair from the normal ratio of 90:10 to 70:30. This form of alopecia generally begins approximately 3 months after a major illness or other stress (e.g., surgery, parturition, rapid weight loss, nutritional deficiency, high fever, or hemorrhage) or hormonal derangement; it also has been reported after the initiation of treatment with certain medications.

The term “alopecia areata” refers to a common condition of undetermined etiology characterized by circumscribed, nonscarring, usually asymmetrical areas of baldness on the scalp, eyebrows, and beaded portion of the face. Hairy skin anywhere on the body may be affected. It is thought to be an autoimmune disease occurring on areas of the body (most commonly the scalp) where the person's immune system attacks hair follicles, thereby suppressing and arresting hair growth.

Those suffering from hair loss often experience embarrassment and the fear being ridiculed by others because they look different. Some may take to wearing oversized eyeglasses in an attempt to hide the absence of eyelashes and/or eyebrows. Loss of nasal cilia may render some more susceptible to respiratory illnesses.

Therapies for hair loss are designed primarily for scalp applications. These include topical minoxidil (Rogain®), antiandrogen agents, including the androgen-receptor blockers sprionolactone, cyproterone acetate, and flutamide, and the 5α-reductase inhibitor finasteride (Propecia®, Merck & Co.®). Latanoprost, bimatoprost, and travoprost ophthalmic solution are prostaglandin F2 alpha analogues, marketed for the treatment of ocular hypertension that have the common side effect of eyelash hypertrichosis and trichomegaly. Bimatoprost (0.03%) is the only one approved for hypotrichosis of the eyelashes. However, topical prostaglandins may cause unwanted side effects, such as darkening of the iris color, periocular pigmentation; uveitis, deepening of the superior sulcus and fat atrophy; enhancement of the eyelid crease; and a decrease in proptosis. [Starace, M. et al. Dermatol. Ther. (2023) 13: 1243-1253].

### Heat Shock Proteins in Cutaneous Biology

Heat shock proteins are of fundamental importance in cutaneous biology, from protection against UV-induced damage to wound healing and repair. They play important regulatory roles in the control of apoptosis, regulation of steroid aporeceptors, kinases, and other protein remodeling events. They are also implicated in the control of cell growth, and as such, are potential targets for cancer diagnosis and treatment. Currently, emphasis is being placed on the potential use of these proteins in the prevention and treatment of disease.

Cells are repeatedly exposed to environmental or endogenous stresses that can alter normal cell behavior and increase cell vulnerability. In order to ensure tissue integrity and function, cells cope with cellular injuries by adapting their metabolism, protecting essential intracellular constituents, inhibiting cell death signaling pathways and activating those devoted to damage repair.

The molecular chaperones of the heat-shock protein (HSP) family are critical effectors of this adaptive response. They protect intracellular proteins from misfolding or aggregation, inhibit cell death signaling cascades and preserve the intracellular signaling pathways that are essential for cell survival. Most HSPs are rapidly overexpressed in response to cellular injuries including genotoxic stress. DNA damage can dramatically alter cell behavior and contribute to a number of diseases including developmental defects, neurodegenerative disorders, and cancer. Thus, the ability of cells to repair DNA damage is essential for preserving cell integrity. DNA damage activates a coordinated response that includes detecting DNA lesions before their transmission to daughter cells, blocking cell cycle progression and DNA replication and repairing the damage. Although the role of HSPs in proteins homeostasis and cell death, especially apoptosis, has been widely reported, much less is known about their function in DNA repair.

Hsp72 and hsp27 are among the best investigated stress proteins in skin biology. The hsp70 family consists of two major cytoplasmatic members, namely hsp72 (hsp70) that shows strong stress inducibility and hsp73 (hsc70) that is constitutively expressed in all investigated cells and tissues. Proteins of the hsp70 family have an 18-kD peptide-binding domain at the carboxy-terminal end and a 45-kD ATP-binding domain at the amino-terminal end [Jonak, C. et al. Intl J. Cosmetic Sci. (2006) 28: 233-242, citing Osipiuk, J. et al. Acta Crystallogr. D. Biol. Crystallogr. (1999) 55: 1105-1107; Wu, B. et al. Mol. Cell Biol. (1985) 5: 330-341.

Hsp27 is a member of the ‘small heat shock family’. It can be found in various cells and tissues without prior stress stimulation: breast, uterus, cervix, placenta, platelets, epidermis and adnexal structures [Id., citing Cocca, D R et al. J. Natl Cancer Instit. (1993) 85: 1558-70]. Hsp27 is expressed in different tumor tissues and cell lines. As an intracellular protein, it can be found perinuclearly and is translocated to the nucleus after stimulation. Hsp27 and hsp25 (the murine analogue of hsp27) provide their chaperone function as large oligomer complexes [Id., citing Jakob, U. et al. J. Biol. Chem. (1993) 268: 1517-1520; Rogalla, T. et al. J. Biol. Chem. (1999) 274: 18947-18956]. Phosphorylation of hsp27 leads to the formation of tetramers resulting in a decrease of their protective functions. Since tumor necrosis factor alpha and interleukin 1 can mediate hsp27 activities, it is speculated that this stress protein is involved in the pathogenesis of inflammatory skin diseases [Id., citing Kaur, P et al. FEBS Letters (1989) 227: 175-178; Arrigo, AP. Mol. Cell Biol. (1990) 10: 1276-1280; Guesdon, F. et al. J. Biol. Chem. (1993) 268: 4236-4243]. It has been proven experimentally that hsp27 expression in situ and in vitro correlates with human keratinocyte differentiation [Id., citing Kindas-Muggee, I. and Trautinger, F. Cell Growth Differ. (1994) 5: 777-781; Trautinger, F. et al. Brit. J. Dermatol. (1995) 133: 194-202].

### Epidermal HSPs

Like in all other investigated tissues, the expression of HSPs in human skin is stimulated under stress conditions. In contrast to other cells, keratinocytes show significant basal Hsp72 expression without prior stress [Id., citing Trautinger, F. et al. J. Invest. Dermatol. (1993) 101: 334-338]. Hsp72 is expressed throughout all layers of the epidermis including adnexal structures (hair follicle and sweat gland). Melanocytes, fibroblasts and other (epi)dermal cells are negative for Hsp72 in immunohistochemical staining. Heat shock ex vivo and in vitro results in ‘superinduction’ of Hsp72 in keratinocytes and de novo expression in dermal cells. The expression of Hsp27 correlates with keratinocyte differentiation and increases continuously from the basal layer to the stratum granulosum [Id., citing Kindas-Mugge, I. and Trautinger, F. Cell Growth Differ. (1994) 5: 777-81; Trautinger, F. et al. Brit. J. Dermatol. (1995) 133: 194-202]. Hsp27 is not detectable immunohistochemically in the stratum corneum. It has been suggested that this finding could be explained either by degradation of hsp27 in corneocytes or by its incorporation into the cornified cell envelope matrix making the antigen inaccessible for antibody staining.

In basal cell carcinomas and squamous cell carcinomas, Hsp27 is absent or sparsely expressed, despite in well-differentiated areas of the tumors [Id., citing Trautinger, F. et al. Brit. J. Dermatol. (1995) 133: 194-202]. Hsp27 overexpression in a transfected squamous carcinoma cell line (A431) results in a delay of tumor development and reduction of tumorigenicity after injection into nude mice [Id., citing Kindas-Mugge, L. et al. Cell Growth Differ. (1996) 7: 1167-1174]. Although Hsp27 has also been described to have a role in thermotolerance, this findings support the hypothesis that in the epidermis Hsp27 is involved mainly in the regulation of cell growth, differentiation and tumorigenicity [Id., citing Welsh, M J and Gaestel, M. Ann. N.Y. Acad. Sci. (1998) 851: 28-35].

### Heat Shock Proteins and UV-Induced Cell Death

Heat-induced inhibition of UVB-induced cell death has been described for in vitro and in situ settings in murine and human skin [Id., citing Mattin, EV., J. Biol. Chem. (1992) 267: 23189-23196; Maytin E V et al. (1993) Cancer Res. (1993) 53: 4952-4959; Maytin, E V et al. J. Invest. Dermatol. (1994) 103: 547-553; Kane, KS, and Maytin, EV. J. Invest. Dermatol. (1995) 104: 62-67; Trautinger, F. et al. J. Invest. Dermatol. (1995) 105: 160-162]. In cell culture using several different assay systems to assess cell viability and function, it has been demonstrated that hyperthermia can reduce damage from UVB. Mild heat conditions have been used and maximal effects were observed when the recovery period between heat and UVB was 6 h. The protective effect disappeared 12 h after heat treatment [Id., citing Trautinger, F. et al. J. Invst. Dermatol. (1995) 105: 160-162]. Investigators have addressed the question of whether hsps are responsible for this effect. Inhibition of protein and mRNA synthesis as well as specific inhibition of Hsp72 block the development of heat-induced UVB tolerance [Id., citing Trautinger, F. et al. J. Invest. Dermatol. (1995) 105: 1600-1662; Simon, M M et al. J. Clin. Invest. (1995) 95: 926-933]. Formation of sunburn cells (SBC), resembling UV-induced apoptotic epidermal keratinocytes, was used as an endpoint in a mouse study [Id., citing Kane, KS and Maytin, EV. J. Invst. Dermatol. (1995) 104: 62-67]. The number of SBC in heat-treated skin (41° C., 3 h) was significantly reduced compared with a sham control. These results were later also confirmed in human skin [Id., citing Trautinger, F. et al. J. Invest. Dermatol. (1996) 107: 442-443]. Hsp72 is among the gene products responsible for this effect. Indeed, constitutive expression of Hsp72 is an inherent protective mechanism in epidermal cells, and overexpression of Hsp72 is at least in part involved in heat-induced UVB resistance.

Since UV radiation is one of the most abundant and potentially harmful environmental factors for human skin Roh et al. [Ann. Dermatol. (Seoul) 20(4): 184-189] asked whether sun light exposure induces HSP. The expression of HSP was examined in human epidermal cell lines (normal human keratinocyte (NHK), epidermoid carcinoma A431 cells, normal human melanocytes, and SK30 malignant melanoma cells), in human dermal fibroblasts (HDF), which are the main cellular components of the dermis at baseline, and after heat treatment, UVA irradiation, and UVA+UVB irradiation. HSP70 was detected by immunoblotting using a monoclonal mouse anti-human HSP70 antibody (Santa Cruz Biotechnology, Santa Cruz, CA) detected by peroxidase-conjugated goat anti-mouse immunoglobulins (Tagoimmunologicals®, Camarillo CA). Each of the examined cell types constitutively expressed high amounts of HSP70 without stress conditions. In summary, epidermal cells (NHK, A431 cells, NHMs and SK30 cells) showed high baseline HSP70 expression and no additional upregulation after UV irradiation, whereas HDF showed very low baseline HSP70 expression and high up-regulation of expression after UV irradiation.

### Role of Transient Receptor Potential (TRP) Channels in Skin

TRP channels in skin are crucial for achieving temperature sensitivity to maintain internal temperature balance and thermal homeostasis, as well as to protect skin cells from environmental stresses such as infrared (IR) or near-infrared (NIR) radiation via Hsp production. [Hsu, W-L and Yoshioka, T. Biophysics (2015) 11: 25-32].

Many kinds of thermosensitive TRP channels are activated by heat, potentially inducing hsps to respond to a wide variety of physiological and environmental insults. Mammalian TRP channels consist of six-transmembrane cation-permeable channels that are classified into six subfamilies based on amino acid sequence homology: TRPC, TRPV, TRPM, TRPP, TRPA, and TRPML [Id., citing Venkatachalam, K. and Montell, C. Ann. Rev. Biochem. (2007) 76: 387-417]. The functional properties of these six subfamilies of TRP channels are listed and exhibited in Zheng, J. Compr. Physiol. (2013) 3(1): 221-242. Four heat-activated channels, TRPV1-4, and two cold-activated channels, TRPM8 and TRPA1, belong to temperature-sensitive transient receptor potential channels, and are expressed in dorsal root ganglion sensory neurons, skin, and other cells [Id., citing Clapham, DE. Nature (2003) 426: 517-524]. Temperature sensitivity is crucial to protect against skin damage as well as to maintain internal temperature balance or thermal homeostasis [Id., citing Toth, B I, et al. Br. J. Pharmacol. (2014) 171: 2568-2581]. Thermo TRP channels, TRPV1,3,4, TRPM8, and TRPA1, are known to be expressed in human keratinocytes (KCs); two of them, TRPV1 and TRPA1, are expressed in human skin fibroblasts (HDFs) [Id., citing Venkatachalam, K. and Montell, C. Ann. Rev. Biochem. (2007) 76: 387-417; Toth, B I et al. Br. J. Pharmacol. (2014) 171: 2568-2581], which are major regulators of skin cell proliferation and differentiation, in the skin barrier, and in immune functions responding to skin injury [Id., citing Toth, B I et al. Br. J. Pharmacol. (2015) 171: 2568-2581].

Although the TRPC family does not belong to thermosensitive TRP channels, it controls calcium entry under epidermal receptor stimulation, which is an important part of a functional system for maintaining skin homeostasis [I., citing Beck, B. et al. J. Invest. Dermatol. (2006) 126: 1982-1993].

### TRPV Subfamily

Among the six subfamilies of TRPV channels, TRPV1, TRPV3, and TRPV4 are known to be found in skin cells, especially in keratinocytes (KCs). TRPV1-type channels are activated by heat, low pH, and pro-inflammation [Id., citing Venkatachalam, K. and Montell C. Ann. Rev. Biochem. (2007) 76: 387-417]. It was also reported that TRPV1-type channels are sensitive to some physical stresses such as high (>42° C.) temperature, membrane stretching, and several chemicals (ethanol, lidocaine, mono-acylglycerols, and 2-Aminoethoxydiphenyl borate, 2APB) [Id., citing Vay, L. et al. Br. J. Pharmacol. (2012) 165: 787-801]. Ion selectivity, which is expressed as PCa/PNa, is approximately 10 for chemical stimulation and 4 for physical stimulation [Id., citing Venkatachalam, K. and Montell C. Ann. Rev. Biochem. (2007) 76: 387-417]. TRPV1 is also expressed in dermal fibroblasts [Id., citing Lan, C C et al. J. Dermatol. Sci. (2013) 72: 290-295]. The TRPV1 channel in skin was found to be essential for the control of skin growth as well as for barrier functions, cutaneous immunological functions, skin pathology and several cutaneous diseases [Id., citing Toth, B I et al. Br. J. Pharmacol. (2014) 171: 2568-2581], and skin aging [Id., citing Lan, C C. et al. J. Dermatol. Sci. (2013) 72: 290-295]. TRPV1 can be activated directly by OAG, a membrane-permeable diacylglycerol (DAG) analog, although the 1-oleoyl-2-acetyl-sn-glycerol (OAG)-induced calcium response is one-fifth of the capsaicin-induced signal [Id., citing Woo, D H et al. Mol. Pain (2008) 4: 42]. The activation of TRPV1 by membrane-permeable OAG does not mean it is activated by G protein-coupled receptor (GPCR), because DAG produced by the hydrolysis of phosphatidylinositol 4,5-bisphosphate (PIP2) by phospholipase c (PLC) R is phosphorylated to phosphatidic acid (PA) by DAG kinase immediately [Id., citing Sandal, M. et al. Curr. Protein Pept. Sci. (2013) 14: 650-657]. PIP2 appears to bind to the TRPV1 channel directly, causing channel inhibition that is relieved by PLC 0-catalyzed PIP2 hydrolysis. The C-terminal of TRPV1 transcription disrupts the PIP2 binding site and impairs thermal responsiveness. Moreover, TRPV3 is sensitive to warm temperature (33° C.-39° C.), and PCa/PNa is around 3. Heat-activated TRPV3 current displays strong outward rectification, striking thermal hysteresis, and sensitization following repeated activation [Id., citing Ramsey, I S et al. Ann. Rev. Physiol. (2006) 68: 619-647]. The TRPV3 channel's roles in the skin include nociception, skin integrity, wound healing, hair growth, and sebocyte functions. Similar to the TRPV3 channel, the TRPV4 channel covers a temperature range (27° C.-34° C.), and the ratio of PCa/PNa is close to 6. Unlike TRPV1-TRPV3, TRPV4 is apparently insensitive to activation by 2APB [Id., citing Chung, M K et al. J. Neurosci. (2004) 24: 5177-5182; Mergler, S. et al. J. Cell Physiol. (2011) 226: 1828-2842]. The TRPV4 channel is involved in mechano-sensation, osmo-sensation, nociception, modulation of cell migration, the shear stress sensor, and control adherence junction in the skin cochlea [Id., citing Nilius, B. and Owsianik, G. Genome Biol. (2011) 12: 218].

### TRPA1

The physiological functions of TRPA1 are summarized as follows: thermo-sensation, the most versatile chemo-sensor, mechanical sensation, nociception and, olfactory transduction [Id., citing Nilius, B. and Owsianik, G. Genome Biol. (2011) 12: 218].

TRPA1, well known as a noxious cold pain sensor, is also expressed highly in KCs [Id., citing Donnerer, J. et al. Pharmacology (2012) 89: 7-12] and in HDFs [Id., citing Nilius, B. and Owsianik, G. Genome Biol. (2011) 12: 218]. The role of this channel may be essentially different from those of other TRPV channels because it is directly activated by intracellular Ca2+ through binding to the N-terminal EF-hand domain of TRPA1 [Id., citing Zayats, V. et al. J. Mol. Model. (2013) 19: 4689-4700]. The mechanism underlying this action was suggested to be activation and/or sensitization of TRPA1 channels by GPCR coupled to PLC R signaling, such as bradykinin receptors [Id., citing Wilson, S R et al. Nat. Neurosci. (2011) 14: 595-602]. In addition, since TRPA1 is involved in inflammatory pain (increased sensitivity to painful stimuli), TRPA1 may underlie some components of inflammatory hyperalgesia (increased sensory neurons for cold hyperalgesia). Thus, TRPA1 is most likely a chemical sensor for injury and inflammation [Id., citing Obata, K. et al. J. Clin. Invest. (2005) 115: 2393-2401]. Many TRPA1 ligands, such as mustard oil, acrolein, formalin, and iodoacetamide, can activate TRPA1 extracellularly, while acetaldehyde, H₂O₂, 15d-prostaglandin J2, and prostaglandin A2 (PGA2), all of which are called reactive electrophilic species (RES), work intracellularly [Id., citing Bang, S. and Hwang, SW. J. Gen. Physiol. (2009) 133: 257-62]. These RESs are generated under oxidative stress with the chemical reactivity being transferred from ROS to RES [Id., citing Bang, S. and Hwang, SW. J. Gen. Physiol. (2009) 133: 257-262]. It is likely that these ROS promote disulfide formation between vicinal thiol residues, resulting in TRPA1 dysfunction [Id., citing Bang, S. and Hwang, SW. J. Gen. Physiol. (2009) 133: 257-262]. However, TRPA1 activation is recovered by the application of dithiothreitol (DTT), which reduces the disulfide bond to the —SH group of cysteine in TRPA1 [Bang, D. et al. Mol. Biosyst. (2009) 5: 750-756].

The sensitivity of TRPA1 to intracellular Ca²⁺ and ROS accelerates cell depolarization, which will activate voltage-dependent Ca²⁺ channels, leading to skin aging, carcinogenesis, and apoptosis [Id., citing Nagata, K. et al. J. Neurosi. (2005) 25: 4052-4061]. Thus, TRPA1 may help regulate the proliferation and differentiation of KCs and skin inflammation [Id., citing Andrade, E L et al. Pharmacol. Ther. (2012) 133: 189-204]. Some functional properties of TRPA1 depend on its ability to interact with TRPV1. The coexistence of TRPA1 with TRPV1 seems to be important for the action of an endogenous mediator, such as bradykinin or calcium [Id., citing Bessac, B F and Jordt, SE. Physiology (Bethesday) 2008 23: 360-370]. Akopian et al. also found that TRPV1 can regulate the mediator of TRPA1 by Ca²⁺ through the PIP2 signaling pathway [Id., citing Akopian, A N et al. J. Physiol. (2007) 583: 175-193].

### TRPM8

The TRPM channel controls certain skin functions, especially those related to melanocyte biology. TRPM8 displays a cold receptor of the body, and its activation can be modulated by many cooling compounds and many other odorant agents isolated from plants, e.g., linalool, geraniol, and hydroxycitronellal [Id., citing Behrendt, H J et al. Br. J. Pharmcol. (2004) 141: 737-745; Harteneck, C. Naunyn Schmiedebergs Arch. Pharmacol. (2005) 371: 307-314]; and the selectivity of Ca²⁺ is about three times higher than that of Na⁺ [Id., citing Venkatachalam, K. and Montell C. Ann. Rev. Biochem. (2007) 76: 387-417]. These results were confirmed electrophysiologically using whole-cell patch clamp experiments [Id., citing Voets, T. et al. Nature (2004) 430: 748-753]. It was also confirmed that the TRPM8 channel was activated by depolarization, which means that the TRPM8 channel is very sensitive to changes in the cell's ionic balance. Thus, it is natural to consider that the TRPM8 channel is involved in body temperature regulation [Id., citing Gavva, N R et al. Mol. Pain (2012) 8: 36].

### TRPC6 and TRPC7

The TRPC subfamily was established as the first recognized mammalian TRP channel [Id., citing Hardie, RC. J. Physiol. (2007) 578: 9-24]. According to the results obtained by Sakura et al., the TRPC subfamily is divided into three groups on the basis of sequence alignments: TRPC1/4/5, TRPC3/6/7, and TRPC2 [Id., citing Sakura, H. and Ashcroft, F M. Diaetologia (1997) 40: 528-532]. TRPC1/4/5/7 channels are expressed in HaCaT, an immortal human keratinocyte line [Id., citing Beck, B. et al. Cell Calcium (2008) 43: 492-505], while Cai et al. detected TRPC1/C5/C6/C7 in gingival KCs [Id., citing Cai, S. et al. J. Dermatol. Sci. (2005) 40: 21-28]. TRPC7 is also a receptor-operated, DAG-activated channel in KCs, mediating a PLC 04-activated transducer current in intrinsic photosensitivity [Id., citing Beck, B. et al. J. Invest. Dermatol. (2006) 126: 1982-1993; Hardie, RC. Handb. Exp. Pharmacol. (2014) 223: 795-826]. As was previously described, most of the TRPC subfamily shows similar characteristics for activation by DAG and inhibition by SKF96365. Thus, it is reasonable to discuss the characteristics of TRPC6 and TRPC7 as representative of the TRPC subfamily in KCs, since these channels are activated by membrane-permeable DAG [Id., citing Beck, B. et al. Cell Calcium (2008) 43: 492-505]. Muller et al. suggested that TRPC6 is a specific channel for inducing KCs differentiation [Id., citing Muller, M. et al. J. Biol. Chem. (2008) 283: 33942-33954]. Eventually, several members of the canonical TRPC subfamily were identified in the skin, where they mostly control the growth and differentiation of KCs under physiological and pathological conditions [Id., citing toth, B I et al. Br. J. Pharmacol. (2014) 171: 2568-2581. These TRPC channels in the skin are considered to be significantly involved in skin cell growth, barrier function, and cutaneous diseases. Based on the above discussion, as TRP channels open through oxidative stress, intracellular calcium elevation induces ROS and ATP production in mitochondria resulting in disulfide bonds formation to protein dysfunction.

### The Skin as an Immune Organ

The skin harbors a highly specialized immunological niche crucial for the maintenance of tissue homeostasis, defense, and repair. It protects the host from invasion by employing physical barriers, biomolecules, and an intricate network of resident immune and non-immune cells and skin structures. In the absence of a challenge, resident immune cells promote skin physiological functions.

### Physical Barriers

The barrier function of the epidermis is mainly mediated by corneocytes in the stratum corneum. These cells are organized in a “bricks and mortar” manner, interspersed by lipids such as ceramides, cholesterol, and free fatty acids [Nguyen, A V and Soulika, A M, Intl J. Molec. Sci. (2019) 20 (8): 1811, citing Mnon, G K et al. Int. J. pharm. (2012) 435: 3-9]. Each corneocyte contains a lipid envelope linked to keratin filament bundles that fill the intracellular compartments of the corneocyte, thus strengthening its rigidity [Id., citing Madison, KC. J. Investig. Dermatol. (2003) 121: 231-241]. The stratum corneum is composed of three layers and it is both an outside-in barrier to prevent the entry of foreign substances and microorganisms, and an inside-out barrier to prevent water loss [Id., citing Kubo, A. et al. Sci. Rep. (2013) 3: 1731].

The formation of the physical barrier of the skin function depends on junction adhesion molecules and tight junction proteins. Junctional adhesion molecules (JAMs), claudins, zonula occludens-1 (ZO-1), and occludins are found in epidermal layers.

### Biomolecules of the Skin

The main classes of biomolecules that participate in skin defense by disrupting bacterial membranes are antimicrobial peptides (AMPs) and lipids [Id., citing Niyonsaba, F. et al. Exp. Dermatol. (2017) 26: 989-998; Niyonsaba, F. et al. Crit. Rev. Immunol. (2006) 26: 545-576; Van Smeden, J. and Bouwstra, JA. Curr. Probl. Dermatol. (206) 49: 8-26]

AMPs are amphipathic peptides that are expressed constitutively or induced after cell activation in response to inflammatory or homeostatic stimulation. The most thoroughly studied AMP families in human skin are the defensins and the cathelicidins, which are produced by a variety of cells in the skin such as keratinocytes, fibroblasts, dendritic cells, monocytes, and macrophages, and sweat and sebaceous glands [Id., citing Niyonsaba, F. et al. Exp. Dermatol. (2017) 26: 989-998; Niyonsaba, F. et al. Crit. Rev. Immunol. (2006) 26: 545-576]. AMPs are produced as propeptides and become active after proteolytic cleavage. In contrast to defensin family members, which are produced by distinct genes, there is only one gene associated with cathelicidins (cathelicidin antimicrobial peptide—CAMP) [Id., citing Pfalzgraff, A. et al. Front. Pharmacol. (2018) 9: 281]. A number of active peptides can be generated via proteolytic cleavage of the inactive CAMP product, but the most studied cathelicidin is LL-37 [Id., citing Ann. Dermatol. (2012) 24: 126-135]. AMPs may act synergistically and have broad activity against microbial species [Id., citing Clausen, M L and Agner, T. Curr. Probl. Dermatol. (2016) 49: 38-46; Hanson, M A et al. eLife (2018) 493817].

AMPs have roles in modulating host immune responses. Human LL-37 was shown to induce differentiation of monocyte-derived dendritic cells, subsequent cytokine production, and expression of the co-stimulatory molecule CD86 [Id., citing Davidson, D J et al. J. Immunol. (2004) 172: 1146-1156]. LL-37 and β-defensins can also serve as alarmins for keratinocytes by inducing their proliferation and migration [Id., citing Tokumaru, S. et al. J. Immunol. (2005) 175: 4662-4668]. Furthermore, human LL-37 exerts its alarmin effects on immune cells in a synergistic manner with other inflammatory mediators, such as IL-1β [Id., citing Yu, J. et al. J. Immunol. (2007) 179: 7684-7691]. Human α- and β-defensins serve as chemoattractants for activated neutrophils, memory and naïve T cells and immature dendritic cells [Id., citing Zhang, L-J and Gallo, R L. Curr. Biol. (2016) 26: R14-R19], while β-defensins 3 and 4 can recruit monocytes and macrophages [Id., citing Oppenheim, J J and Yang, D J C. Curr. Opin. Immunol. (2005) 17: 359-365].

In addition, AMP functions have been associated with the processes of aging and memory [Id., citing Reinholz, M. et al. An. Dermatol. (2012) 24: 126-135; Zhang, L-J. and Gallo, R L. Curr. Biol. (2016) 26: R14-R19; Lezi, E. et al. Neuron (2018) 97: 125-138]. LL-37 has been shown to exert proangiogenic effects and may also play a role in tissue repair [Id., citing Koczulla, R. et al. J. Clin. Invest. (2003) 111: 1665-1672].

Lipids such as sphingomyelin, glucosylceramides, and phospholipids are intermediate molecules, readily converted into sphingosine and dihydrosphingosine, which exert antimicrobial activity against certain bacterial strains such as Staphylococcus aureus, Streptococcus pyogenes, Micrococcus leutus, and Proprionibacterium acnes [Id., citing Bibel, D J et al. J. Investig. Dermatol. (1992) 98: 269-273]. These lipids are stored in lamellar bodies found in corneocytes in the stratum corneum [Id., citing Van Smeden, J. Biochim. Biophys. Acta (2014) 1841: 295-313].

Sebocytes residing in the sebaceous glands produce sebum, which is rich in lipids such as triacylglycerol, wax esters, non-esterized fatty acids, and squalene. There is a consensus that sebum serves as a “seal” for the hair follicles, thus preventing entry of microbes into the deeper layers of the skin. The AMP dermcidin is expressed by sebocytes, suggesting that sebum exerts defensive functions [Id., citing Dahlhoff, M. et al. J. Dermatol. Sci. (2016) 81: 124-126]. Furthermore, sebum can be further processed into free fatty acids by skin commensal bacteria [Id., citing Kendall, A C et al. Prog. Lipid Res. (2013) 52: 141-164; Bibel, D J et al. J. Invst. Dermatol. (1989) 92: 632-638], and in humans, sebum-derived free fatty acids induce β-defensin 2 expression by sebocytes, further suggesting that sebaceous glands serve an innate defensive role [Id., citing Nakatsuji, T. et al. J. Investig. Dermatol. (2010) 130: 985-994].

The pH of human skin is 5.4-5.9, which makes the skin an inhospitable environment for potential pathogens [Id., citing Schmid-Wendtner, M H and Korting, HC. Skin Pharmacol. Physiol. (2006) 19: 296-302; Fluhr, J W et al. J. Investig. Dermatol. (2001) 117: 44-51]. Furthermore, the dramatic difference in pH levels between the skin (pH 5.4-5.9) and the blood (pH=7.4) serves as a secondary defensive mechanism in the event that microbes breach the skin tissue and enter the circulation.

There are various ways that the skin maintains a low pH.

Filaggrin, a filament-associated protein that binds keratin fibers, is broken down into histidine, which is further processed by histidase, expressed by corneocytes into the acidic metabolite trans-urocanic acid [Id., citing Scott, IR. Biochem. J. (1981) 194: 829-838]; this has been implicated in the acidification of the stratum corneum [Id., citing Krien, P M and Kermici, M. J. Investig. Dermatol. (2000) 115: 414-420]. Fatty acids produced in the stratum corneum also alter the acidity of the skin [Id., citing Bibel, D J et al. J. Investig. Dermatol. (1989) 92: 632-638; Fluhr, J W et al. J. Investig. Dermatol. (2001) 117: 44-51]. In addition, sweat glands produce acidic electrolytes and lactic acid, which lowers the pH of the skin [Id., citing Wilke, K. et al. Int. J. Cosmet. Sci. (2007) 29: 169-179] and promotes epidermal turnover [Id., citing Thueson, D O et al. Dermatol. Surg. (1998) 24: 641-645].

The physiological pH of the skin is hospitable for commensal bacteria such as Staphylococcus epidermidis, preventing pathogenic strains such as Staphylococcus aureus from establishing infections in the host [Id., citing Korting, H C et al. Acta Derm. Venereol. (1990) 70: 429-431; Elias, P M. Semin. Immunopathol. (2007) 29: 3-14].

### Immune Cells of the Skin

Skin-resident immune cells promote tissue function in homeostasis and act as sentinels by actively sampling environmental antigens. Both myeloid and lymphoid cell subsets are found in the skin in steady state. Some of these resident immune cells migrate to lymph nodes to either induce peripheral tolerance to tissue self-antigens or initiate robust immune responses. In the event of a challenge, such as infections or tissue injury, immune cells resident in the skin and those infiltrating from the periphery interact to create an intricate defense network to resolve the insult and restore the tissue to its original state.

### Myeloid Cells

Skin-resident myeloid cells include Langerhans cells, dermal dendritic cells, macrophages, mast cells, and eosinophils. Neutrophils are rarely found in healthy skin and thus are not “skin-resident cells.” However, neutrophils populate the skin in inflammatory conditions and after a wound.

Skin-resident myeloid cells contribute to skin homeostasis by secreting growth factors needed for the survival of keratinocytes, fibroblasts, and endothelial cells. In addition, they maintain optimal tissue function by phagocytosing debris and apoptotic cells, supporting vasculature integrity, and promoting tolerance.

In inflammatory conditions, myeloid cells respond immediately and produce pro-inflammatory mediators that drive the activation of cells in the local vicinity and infiltration of the affected site by peripheral immune cells. Skin myeloid cells also serve as a liaison between the innate and adaptive immune system.

### Langerhans Cells

Langerhans cells (LCs) are the sole myeloid cell type in the epidermis. Phenotypically, LCs are characterized by high expression of MHC class II and the presence of langerin+Birbeck granules [Id., citing Deckers, J. et al. Front. Immunol. (2018) 9: 93; Kaplan, DH. Nat. Immunol. (2017) 18: 1068-1075]. LC maintenance depends on keratinocyte-derived IL-34 [Id., citing Kaplan, DH. Nat. Immunol. (2017) 18: 1068-1075; Ginhoux, F. et al. Science (2010) 330: 841-5; Greter, M. et al. Immunity (2012) 37: 1050-1060; Wang, Y. et al. Nat. Immunol. (2012) 13: 753-760], one of the ligands for the colony stimulating factor-1 receptor (CSF-1R). CSF-1R is constitutively expressed on LCs and global deletion of this receptor results in a total lack of LCs [Id., citing Wang, Y. et al. Nat. Immunol. (2012) 13: 753-760; Wang, Y. and Colonna, M. Eur. J. Immunol. (2014) 44: 1575-1581; Ginhoux, F. et al. Nat. Immunol. (2006) 7: 265-273].

LCs are derived from two sources, the extra-embryonic yolk sac and fetal liver monocytes. LCs renew from a local progenitor cell [Id., citing Merad, M. et al. Nat. Immunol. (2002) 3: 1135-1141]. In homeostasis, LCs anchor themselves within the epidermis through interactions between epithelial cell adhesion molecules (EpCAM) or E-cadherin expressed on LCs and E-cadherin expressed by keratinocytes [Id., citing Deckers, J. et al. Front. Immunol. (2018) 9: 93; Choi, H W et al. Clin. Exp. Dermatol. (2018) 43: 291-295; Mayumi, N. et al. Eur. J. Immunol. (2013) 43: 270-280]. Furthermore, autocrine and paracrine TGFβ signaling restricts LCs in the epidermis by regulating the expression of E-cadherin on LCs [Id., citing Bobr, A. et al. Proc. Natl Acad. Sci. USA (2012) 109: 10492-10497; Kel, J M et al. J. Immunol. (2010) 185: 3248-3255] and increases phagocytic behavior in LCs during steady state [Id., citing Bauer, T. et al. J. Exp. Med. (2012) 209: 2033-2047]. While anchored, LCs sample antigens and, upon activation, they can extend their processes from the cell body outward to the stratum corneum or below toward the stratum basale [Id., citing Deckers, J. et al. Front. Immunol. (2018) 9: 93]. LCs participate in tight junction formation and thus can sample the microenvironment without damaging the barrier [Id., citing Decker, J. et al. Front. Immunol. (2018) 9: 93].

LCs are migratory cells and continually travel to the skin draining lymph nodes to promote tolerance in homeostasis [Id., citing Ghigo, C. et al. J. Exp. Med. (2013) 210: 1657-1664] or to initiate adaptive immune responses [Id., citing Romani, N. et al. J. Exp. Med. (1989) 169: 1169-1178; Romani, N. et al. J. Exp. Med. (1989) 93: 600-609; Schuler, G. and Steinman, R M. J. Exp. Med. (1985) 161: 526-546].

The migration of LCs (and also of dermal dendritic cells) through the dermis is mediated via CXCR4 signaling after binding to its cognate chemokine CXCL12, which is produced by dermal fibroblasts [Id., citing Kabashima, K. et al. Am. J. Pathol. (2007) 171: 1249-1257].

In the presence of inflammatory mediators and other activators such as pathogen-associated molecular patterns (PAMPs) and danger-associated molecular patterns (DAMPs), LCs upregulate co-stimulatory molecules and migrate from the epidermis en masse to the draining lymph nodes, where they prime adaptive immune responses in a manner equivalent to that of conventional dendritic cells [Id., citing Deckers, J. et al. Front. Immunol. (2018) 9: 93; Wang, B. et al. Immunology (1996) 88: 284-288; Griffiths, C E et al. Cytokine (2005) 32: 67-70; Kashem, S W et al. Immunity (2015) 42: 356-366; Nakajima, S. et al. J. Allergy Clin. Immunol. (2012) 129: 1048-1055]. TGFβ signaling is disrupted during inflammation, thus also promoting LC migration from the epidermis [Id., citing Bobr, A. et al. Proc. Natl Acad. Sci. USA (2012) 109: 10492-10497]. LCs are professional antigen-presenting cells and activate both CD8+ cytotoxic T lymphocytes and CD4+ helper T lymphocytes. In humans, efficient antigen presentation by LCs is dependent upon caveolin-1, a scaffold protein that serves a multitude of functions including transport of lipids, signal transduction, and membrane trafficking [Id., citing Polak, M E et al. J. Investig. Dermatol. (2014) 134: 695-703; Liu, P. et al. J. Biol. Chem. (2002) 277: 41295-41298]. LCs can also exert immunoregulatory and tolerogenic functions [Id., citing Deckers, J. et al. Front. Immunol. (2018) 9: 93; Flacher, V. et al. EMBO Mol. Med. (2014) 6: 1191-1204; Shklovskaya, E. et al. Proc. Natl Acad. Sci. USA (2011) 108: 18049-18054].

### Dermal Dendritic Cells

Dendritic cells that reside in the dermis are known as dermal dendritic cells (dDCs). In a similar manner to LCs, dDCs migrate to the lymph nodes, and are professional antigen-presenting cells efficient at priming adaptive immune responses [Id., citing Hain, T. et al. J. Investig. Dermatol. (2018) doi: 10.1016/j.jid/2018.08.022; Tomura, M. et al. Sci. Rep. (2014) 4: 6030]. dDCs can induce tolerance to topically applied antigens encountered in the hair follicles [Id., citing Tordesillas, L. et al. Nat. Commun. (2018) 9: 5238].

Unlike LCs, all dDCs are derived from progenitors originating from the bone marrow, with replenishment occurring roughly every seven days [Id., citing Kim, T G et al. Intl J. Mol. Sci. (2017) 19: 42; Ober-Blobaum, J L, et al. Methods Mol Biol. (2017) 1559: 37-52]. The two main subsets of dDCs are the conventional dendritic cell type 1 (cDC1) and the conventional dendritic cell type 2 (cDC2) [Id., citing Ober-Blobaum, J L, et al. Methods Mol Biol. (2017) 1559: 37-52; Kashem, S W et al. Annu. Rev. Immunol. (2017) 35: 466-499].

dDCs are implicated in maintaining homeostatic interactions between the host and skin-resident commensal bacteria. Plasmacytoid DCs (pDCs) are a DC subset, found in the skin exclusively under inflammatory conditions. pDCs are mass producers of IFNα, which is essential for viral defense [Id., citing Kashem, S W et al. Annu. Rev. Immunol. (2017) 35: 466-993].

Human dDCs are subdivided into cDC1 (CD141+), cDC2 (CDlc+), and CD14+ dDCs [123]. cDCls co-express CD304 (neuropilin-1), XCR1, and CD370 (CLEC9A). Unlike murine dDCs, human dDCs do not express langerin [Id., citing Kashem, S W et al. Annu. Rev. Immunol. (2017) 35: 466-499; Haniffa, M. et al. Immunity (2012) 37: 60-73]. Human cDC1s can cross-present in a similar manner to murine cDC1s [Jongbloed, S L et al. J. Exp. Med. (2010) 207: 1247-1260] and are potent in inducing Th1 responses [Id., citing Segura, E. et al. J. Exp. Med. (2012) 209: 653-660]. cDC2s and CD14+ dDCs co-express CD11b and CX3CR1. Both human cDC1s and cDC2s are capable of inducing Th2 responses [Id., citing Segura, E. et al. J. Exp. Med. (2012) 209: 653-660]. Human pDCs express CD304, CD303 and CD123 and like their murine counterparts, they are only found in inflamed skin. Human peripheral blood pDCs upregulate the expression of B cell maturation antigens upon activation via TLR9 signaling, suggesting that pDCs play a role in the maintenance of plasma cells, in addition to their inflammatory functions [Id., citing Schuh, E. et al. J. Immunol. (2017) 198: 3081-3088].

### Macrophages

Macrophages are found in the dermal layer of the skin and require IL-34 for survival [Id., citing Wang, Y. et al. Eur. J. Immunol. (2014) 44: 1575-1581; Ginhoux, F. and Jung, S. Nat. Rev. Immunol. (2014) 14: 392-404]. Two sources of dermal macrophages have been identified so far. The first source is embryo-derived progenitors that seed the skin prenatally and are self-renewing in a similar fashion to LCs [Id., citing Guilliams, M. et al. Nat. Rev. Immunol. (2014) 14: 571-578]. The second and major source of dermal macrophages is circulating monocytes (monocyte-derived macrophages) that mature once they reach the skin. This population replenishes roughly every 10 days [Id., citing Sheng, J. et al. Immunity (2015) 43: 382-393; Baranska, A. et al. J. Exp. Med. (2018) 215: 1115-33]. Monocytes that give rise to dermal macrophages express lymphocyte antigen 6C (Ly6C), and home to the skin in a CCR2-dependent manner [Id., citing Malissen, B. et al. Nat. Rev. Immunol. (2014) 14: 417-428]. As monocytes mature into skin-resident macrophages, the expression of CCR2 is downregulated [Id., citing Tamoutounour, S. et al. Immunity (2013) 39: 925-938]. CD64 expression is prominent on dermal macrophages and is used as a marker to differentiate them from the dDCs [Id., citing Baranska, A. et al. J. Exp. Med. (2018) 215: 1115-1133; Tamoutounour, S. et al. Immunity (2013): 39: 925-938]. CD36, DC-SIGN, and IL-10 are highly expressed by macrophages isolated from healthy skin, suggesting that they adapt an immunoregulatory phenotype [Id., citing Tamoutounour, S. et al. Immunity (2013): 39: 925-938; Lonati, A. et al. J. Investig. Dermatol. (1996) 106: 96-101]. In steady state, macrophages remove cellular debris [Id., citing Malissen, B. et al. Nat. Rev. Immunol. (2014) 14: 417-428, Tamoutounour, S. et al. Immunity (2013): 39: 925-938], and have also been implicated in homeostatic hair regeneration [Id., citing Yanez, D A et al. Pflugers Arch. (2017) 469: 455-463; Eichmullelr, S. et al. J. Histochem. Cytochem. (1998) 46: 361-370].

Macrophages are plastic, and one way to categorize their effector functions is as pro-inflammatory “M1” or anti-inflammatory/pro-repair “M2.” M1 macrophages express inducible nitric oxide synthase (iNOS), and secrete inflammatory cytokines such as TNFα, IL-1β, and IL-6 [Id., citing Mills, CD. Front. Immunol. (2015) 6: 212; Mills, C D and Ley, K. J. Innate Immun. (2014) 6: 716-726]. M2, or “alternatively activated” macrophages, adopt an anti-inflammatory and/or pro-repair phenotype. M2 macrophages can be further subdivided into M2a, M2b, M2c, and M2d. M2a macrophages are known to be pro-fibrotic due to TGFβ production [Id., citing Ohji, M. et al. Curr. Eye Res. (1993) 12: 703-709]. M2b macrophages express the co-stimulatory molecule CD86 and as such they are the most pro-inflammatory of the M2 subsets. M2c macrophages are induced by IL-10 or TGFβ, express the Mer tyrosine kinase (MerTK) and promote neovascularization [Id., citing Martinez, F O et al. Front. Biosci. (2008) 13: 453-461] and have high scavenging and debris clearing activity. M2d macrophages are responsive to IL-6 and exhibit a few of the properties of tumor-associated macrophages such as secretion of IL-12 and IL-10 [Id., citing Martinez, F O et al. Front. Biosci. (2008) 13: 453-461, Ferrante, C J and Leibovich, SJ. Ad. Wound Care (New Rochelle) (2012) 1: 10-16; Arora, S. et al. Immunobiology (2018) 223: 383-396; Wang, Q. et al. Cell Res. (2010) 20: 701-712]. M2d cells are also known to express high levels of the adenosine receptor A2AR in the presence of LPS [Id., citing Ferrante, C J et al. Inflammation (2013) 36: 921-931]. A2AR signaling has been shown to attenuate pro-inflammatory cytokine production [Id., citing Koroskenyi, K. et al. Biochim. Biophys. Acta (2016) 1863: 1461-1471], suggesting that A2AR-expressing M2d cells may be involved in the resolution of the inflammatory response.

### Mast Cells

Mast cells are commonly found in the dermal layer. In humans, mast cells are found in all areas of the skin but are most numerous in the arms and the legs [Id., citing Janssens, A S et al. J. Clin. Pathol. (2005) 58: 285-289]. The density of mast cells in the papillary dermis increases with age and they are most often localized in the proximity of PGP9.5+ nerve fibers expressing vasoactive intestinal peptide (VIP), which was shown to suppress mast cell degranulation [Id., citing Pilkington, S M et al. Br. J. Dermatol. (2018) doi: 10.111.bjd.17268]. This has been associated with the reduction of the amount of extracellular matrix remodeling in the skin observed during the later stages of life.

Mast cells contain granules containing preformed mediators such as histamine, sulfated proteoglycans, serotonin, and tryptase and/or chymase. In both humans and mice, mast cells resident in the skin express both tryptase and chymase, whereas other tissue-resident mast cells express only tryptase [Id., citing Olivera, A. et al. J. Allergy Cli. Immunol. (2018) 142: 381-383].

Mast cells are classically known for their involvement in allergic reactions as they produce and release copious amounts of histamine when their FcF receptors are crosslinked via IgE-antigen complexes [Id., citing Shi, L B et al. Pestic. Biochem. Physiol. (2018) 148: 159-165; Hirano, T. et al. Sci. Rep. (2018) 8: 14237]. They also make large amounts of prostaglandin D2 (PGD2), a lipid-derived inflammatory mediator.

Mast cells are mass producers of leukotrienes (LTs), which are short-lived lipid inflammatory mediators synthesized via the 5-lipoxygenase (5-LO) pathway.

A variety of cytokines and growth factors are produced by mast cells either constitutively or in response to a stimulus [Id., citing Mukai, K. et al. Immunol. Rev. (2018) 282: 121-150]. Many of these cytokines and growth factors such as TNFα and vascular endothelial growth factor (VEGF) may be pre-formed and packaged in mature mast cell granules [Id., citing Wernersson, S. and Pejler, G. Nat. Rev. Immunol. (2014) 14: 478-494; Gordon, JR, Galli, SJJN. Nature (1990) 346: 274; Grutzkau, A. et al. Mol. Biol. Cell (1998) 9: 875-884]. Proper formation of mast cell granules is mediated mostly by proteoglycan serglycin [Id., citing Braga, T. et al. Biochem. J. (2007) 403: 49-57].

Dermal mast cells can prime adaptive responses during cutaneous infections. Mast cell-derived IL-1β induces production of histamine and IL-8 in human mast cells, suggesting that IL-1β is part of a positive feedback loop for mast cell activation [Id., citing Subramanian, N. and Bray, MA. J. Immunol. (1987) 138: 271-275; Kim, G Y et al. J. Immunol. (2010) 184: 3946-3954]. TNFα produced by mast cells is known to drive migration of dermal dendritic cells to draining lymph nodes in a murine model of hapten-induced contact hypersensitivity [Id., citing Suto, H. et al. J. Immunol. (2006) 176: 4102-4112]. Mast cell-derived TNFα is also crucial for maintenance of tolerance toward allogeneic skin grafts in mice [Id., citing De Vries, V C et al. Immunity (2011) 35: 550-561]. Studies revealed that mast cells are able to form immunological synapses with 76 T lymphocytes when challenged with dengue virus [Id., citing Mantri, CK and St. John, A L. J. Clin. Investig. (2018) doi: 10.1172/JCI122520197] and can acquire MHC class II expression via vesicle transfer from dendritic cells after administration with dinitrofluorobenzene [Id., citing Dudeck, J. et al. J. Exp. Med. (2017) 214: 3791-3811].

### Eosinophils

Eosinophils are skin-resident cells [Id., citing Yu, Y R et al. PLoS ONE (2016) 11: e0150606; Ramirez, G A et al. Biomed. Res. Int. (2018) 99095275], but not much is known about their role in tissue homeostasis. Eosinophilic granules are loaded with potent and toxic proteins: major basic protein (MBP), eosinophil peroxidase (EPO), eosinophil protein X/eosinophil-derived neurotoxin (EPX/EDN), and eosinophil cationic protein (ECP) [Id., citing Long, H. et al. Clin. Rev. Allergy Immunol. (2016) 50: 189-213].

Like mast cells, eosinophils produce inflammatory lipid mediator leukotrienes (LTs) and prostaglandin D2 (PGD2) [Id., citing Luna-Gomes, T. et al. J. Immunol. (2011) 187: 6518-6526], with the latter being crucial for eosinophilic infiltration of the skin in hypersensitivity reactions such as atopic dermatitis [Id., citing He, R. et al. J. Allergy Clin. Immunol. (2010) 126: 784-790]. In addition, eosinophils generate extracellular DNA traps (ETs) containing eosinophil granules [Id., citing Ueki, S. et al. Blood (2013) 121: 2074-2083; Yousefi, S. et al. Nat. Med. (2008) 14: 949-953]. These traps are believed to play a role in antibacterial defense.

Eosinophils are classically known to promote host defense against parasitic infections [Id., citing Jacobesen, E A et al. Blood (2012) 120: 3882-3890]. The roles of eosinophils in dermatoses, or skin diseases associated with eosinophilia such as allergic contact dermatitis and urticaria, is emerging [Id., citing Long, H. et al. Clin. Rev. Allergy Immunol. (2016) 50: 189-213; Foster, E L et al. PLoS ONE (2011) 6: e22029; Oyoshi, M K et al. Proc. Natl Acad. Sci. USA (2012) 109: 4992-7; Wang, S F et al. Med. Inflamm. (2016) 2016: 5032051]. In eosinophilic dermatoses, extensive eosinophilic degranulation in the skin results in local tissue damage.

### Lymphoid Immune Cells

The skin harbors different types of lymphoid cells, all of which are important in both steady state and inflammatory responses. Both human and murine skin contain γδ T lymphocytes and αβ T lymphocytes, along with natural killer T cells. γδ T cells are the dominant T cell population in murine skin, while αβ T cells are the dominant T cell population in human skin [Id., citing Mestas, J. and Hughes, CC. J. Immunol. (2004) 172: 2731-8; Elbe, A. et al. Semin. Immunol. (1996) 8: 341-349].

### αβ T Lymphocytes

In both mice and humans, αβ T lymphocytes are found in the epidermis and dermis [Id., citing 224], and traffic to the skin from the periphery via cutaneous lymphocyte antigen (CLA) interactions with E-selectin (expressed on endothelial cells), which is upregulated under inflammatory conditions [Id., citing Kantele, A. et al. J. Immunol. (1999) 162: 5173-5177; Groves, R W et al. Br. J. Dermatol. (1991) 124: 117-123]. αβ T lymphocytes in the skin are resident memory T cells (T_{RM}), which are long-lived and distinct from their circulating counterparts [Id., citing Seidel, J A et al. Clin. Exp. Immunol. (2018) 194: 79-92].

Most T_{RM} in the skin are derived from antigen-specific effector T cells, which previously infiltrated the tissue as a result of an infection. After resolution, these T_{RM} cells seed all areas of the skin but are denser in areas of antecedent infection [Id., citing Clark, RA. Sci. Trans. Med. (2015) 7: 269rv261]. Following a skin infection, T_{RM} cells are also found in distal organs such as the lung and gastrointestinal tract [Id., citing Clark, RA. Sci. Transl. Med. (2015) 7:269rv261].

T_{RM} express lower levels of CD28 than effector memory T cells, but can mount robust local recall responses [Id., citing Seidel, J A et al. Clin. Exp. Immunol. (2018) 194: 79-92] without emigrating from the tissue to do so [Id., citing Gebhardt, T. et al. Nat. Immunol. (2009) 10: 524-530]. T_{RM} also exert sentinel-like functions by promoting recruitment of other memory T cells from the periphery to sites of infection [Id., citing Ariotti, S. et al. Science (2014) 346: 101-105; Schenkel, J M et al. Nat. Immunol. (2013) 14: 508-513].

The most studied skin T_{RM} are CD8+ T cells. CD8+T_{RM} cells are usually found in the epidermis and may dislocate dendritic epidermal T cells [Id., citing Mackay, L K et al. Nat. Immunol. (2013) 14: 1294-1301]. All CD8+T_{RM} express CD69, an early marker of lymphocyte activation, and a high proportion also express CD103 [Id., citing Mackay, L K et al. Nat. Immunol. (2013) 14: 1294-1301]. CD103 is required for the development of CD8+T_{RM} cells in the skin [Id., citing Mackay, L K et al. Nat. Immunol. (2013) 14: 1294-1301] and mediates adhesion interactions with keratinocytes via an E-cadherin-independent manner [Id., citing Jenkinson, S E et al. Immunology (2011) 132: 188-96].

CD4+T_{RM} cells also make up a significant portion of the skin-resident lymphocyte population and are found in both the epidermis and dermis [Id., citing Mackay, L K et al. Nat. Immunol. (2013) 14: 1294-1301]. Although the function of CD4+ T cells in the skin is not studied to the same extent as CD8+T_{RM}, they are often the dominant αβ T cell subset. During steady state, clusters of antigen-presenting cells with CD4+ memory T cells are found around the hair follicles in both murine and human skin, and cells in these clusters circulate between the skin and periphery [Id., citing Collins, N. et al. Nat. Commun. (2016) 7: 11514]. These clusters are formed because keratinocytes in the hair follicles produce IL-7 and IL-15, which are required for homeostatic maintenance of the T cell populations [Id., citing Adachi, T. et al. Nat. Med. (2015) 21: 1272-1279].

### γδ T Lymphocytes:

Unlike the αβ T lymphocytes, γδ T cells do not undergo the same stringent negative selection process during development and are released from the thymus in waves, with the first wave of γδ T cells seeding the dermis of the skin [Id., citing Sutoh, Y. et al. Front. Immunol. (2018) 9: 1059; Xiong, N. et al. Immunity (2004) 21: 121-131; Xiong, N. and Raulet, DH. Immunol. Rev. (2007) 215: 15-31].

The functions of dermal γδ T cells in inflammatory skin conditions are well documented. This population of dermal T cells is inclined to produce IL-17 [Id., citing Gray, EE. Et al. J. Immunol. (2011) 186: 6091-6095], indicating their significance in cutaneous diseases such as psoriasis [Id., citing Van der Fits, L. et al J. Immunol. (2009) 182: 5836-5845; Mabuchi, T. et al. J. Investig. Dermatol. 2(2009) 182: 5836-5845].

γδ T cells in the skin can also play a protective role in cutaneous defense; a previous study showed that γδ T cell-deficient mice exhibited larger lesions and reduced IL-17 production in response to Staphylococcus aureus compared to wild type (WT) control mice [Id., citing Cho, J S et al. J. Clin. Investig. (2010) 120: 1762-73].

γδ T cells are not MHC-restricted [Id., citing Champagne, E. Arch. Immunol. Ther. Exp. (2011) 59: 117-37] and can recognize soluble antigens [Id., citing Champagne, E. Arch. Immunol. Ther. Exp. (2011) 59: 117-137; Born, WK and O'Brien, R L. Arch. Immuol. Ther. Exp. (2009) 57: 129-135], antigens derived from damaged or stressed cells [Id., citing O'Brien, R L and Born, W. Semin. Immunol. (1991) 3: 81-87; O'Brien, R I et al. Proc. Nat. Acad. Sci. USA (1992) 89: 4348-4352; Heng, MK. Madame Curie Bioscience Database, Landes Bioscience: Austin TX, SA (2013); pp. 2000-2013], or antigens complexed with non-classical MHC molecules such as CD1b, CD1c, and CDld [Id., citing Cui, Y. et al. Biol. Direct. (2009) 4: 47; Luoma, A M et al. Trends Immunol (2014) 35: 613-621] or MHCI-related chain A/B (MICA/MICB) [Id., citing Champsaur, M. and Lanier, LL. Immunol. Rev. (2010) 235: 267-285]. One study suggested that γδ T cells also recognize butyrophilin-like Btnl/BTNL proteins, which are part of the B7 superfamily that regulates immune responses via costimulatory or coinhibitory signals [Id., citing Melandri, D. et al. Nat. Immunol. (2018) 19: 1352-1365].

### B Lymphocytes

B cells are rather sparse in the skin in steady state and it is unclear whether they are indeed resident to the skin [Id., citing Nihal, M. et al. J. Mol. Diagn. (2000) 2: 5-20; Geherin, S A et al. J. Immunol. (2012) 188: 6027-6035; Egbuniwe, I U, et al. Trends Immunol. (2015) 36: 102-111]. However, the roles of B lymphocytes in skin inflammatory conditions are well documented. In humans, B cells are found in elevated levels in cutaneous diseases such as atopic eczema, cutaneous leishmaniasis, and cutaneous sclerosis [Id., citing Simon, D. et al. J. Allergy Clin. Immunol. (2008) 121: 122-128; Geiger, B. et al. Br. J. Dermatol. (2010) 162: 870-874; Lafyatis, R. et al. Arthritis Rheum. (2009) 60: 578-583]. B cells are found in the reticular dermis over the course of these diseases and are associated with increased levels of IgM, IgE, and IgG. In a similar manner to T lymphocytes, B lymphocytes traffic to the skin tissue via cutaneous lymphoid antigen (CLA) interactions [Id., citing Postigo, A A et al. J. Clin. Investig. (1994) 94: 1585-1596]. B cells also play a role in delayed-type hypersensitivity reactions in the skin.

### Non-Immune Cells

Pattern recognition receptors (PRR) are expressed by most cells of the skin and have been characterized on keratinocytes, fibroblasts, adipocytes, melanocytes, and endothelial cells [Id., citing Chen, L. and DiPietro, LA. Adv. Wound Care (2017) 6: 344-355]. Activation of these receptors results in the production of cytokines and chemokines by non-immune skin cells, thus participating in the local immune response [Id., citing Ahn, J H et al. Exp. Dermatol. (2008) 17: 412-417; Faure, E. et al. J. Biol. Chem. (2000) 275: 11058-11063; Taylor, K R et al. J. Biol. Chem. (2004) 279: 17079-17084; Song, M J et al. Biochem. Biophys. Res. Commun. (2006) 346: 739-745].

Keratinocyte-derived inflammatory responses have been extensively studied. These cells express almost all intracellular and extracellular PRRs and produce a variety of cytokines, chemokine and AMPs to protect the host against infection [Id., citing Bitschar, K. et al. J. Dermatol. Sci. (2017) 87: 215-220; Pasparakis, M. et al. Nat. Rev. Immunol. (2014) 14: 289; Schittek, B. Curr. Probl. Dermatol. (2011) 41: 54-67]. Keratinocytes both in the epidermis and skin elements are in constant interaction with local immune cells and produce factors crucial in homeostasis and in tissue repair [Id., citing Wang, Y. et al. Nat. Immunol. (2012) 13: 753-760; Adachi, T. et al. Nat. Med. (2015) 21: 1272-1279; Chodaczek, G. et al. Nat. Immunol. (2012) 13: 272-282; Takashima, A. et al. J. Investig. Dermatol. (1995) 105: 505-535]. Other studies demonstrated that keratinocytes produce IL-33 in response to hypo-osmotic stress [Id., citing Pietka, W. et al. J. Invest. Dermatol. (2018 doi 10.1016/j.jId.2018.07.023], and that human and murine keratinocytes produce IL-6 and IL-1β mediated by NFκB signaling in response to UVB irradiation [Id., citing Tang, S C et al. J. Dermatol. Sci. (2017) 86: 238-248].

Fibroblasts immunomodulatory functions have also been well delineated. They express PRRs, synthesize many cytokines, and were shown to produce AMPs [Id., citing Bautista-Hernandez, L A et al. Eur. J. Microbiol. Immunol. (2017) 7: 151-157]. Dermal fibroblasts and keratinocytes produce serum amyloid A in response to PRR signaling [Id., citing Morizane, S. et al. Clin. Exp. Dermatol. (2018) doi: 10.111/ced.13604], which is believed to induce the production of pro-inflammatory cytokines from various immune cells [Id., citing Eklund, K K et al. Crit. Rev. Immunol. (2012) 32: 335-348]. A recent investigation showed that human fibroblasts cultured in vitro produced massive amounts of TNFα, IL-1β, IL-6, IL-8, and IL-25 when subjected to thermal stress [Id., citing Jiang, L. et al. Biomed. Pharmacother. (2018) 107: 24-33].

### Influence of Circadian Oscillations in Skin on Immune Response, Skin Homeostasis, Stress Mediation, and Aging

The circadian clock, an intrinsic autonomous clock that controls the body's divergent functions during day and night, affects the expression of multiple genes that mediate skin stem cell metabolism and proliferation, DNA repair, stimulus response and immunity. [Duan, J. et al. FEBS Lett (2021) 595 (19): 2413-2436] Circadian rhythm is closely linked to immunological processes and skin homeostasis, and its dysynchrony can be linked to the perturbation of the skin. [Salazar, A. and von Hagen, J. Intl Jm Molec. Sci. (2023) 24: 5635].

The mammalian circadian clock at a cellular level consists of at least 3 overlapping feedback loops (FIG. 3) [Id., citing Sherratt, M J et al. Matrix Bio. (2019) 84: 97-110; Lyons, A B et al. J. Clin. Aesthet. Dermatol. (2019) 12: 42-45; Curtis, A M et al. Immunity (2014) 40: 178-186].

In the first loop, the core circadian clock proteins BMAL1 (basic helix-loop-helix ARNT like 1; also called ARTNL, aryl hydrocarbon receptor nuclear translocator-like) dimerizes either with CLOCK (circadian locomotor output cycles kaput) or with NPAS2 (neuronal PAS domain protein 2) and then triggers the expression of PER (period circadian regulator; PER1-3), CRY (cryptochrome circadian regulator; CRY1-2), ROR (or RAR, related orphan receptor), NR1D1 (nuclear receptor subfamily 1 group D member 1; also called REV-ERE), DBP (D-box binding PAR Bzip transcription factor), and other clock controlled genes by binding to their E-box elements (5′-CACGTG-3′) in the promoter region. On reaching a critical concentration, the proteins PER and CRY dimerize to inhibit their own expression by preventing the binding of BMAL1: CLOCK to DNA, which results in an oscillation of these proteins [Id., citing Sherratt, M J et al. Matrix Biol. (2019) 84: 97-110; Lyons, A B et al. J. Clin. Aesthet. Dermatol. (2019) 12: 42-45; Curtis, A M et al. Immunity (2014) 40: 178-186; Richards, J. and Gumz, ML. Am. J. Physiol. Regul. Integr. Comp. Physiol. (2013) 304: R1053-R1064].

In the second loop, the protein ROR binds to the RORE element 5′-(A/G)GGTCA-3′ in the promoter of BMAL1, CLOCK, and NFIL3 (nuclear factor, interleukin 3 regulated), resulting in their transcription. The binding of ROR to the RORE element is inhibited by NR1D1, and potentially also by related proteins from this family. This completes the second loop.

The protein DBP, whose expression is under the control of BMAL1:CLOCK from the first loop binds to the D box (5′-TTATG(T/C)AA-3′) in the promoter region of PER. This binding is negatively regulated by NFIL3 from the second loop. Taken together, this is considered the third loop.

Similar to PER1/2, CRY1 is regulated by a combinatorial mechanism involving both E-box and RORE, giving rise to a phase distinct from DBP and REV-ERB. The DEC loop is ancillary to the core circadian loops, and is characterized by the expression of DEC and other circadian controlled genes, which are under the control of BMAL1: CLOCK. DEC, in turn, inhibits the binding of BMAL1: CLOCK to the E-box element, thereby regulating its own expression [Id., citing Ono, D. et al. Sci. Rep. (2021) 11: 19240; Honma, S. et al. Nature (2002) 419: 841-844].

Posttranslational modifications (PTMs) and proteasomal degradation of these core components of the molecular clock machinery are essential to maintain the oscillatory nature of these proteins and the proteins they regulate. These PTMs include phosphorylation, glycosylation, ubiquitination, acetylation, and SUMOylation, as reviewed by Hirano et al. [Id., citing Hirano, A. et al. Nat. Struct. Mol. Biol. (2016) 23: 1053-1060]. In some cases, a further level of complexity is introduced in the form of crosstalk between these mechanisms; for instance, O-linked β-N-acetylglucosamine (O-GlcNAc) competing for the same serine and threonine residues as kinases for phosphorylation [Id., citing Hirano, A. et al. Nat. Struct. Mol. Biol. (2016) 23: 1053-1060; Hardin, P E and Panda, S. Curr. Opin. Neurobiol. (2013) 23: 724-731]

### Zeitgebers and the Circadian Clock in Mammalian Skin

The intracellular molecular clock oscillates in response to environmental signals known as ‘Zeitgebers’, derived from German and directly translating to ‘time giver’. As a result, in the study of circadian rhythm, time in days is often divided into ‘zeitgeber time’. Light is the primary zeitgeber. It is detected via the optical nerve, which then transmits signals of perceived light to the hypothalamic suprachiasmatic nucleus (SCN) [Id., citing Curtis, A M et al. Immunity (2014) 40: 178-186]. This information is used to entrain the functional molecular clocks in peripheral tissues via the autonomic nervous system and the hypothalamus pituitary adrenal axis via hormones including glucocorticoids and catecholamines (epinephrine and norepinephrine) [Id., citing Kalsbeek, A. et al. Mol. Cell Endocrinol. (2012) 349: 20-29]. The hormones prolactin, growth hormone, and melatonin have been implicated in circadian signaling. Other zeitgebers that have been exploited in in vivo studies include sleep wake cycles, feeding and fasting regimes, and temperature. These zeitgebers are linked to the presence of light, as well as circulating levels of melatonin [Id., citing Sato, K. et al. J. Pineal. Res. (2020) 688: e12639; Slominski, A T et al. J. Investig. Dermatol. (2018) 138: 490-499; Kleszcynski, K. et al. Dermato-endocrinology (2011) 3: 27-31].

It has been shown that light can entrain the circadian clock in these peripheral tissues even after SCN ablation or scarring, although oscillations were found to be lower in these cases [Id., citing Buhr, E D et al. Curr. Biol. (2019) 29: 3478-3487; Welz, P S et al. Cell (2019) 177: 1436-1447; Koronowski, K B et al. Cell (2019) 177: 1448-1462]. The peripheral clocks also can communicate with each other, achieving entrainment independent of the SCN. The potential of UV, visible, and infra-red light to cause DNA damage, oxidative stress, lipid peroxidation, etc., in skin cells has been well characterized. Furthermore, UV light is also able to stimulate the production of melanin and melanocyte stimulating hormone (MSH) in the skin. The knowledge that skin cells have been known to interact with light makes it even more plausible that they can be directly entrained by light exposure.

### Influence of the Circadian Clock on the Immune Response of the Skin

The components of the circadian clock machinery are crucial to the development and functioning of a robust immune system. Indeed, most immune cell lineages have intrinsic clocks that govern their maturation, migration, differentiation, and function. An example is NFIL3, which is responsible for the development and maintenance of a population of interferon-gamma (IFN-7) producing group 1 innate lymphoid cells and NK cells [Id., citing Greenberg, E N et al. Proc. Natl Acad. Sci. USA (2020) 117: 5761-5771]. This, in turn, can be linked to the rhythmic activation of IFN-sensitive gene pathways in the skin, including a key transcription factor IFN regulatory factor 7 (Irf7) via Toll-like receptor 7 (TLR7). Similarly, ROR-α expression is thought to be increased in activated phenotype Treg cells in mouse skin [Id., citing Malhotra, N. et al. Sci. Immunol. (2018) 3: eaao6923]. Activated Treg cells expressing ROR-α have been shown to attenuate the function of group 2 innate lymphoid cells that reside in the skin. This has been shown to limit allergic skin inflammation in models of atopic dermatitis mediated by type II cytokines including IL-4, IL-5, and IL-13.

The circadian clock determines the rhythm with which immune cells circulate or migrate into tissues. The adhesion molecules ICAM-1 and VCAM-1 on endothelial cells vary based on the degree of inflammation, as well as rhythmicity, and act as homing signals for leukocytes in homeostasis, as well as inflammation [Id., citing He, W. et al. Immunity (2018) 49: 1175-1190]. Moreover, in skin, CD44 appears to be the adhesion molecule that varies in a circadian manner and acts as a honing signal for leucocytes in endothelial cells that constitute the capillaries of the dermis [Id., citing He, W. et al. Immunity (2018) 49: 1175-1190; Zoller, M. et al. J. Leukoc. Biol. (2007) 82: 57-71]. This circadian rhythmic variation prevents overactivation of the immune system when an external challenge is unlikely, and preparation of the immune system in more active phases of the day when a host is more likely to be faced with a challenge of a pathogen [Id., citing He, W. et al. Immunity (2018) 49: 1175-1190].

Although the immune system remains constantly vigilant and primed to mount a response to antigens, research into the influence of circadian rhythm on the immune system suggests an existence of a partition of the day into two phases. The first phase is one of heightened vigilance during waking hours where most activity occurs and an immune onslaught is most likely. This is followed by a recovery phase where resolution of inflammation and tissue repair occurs in the entire organism including the skin [Id., citing Curtis, A M et al. Immunity (2014) 40: 178-186]. The modulation of the two immunological pathways in skin has been shown to be influenced by glucocorticoids and nutrient intake, which act as zeitgebers. Of these, glucocorticoids have been studied in greater depth and have been linked to the central clock in the SCN [Id., citing San Phan, T. et al. J. Sci. Adv. (2021) 7: eabe0337; Palomino-Segura, M. and Hidalgo, A. J. Exp. Med. (2021) 218: e20200798; Waggoner, SN, Curr. Allergy Asthma Rep. (2020) 20: 2; Oster, H. et al. Endocr. Rev. (2016) 38: 3-45]. The secretion of adrenocorticotropin (ACTH) from the anterior pituitary gland is under the control of the SCN [Id., citing Curtis, A M et al. Immunity (2014) 40: 178-186]. However, ablation of the adrenal glands does not lead to loss of circadian oscillation in the skin and other peripheral tissue. In addition to the fact that skin can be directly entrained, this retention of circadian rhythm could be explained by the fact that keratinocytes of the epidermal layer in the skin are capable of regulating immune function by de novo synthesis of glucocorticoids via 11β-hydroxylase (Cyp11b1), in addition to reactivation of inactive glucocorticoids via the enzyme 11β-hydroxysteroid dehydrogenase type 1 (HSD11B1) [Id., citing San Phan, T. et al. J. Sci. Adv. (2021) 7: eabe0337; Buhr, E D et al. Curr. Biol. (2019) 29: 3478-3487].

Inflammation can disrupt the local, peripheral circadian clocks, as well as the central clock in the SCN. Recent reports implicate the NF-kB pathway in playing a central role in causing these perturbations [Id., citing Shen, Y. et al. PLoS Genet. (2021) 17: e1009933; Hong, H K et al. Genes Dev. (2018) 32: 1367-1379; Haspel, J A, et al. Nat. Commun. (2014) 5: 4753]. In addition to this pathway, researchers have shown that TNF-α, IFN-γ, IL-1, and LPS are capable of disrupting the oscillations of a core clock gene and the genes that they control [Id., citing Curtis, A M et al. Immunity (2014) 40: 178-186; Waggoner, SN. Curr. Allergy Asthma Rep. (2020) 20: 2; Abreu, M. et al. Sci. Rep. (2018) 8: 11474; Yoshida, K. et al. Scand. J. Rheumatol. (2013) 42: 276-280; Bashir, M M et al. Arch. Dermatol. Res. (2009) 301: 87-91; Cavadini, G. et al. Proc. Natl Acad. Sci. USA (2007) 104: 12843-12848; Cermakian, N., et al. Chronobiol. Int. (2013) 30: 870-888; Castanon-Cervantes, O. et al. J. Immunol. (2010) 185: 5796-5805; Okada, K. et al. J. Surg. Res. (2008) 145: 5-12]. In humans, epidemiological studies have also associated shift work, where the circadian clock is assumed to be disrupted, with higher risk of psoriasis [Id., citing Paganelli, R. et al. Clin. Mol. Allergy (2018) 16: 1; Wu, G. et al. Proc. Natl Acad. Sci. USA (2018) 115: 12313-12318]. These inflammatory reactions brought on by circadian disruptions are also likely to compromise the integrity of the skin, since, in human keratinocytes, TIMP3, which is a broad spectrum inhibitor of extracellular matrix (ECM)-degrading enzymes (MMPs, ADAM, ADAMTS), is likely to be under CLOCK control [Id., citing Sherratt, M J et al. Matrix Biol. (2019) 84: 97-110; Palomino-Segura, M. and Hidalgo, A. J. Exp. Med. (2021) 218: e20200798; Waggoner, SN. Curr. Allergy Asthma Rep. (2020) 20: 2; Scheiermann, C. et al. Nat. Rev. Immunol. (2018) 18: 423-437; Matsui, M S et al. Int. J. Mol. Sci. (2016) 17: 801; Fan, D. and Kassiri, Z. Front. Physiol. (2020) 11: 661; Park, S. et al. FASEB J. (2018) 32: 1510-23; Yeom, M. et al. Molecules (2018) 23: 745]. Thus, the magnitude of immune responses in the skin are profoundly impacted by the circadian system. In turn, disruption of the circadian rhythm of the skin leads to immune hyperactivity or an aberrant immune response that can manifest as pathologies such as dermatitis or psoriasis.

### Influence of the Circadian Clock on Skin Homeostasis and Stress Mediation

Circadian oscillations are observed in keratinocytes and melanocytes of the epidermis and the fibroblasts of the dermis [Id., citing Sandu, C. et al. Cell. Mol. Life Sci. (2015) 72: 2237-2248; Sandu, C. et al. Cell Mol. Life Sci. (2012) 69: 3329-3339]. The circadian clock machinery responsible for oscillations impact the metabolic processes of these cells and has an impact of tissue homeostasis. The epidermis is generated from epidermal stem cells in the basal layer that undergo asymmetric cell division, giving rise to either daughter stem cells or keratinocytes that will undergo a process of differentiation and desquamation to form the horny layer of the stratum corneum. It takes approximately 14 days for epidermal stem cells to end up as part of the stratum corneum. During this 2-week period, the process of differentiation does not occur continuously, but instead appears to occur in five sequential 24-h cyclic phases coordinated by the circadian clock. When studied via gene expression, each phase lasts for 4-5 h [Id., citing Sherratt, M J et a. Matrix Biol. (2019) 84: 97-110; Janich, P. et al. Cell Stem Cell (2013) 13: 745-753; Eckhart, L. et al. Biochim. Biophys. Acta (2013) 1833: 3471-3480]. In differentiated keratinocytes, the genes upregulated in the first three phases under circadian control remained similar to their undifferentiated counterparts, but included genes associated with DNA damage protection and repair, indicating constant vigilance against assault to the genetic code. In the next two phases, differentiated keratinocytes seem to shift their focus to building a defensive barrier with genes for differentiation and keratin organization being upregulated. This is likely to include the surface lipids of the skin that are under clock control and contribute to the skin barrier [Id., citing Janich, P. et al. Cell Stem Cell (2013) 13: 745-753; Jia, Y et al. Exp. Dermatol. (20019) 28: 858-862]. The differentiation process is not only dependent on the expression of the clock genes, but also their amplitude.

Melanocytes and dermal fibroblasts have also shown to possess functioning circadian clock machinery, but the amplitude of oscillations appear smaller than that of keratinocytes. Despite this, the circadian clock plays a functional role in melanocytes by controlling the abundance of melanosomes, as well as the expression of melanin synthesis enzyme, Tyrosinase and the phosphorylation of melanocyte inducing transcription factor (MITF), which increases when BMAL1 or PER1 are silenced [Id., citing Hardman, J A et al. J. Investig. Dermatol. (2015) 135: 1053-1064; Slominski, A T et al. J. Investig. Dermatol. (2015) 135: 943-945]. The protein OPN4 (Melanopsin) has been shown to affect the molecular clock components and their responsiveness to classical clock activators in melanocytes. Knocking out OPN4 in melanocytes resulted in rapid cell cycle progression and increased cellular proliferation, which correlated with the altered gene expression of MITF and the core circadian clock components [Id., citing De Assis, L V M et al. Curr. Issues Mol. Biol. (2021) 43: 1436-1450]. The impact of the function of the circadian clock on dermal fibroblasts is yet to be characterized. However, the influence of circadian rhythm on the synthesis and secretion of Type I collagen, a major component of the ECM of the dermis, is already known [Id., citing Chang, J. et al. Nat. Cell Biol. (2020) 22: 74-86]. Furthermore, the efficiency of migration and adhesion of fibroblasts modulated via actin dynamics was found to be circadian regulated [Id., citing Hoyle, N P et al. Sci. Transl. Med. (2017) 9: eaa12774].

Not only is normal function of skin impacted by circadian oscillations, but also the ability of skin to deal with stress is influenced by the cellular clock. As described above, the genes involved in DNA damage protection and repair in the epidermis are under clock control, as well as genes that mediate oxidative stress responses, in particular NRF2, the peroxiredoxins, glutathione peroxidase, and sestrins [Id., citing Ishii, T. et al. Free Radic. Biol. Med. (2018) 119: 34-44; Ndiaye, M A et al. Antioxid. Redox. Signal. (2014) 20: 2982-2996; Edgar, R S et al. Nature 92012]485: 459-64; Avitabile, D. et al. Intl J. Biochem. Cell Biol. (2014) 53: 24-34; Kolinjivadi, A M et al. J. Endocr. Re. at. Cancer (2021) 28: R55-R66; Kondratov, R V et al. Aging (2009) 1: 979-987]. In human skin, the activity of the DNA repair enzyme 8-oxoguanine DNA glycosylase (OGG1) was higher at night [Id., citing Manzella, N. et al. Sci. Rep. (2015) 5: 13752]. This is particularly important in the case of melanocytes, since they can accumulate DNA damage long after UV exposure, via melanin excitation [Id., citing Lyons, A B et al. J. Clin. Aesthet. Dermatol. (2019) 12: 42-45; Premi, S. et al. Science (2015) 347: 842-847].

The evolutionary conserved hormone melatonin is also responsible for combating DNA damage and oxidative stress, as well as maintaining skin homeostasis [Id., citing Slominski, A T et al. J. Investig. Dermatol. (2018) 138: 490-499]. This correlates with the finding that the circadian rhythm of melanin secretion is disrupted in psoriatic patients [Id., citing Mozzanica, N. et al. Acta Derm. Venereol. (1988) 68: 312-16]. Since this molecule and its related metabolites are free radicle scavengers, it is capable of stress mediation [Id., citing Slominski, A T et al. J. Investig. Dermatol. (2018) 138: 490-499; Ndiaye, M A et al. Antiox. Redox Signal. (2014) 20: 2982-2996]. Skin pigmentation and hair growth are also controlled by melatonin; its activity may also be influenced by the skin's circadian clock [Id., citing Hardman, J A et al. J. Investig. Dermatol. (2015) 135: 1053-1064; Slominski, A T et al. J. Investig. Dermatol. (2015) 135: 943-45; Al-Nuaimi, Y. et al. J. Investig. Dermatol. (2014) 134: 610-619]. Melatonin has also been implicated in the control of skin and body temperature in a circadian manner. In rat skin, the circadian clock machinery dermal fibroblasts is capable of using melatonin as an internal signal to fine-tune its oscillations, with temperature being used as an external queue [Id., citing Cuesta, M. et al. J. Biol. Rhythms (2017) 32: 257-273].

### The Circadian Clock and Seasonality

Physiological changes in human skin are a consequence of seasonality triggered by several parameters such as light irradiation of various wavelengths with changing intensities over the year, temperature, temperature shifts between indoors and outdoors, humidity, and sweat resulting in, for example, a decrease in pH due to acidification and wind accelerated evaporation, thus modulating trans-epidermal water loss (TEWL). The seasonal temperature changes modulate blood microcirculation and thus the accessibility to nutrients, which contributes to physiological skin changes.

### Intersection of Circadian Rhythms and Aging

The role of circadian rhythm and its relationship with skin aging is only now beginning to be understood. The rhythmicity of multiple features are altered with age. This includes sleep, body temperature cycles, and locomotor activity [Id., citing Duan, J. et al. FEBS Lett. (2021) 595: 2413-2436; Hood, S. and Amir, S. J. Clin. Investig. (2017) 127: 437-446]. For circadian rhythm, the amplitude of oscillation of clock controlled genes decreases with age in peripheral tissue, but not in the SCN, particularly when Per2 is being tracked [Id., citing Buijink, M R et al. J. Biol. Rhythms (2020) 35: 167-179]. This indicates that age-related circadian alteration in peripheral clocks are independent of the SCN clock. In aged skin, this problem is further compounded by the presence of senescent cells. Senescent cells show dampened circadian rhythmicity and are less efficient in the transmission of circadian signals to their clocks [Id., citing Kunieda, T. et al. Circ. Res. (2006) 98: 532-539]. Therefore, senescence is implicated as the mechanism by which aging impairs entrainment of peripheral circadian clocks [Id., citing Kunieda, T. et al. Cir. Res. (2006) 98: 532-539; Ahmed, R. et al. Front. Neurosci. (2021) 15: 638122]. Furthermore, aged dermal fibroblasts secrete a unique aging-associated set of proteins, distinct from the canonical senescence-associated secretory phenotype. Among these include multiple candidates involved in inflammatory signaling and maintenance or alteration of the tissue microenvironment [Id., citing Waldera Lupa, D M et al. J. Investig. Dermatol. (2015) 135: 195419-68]. Thus, there is a high likelihood that aging via senescence is directly capable of disrupting the above mentioned immunological and stress mediatory pathways in skin. Since the rigidity of the tissue microenvironment is also impacted, aging also likely dampens the circadian clock of keratinocytes (which prefer a softer matrix) and fibroblasts (which prefer a firmer matrix) [Id., citing Williams, J. et al. J. Cell Sci. (2018) 131: jcs208223; Yang, N. et al. Nat. Commun. (2017) 8: 14287].

The circadian oscillations of epidermal cells remain robust even under aged conditions, but they are rewired to adapt to the stressors associated with an aged environment and remain committed to development and maintenance of the skin barrier through daily rhythmic cell division, in spite of DNA damage they many have incurred [Id., citing Solanas, G. et al. Cell (2017) 170: 678-692].

### Plant-Derived Exosomes Affect a Human Keratinocyte Cell Line In Vitro

Plant-derived secondary metabolites are natural substances including alkaloids, flavonoids, polyphenols, terpenoids, and quinones; these are widely used as cosmeceuticals (cosmetic products that contain bioactive ingredients with potential therapeutic benefits for the skin) because they exert beneficial effects on the human skin, such as antiaging, moisturizing, whitening, regeneration, and nutritional supply. [Cho, J H et al. Applied Biological Chemistry (2022) 65: 8]. Typical examples are ginseng (Panax ginseng) and green tea (Camellia sinensis). Ginseng has several ginsenosides as representative active ingredients and exerts antiaging, anti-inflammatory, and antioxidative effects [Id., citing Kang, T H et al. J. Ethnopharmacol. (2009) 123: 446-451; Hong, C E and Lyu, SY. Immune Netw. (2011) 11: 42-49; Park, H J et al. J. Ginseng Res. (2012) 36: 225-414-415]. Green tea has reportedly been effective in antioxidation, photoprotection, and improvement in skin-related conditions owing to the active component of flavonoids including catechin and polyphenols [Id., citing Yarnell, E. and Abascal, K. Altern. Compl. Ther. (2012) 18: 141-144; Meetham, P. et al. Rev. Bras. Farmacogn. (2018) 28: 214-217; Koch, W. et al. Molecules (2019) 24: 4277].

To assess whether plant exosomes exert effects on human cells, extracts and exosomes from ginseng and green tea were used to treat keratinocytes in vitro and the change in transcriptosomes was compared to analyze the differential effect on cells. To prepare plant extracts, the leaf part of Green tea (1 kg) and the root part of Ginseng plants were hot air dried at 45° C. for 24 h, finely ground using a blender and 25 g then extracted in 25 L of distilled water at 80° C. for 3 hr and filtered through a 0.45 um mesh filter. Exosomes were prepared by ultracentrifugation or an aqueous two-phase extraction system (ATPS).

A Human Epidermal Keratinocyte (HaCaT) cell line was cultured in Dulbecco Modified Eagles Medium supplemented with 10% fetal bovine serum (FBS), 1% penicillin and 1% streptomycin at 37 C, 5% CO2. Keratinocytes were seeded at a density of 1×10⁵ in a 35 mm cell culture plate and incubated for 24 h. After incubation, cells were treated with fresh media containing 2% of a water extract or 1×10⁸/mL plant exosomes and mock-treated with distilled water as a control for 6 h. Extracts are normally treated for >24 h, so this 6 hr treatment window was shorter than normally required. Experiments were conducted when cell density reached 70-80% confluency. Exosomes showed a Gaussian distribution of nanoparticles within the range of 87-226 nm, inclusive, for ginseng and 108-234 nm, inclusive, for green tea.

The exosome treatment group had significantly higher numbers of expressed genes than the extract treatment or control groups. In the extract treatment group, 386 genes and 420 genes were upregulated for ginseng and green tea, respectively and 390 and 460 genes were downregulated. In the exosome treatment group, 877 and 843 genes were upregulated and 658 and 620 genes were downregulated for ginseng and green tea, respectively. This result implies that exosomes were effectively absorbed to keratinocytes. A previous study had reported that cabbage-derived exosomes were effectively absorbed to human cells by penetrating into them [Id., citing You, J Y et al. Bioact. Mat. (2021) 6: 4321-4332].

Expression patterns of genes related to skin aging, regenerating, barriers, and moisturizing were analyzed. Gene expression level of MMP12, MMP13, and NOTCH3, which are associated with skin aging, reportedly increase when exposed to light stress such as UVB and cause decomposition of collagen and skin aging [Id., citing Tewari, A. et al. J. Invest. Dermatol. (2014) 134: 2598-2609; Pittayapruek, P. et al. Int. J. Mol. Sci. (2016) 17: 868; Rossi, M. and Abdelmohsen, K. Cells (2021) 10: 1740]. Expression of these genes significantly decreased in the exosome treatment groups of ginseng and green tea, with no notable changes in the extract treatment groups. However, a previous study reported that the gene expression was decreased by >twofold change when keratinocytes were treated with compound K, obtained from ginseng extract, for 24 h [Id., citing Kim, S. et al. Biochem. Biophys. Res. Commun. (2004) 316: 348-55]. Analyzing the expression of genes related to skin regeneration revealed that fibroblast growth factor 12 (FGF12), which suppresses radiation damage to skin tissue, increased specifically in the ginseng exosome treatment group [Id., citing Nakayama, F. et al. J. Biol. Chem. (2011) 286: 25823-34]. A gene encoding a heparin sulfate sulfotransferase enzyme (HS3ST3A1), which facilitates skin regeneration by affecting skin regeneration signal delivery, and a lysyl oxidase (LOX) gene, which considerably influences healing of skin wounds by facilitating ECM stabilization through ECM formation, development, maturation and remodeling showed an increase in the exosome treatment group [Id., citing Patel, V N et al. Dev. Cell (2014) 29: 662-673; Cai, L. et al. Tissue Eng. Regen. Med. (2017) 14: 15-30]. A vimentin (VIM) gene, which affects the differentiation and migration of keratinocytes; ELOVL3, whose protein product belonged to a highly conserved family of microsomal enzymes involved in the formation of very long chain fatty acids (VLCFA), which affects lipid acid biosynthesis; and a keratin 1 (KRT1) gene whose protein product is a fibrillary protein that form skin, hair, and nails, are all recognized as genes that construct skin barrier and prevent water loss [Id., citing Velez-delValle, C. et al. Sci. Rep. (2016) 6: 24389; Cheng, F. et al. Proc. Nat. Acad. Sci. USA (2016) E4320-E4327; Westerberg, R. et al. J. Biol. Chem. (2004) 279: 5621-5629; Roth, W. et al. J. Cell Sci. (2012) 125: 5269-5279]. These genes responded particularly to the exosome treatment group and showed the highest increase in response to ginseng exosomes.

The Medicinal Plant Aloe vera: Pharmacological Properties

Aloe vera (Aloe barbadensis Miller, family Xanthorrhoeaceae) is a perennial green herb with bright yellow tubular flowers that is extensively distributed in hot and dry areas of North Africa, the Middle East of Asia, the Southern Mediterranean, and the Canary Islands. The colorless mucilaginous gel from Aloe vera leaves has been extensively used for pharmacological and cosmetic applications. Traditionally, this medicinal plant has been employed to treat skin problems (burns, wounds, and anti-inflammatory processes). Moreover, Aloe vera has shown other therapeutic properties including anticancer, antioxidant, antidiabetic, and antihyperlipidemic effects. Aloe vera contains more than 75 different compounds, including vitamins (vitamin A, C, E, and B12), enzymes (i.e., amylase, catalase, and peroxidase), minerals (i.e., zinc, copper, selenium, and calcium), sugars (monosaccharides such as mannose-6-phosphate and polysaccharides such as glucomannans), anthraquinones (aloin and emodin), fatty acids (i.e., lupeol and campesterol), hormones (auxins and gibberellins), and others (i.e., salicylic acid, lignin, and saponins) [Sanchez, M. et al. Molecules (2020) 25: 1324, citing Surjushe, A. et al. Indian J. Dermatol. (2008) 53: 163-166; Malik, I and Zarnigar, HN. Int. Res. J. Phar. (2003) 4: 75-79; Maan, A A et al. J. Herb. Med. (2018) 12: 1-10].

### Skin Protection

Most in vitro studies on skin protection study the ability of Aloe vera and active compounds in wound healing. The immortalized human keratinocyte HaCaT cell line, the primary normal human epidermal keratinocytes HEKa cell line, and fibroblast cell lines are the most used. These studies have revealed that Aloe vera and its major compounds (aloesin, aloin, and emodin) exert their protective action mainly through antioxidant and anti-inflammatory mechanisms.

The most common models for in vivo studies are genetically modified animals (BALB/c mice, HR-1 hairless mice and SKH-1 hairless mice) and UV and X-ray skin damage in animals. Most of these in vivo studies have been done with Aloe vera extracts and gel. Application of topical Aloe vera favored wound healing in animal models with dermal incisions by reducing inflammatory cell infiltration, increasing CD4+/CD8+ ratio lymphocytes, and improving epidermal thickness and collagen deposition [Id., citing Brandao, M L et al. Acta Cir. Bras. (2016) 31: 570-577; Oryan, A. et al. Annals Plast. Sug. (2016) 77: 37-46; Takzaree, N. t al. J. Physiol. Pharm. (2016) 94: 1285-90; Yos Adi Prakoso, K. J. Trop. Med. (2018) 2018: 6218303]. Clinical trials have demonstrated that Aloe vera facilitated rapid tissue epithelialization and granulation in burns [Id., citing Irani, P S and Varaie, S. Iran. J. Med. Sci. (2016) 41: S3], promoted healing of cesarean wounds [Id., citing Molazem, Z. et al. Global J. Health Sci. (2015) 7: 203], and accelerated wound healing of split-thickness skin graft donor sites [Id., citing Burusapat, C. et al. Plast. Reconstr. Surg. (2018) 142: 217-236]. Furthermore, Aloe vera has been investigated in randomized, double-blind, placebo-controlled studies for its benefits to maintain healthy skin. The daily oral intake of 40 μg of Aloe sterol (cycloartenol and lophenol) for at least 12 weeks improved skin elasticity in men under 46 years exposed to the sunlight but who do not use sunscreen to protect themselves [Id., citing Tanaka, M. et al. Clin. Cosmet. Invest. Dermat. (2016) 9: 435-442], reduced facial wrinkles in Japanese women over 40 years old by stimulating hyaluronic acid and collagen production [Id., citing Tanaka, M. et al. Clinical Cosmet. Invest. Dermat. (2015) 8: 95-104], and increased gross elasticity, net elasticity, and biological elasticity in women aged 30-59 [Id., citing Tanaka, M. et al. Skin Pharmacol. Physiol. (2016) 29: 309-317].

### Anti-Inflammatory and Anti-Oxidant Activity

Most recent studies on anti-inflammatory activity of Aloe vera are focused on the action mechanism of isolated compounds in murine macrophage RAW264.7 cells and mice stimulated with LPS. Hence, the potential anti-inflammatory effect of aloin was reported to be related to its ability to inhibit cytokines, ROS production, and the JAK1-STAT1/3 signaling pathway [Id., citing Ma, Y. et al. Int. J. Mol. Med. (2018) 42: 1925-1934; Jiang, K. et al. Int. Immunopharm. (2018) 64: 140-50]. Moreover, aloe-emodin sulfates/glucuronides (0.5 μM), rhein sulfates/glucuronides (1.0 M), aloe-emodin (0.1 M), and rhein (0.3 μM) inhibited pro-inflammatory cytokines and nitric oxide production, iNOS expression, and MAPKs phosphorylation [Id., citing Li, C Y et al. Am. J. Chin. Med. (2017) 45: 847-861].

The antioxidant activity of Aloe vera has been attributed, at least in part, to anthraquinones and related compounds (10 μM) which possess peroxyl radical scavenging activity and reducing capacity [Id., citing Sun, Y N et al. Nat. Prod. Res. (2017) 31: 2810-2813].

The present disclosure provides compositions containing exosome-like nanoparticles derived from plants that can be formulated for delivery to human skin. The plant exosomes are derived from plants of the family Asphodelaceae comprising the genus Aloe; or the plant is from Family Papaveraceae and is a Celandine plant; or the plant is from Family Passifloraceae and is a Passiflora ligularis or a Passiflora edulis plant. Plants can be subjected to two or more abiotic environmental stresses selected from high/low temperature, salinity, drought, light stresses, flooding, physical wounding, or heavy metal pollutants, which produce secondary plant stresses comprising oxidative stress and osmotic stress. The plants' response to these abiotic stresses is to generate exosomes comprising cargo comprising nucleic acids (e.g., miRNAs), plant proteins including heat shock proteins, and lipids that in vivo would enable adjustments of its signaling pathways and metabolism to ensure its growth and development in a challenging environment. The present disclosure provides that instead, the plant exosomes will be harvested for cosmetic, cosmeceutical or therapeutic applications in human subjects. We have demonstrated that plant exosomes can bind and release a signature cargo into human skin keratinocytes. The released cargo can promote survival of keratinocytes, fibroblasts and endothelial cells resident in the skin; reduce oxidative stress, modulate inflammation, modulate perturbations of the circadian clock of the skin comprising immune responses comprising inflammation; restore youthful appearance to the skin by producing collagen and elastin components that give skin its turgor, and maintain the integrity of the statum corneum and prevent water loss. The nanoparticles offer advantageous properties stemming from the tunable delivery of the signature cargo comprising natural biochemicals derived from the origin plants with no evidence of plant-induced inflammation or toxicity.

### SUMMARY OF THE INVENTION

According to one aspect, the present disclosure provides a composition comprising a purified population of plant-derived exosome-like nanoparticles isolated from tissue of a vascular plant, wherein size of the exosome-like nanoparticles is about 50 nm-500 nm inclusive; wherein the exosome-like nanoparticles comprise a tuned cargo comprising a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase, or a combination thereof; wherein the tuned cargo of the plant-derived exosome-like nanoparticles is produced by exposure of the plant to combinations of abiotic stress conditions that cause the plant to modulate its signaling pathways and metabolism to ensure its survival in a challenging environment.

According to some embodiments of the composition, the plant is of Family Asphodelaceae or is an Aloe vera plant. According to some embodiment, the plant is from Family Papaveraceae and is a Celandine plant. According to some embodiments, the plant is from Family Passifloraceae and is a Passiflora ligularis or a Passiflora edulis plant. According to some embodiments, the plant is from the Family Rubiaceae and is a Morinda citrifolia plant.

According to some embodiments, the plant tissue includes roots, stems, leaves, flowers, seeds, fruits, a liquid extract of the plant tissue, a nut milk, or a combination thereof.

According to some embodiments, the HSP chaperones induced under the abiotic stress conditions include HSP100, HSP90, HSP70, HSP60 a small HSP; or a combination thereof; and the Hsfs induced under the abiotic stress conditions include HsfA, HsfB or HsfC, or a combination thereof.

According to some embodiments, primary abiotic stress conditions, which include high/low temperature; salinity; drought; dehydration; flooding; heavy metal chemical pollutants; light stresses or physical wounding, produce secondary stresses comprising oxidative stress and osmotic stress.

According to some embodiments, the tuned cargo of the plant-derived exosomes is a result of exposure of the plant to two high temperature abiotic stress conditions.

According to some embodiments, the tuned protein cargo of the plant-derived exosomes correlates to a protein signature comprising human proteins including a keratin; semaphorin receptor plexin-B1 mitogen-activated protein kinase kinase 2 (MEKK2), diacylglycerol kinase; T cell receptor beta chain; a fez family zinc finger protein or a combination thereof.

According to some embodiments, the tuned protein cargo of the plant-derived exosomes can modulate bioactivities of mammalian cells directly or indirectly.

According to some embodiments, the mammalian cells are human cells; and the bioactivities comprise one or more correlated signaling pathways in the human cells. According to some embodiments, the human mammalian cells are cells of human skin.

According to some embodiments, the correlated human signaling pathways includes PI3K signaling, ERK/MAPK signaling; insulin growth factor 1 receptor (IGF1R) signaling, VEGFA/VEGFR2 signaling; leptin signaling; cytokine signaling; interleukin signaling, semaphorin signaling; sirtuin signaling; LRP1 signaling, or a combination thereof.

According to some embodiments, administration of the composition comprising the exosome-like nanoparticles comprising the tuned cargo modulates: collagen production in human dermal fibroblasts in vitro; or elastin production in human dermal fibroblasts in vitro; or hyaluronic acid production in human dermal fibroblasts in vitro; or interferon a2 production in mammalian PBMCs exposed to a microbial agent in vitro; or VEGFA production in human dermal fibroblasts in vitro; or a combination thereof.

According to some embodiments, the composition is a nutraceutical composition comprising a dietary amount of the purified plant-derived exosomes comprising the tuned cargo; or the composition is a cosmetic composition comprising a cosmetic amount of the purified plant-derived exosomes and a cosmetically acceptable carrier; or the composition is a cosmeceutical composition comprising a cosmeceutical amount of the purified plant-derived exosomes and a cosmeceutically acceptable carrier; or the composition is a therapeutic composition; comprising a therapeutic amount of the purified plant-derived exosomes and a pharmaceutically acceptable carrier.

According to another aspect, the present disclosure provides a method for improving appearance of human skin comprising: exposing a vascular plant to combinations of abiotic stress conditions that cause the plant to modulate its signaling pathways and metabolism to ensure its survival in a challenging environment; purifying from tissue of the vascular plant exposed to the combinations of abiotic conditions a population of plant-derived exosome-like nanoparticles (plant-derived exosomes) comprising a tuned cargo, wherein size of the plant-derived exosomes is about 50 nm-500 nm inclusive; preparing a composition comprising about 1×10E8 to about 1×10E12, inclusive, abiotically stressed plant-derived exosome-like nanoparticles containing the tuned cargo and a cosmetically acceptable carrier; and applying the composition topically to human skin; wherein the tuned cargo of the plant-derived exosomes comprises: a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase; or a combination thereof.

According to some embodiments of the method, the plant is a plant of Family Asphodelaceae or an Aloe vera plant; or the plant is from Family Papaveraceae and is a Celandine plant; or the plant is from Family Passifloraceae and is a Passiflora ligularis or a Passiflora edulis plant. According to some embodiments, the plant is from the Family Rubiaceae and is a Morinda citrifolia plant.

According to some embodiments, the plant tissue includes roots, stems, leaves, flowers, seeds, fruits, a liquid extract of the plant tissue, a nut milk or a combination thereof.

According to some embodiments, the HSP chaperone induced under the abiotic stress conditions include HSP100, HSP90, HSP70, HSP60, a small HSP or a combination thereof; and the Hsf induced under the abiotic stress conditions include HsfA, HsfB or HsfC, or a combination thereof.

According to some embodiments, primary abiotic stress conditions including high/low temperature; salinity; drought; dehydration; flooding; heavy metal chemical pollutants; light stresses or physical wounding produce secondary stresses comprising oxidative stress and osmotic stress.

According to some embodiments, the tuned protein cargo of the plant-derived exosome-like nanoparticles correlates to a protein signature comprising human proteins including a keratin; semaphorin receptor plexin-B1 mitogen-activated protein kinase kinase 2 (MEKK2), diacylglycerol kinase; T cell receptor beta chain; fez family zinc finger protein or a combination thereof.

According to some embodiments, the tuned protein cargo of the plant-derived exosomes can modulate bioactivities of mammalian cells directly or indirectly.

According to some embodiments, the bioactivities comprise one or more correlated signaling pathways in the human cells.

According to some embodiments, the correlated signaling pathways in the human cells include PI3K signaling, ERK/MAPK signaling; insulin growth factor 1 receptor (IGF1R) signaling, VEGFA/VEGFR2 signaling; leptin signaling; cytokine signaling; interleukin signaling, semaphorin signaling; sirtuin signaling; LRP1 signaling, or a combination thereof.

According to some embodiments, the composition comprising the tuned cargo of the exosome-like plant nanoparticles when applied to the skin may modulate gene expression in immune cells, keratinocytes, melanocytes or fibroblasts in the skin; modulate a signaling pathway that contributes to inflammation, immune dysfunction or both in the skin; modulate circadian rhythms of the skin and its components; rejuvenate appearance of the skin by improving youthful appearance of skin; reducing appearance of wrinkles by stimulating hyaluronic acid and collagen production; improving skin clarity; improving skin texture; improving skin luminosity; improving skin radiance; or a combination thereof.

According to some embodiments, the tuned protein cargo of the plant-derived exosome-like nanoparticles correlates to a protein signature comprising a huma protein in a signaling pathway in human skin, wherein the signaling pathway is a PI3K/AKT/mTOR pathway, an MAPK pathway, an IGF-1R pathway, a sirtuin pathway, an LRP1 pathway, or a combination thereof.

According to another aspect, the present disclosure provides a method for promoting hair health, comprising: exposing a vascular plant to combinations of abiotic stress conditions that cause the plant to modulate its signaling pathways and metabolism to ensure its survival in a challenging environment; purifying from tissue of the vascular plant exposed to the combinations of abiotic conditions a population of plant-derived exosome-like nanoparticles (plant-derived exosomes), comprising a tuned cargo wherein size of the plant-derived exosomes is about 50 nm-500 nm inclusive; preparing a composition comprising about 1×10E8 to about 1×10E12, inclusive, abiotically stressed plant-derived exosome-like nanoparticles containing the tuned cargo and a cosmetically acceptable carrier; and applying the composition topically to a subject in need thereof; wherein the tuned cargo comprises: a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase; or a combination thereof, wherein the composition increases proliferation of dermal papillae cells and hair follicle stem cells and increases hair growth.

According to some embodiments, applying topically includes applying to scalp, eyebrows, eyelashes or a combination thereof of the subject.

According to some embodiments, the method decreases hair loss; increases hair density; increases appearance of hair thickness; improves scalp health; improves hair shine; improves hair volume or body; or a combination thereof.

According to some embodiments, the composition comprising the tuned cargo of the exosome-like plant nanoparticles, when applied topically, modulates gene expression in the dermal papillae cells, hair follicle stem cells, or a combination thereof.

According to some embodiments, the composition comprising the tuned cargo of the exosome-like plant nanoparticles, when applied topically, modulates gene expression in human follicle dermal papilla cells (HFDPCs).

According to some embodiments, the composition comprising the tuned cargo of the exosome-like plant nanoparticles, when applied topically, modulates gene expression of IL-6 in HFDPCs 24 hours after application.

According to some embodiments, the composition comprising the tuned cargo of the exosome-like plant nanoparticles, when applied topically, modulates gene expression of CORIN, LEP, IL1B, IL-6, SRD5A2, BMP4, TGFB1, IGF1, HEY1, or a combination thereof in HFDPCs 72 hours after application.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of skin anatomy.

FIG. 2 is a schematic of cell types in the layers of the skin from the skin surface to the dermis, including the stratum corneum, the stratum lucidum, stratum granulosum, stratum germinativum, and stratum basale. Each contains living cells with specialized functions.

FIG. 3 is a schematic of the molecular structure of the mammalian circadian clock. The circadian clock consists of three core feedback loops. The newly described feedback loop has also been depicted. Loop 1: basic helix loop helix ARNT like 1 (BMAL1) gene dimerizes with Clock Circadian Regulator (CLOCK) gene or Neuronal PAS Domain Protein 2 (NPAS2) gene. These dimers bind to the promoter region E-box elements (5′-CACGTG-3′), triggering Period 1-3 (PER1-3), crystalline (CRY), retinoic acid-related orphan receptor (ROR), nuclear receptor subfamily 1, group D, member 1 (NR1D1), and transcription factor albumin D site-binding protein (DBP) gene expression. In mammals, DBP rhythmically activates transcription of various genes through a DNA cis-element, D-box. PER and CRY dimerize on reaching a critical concentration, inhibiting their own expression, causing oscillation of the expression of these proteins. Loop 2: The transcription of BMAL1, CLOCK, and NFIL3 (a rhythmically expressed transcription factor) is triggered when ROR binds to the RORE element 5′-(A/G)GGTCA-3′ in their promoter region. This binding of ROR to RORE is inhibited by NR1D1. Loop 3: PER transcription is initiated when the D box element (5′-TTATG(T/C)AA-3′) in its promoter region is bound to by DBP (Loop 1). This binding is negatively regulated by NFIL3 (Loop 2). DEC loop: The DEC protein that gives this loop its name, is responsible for its own oscillatory expression by inhibiting the binding of BMAL1: CLOCK to E-box elements (5′-CACGTG-3′) in its promoter. (taken from Salazar, A. and von Hagen, J., Intl J. Molec. Sci. (2023) 24: 5635)

FIG. 4 is a schematic of the mitogen activated protein kinase (MAPK) signaling pathways [taken from Soares-Silva, M. et al. Front. Microbiol. (2016) 7: 183]. The major MAPK pathways involved in inflammatory diseases are extracellular regulating kinase (ERK), p38 MAPK, and c-Jun NH2-terminal kinase (JNK). All three MAPK pathways may be activated by TGF-β, and signaling through these cascades can further regulate the expression of Smad proteins and mediate Smad-independent TGF-β responses. Smaroteins are key signal transducers for the TGF-β superfamily. These three MAPK pathways are all involved in TGF-β-induced fibrosis. [He, W. and Dai, C. Curr. Pathobiol. Rep. (2015) 3: 183-92, citing Tsou, P S et al. Am. J. Physiol. Cell Physiol. (22014) 307: C2-13; Kamato, D. et al. Cell Signal (2013) 25: 2017-24; Pannu, J. et al. J. Biol. Chem. (2007) 282: 10405-13; Yu, L. et al. J. Biol. Chem. (2002) EMBO J. 21: 3749-59]. Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-β can signal in a noncanonical manner via the MAPK family.

FIG. 5 is a schematic of the PI3K/Akt/mTOR pathway (Taken from Porta, et al. Front. Oncol. (2014) 4: art. 64). Activation of growth factor receptor protein tyrosine kinases results in autophosphorylation on tyrosine residues. P13K is then recruited to the membrane by directly binding to phosphotyrosine consensus residues of growth factor receptors or adaptors through one of the two SH2 domains in the adaptor subunit. This leads to allosteric activation of the catalytic (CAT) subunit of PI3K. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) [Id., citing Fruman, D A et al., Phosphoinositide kinases. Annu. Rev. Biochem. (1998) 67: 481-507, Fresno-Vara, J A, et al., PI3K/Akt signaling pathway and cancer. Cancer Treat. Rev. (2004) 30: 193-204]. Akt (also known as protein kinase B) is activated in response to stimulation of tyrosine kinase receptors, such as platelet-derived growth factor (PDGF), insulin-like growth factor, and nerve growth factor. Stimulation of Akt has been shown to be dependent on phosphatidylinositol 3-kinase (PI3-kinase) activity. Mammalian target of rapamycin (mTOR) is an atypical serine/threonine kinase that is present in two distinct complexes. The first, mTOR complex 1 (mTORC1), is composed of mTOR, regulatory protein associated with mTOR (Raptor), GβL (also known as mammalian lethal with Sec13 protein 8 or mLST8), and DEP domain containing m-TOR Interacting Protein (DEPTOR) and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumor-suppressor genes mutated in the tumor syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1. Most upstream inputs are funneled through Akt and TSC1/2 to regulate the nucleotide-loading state of Rheb. The second mTOR complex (mTORC2) is sensitive to growth factors, not nutrients, and is associated with rapamycin-insensitivity mTORC2 is thought to modulate growth factor signaling by phosphorylating the C-terminal hydrophobic motif of some cAMP-dependent, cGMP-dependent and protein kinase C (AGC) kinases such as Akt and SGK [Jhanwar-Uniyal, M. et al. Adv. Biol. Regul. (2015) 57: 64-74].

FIG. 6 is a schematic of the IGF-1 signaling pathway [taken from Jams, W T and Lovly, CM, Clin. Cancer Res. (2015) 21 (19): 42770-77]. Insulin-like growth factors (IGFs) bind specifically to the IGF1 receptor on the cell surface of targeted tissues. Ligand binding to the α subunit of the receptor leads to a conformational change in the β subunit, resulting in the activation of receptor tyrosine kinase activity. Activated receptor phosphorylates several substrates, including insulin receptor substrates (IRSs) and Src homology collagen (SHC). Phosphotyrosine residues in these substrates are recognized by certain Src homology 2 (SH2) domain-containing signaling molecules. These include an 85 kDa regulatory subunit (p85) of phosphatidylinositol 3-kinase (PI 3-kinase), growth factor receptor-bound 2 (GRB2) and SH2-containing protein tyrosine phosphatase 2 (SHP2/Syp), which lead to the activation of downstream signaling pathways, PI 3-kinase pathway and Ras-mitogen-activated protein kinase (MAP kinase) pathway.

FIG. 7, taken from Su, J. et al. Front. Immunol. (2021) 11: 593564, is a schematic representation of leptin and leptin receptor signal transduction pathways and functions. Stimulation of the leptin receptor by leptin can activate Janus (JAK2) kinase, resulting in tyrosine phosphorylation of the receptor and downstream proteins, including Signal transducer and Activator of Transcription 3 (STAT3), a member of the signal transducers and activators of transcription (STAT) transcription factor family, which functions together with the janus kinases (JAK) in JAK-STAT signaling networks commonly activated by cytokines, protein tyrosine phosphatase 2 (SHP-2), an SH2 domain-containing protein tyrosine phosphatase, Insulin Receptor Substrate 2 (IRS2, an adaptor molecule that plays a fundamental role in insulin and IL-4, IL-7 and IL-9 signaling), and phosphoinositide 3-kinase (PI3K) that play a role in regulating transcription of genes essential for energy intake and lipid metabolism. ACC (acetyl-CoA carboxylase); AMPK (adenosine monophosphate kinase); CPTI (carnitine palmitoyltransferase 1); ERK (extracellular signaling-regulated kinase); FAS (fatty acid synthase); SCD1 (stearoyl-coenzyme A desaturace 1, enzyme in fatty acid metabolism); TG (triglycerides); PL (phospholipids); CE (cholesterol esters)

FIG. 8 is a bar graph showing HSP70 gene expression in purified aloe-derived exosomes before treatment and after heat stress treatment compared to the whole plant and a pre-treatment control.

FIG. 9A is a bar graph of collagen 1 (ng/mL) in human dermal fibroblasts against a media control; treatment samples (Aloe 1, Aloe 2, and adipose stromal stem cells (ASC)); and a TGFb positive control showing that Aloe-derived purified exosomes can induce collagen I synthesis in human dermal fibroblasts. FIG. 9B is a bar graph showing percent change of collagen in human dermal fibroblasts treated as in FIG. 9A.

FIG. 10A is a bar graph of VEGF-A concentration (ng/mL) in human dermal fibroblasts treated with 5% FBS (positive control), Aloe exosomes (1×10E9) Aloe Exosomes (1×10E8), and Aloe exosomes (1×10E7). FIG. 10B shows VEGFA concentration fold change; vs. 5% FBS positive control, Aloe exosomes (1×10E9) Aloe Exosomes (1×10E8), and Aloe exosomes (1×10E7). FIG. 10C shows VEGFA concentration (ng/mL) versus Il-8 (1000 ng/mL). IL-8 (100 mg/mL), IL-8 (10 ng/mL), IL-8 (1 ng/mL), IL8 (0.1 ng/mL), and 10% FBS positive control

FIG. 11A, FIG. 11B, and FIG. 11C show bar graphs of fold change in production of elastin (blue), hyaluronic acid (orange) and collagen 1 (gray) in human skin dermal fibroblasts treated with Aloe exosomes FIG. 11A Prep 1 [1×10E8, 3.33×10E7, 1.11×10E7], and Prep 2 (1.00×10E8, 3.33E7, 1.11×10E7), FIG. 11B Prep 3 [1×10E8, 3.33×10E7, 1.11×10E7], and Prep 4 (1.00×10E8, 3.33×10E7, 1.11×10E7), and FIG. 11C Prep 5 [1×10E8, 3.33×10E7, 1.11×10E7] compared to a media control.

FIG. 12 shows average cytokine production (pg/mL interferon a2 (INFa2) in PBMCs in vitro in response to bacterial exposure. Treatment groups from left to right are: media controls (dark green=media only, black=media plus dextran)−Media+P. acnes; adipose stromal stem cells (ASC) (yellow) Aloe exosomes (light green=prep 1; and light blue=prep II).

FIG. 13A, FIG. 13B, FIG. 13C, FIG. 13D, FIG. 13E and FIG. 13F show delivery of exosomes containing fluorescent-labeled RNA cargo into cell cytoplasm of a monolayer of human skin fibroblasts in vitro at 4 degrees C. (TOP ROW) and at 37 degrees C. (BOTTOM row). Top Row, incubation at 4° C.: FIG. 13A (cells alone (no addition of labeled exosomes) incubated at 4° C.), FIG. 13B (addition of Aloe exosomes at 4° C.), FIG. 13C (addition of adipose MSC exosomes at 4° C.). At 4° C., minimal Aloe and MSC exosome fluorescence were expected, since 4° C. impedes exosome binding and internalization. Bottom Row, incubation at 37° C.: FIG. 13D (cells alone incubated, at 37° C., no addition of labeled exosomes), FIG. 13E (addition of aloe exosomes at 37° C.) and FIG. 13F (addition of adipose MSC exosomes at 37° C.). At 37° C., MSC exosome fluorescence is readily visible (blue arrows) when compared to the negative control at 4° C. shown in FIG. 13C, indicating successful labeled RNA cargo delivery.

FIG. 14A, FIG. 14B, and FIG. 14C show exosome attachment to human dermal fibroblast cells in suspension culture. Exosomes comprised fluorescent-labeled RNA. FIG. 14A, Cells alone (negative control), green; HS aloe exosomes (red). FIG. 14B cells plus labeled heat shock MSC exosomes. FIG. 14C, merging of left and right panels, showing overlay of labeled HS aloe and HS MSC peaks.

FIG. 15A and FIG. 15B, show bar graphs from two experiments showing effect of heat-shocked (HS) human adipose stromal stem cell (ASC) exosomes and HS aloe exosomes on cell proliferation of hair dermal papillae, compared to media controls (control media, test media).

FIG. 16 is a bar graph showing cell counts (×1000) vs. exosome preparation (left to right, control media; test media; Aloe-exosomes (1.5×10E9; 3.0×10E9; 6.0×10E9); adipose stromal stem cells (ASCs) (1.5×10E8; 3.0×10E8; 6.0×10E8); amniotic fluid (AF) (1.5×10E8; 3.0×10E8; 6.0×10E8). Overall, all test concentrations of exosomes showed greater cell proliferation than the Test Media alone. All but 1.5 and 3.0 for ASC and 6.0 for AF showed equal to or greater cell proliferation than the Control growth media.

FIG. 17A and FIG. 17B are bar graphs showing in vitro antioxidant activity of Aloe-derived exosomes and human ASC exosomes by 2,2′-azino-bis-(3-ethylbenzothiazoline-6-sulfonic) acid (ABTS) (FIG. 17A) and oxygen radical absorbance capacity (ORAC) (FIG. 17B) assays.

FIG. 18, taken from Revollo, JR and Li, X. Trends Biochem. Sci. (2013) 38 (3): 160-67, schematically depicts the enzymatic reaction catalyzed by Sirt1. Sirt1 catalyzes the deacetylation of several proteins by consuming nicotinamide adenine dinucleotide (NAD+), generating nicotinamide (NAM) and 2′-O-Acetyl-ADP-Ribose. NAM is recycled back into NAD+ by the enzymes nicotinamide phosphoribosyltransferase (NAMPT), nicotinamide mononucleotide adenylyl transferase (NMNAT), and the nicotinamide mononucleotide (NMN) intermediate.

FIG. 19A is a photograph of the scalp of Subject 1, a female with hair loss from salon coloring treatments every two weeks, showing hair loss before treatment, FIG. 19B is a photograph of the same area of the scalp after 5 weeks of treatment.

FIG. 20A is a photograph of the scalp of Subject 2, a female with hair loss due to androgenetic alopecia, showing hair loss before treatment, FIG. 20B is a photograph of the same area of the scalp after 8 weeks of treatment.

FIG. 21A is a photograph of the scalp of Subject 3, a male with hair loss post COVID-19, showing hair loss before treatment, FIG. 21B is a photograph of the same area of the scalp after 15 weeks of treatment.

FIG. 22A is a photograph of the scalp of Subject 4, a male with hair loss of unknown pathology, showing hair loss before treatment, FIG. 22B is a photograph of the same area of the scalp after 20 weeks of treatment.

FIG. 23A a photograph of the scalp of Subject 5, a male with alopecia areata, showing hair loss before treatment, FIG. 23B is a photograph of the same area of the scalp after 12 weeks of treatment.

FIG. 24A a photograph of the scalp of Subject 6, a 58 year old male with androgenetic alopecia and stress, showing hair loss before treatment, FIG. 24B is a photograph of the same area of the scalp after 20 weeks of treatment.

FIG. 25 is a bar graph of a lactate dehydrogenase (LDH) cytotoxicity assessment of human follicle dermal papilla cells (HFDPCs) 24 hours after treatment with 1.5 billion native, not heat shocked, aloe-derived exosomes (TM1), 1.5 billion engineered, heat shocked, aloe-derived exosomes (TM2), 5 billion engineered, heat shocked, aloe-derived exosomes (TM3), and 5 billion engineered, heat shocked) human adipose stromal stem cell (ASC)-derived exosomes (TM4) compared to an untreated negative control (UNT), a media+serum positive control (Positive Ctrl), a Triton X-100® treated positive control (Triton), and a PBS treated vehicle control (Vehicle Ctrl).

FIG. 26 is a qPCR amplification curve for the selected endogenous control gene, peptidylprolyl isomerase A (PPIA) for gene expression analysis of human follicle dermal papilla cells (HFDPCs) 24 hours after treatment with 1.5 billion native, not heat shocked, aloe-derived exosomes, 1.5 billion engineered, heat shocked, aloe-derived exosomes, 5 billion engineered, heat shocked, aloe-derived exosomes, and 5 billion engineered, heat shocked, human adipose stromal stem cell (ASC)-derived exosomes, compared to an untreated negative control, a media+serum positive control, and a PBS treated vehicle control. Each line in the amplification curve represents a different sample across treatment groups.

FIG. 27A, FIG. 27B, and FIG. 27C are qPCR amplification curves showing poor quality data for different genes expressed in human follicle dermal papilla cells (HFDPCs) 24 hours after treatment with 1.5 billion native, not heat shocked, aloe-derived exosomes, 1.5 billion engineered, heat shocked, aloe-derived exosomes, 5 billion engineered, heat shocked, aloe-derived exosomes, and 5 billion engineered, heat shocked, human adipose stromal stem cell (ASC)-derived exosomes, compared to an untreated negative control, a media+serum positive control, and a PBS treated vehicle control. Each line in the amplification curve represents a different sample across treatment groups. FIG. 27A shows a poor quality amplification curve for the WIF1 gene. FIG. 27B shows a poor quality amplification curve for the WNT3A gene. FIG. 27C shows a poor quality amplification curve for the PROM1 gene.

FIG. 28A and FIG. 28B are exemplary qPCR amplification curves showing high quality data for different genes expressed in human follicle dermal papilla cells (HFDPCs) 24 hours after treatment with 1.5 billion native, not heat shocked, aloe-derived exosomes, 1.5 billion engineered, heat shocked, aloe-derived exosomes, 5 billion engineered, heat shocked, aloe-derived exosomes, and 5 billion engineered, heat shocked, human adipose stromal stem cell (ASC)-derived exosomes, compared to an untreated negative control, a media+serum positive control, and a PBS treated vehicle control. Each line in the amplification curve represents a different sample across treatment groups. FIG. 28A shows a high quality amplification curve for the BMP6 gene. FIG. 28B shows a high quality amplification curve for the SRD5A2 gene.

FIG. 29A, FIG. 29B, and FIG. 29C are exemplary qPCR amplification curves showing a high fold-change for different genes expressed in human follicle dermal papilla cells (HFDPCs) 24 hours after treatment with 1.5 billion native, not heat shocked, aloe-derived exosomes, 1.5 billion engineered, heat shocked, aloe-derived exosomes, 5 billion engineered, heat shocked, aloe-derived exosomes, and 5 billion engineered, heat shocked, human adipose stromal stem cell (ASC)-derived exosomes when compared to control groups (vehicle or untreated control groups). Each line in the amplification curve represents a different sample across treatment groups. FIG. 29A shows a high fold-change amplification curve for the DKK1 gene. FIG. 29B shows a high-fold change amplification curve for the IL1B gene. FIG. 29C shows a high fold-change amplification curve for the MKI67 gene.

FIG. 30 is a bar graph of a lactate dehydrogenase (LDH) cytotoxicity assessment of human follicle dermal papilla cells (HFDPCs) 72 hours after treatment with 5 billion engineered (heat shocked) aloe exosomes (TM 1), 5 billion native (not heat shocked) aloe-derived exosomes (TM 2), 5 billion engineered (heat shocked) human adipose stromal stem cell (ASC)-derived exosomes (TM 3), and 5 billion native (not heat shocked) human ASC-derived exosomes (TM 4), compared to an untreated control (UNT), a media+serum positive control (Positive Ctrl), a Triton X-100@treated positive control (Triton), and a PBS treated vehicle control (Vehicle Ctrl).

FIG. 31 is a qPCR amplification curve for the selected endogenous control gene, Hypoxanthine Phosphoribosyltransferase 1 (HPRT1) for gene expression analysis of human follicle dermal papilla cells (HFDPCs) 72 hours after treatment with 5 billion engineered (heat shocked) aloe exosomes, 5 billion native (not heat shocked) aloe-derived exosomes, 5 billion engineered (heat shocked) human adipose stromal stem cell (ASC)-derived exosomes, and 5 billion native (not heat shocked) human ASC-derived exosomes, compared to an untreated control, a positive control, negative control, and a PBS treated vehicle control. Each line in the amplification curve represents a different sample across treatment groups.

FIG. 32A, FIG. 32B, and FIG. 32C are qPCR amplification curves showing poor quality data for different genes expressed in human follicle dermal papilla cells (HFDPCs) 72 hours after treatment with 5 billion engineered (heat shocked) aloe exosomes, 5 billion native (not heat shocked) aloe-derived exosomes, 5 billion engineered (heat shocked) human adipose stromal stem cell (ASC)-derived exosomes, and 5 billion native (not heat shocked) human ASC-derived exosomes, compared to an untreated control, a positive control, negative control, and a PBS treated vehicle control. Each line in the amplification curve represents a different sample across treatment groups. FIG. 32A shows a poor quality amplification curve for the PROM1 gene. FIG. 32B shows a poor quality amplification curve for the WIF1 gene. FIG. 32C shows a poor quality amplification curve for the WNT3A gene.

FIG. 33A, FIG. 33B, FIG. 33C, and FIG. 33D are exemplary qPCR amplification curves showing high quality data for different genes expressed in human follicle dermal papilla cells (HFDPCs) 72 hours after treatment with 5 billion engineered (heat shocked) aloe exosomes, 5 billion native (not heat shocked) aloe-derived exosomes, 5 billion engineered (heat shocked) human adipose stromal stem cell (ASC)-derived exosomes, and 5 billion native (not heat shocked) human ASC-derived exosomes, compared to an untreated control, a positive control, negative control, and a PBS treated vehicle control. Each line in the amplification curve represents a different sample across treatment groups. FIG. 33A shows a high quality amplification curve for the LEF1 gene. FIG. 33B shows a high quality amplification curve for the TCF4 gene. FIG. 33C shows a high quality amplification curve for the TGFB1 gene. FIG. 33D shows a high quality amplification curve for the WNT5A gene.

FIG. 34A, FIG. 34B, and FIG. 34C are exemplary qPCR amplification curves showing a high fold-change for different genes expressed in human follicle dermal papilla cells (HFDPCs) 72 hours after treatment with 5 billion engineered (heat shocked) aloe exosomes, 5 billion native (not heat shocked) aloe-derived exosomes, 5 billion engineered (heat shocked) human adipose stromal stem cell (ASC)-derived exosomes, and 5 billion native (not heat shocked) human ASC-derived exosomes, compared to an untreated control, a positive control, negative control, and a PBS treated vehicle control when compared to control groups (vehicle or untreated control groups). Each line in the amplification curve represents a different sample across treatment groups. FIG. 34A shows a high fold-change amplification curve for the BMP2 gene. FIG. 34B shows a high-fold change amplification curve for the IL6 gene. FIG. 34C shows a high fold-change amplification curve for the LEP gene.

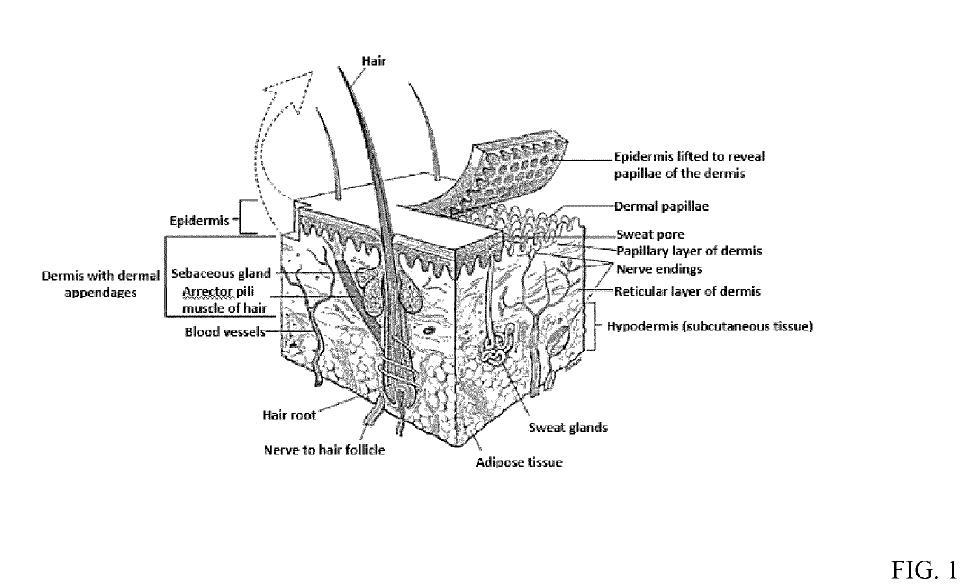

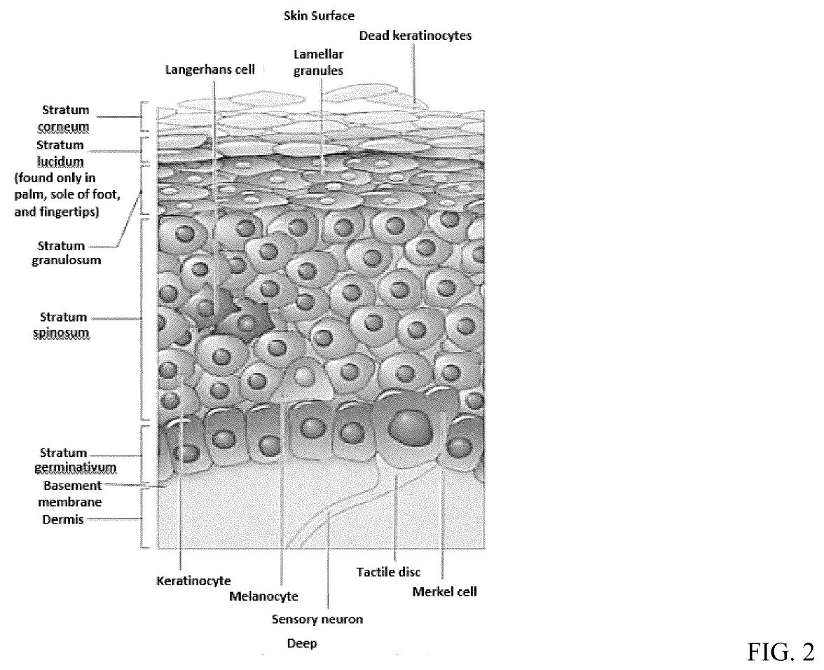

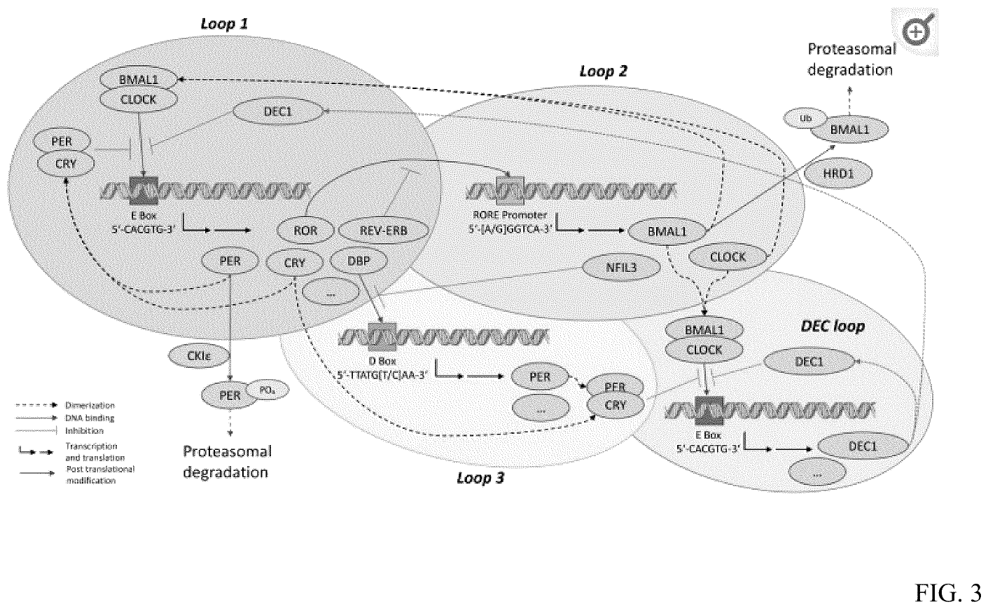

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, about 50% means in the range of 40%-60%.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “active” refers to the ingredient, component or constituent of the compositions of the described invention responsible for the intended cosmetic, cosmeceutical or therapeutic effect.

The term “adaptive immunity” as used herein refers to a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

“Administering” when used in conjunction with a cosmetic, cosmeceutical or therapeutic means to give or apply a cosmetic, cosmeceutical or therapeutic directly into or onto a target organ, tissue or cell, or to administer the cosmetic, cosmeceutical or therapeutic to a subject, whereby the cosmetic, cosmeceutical or therapeutic positively impacts the organ, tissue, cell, or subject to which it is targeted. Thus, as used herein, the term “administering”, when used in conjunction with EVs or compositions thereof, can include, but is not limited to, providing EVs into or onto the target organ, tissue or cell; or providing EVs systemically to a subject, whereby the therapeutic reaches the target organ, tissue or cell. “Administering” may be accomplished by oral, parenteral or topical administration or by such methods in combination with other known techniques.

The term “allogeneic” as used herein refers to being genetically different although belonging to or obtained from the same species.

The term “allograft” as used herein refers to a transplant of tissue from an allogeneic donor of the same species.

The term “allograft immunity” as used herein refers to any immune response by the host against a transplanted tissue.

The term “amino acid” is used to refer to an organic molecule containing both an amino group and a carboxyl group; those that serve as the building blocks of naturally occurring proteins are alpha amino acids, in which both the amino and carboxyl groups are linked to the same carbon atom. The terms “amino acid residue” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=lsoleucine; L=Leu=Leucine; K=Lys=Lysine; M=Met=Methionine; F=Phe=Phenyalanine; P=Pro=Proline; S=Ser-Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The following represent groups of amino acids that are conservative substitutions for one another:
Alanine (A), Serine (S), Threonine (T);
Aspartic Acid (D), Glutamic Acid (E);
Asparagine (N), Glutamine (Q);
Arginine (R), Lysine (K);
Isoleucine (I), Leucine (L), Methionine (M), Valine (V); and
Phenylalanine (F), Tyrosine (Y), Tryptophan (W).

The term “anesthetic agents” as used herein refers to agents that resulting in a reduction or loss of sensation. Non-limiting examples of anesthetic drugs that are suitable for use in the context of the described invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “anti-acne” as used herein refers to agents that alleviate the symptoms of acne. The term “acne” as used herein refers to an inflammatory disease of the sebaceous glands, characterized by comedones and pimples. Examples of anti-acne agents include, without limitation, keratolyses, such as salicylic acid, sulfur, glycolic, pyruvic acid, resorcinol, and N-acetylcysteine; and retinoids such as retinoic acid and its derivatives (e.g., cis and trans, esters).

The term “angiogenesis” as used herein refers to the formation of new blood vessels from pre-existing vasculature. Angiogenesis is central to a number of physiological conditions, from embryogenesis to wound healing, and is a hallmark of pathological conditions, such as tumorigenesis. [Abhinand, C S et al. J. Cell Commun. Signal. (2016) 10 (4): 347-54].

The terms “animal,” “patient,” and “subject” as used herein include, but are not limited to, humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals.

The term “annexins” as used herein refers to Ca+2-dependent proteins that bind to membrane phospholipids in plants. [Laohavisit, A. and Davies, J M. New Phytologist (2011) 189: 40-53]. They have been found to be stimulated by abiotic stress, including light, salinity, heat, cold, drought; oxidative and mechanic stress. [Saad, R B et al. Plant Signaling & Behavior (2020) 15 (1): e1699264; Baucher, M. et al. Plant Signaling & Behavior (2012) 7 (4): 524-8] Annexin expression also has been shown to be upregulated during pathogen attack or symbiotic interaction [Baucher, M. et al. Plant Signaling & Behavior (2012) 7 (4): 524-8, citing de Carvadho, N F, et al. Mol. Plant Microbe Interact. (1998) 11: 504-13; Manthey, K. et al. Mol. Plant Microbe Interact (2004) 17: 1063-77; Vandeputte, O. et al. Mol. Plant Pathol. (2007) 8: 185-94]. The dynamic properties of plant annexins together with their expression patterns suggest that they are involved in cell signaling processes and therefore in plant development al adaptation to environmental changes.

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetatnet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-dandruff agents” as used herein refers to agents that reduce, eliminate or prevent a scurf from forming on skin, especially of the scalp, that comes off in small white or grayish scales. Exemplary anti-dandruff ingredients usable in context of the described invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrate and any possible stereo isomers and derivatives thereof such as anthralin, piroctone olamine (Octopirox), selenium sulfide, and ciclopiroxolamine, and mixtures thereof.

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

The term “anti-irritant” as used herein refers to an agent that prevents or reduces soreness, roughness, or inflammation of a bodily part.

The term “anti-oxidant agent” as used herein refers to a substance that inhibits oxidation or reactions promoted by oxygen or peroxides. Non-limiting examples of antioxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, ó-hydroxy-ZjSjTjδ-tetramethylchroman-1-carboxylic acid (commercially available under the tradename Trolox®), gallic acid and its alkyl esters, especially propyl gallate, uric acid and its salts and alkyl esters, sorbic acid and its salts, lipoic acid, amines (e.g., N5N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®), sulfadiazine, and Leucovorin.

The term “antipruritic agents” as used herein refers to those substances that reduce, eliminate or prevent itching. Suitable antipruritic agents include, without limitation, pharmaceutically acceptable salts of methdilazine and trimeprazine.

The term “anti-skin atrophy actives” refers to substances effective in replenishing or rejuvenating the epidermal layer by promoting or maintaining the natural process of desquamation. The term “anti-wrinkle agent” as used herein an agent intended to reduce the appearance of wrinkles in the skin, e.g., retinols, Bakuchiol, vitamin C (ascorbic acid), niacinamide; tranexamic acid; azelaic acid.

Non-limiting examples of antiwrinkle and antiskin atrophy actives which can be used in context of the described invention include retinoic acid, its prodrugs and its derivatives (e.g., cis and trans) and analogues; salicylic acid and derivatives thereof, sulfur-containing D and L amino acids and their derivatives and salts, particularly the N-acetyl derivatives, an example of which is N-acetyl L-cysteine; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

The term “antigen” as used herein, is meant to refer to a molecule containing one or more antigenic determinants or epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response. These modifications may be deliberate, as through site-directed mutagenesis, or may be accidental, such as through mutations of the host that produces the antigens.

The term “antigen presentation” as used herein, generally refers to the display of antigen on the surface of a cell, e.g., in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system and thereby inducing an effective cellular immune response against the antigen or antigens being presented. Examples of professional APCs are dendritic cells and macrophages, though any cell expressing MHC Class I or II molecules can potentially present peptide antigen. An APC can be an irradiated population of PBMCs. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The term “Argonaute 2” or “AGO2” as used herein refers to an RNA binding protein that can shuttle between the cytoplasm and nucleus in a context-dependent fashion [Sharma, N R et al. J. Biol. Chem. (2016) 291: 2302-9] and is a key effector of RNA-silencing pathways. It is a major component of the RNA-induced silencing complex (RISC).

The term “astringents” are generally protein precipitants that have such low cell penetrability that the action essentially is limited to the cell surface and interstitial spaces. Astringents are locally applied. The astringent action is accompanied by contraction and wrinkling of the tissue and by blanching. Astringents are used therapeutically to arrest hemorrhage by coagulating the blood, to promote healing, to toughen the skin or to decrease sweating. The principal components of astringents are salts of aluminum, zinc, manganese, iron or bismuth.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “biomarker” (or “biosignature”) as used herein refers to a peptide, a protein, a nucleic acid, an antibody, a gene, a metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “cargo” as used herein refers to a load or that which is conveyed. With respect to exosomes and/or extracellular vesicles, the term cargo refers to a substance encapsulated in the exosome and or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or an extracellular vesicle.

The term “cargo profile” as used herein refers to measurements of cargo components that characterize a population of extracellular vesicles.

The term “carrier” as used herein describes a material that does not cause significant irritation to a mammal and does not abrogate the biological activity and properties of the actives of the composition. Carriers must be of sufficiently high purity and of sufficiently low toxicity to render them suitable for administration to the mammal being treated. The carrier can be inert, or it can possess pharmaceutical benefits, cosmetic benefits or both.

The term “caustic agents” as used herein refers to substances capable of destroying or eating away epithelial tissue by chemical action. Caustic agents can be used to remove dead skin cells. For example, beta-hydroxy acids, naturally derived acids with a strong kerolytic effect, are useful for problem skin, acne or peeling.

“Cluster of Differentiation” or “cluster of designation” (CD) molecules are utilized in cell sorting using various methods, including flow cytometry. Cell populations usually are defined using a “+” or a “−” symbol to indicate whether a certain cell fraction expresses or lacks a particular CD molecule.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences.

The term “compatible” as used herein means that the components of a composition are capable of being combined with each other in a manner such that there is no interaction that would substantially reduce the efficacy of the composition under ordinary use conditions.

The term “complementary DNA” or “cDNA” as used herein refers to synthetic DNA that has been transcribed from a specific mRNA through a reaction using the enzyme reverse transcriptase. While DNA is composed of both coding and non-coding sequences, cDNA contains only coding sequences.

The term “condition” as used herein includes a variety of conditions related to skin or mucosal membranes. This term is meant to include disorders or diseases, the promotion of healthy epithelium; dry skin; and inflammation caused by any underlying mechanism or disorder.

The term “conditioned medium” (or plural, media), as used herein refers to spent culture medium harvested from cultured cells containing metabolites, growth factors, RNA and proteins released into the medium by the cultured cells.

The term “contact” and its various grammatical forms as used herein refers to a state or condition of touching or of immediate or local proximity.

The term “cosmeceutical” as used herein refers to a topical preparation sold as a cosmetic that has performance characteristics that affect the skin positively beyond the time of its application.

The term “cosmetic composition” as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The terms “cosmetic signature”, cosmeceutical signature” and “therapeutic signature” as used herein respectively refer to a specific and complex combination of biomarkers that reflect a biological state that leads to a specific cosmetic, cosmeceutical or therapeutic effect.

The term “cosmetically acceptable carrier” as used herein refers to a substantially non-toxic carrier, conventionally useable for the topical administration of cosmetics, with which compounds will remain stable and bioavailable.

The terms “cosmetic amount”, cosmeceutical amount” or “pharmaceutical amount” as used herein refer to the amount of any of the compositions of the invention that result in a beneficial effect following administration to a subject. The cosmetic, cosmeceutical, or pharmaceutical effect can improve physical appearance and aesthetics, treating, a condition, disease or disorder, or any other beneficial effect. The concentration of the active(s) is selected so as to exert its cosmetic, cosmeceutical or pharmaceutical effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound(s), composition or other active ingredient(s) employed, the particular carrier utilized, and like factors. A skilled artisan can determine an effective amount of the inventive compositions by determining the unit dose. As used herein, a “unit dose” refers to the amount of inventive composition required to produce a response of 50% of maximal effect (i.e. ED50). The unit dose can be assessed by extrapolating from dose-response curves derived from in vitro or animal model test systems.

The term “cross-dressing” as used herein refers to a third pathway for cross-presentation. In cross-dressing, dendritic cells acquire preformed MHC class I molecules in complex with antigens from other cells by the process of trogocytotis (meaning the transfer of cell membrane patches or individual proteins between cells [Yewdell, J W and Dolan, BP, Nature (2011) 471 (7340): 581-582, citing Joly, E. and Hudrisier, D. Nature Immunol. (2003) 4: 815; Herrera O B et al. J. Imunol. (2004) 173: 4828-4837] or through gap junctions. This allows antigen presentation by acceptor dendritic cells to occur immediately, without any processing. Cross-dressing is used to activate memory T cells, but not naïve T cells, in response to viral infection [Id., citing Wakins, L M and Bevan, MJ. Nature (2011) 471: 629-632].

The term “cross-presentation” as used herein refers to a process by which proteins taken up by dendritic cells from the extracellular milieu can give rise to peptides presented by MHC class I molecules. It enables antigens from extracellular sources to be presented by MHC class I molecules and to activate CD8 T cells.

The term “cross-priming” as used herein refers to activation of CD8 T cells by dendritic cells in which the antigenic peptide presented by MHC class I molecules is derived from an exogenous protein (i.e., by cross-presentation), rather than produced within the dendritic cells directly (compare direct presentation).

The term “culture medium” (or plural, media), as used herein refers to a substance containing nutrients in which cells or tissues are cultivated for controlled growth.

The term “cytokine” as used herein refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (TNF)-related molecules, including TNFα and lymphotoxin; immunoglobulin super-family members, including interleukin 1 (IL-1); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term damage-associated molecular patterns” or “DAMPS” as used herein refers to molecules released by stressed or dying cells that bind to pattern recognition molecules (PRMs) and induce inflammation.

The term “dendritic cells (DC)” as used herein refers to professional antigen presenting cells, which induce naïve T cell activation and effector differentiation. [Patente, T A, et al., Frontiers Immunol. (2019) doi.org/10.3389/fimmu.2018.03176]. Human DC are identified by their high expression of major histocompatibility complex (MHC) class II molecules (MHC-II) and of CD11c, both of which are found on other cells, like lymphocytes, monocytes and macrophages [Id., citing Carlens J, et al. J Immunol. (2009) 183:5600-5607; Drutman S B, et al. J Immunol. (2012) 188:3603-3610; Hochweller K, S et al. Eur J Immunol. (2008) 38:2776-2783; Huleatt J W, Lefrangois L. J Immunol. (1995) 154:5684-5693; Rubtsov A V, et al. Blood (2011) 118:1305-15; Probst H C, et al. Clin Exp Immunol. (2005) 141:398-404; Vermaelen K, Pauwels R. Cytometry (2004) 61A:170-177].

As used herein, the term “derived from” is meant to encompass any method for receiving, obtaining, or modifying something from a source of origin.

As used herein, the terms “detecting”, “determining”, and their other grammatical forms, are used to refer to methods performed for the identification or quantification of a biomarker, such as, for example, the presence or level of miRNA, or for the presence or absence of a condition in a biological sample. The amount of biomarker expression or activity detected in the sample can be none or below the level of detection of the assay or method.

The term “differentiation” as used herein refers to a process of development with an increase in the level of organization or complexity of a cell or tissue, accompanied by a more specialized function.

The term “direct presentation” as used herein refers to a process by which proteins produced within a given cell give rise to peptides presented by MHC class I molecules. This may refer to APCs (such as dendritic cells), or to nonimmune cells that will become the targets of CTLs.

When used to describe the expression of a gene or polynucleotide sequence, the terms “down-regulation”, “disruption”, “inhibition”, “inactivation”, and “silencing” are used interchangeably herein to refer to instances when the transcription of the polynucleotide sequence is reduced or eliminated. This results in the reduction or elimination of RNA transcripts from the polynucleotide sequence, which results in a reduction or elimination of protein expression derived from the polynucleotide sequence (if the gene comprised an ORF). Alternatively, down-regulation can refer to instances where protein translation from transcripts produced by the polynucleotide sequence is reduced or eliminated. Alternatively, still, down-regulation can refer to instances where a protein expressed by the polynucleotide sequence has reduced activity. The reduction in any of the above processes (transcription, translation, protein activity) in a cell can be by about 20%, 30%, 40%, 50%, 60%, 70%, 80%, 90%, 95%, or 100% relative to the transcription, translation, or protein activity of a suitable control cell. Down-regulation can be the result of a targeting event as disclosed herein (e.g., indel, knock-out), for example.

The term “Drosha” as used herein refers to a nuclear RNase III that cleaves primary miRNAs to release hairpin-shaped pre-miRNAs that are subsequently cut by the cytoplasmic RNase III Dicer to generate mature miRNAs.

The terms “drying agent” or “desiccant” as used herein refers to a substance that has an affinity for water such that it will extract the water from other materials.

The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds. Common emollients used in the manufacture of cosmetics include, but are not limited to, butters, such as Aloe Butter, Almond Butter, Avocado Butter, Cocoa Butter, Coffee Butter, Hemp Seed Butter, Kokum Butter, Mango Butter, Mowrah Butter, Olive Butter, Sal Butter, Shea Butter, glycerin, and oils, such as Almond Oil, Aloe vera Oil, Apricot Kernel Oil, Avocado Oil, Babassu Oil, Black Cumin Seed Oil, Borage Seed Oil, Brazil Nut Oil, Camellia Oil, Castor Oil, Coconut Oil, Emu Oil, Evening Primrose Seed Oil, Flaxseed Oil, Grape Seed Oil, Hazelnut Oil, Hemp Seed Oil, Jojoba Oil, Kukui Nut Oil, Macadamia Nut Oil, Meadowfoam Seed Oil, Mineral Oil, Neem Seed Oil, Olive Oil, Palm Oil, Palm Kernel Oil, Peach Kernel Oil, Peanut Oil, Plum Kernel Oil, Pomegranate Seed Oil, Poppy Seed Oil, Pumpkin Seed Oil, Rice Bran Oil, Rosehip Seed Oil, Safflower Oil, Sea Buckthorn Oil, Sesame Seed Oil, Shea Nut Oil, Soybean Oil, Sunflower Oil, Tamanu Oil, Turkey Red Oil, Walnut Oil, Wheatgerm Oil

As used herein “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent. Common types of emulsions are oil-in-water, where oil is the dispersed liquid and an aqueous solution, such as water, is the dispersion medium, and water-in-oil, where, conversely, an aqueous solution is the dispersed phase. It also is possible to prepare emulsions that are nonaqueous. Creams of the oil-in-water type include hand creams and foundation creams. Water-in-oil creams include cold creams and emollient creams.

Creams may be diluted only with suitable diluents specified in the appropriate entries, and diluted creams must be freshly prepared without the application of heat. Creams should be stored in a cool place and supplied in well-closed containers that prevent evaporation and contamination of the contents. When making a natural cream, however, butters first are melted. The vessel is removed from the heat and the oils are added. When the solution is 100 degrees F., the balance of the liquid portion of the formula then is slowly added while continuously stirred.

The term “endogenous” as used herein refers to that which is naturally occurring, incorporated within, housed within, adherent to, attached to, or resident in.

As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

The term “entrainment” as used herein refers to a process of coordinating the internal circadian clock to external rhythmic time-cues (Zeitgeber), mainly light. The organization of circadian phases is dependent on the geographical and seasonal context and individual clock properties (personal chronotypes). [Schmal, C. et al. Front. Physiol. (2020) 11: 272].

The term “ESCRT machinery” as used herein refers to an evolutionarily conserved, multi-subunit membrane remodeling complex originally identified in yeast for its essential role in the biogenesis of intraluminal vesicles (ILVs) upon a class of endosome called the multivesicular body (MVB), whose role in mammalian cells includes a number of topologically equivalent membrane modeling events, see Olmos, Y., Oarlton, JG. Curr. Opin. Cell Biol. (2016) 38: 1-11, citing Katzmann, D J., et al. Cell (2001) 106: 145-155; Babst, M., et al. Dev. Cell (2002) 3: 283-289; Babst, M., et al., Dev. Cell (2002) 3: 272-282]

The term “exogenous” as used herein refers to that which is non-naturally occurring, or that is originating or produced outside of a specific EV, cell, organism, or species.

The term “expand” and its various grammatical forms as used herein refers to a process by which dispersed living cells propagate in vitro in a culture medium that results in an increase in the number or the amount of viable cells.

As used herein, the term “expression” and its various grammatical forms refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

The term “extracellular vesicles (EVs)” as used herein refers to nanosized, membrane-bound vesicles released from mammalian cells that can transport cargo-including DNA, RNA, and proteins-between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (m) in size, and contain cytoplasmic cargo (Zaborowski, M P., et al. BioScience (2015) 65 (8): 783-797, citing Heijnen, H F., et al. Blood (1999) 94: 3791-3799). Another EV subtype, exosomes, is formed by the fusion between multivesicular bodies and the plasma membrane, by which multivesicular bodies release smaller vesicles (exosomes) whose diameters range from 40 to 120 nm (Id., citing El Andaloussi, S., et al. Nature Reviews Drug Discovery (2013) 12: 347-357; Cocucci, E. and Meldolesi J. Trends in Cell Biology (2015) 25: 364-372). Dying cells, release vesicular apoptotic bodies (50 nm-2 m) that can be more abundant than exosomes and or extracellular vesicles or MVs under specific conditions and can vary in content between biofluids (Id., citing Thery, C., et al. J. Immunology (2001) 1666: 7309-7318; El Andaloussi, S., et al. Nature Reviews Drug Discovery (2013) 12: 347-357). Membrane protrusions can also give rise to large EVs, termed oncosomes (1-10 m), which are produced primarily by malignant cells in contrast to their nontransformed counterparts (Id., citing Di Vizio, D., et al. Am. J. Pathol. (2012) 181: 1573-1584; Morello, M., et al. Cell Cycle (2013) 12: 3526-3536).

The terms “formulation” and “composition” are used interchangeably herein to refer to a product of the described invention that comprises all active and inert ingredients.

As used herein the term “fragment” and its other grammatical forms are meant to refer to portions of a nucleic acid, polynucleotide or oligonucleotide shorter than the full sequence of a reference molecule. The sequence of bases in a fragment is unaltered from the sequence of the corresponding portion in the molecule from which it arose; there are no insertions or deletions in a fragment in comparison with the corresponding portion of the molecule from which it arose. As contemplated herein, a fragment of a nucleic acid or polynucleotide, such as an oligonucleotide, is 15 or more bases in length, or 16 or more, 17 or more, 18 or more, or 19 or more, or 20 or more, or 21 or more, or 22 or more, or 23 or more, or 24 or more, or 25 or more, or 26 or more, or 27 or more, or 28 or more, or 29 or more, 30 or more, 50 or more, 75 or more, 100 or more bases in length, up to a length that is one base shorter than the full length sequence.

The term “free radical” as used herein refers to a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms. Oxidative injury may lead to widespread biochemical damage within the cell. The molecular mechanisms responsible for this damage are complex. For example, free radicals may damage intracellular macromolecules, such as nucleic acids (e.g., DNA and RNA), proteins, and lipids. Free radical damage to cellular proteins may lead to loss of enzymatic function and cell death. Free radical damage to DNA may cause problems in replication or transcription, leading to cell death or uncontrolled cell growth. Free radical damage to cell membrane lipids may cause the damaged membranes to lose their ability to transport oxygen, nutrients or water to cells.

The term “gene” as used herein refers to a DNA polynucleotide sequence that expresses an RNA (RNA is transcribed from the DNA polynucleotide sequence) from a coding region, which RNA can be a messenger RNA (encoding a protein) or a non-protein-coding RNA. A “gene” can refer to the coding region alone or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source but arranged in a manner different than that found in nature. A “foreign” or “heterologous” gene refers to a gene that is introduced into the host organism by gene transfer. Foreign/heterologous genes can comprise native genes inserted into a non-native organism, native genes introduced into a new location within the native host, or chimeric genes. A homologous gene is a type of gene that is inherited by two different species that evolved from the same ancestor. A paralogue gene is one of a set of homologous genes that have diverged from each other as a consequence of genetic duplication. A “transgene” is a gene that has been introduced into the genome by a gene delivery procedure (e.g., transformation). A “codon-optimized” open reading frame has its frequency of codon usage designed to mimic the frequency of preferred codon usage of the host cell.

The term “growth factor” as used herein refers to extracellular polypeptide molecules that bind to a cell-surface receptor triggering an intracellular signaling pathway, leading to proliferation, differentiation, or other cellular response. These pathways stimulate the accumulation of proteins and other macromolecules, e.g., by increasing their rate of synthesis, decreasing their rate of degradation, or both.

The term “hair health” as used herein refers to shiny hair with a smooth texture and clean-cut ends or tapered tips. Hair consists of an outer hydrophobic lipid epicuticle, a layer of flattened overlapping cuticle cells surrounding the elongated polyhedral cortical cells. The normal cuticle, which has a smooth appearance, allowing light reflection and limiting friction between the hair shafts, is responsible for the luster and texture of the hair [Sinclair, RD. J. Investig. Dermatology Symposium Proceedings (2007) 12: 2-5, citing Draelos, ZD. Dermatol. Clin. (1991) 9: 199-227]. The cortical layer determines many of the mechanical properties of the hair. It consists of closely packed spindle-shaped cortical cells filled with keratin filaments that are orientated parallel to the longitudinal axis of the hair shaft, and an amorphous matrix of high sulfur proteins [Id., citing Dawber, R. Clin. Dermatol. (1996) 4: 105-112]. The outer, intensely hydrophobic layer and the cortex confer the physical properties of luster (shine) and volume (body) that contribute to the appearance of hair health.

The term “heat shock proteins” or “HSPs” as used herein refers to highly conserved multimolecular complexes expressed constitutively under normal growth conditions in cells that act as molecular chaperones, which play a regulatory role in the folding of proteins, intracellular transport of proteins in cytosol, endoplasmic reticulum and mitochondria, repair or degradation of proteins and refolding of misfolded proteins. In addition to being constitutively expressed, these proteins can be induced by a range of environmental, pathological, or physiological stimuli.

The term “homolog” as used herein refers to being similar in form or structure, but not necessarily in function; homology suggests evolutionary relatedness.

The term “hormone” as used herein refers to natural substances produced by organs of the body that travel by blood to trigger activity in other locations or their synthetic analogs. Suitable hormones for use in the context of the described invention include, but are not limited to, calciferol (Vitamin D3) and its products.

The term “hypopigmenting agents” as used herein refers to substances capable of depigmenting the skin. Suitable hypopigmenting agents include, but are not limited to, hydroquinones, mequinol, and various protease inhibitors including serine protease inhibitors, active soy and retinoic acid.

The terms “immune response” and “immune-mediated” are used interchangeably herein to refer to any functional expression of a subject's immune system, against either foreign or self-antigens, whether the consequences of these reactions are beneficial or harmful to the subject.

The term “immune system” as used herein refers to a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other but rather work together to elicit effective immune responses.

The term “immunogen” and its various grammatical forms as used herein refers to a substance that elicits an immune response

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

The term “inflammasome” as used herein refers to a pro-inflammatory protein complex that is formed after stimulation of the intracellular nucleotide-binding oligomerization domain (NOD)-like receptors. Production of an active caspase in the complex processes cytokine proteins into active cytokines.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory cytokines” or “inflammatory mediators” as used herein refers to the molecular mediators of the inflammatory process, which may modulate being either pro- or anti-inflammatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

The term “innate immunity” as used herein refers to a nonspecific fast response to pathogens that is predominantly responsible for an initial inflammatory response before adaptive immunity is induced. These include such mechanisms as anatomical barriers, antimicrobial peptides, the complement system and the chemokine/cytokine system; macrophages and neutrophils carrying nonspecific pathogen-recognition receptors, and a number of specialized cell types, including innate lymphoid cells (ILCs, including natural killer (NK) cells) mast cells and dendritic cells (DCs). Innate immunity is present in all individuals at all times, does not increase with repeated exposure to a given pathogen, and discriminates between groups of similar pathogens, rather than responding to a specific pathogen.

The term “insulin-like growth factor 1” or IGF-1” as used herein refers to a hormone similar in molecular structure to insulin that has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase non-receptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling. IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 can regulate cell growth and development, especially in nerve cells, as well as cellular DNA synthesis. IGF-1 was shown to increase the expression levels of the chemokine receptor CXCR4 (receptor for stromal cell-derived factor-1, SDF-1) and to markedly increase the migratory response of mesenchymal stem cells (MSCs) to SDF-1 (Li, Y, et al. 2007 Biochem. Biophys. Res. Communic. 356(3): 780-784). The IGF-1-induced increase in MSC migration in response to SDF-1 was attenuated by PI3 kinase inhibitor (LY294002 and wortmannin) but not by mitogen-activated protein/ERK kinase inhibitor PD98059.

The term “integrin” as used herein refers to heterodimeric cell surface proteins involved in cell-cell and cell-matrix interactions. They are important in adhesive interactions between lymphocytes and antigen-presenting cells and in lymphocyte and leukocyte adherence to blood vessel walls and migration into tissues.

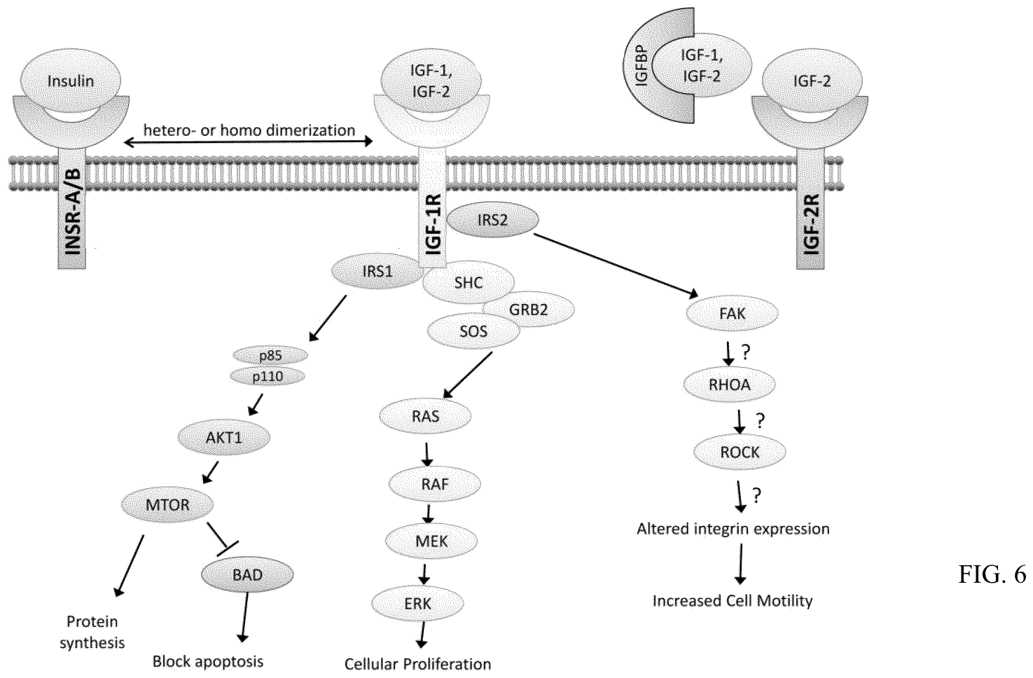

The term “Insulin-Like Growth Factor (IGF) signaling pathway” refers to a complex and tightly regulated network which is critical for cell proliferation and survival [Jams, W T and Lovly, CM. Clin. Cancer Res. (2015) 21 (19): 4270-77, citing (1). This pathway (FIG. 6) is composed of three receptor tyrosine kinases—insulin-like growth factor-1 receptor (IGF-1R), insulin-like growth factor-2 receptor (IGF-2R), and insulin receptor (INSR); three ligands—insulin, IGF-1, and IGF-2 (Id, citing 2, 3); and six serum Insulin-like Growth Factor Binding Proteins (IGFBP's), which serve as regulators of the pathway by determining ligand bioavailability (Id., citing 4). The most prevalent of the IGFBP's is IGFBP3 (5). Both IGF-1 and IGF-2 exert their effects through autocrine, paracrine, and endocrine mechanisms, and both can activate IGF-1R signaling. For simplification, IGF-1 ligand only is shown binding to IGF-1R. IGF-1 binding to IGF-1R promotes receptor homodimerization or heterodimerization with INSR. Ligand-activated IGF-1R first binds to intracellular adaptor proteins, such as insulin receptor substrate1 (IRS1) and SHC. These adaptor proteins transmit signals through the phosphatidyl-inositol-3 kinase (PI3K)-AKT1-mammalian target of rapamycin (MTOR) pathway and through the mitogen activated protein kinase (MAPK) pathway. Activated IGF-1R promotes cellular motility through activation of IRS2, which alters integrin expression through poorly understood mechanisms involving the small G protein RHOA, focal adhesion kinase (FAK), Rho-kinase (ROCK), PI3K, and other signaling molecules. Targets for potential monotherapy and combinatorial therapeutic strategies are noted in the figure. TKI: tyrosine kinase inhibitor. mAb: monoclonal antibody.

The term “interferons” (“IFNs”) as used herein refers to several related families of cytokines originally named for their interference of viral replication. IFN-α and IFN-β are antiviral in their effects; IFN-γ has other roles in the immune system.

The term “IFN-α, IFN-β” and “type I interferons” as used herein refers to antiviral cytokines produced by a wide variety of cells in response to infection by a virus and which also help healthy cells resist viral infection. They act through the same receptor, which signals through a Janus-family tyrosine kinase. Type I interferons are inducible and are synthesized by many cell types after infection by diverse viruses. Almost all types of cells can produce IFN-α and IFN-β in response to activation of several innate sensors. The major producers of IFN-α and -β are macrophages and DCs. [Janeway's Immunology, 9th Ed. (2017) Garland Science, New York, at 122-125]

The term “IFNγ” as used herein refers to a cytokine of the interferon structural family produced by effector CD4TH1 cells, CD8 T cells, and NK cells. Its primary function is the activation of macrophages. It acts through a different receptor from that of the type I interferons. IFN-7 is produced by activated Th1 cells and ILC1s, including NK cells, which are activated mainly through TLRs.

The term “IFNλ” or “type III interferons” as used herein refers to a family that includes IL-28A, IL-28B, and IL-29, which bind a common receptor expressed by a limited set of epithelial tissues.

The term “interferon regulatory factor (TRF) as used herein refers to a family of nine transcription factors that regulate a variety of immune responses. For example, IRF3 and IRF7 are activated as a result of signaling from some TLRs. Several IRFs promote expression of the genes for type 1 interferons.

The term “interferon stimulating genes” or “ISGs” as used herein refers to a category of gene induced by interferons, which include many that promote innate defense against pathogens, such as oligoadenylate synthetase, PDR and the Mx, IFITs and IFITM proteins.

The term “interferon α receptor” or “IFNAR” as used herein refers to a receptor that recognizes IFN-α and IFN-β to activate STAT1 and STAT2 and induce expression of many ISGs.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin 2 (IL-2), interleukin-1β (IL-1β), interleukin-6 (IL-6), interleukin-8 (IL-8), and interleukin-12 (IL-12).

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “Janus kinase (JAK) family” as used herein refers to enzymes of the JAK-STAT intracellular signaling pathways that link many cytokine receptors with gene transcription in the nucleus. The kinases phosphorylate STAT proteins in the cytosol, which then move to the nucleus and activate a variety of genes.

The term “keratolytics” (desquamating agents) as used herein refers to an agent that acts to remove outer layers of the stratum corneum. Keratolytics are particularly useful in hyperkeratotic areas. The keratolytics include, but are not limited to, benzoyl peroxide, fluorouracil, resorcinol, salicylic acid, tretinoin, and the like.

The term “leptin” as used herein refers to a protein hormone secreted by adipose tissue. The function of leptin is multifaceted, mainly in the regulation of fat and body weight; appetite suppression; increased energy consumption; direct inhibition of fat synthesis and promote its decomposition. Insulin can promote the secretion of leptin; leptin decreases insulin synthesis and secretion by pancreatic beta cells and increases insulin hepatic extraction. As a result, insulin delivery is reduced by leptin. The secretion of leptin has a circadian rhythm, which is characterized by high evening, low morning, and impulsive secretion. Leptin exerts its actions by binding to leptin receptors in various tissues, especially in the CNS. Activation of leptin receptor (LepR) with leptin activates signaling modules such as the Janus kinase/signal transducer and activator of transcription (JAK/STAT) pathway, phosphoinositide 3-kinase (PI3K pathway), the mitogen-activated protein kinase (MAPK) pathway; the extracellular signaling-regulated kinase ½ (ERK1/2) pathway; the adenosine monophosphate kinase (AMPK) pathway; and the PPAR gamma coactivator/peroxisome proliferator-activated receptor (PGC/PPAR) pathway. Tyrosine phosphorylation of long leptin receptor isoform LEPRb induces binding of STATs to LEPRb. Binding of STATs to the phosphorylated residues of LEPR leads to the JAK2 mediated tyrosine phosphorylation and activation of STATs. Activated STATs translocate to the nucleus and induces expression of genes such as suppressor of cytokine signaling 3 (SOCS3) and TIMP metallopeptidase inhibitor 1 (TIMP1). SOCS3 mediates feedback inhibition of leptin pathway by binding to Tyr-986 residue of LEPR. [Liu, Z., et al. Front. Neurosci. (2023) 17: 1238528].

The term “long noncoding RNA” (“incRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A., et al. Front. Physiol. (2018) doi.org/10.3389/fphys.2018.01201, citing Yoon, J H., et al. Semin. Cell Dev. Bio. (2014) 34: 9-14; Ballantyne, MD., et al. Clin. Pharmacol. Ther. (2016) 99: 494-501). Finally, lncRNAs may contain embedded miRNA sequences and serve as a source of miRNAs (Id., citing Piccoli, M T., et al. Cir. Res. (2017) 121: 575-583).

The term “low density lipoprotein receptor related protein 1” or “LRP1” as used herein refers to a transmembrane receptor protein belonging to the LDL receptor family, which plays multifunctional roles in maintaining endocytosis, homeostasis and signal transduction. Accumulating evidence suggests that LRP1 modulates vascular homeostasis mainly by regulating vasoactive substances and specific intracellular signaling pathways, including the plasminogen activator inhibitor 1 (PAI-1) signaling pathway, platelet-derived growth factor (PDGF) signaling pathway, transforming growth factor-β (TGF-β) signaling pathway and vascular endothelial growth factor (VEGF) signaling pathway. [He, Z. et al. Biomedicine & Pharmacotherapy (2021) 139: 111667].

The term “macrophage” as used herein refers to a mononuclear, actively phagocytic cell arising from monocyte stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Macrophages have been classified based on their mode of activation: classically activated/M1 macrophages respond to interferon-gamma (IFN-γ) by releasing pro-inflammatory cytokines and are involved in TH1 cell mediated resolution of acute infection. Alternatively activated/M2 macrophages respond to cytokines from TH2 cells and are involved in wounding and fibrosis. [Ghajar, C M., et al., “The role of the microenvironment in tumor initiation, progression, and metastasis,” In Mendelsohn, J., et al., the Molecular Basis of Cancer, Elsevier Saunders, Philadelphia, citing Pollard, J W, Nat. Rev. Immunol. (2009) 9: 259-270]. The diverse functions of macrophages are executed in a tissue- and context-specific fashion by a number of discrete macrophage subtypes, which aid these developmental processes by remodeling collagen and secreting a host of pro-angiogenic, pro-inflammatory and matrix-degrading factors (Id., citing Qian, BZ, Pollard, J W. Cell (2010) 141: 39-51).

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups—class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1:TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyze peptide binding to MHC class II molecules are related to the MHC class II genes, as are the DOA and DOB genes that encode the subunits of the regulatory HLA-DO molecule. [Janeways Immunobiology. 9th ed., GS, Garland Science, Taylor & Francis Group, 2017. pps. 232-233]. MHC-like molecules, while not encoded by the same gene group as true MHCs, have the same folding and overall structure of MHCs, and specifically MHC class I molecules, and thus possesses similar biological functions such as antigen presentation. MHC Class I-like molecules are nonclassical MHC type molecules, while including Cd1d also include CD1a, CD1b, CD1c, CD1e, and MR1 are also expressed on APCs and can activate various subsets of T cells. [Kumar and Delovitch (2014) “Different subsets of natural killer T cells may vary in their roles in health and disease.” Immunology 142: 321-336]. Other non-classical histocompatibility molecules include MR1, which activate MAIT cells.

The term “mammalian exosomes” as used herein refers to extracellular bilayered membrane-bound vesicles of endosomal origin in a size range of −40 to 160 nm in diameter (˜100 nm on average) generated by all mammalian cells that are actively secreted.

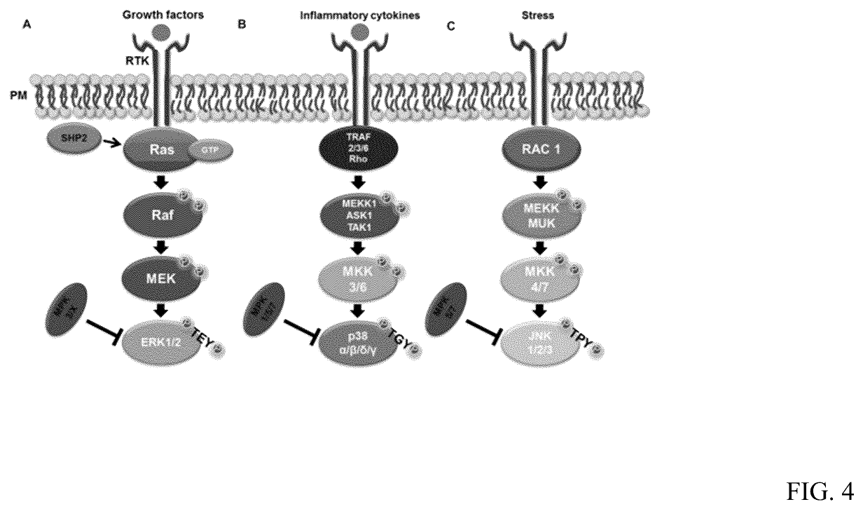

The abbreviation “MAPK” as used herein refers to Mitogen-Activated Protein Kinase (MAPK) signaling, which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses. [Cargnello, M. and Roux, PP, Microbiol. Mol. Biol. Rev. (2011) 75(1): 50-83]. FIG. 4 is a schematic of the MAPK signaling pathways. [taken from Soares-Silva, M., et al. Front. Microbiol. (2016) 7: 183]. The major MAPK pathways involved in inflammatory diseases are extracellular regulating kinase (ERK), p38 MAPK, and c-Jun NH2-terminal kinase (JNK). All three MAPK pathways may be activated by TGF-β, and signaling through these cascades can further regulate the expression of Smad proteins and mediate Smad-independent TGF-β responses. These three MAPK pathways are all involved in TGF-β-induced fibrosis. [He, W. and Dai, C. Curr. Pathobiol. Rep. (2015) 3: 183-92, citing Tsou, P S., et al. Am. J. Physiol. Cell Physiol. (22014) 307: C2-C13; Kamato, D., et al. Cell Signal (2013) 25: 2017-2024; Pannu, J., et al. J. Biol. Chem. (2007) 282: 10405-10413; Yu, L., et al. J. Biol. Chem. (2002) EMBO J. 21: 3749-3759]. Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-β can signal in a noncanonical manner via the MAPK family.

The term “matrix metalloproteinases” as used herein refers to a collection of zinc-dependent proteases involved in the breakdown and the remodeling of extracellular matrix components (Guiot, J., et al. Lung (2017) 195(3): 273-280, citing Oikonomidi, et al. Curr Med Chem. 2009; 16(10): 1214-1228). MMP-1 and MMP-7 seem to be primarily overexpressed in plasma of IPF patients compared to hypersensitivity pneumonitis, sarcoidosis and COPD with a possible usefulness in differential diagnosis (Id., citing Rosas I O., et al. PLoS Med. 2008; 5(4): e93). They are also involved in inflammation and seem to take part to the pathophysiological process of pulmonary fibrosis (Id., citing Vij R, Noth I. Transl Res. 2012; 159(4): 218-227; Dancer R C A., et al. Eur Respir J. 2011; 38(6): 1461-1467). The most studied is MMP-7, which is known as being significantly increased in epithelial cells both at the gene and protein levels and is considered to be active in hyperplastic epithelial cells and alveolar macrophages in IPF (Id., citing Fujishima S., et al. Arch Pathol Lab Med. (2010) 134(8): 1136-1142). There is also a significant correlation between higher MMP-7 concentrations and disease severity assessed by forced vital capacity (FVC) and diffusing capacity of the lungs for carbon monoxide (DLCO) (Id., citing Rosas I O., et al. PLoS Med. 2008; 5(4): e93). Higher levels associated to disease progression and worse survival (>4.3 ng/ml for MMP-7) (Id.). The MMP2 gene provides instructions for making matrix metallopeptidase 2. This enzyme is produced in cells throughout the body and becomes part of the extracellular matrix, which is an intricate lattice of proteins and other molecules that forms in the spaces between cells. One of the major known functions of MMP-2 is to cleave type IV collagen, which is a major structural component of basement membranes, the thin, sheet-like structures that separate and support cells as part of the extracellular matrix.

MMPs play a critical role in neuroinflammation through the cleavage of ECM proteins, cytokines and chemokines. (Ji. R-R., et al, US Neurology, Touch Briefings (2008) 71-74). MMP-2 is constitutively expressed and normally present in brain and spinal cord tissues. In contrast, MMP-9 is normally expressed at low levels, but upregulated in many injury and disease states such as spinal cord injury and brain trauma (Id., citing Rosenberg, GA. Glia (2002) 39: 279-291); it is also induced in the crushed sciatic nerve and causes demyelination, a condition associated with neuropathic pain, by the cleavage of myelin basic protein. (Id., citing Chattopadhyay, S., et al. Brain Behav. Immun. (20007) 21: 561-568). Besides targeting matrix, because MMPs can process a variety of growth factors and other extracellular cytokines and signals, they may contribute to the neurovascular remodeling that accompanies chronic CNS injury. (Id., citing Zhao, B Q., et al. Nat. Med. (2006) 12: 441-445).

The term “microRNA,” “miRNA”, or “miR” as used herein refers to a class of small, non-coding RNA molecules, usually from about 18 to about 28 nucleotides in length. MicroRNAs are partially complementary to one or more messenger RNA (mRNA) molecules, and function in posttranscriptional regulation of gene expression and RNA silencing. A precursor microRNA (miRNA) has two arms: miR-5p and miR-3p (miR-5p/-3p). In the precursor miRNA stem loop structure, the 5p strand is present in the forward (5′-3′) position and the 3p strand (which will be almost complimentary to the 5p strand) is located in the reverse position. In terms of which strand is functional following Dicer cleavage of the stem loop to produce the two mature strands, either the 5p or 3p strand or both can become functional depending on the tissue or cell types. The stability of the mature strand may influence its function and ability to enter the RISC complex to then bind to its target gene—in general, the more stable strand will be functional, and less stable strand will be degraded.

The term “modulate” as used herein means to regulate, alter, adapt, or adjust to a certain measure or proportion.

The term “moisturizing agent” as used herein refers to a substance that adds or restores moisture to the skin.

The term “myeloid” as used herein means of or pertaining to bone marrow. Granulocytes and monocytes, collectively called myeloid cells, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity. [Kawamoto, H. and Minato, N. Intl J. Biochem. Cell Biol. (2004) 36 (8): 1374-1379].

The term “Next Generation Sequencing” or “NGS” as used herein refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

The term “non-steroidal anti-inflammatory agents” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopmac, zidometacin, acematacin, fentiazac, zomepirac, clindanac, oxepinac, felbinac, and ketorolac; fenamates, such as mefenamic, meclofenamic, flufenamic, nifiumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alrninoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

The term “normal healthy subject” as used herein refers to a subject having no symptoms or other evidence of a dermatological condition.

The term “Nucleotide-binding Oligomerization Domain (NOD)-like receptors (NLRs)” as used herein refers to innate sensors that detect microbial products or cellular damage in the cytoplasm or activate signaling pathways and are expressed in cells that are routinely exposed to bacteria, such as epithelial cells, macrophages and dendritic cells. Some NLRs activate NFκB to initiate the same inflammatory responses as the TLRs, while others trigger a distinct pathway that induces cell death and the production of pro-inflammatory cytokines. [Janeway's Immunobiology. 9th ed., GS, Garland Science, Taylor & Francis Group, 2017, at 96].

Subfamilies of NLRs can be distinguished based on the other protein domains they contain. For example, the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins. [Janeway's Immunobiology. 9th ed., GS, Garland Science, Taylor & Francis Group, 2017, at 96]. NOD1 senses 7-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood. [Janeway's Immunobiology. 9th ed., GS, Garland Science, Taylor & Francis Group, 2017, at 96-98]

When NOD1 or NOD2 recognizes its ligand, it recruits the CARD-containing serine-threonine kinase RIP2 (also known as RICK and RIPK2), which associates with the E3 ligases cIAP1, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO. [Janeway's Immunobiology. 9th ed., GS, Garland Science, Taylor & Francis Group, 2017, at 97].

Macrophages and dendritic cells express both TLRs and NOD1 and NOD2 and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins. [Janeway's Immunobiology. 9th ed., GS, Garland Science, Taylor & Francis Group, 2017, at 97].

Other members of the NOD family, including NLRX1 and NLRC5, have been identified, but their function is less well understood. [Janeway's Immunobiology. 9th ed., GS, Garland Science, Taylor & Francis Group, 2017, at 96-98].

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would activate the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3. [Janeway's Immunobiology. 9th ed., GS, Garland Science, Taylor & Francis Group, 2017, at 98-99].

NLR signaling, as exemplified by NLRP3, leads to the generation of pro-inflammatory cytokines and to cell death through formation of an inflammasome, a multiprotein complex. Activation of the inflammasome proceeds in several stages. Aggregation of NLRP molecules triggers autocleavage of procaspase I, which releases active caspase 1—Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxy terminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly IL-1β and IL-18, into their active forms, and induces a form of cell death (pyroptosis) associated with inflammation because of the release of these pro-inflammatory cytokines upon cell rupture. [Janeway's Immunobiology. 9th ed., GS, Garland Science, Taylor & Francis Group, 2017, at 99-100].

A priming step, which can result from TLR signaling, must first occur in which cells inducer and translate the mRNAs that encode the pro-forms of IL-1, IL-18 or other cytokines for inflammasome activation to produce inflammatory cytokines. For example, the TLR-3 agonist poly I:C can be used experimentally to prime cells for triggering of the inflammasome. [Janeways Immunobiology. 9th ed., GS, Garland Science, Taylor & Francis Group, 2017, at 100].

Inflammasome activation also can involve proteins of the PYHIN family, which have an H inversion (HIN) domain in place of an LRR domain. There are four PYIN proteins in humans. Id. at 100. A noncanonical inflammasome (caspase I-independent) pathway uses the protease caspase 11, which therefore is both a sensor and an effector molecule, to detect intracellular LPS. [Janeway's Immunobiology. 9th ed., GS, Garland Science, Taylor & Francis Group, 2017, at 101].

Besides activating effector functions and cytokine production, another outcome of the activation of innate sensing pathways is the induction of co-stimulatory molecules on tissue dendritic cells and macrophages. B7.1 (CD80) and B7.2 (CD86), for example, which are induced on macrophages and tissue dendritic cells by innate sensors such as TLRs in response to pathogenic recognition, are recognized by specific co-stimulatory receptors expressed by cells of the adaptive immune response, particularly CD4 T cells, and their activation by B7 is an important step in activating adaptive immune responses. [Janeway's Immunobiology. 9th ed., GS, Garland Science, Taylor & Francis Group, 2017, at 105].

The term “nutraceutical,” as used herein refers to a non-toxic dietary compound with extra health benefits in addition to the basic nutritional value found in foods. Nutraceuticals can be of either plant or animal origin. According to FDA regulations, most nutraceuticals would be categorized as “dietary supplements”. These are extracts, concentrates, metabolites, constituents or combinations of vitamins, minerals, botanicals, herbs, amino acids or dietary substances “for use by man to supplement the diet by increasing the total dietary intake.” In order to maintain classification as a dietary supplement (and avoid the FDA's strict drug approval process), nutraceutical labeling must maintain that the nutraceutical is not intended to diagnose, treat, cure or prevent any disease.

The term “nut milk” as used herein refers to non-dairy alternatives to traditional animal-derived milk made by soaking a plurality of nuts in water, blending until smooth, and then straining.

The term “organ” as used herein refers to a differentiated structure consisting of cells and tissues and performing some specific function in an organism.

The term “oxidative stress” as used herein refers to a condition where the levels of ROS significantly overwhelm the capacity of antioxidant defenses, leading to potential damage in a biological system. An oxidative stress condition can be caused by either increased ROS formation or decreased activity of antioxidants or both. Under certain circumstances, small transient increases in ROS levels can be employed as a signaling mechanism, leading to physiological cellular responses. [Li, R., et al. React. Oxyg. Species (Apex) (2016) 1 (1): 9-21].

As used herein, the term “paracrine signaling” refers to short range cell-cell communication via secreted signal molecules that act on adjacent cells.

The term “PAMPs” is an abbreviation for pathogen-associated molecular patterns. PAMPS are structural patterns present in components or products common to a wide variety of microbes but not host cells. PAMPS are ligands for pattern recognition molecules (PRMs).

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. The term PRRs includes toll-like receptors (TLRs) and scavenger receptors. Engagement of PRRs induces pro-inflammatory cytokines.

“Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

$$J = {C}_{v e h} \times {K}_{m} D / x \qquad [ Formula 1 ]$$

where
J=rate of absorption
Cᵥₑₕ=concentration of drug in vehicle
Kₘ=partition coefficient, which describes how a solute is distributed between two immiscle solvents);
D=diffusion coefficient, which indicates speed of diffusion and is dependent on molecule size, and other properties of the solute as well as temperature and pressure;
X=path length/thickness.

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Temperature—increased skin temperature increases permeability; and (vi) The composition of the compound and of the vehicle also determines the absorbency of a substance. Most substances applied topically are incorporated into bases or vehicles. The vehicle chosen for a topical application will greatly influence absorption and may itself have a beneficial effect on the skin. Ideally, a vehicle that has use in the present invention is easy to apply and remove, nonirritating, and cosmetically pleasing. In addition, the active must be stable in the chosen vehicle and must be released readily. Factors that determine the choice of vehicle and the transfer rate across the skin are the substance's partition coefficient, molecular weight and water solubility. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460.

The term “peptide” is used herein to refer to two or more amino acids joined by a peptide bond. Solid phase peptide synthesis may be accomplished by techniques familiar to those in the art and provided, for example, in Stewart and Young, 1984, Solid Phase Synthesis, Second Edition, Pierce Chemical Co., Rockford, III; Fields and Noble, 1990, Int. J. Pept. Protein Res. 35:161-214 or using automated synthesizers.

The term “peptidomimetic” as used herein refers to a small protein-like chain designed to mimic a peptide. A peptidomimetic typically arises from modification of an existing peptide in order to alter the molecule's properties.

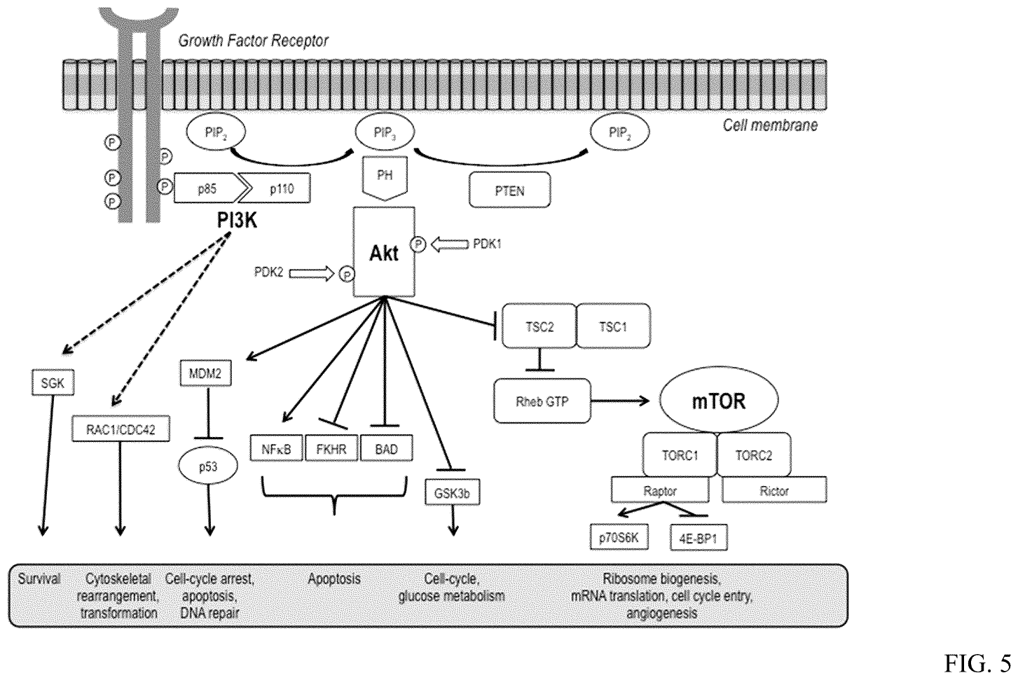

The PI3K/Akt/mTOR signaling pathways are crucial to many aspects of cell growth and survival. [Porta, C., et al. Frontiers in Oncology (2014) doi.10.3389/fpmc.2014.00064]. A schematic of the PI3K/Akt/mTOR pathway is shown in FIG. 5 (Taken from Porta, et al. Front. Oncol. (2014) 4: art. 64).

PI3Ks constitute a lipid kinase family characterized by the capability to phosphorylate inositol ring 3′—OH group in inositol phospholipids. [Id., citing Fruman, D A., et al. Annu. Rev. Biochem. (1998) 67: 481-507]. Class I PI3Ks are heterodimers composed of a catalytic (CAT) subunit (i.e., p110) and an adaptor/regulatory subunit (i.e., p85). This class is further divided into two subclasses: subclass IA (PI3Kα, β, and δ), which is activated by receptors with protein tyrosine kinase activity, and subclass IB (PI3KT), which is activated by receptors coupled with G proteins (Id., citing Fruman, D A., et al. Phosphoinositide kinases. Annu. Rev. Biochem. (1998) 67: 481-507).

Activation of growth factor receptor protein tyrosine kinases results in autophosphorylation on tyrosine residues. P13K is then recruited to the membrane by directly binding to phosphotyrosine consensus residues of growth factor receptors or adaptors through one of the two SH2 domains in the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB) [Id., citing Fruman, D A., et al., Phosphoinositide kinases. Annu. Rev. Biochem. (1998) 67: 481-507, Fresno-Vara, J A., et al., PI3K/Akt signaling pathway and cancer. Cancer Treat. Rev. (2004) 30: 193-204].

Akt. Akt/PKB, on its own, regulates several cell processes involved in cell survival and cell cycle progression. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor [Shimamura, H, et al. J. Am. Soc. Nephrol. (2003) 14: 1427-1434; Datta K., et al., Mol Cell Biol (1995) 15: 2304-2310; Kulik G., et al. Mol Cell Biol (1997) 17: 1595-1606; Yao R and Cooper G M, Science (1995) 267: 2003-2006). Stimulation of Akt has been shown to be dependent on phosphatidylinositol 3-kinase (PI3-kinase) activity (Fruman D A, et al. Annu Rev Biochem (1998) 67: 481-507; Choudhury G G, et al. Am J Physiol (1997) 273: F931-938, Franke T F., et al. Cell (1995) 81: 727-736; Franke T F., et al. Cell (1997) 88: 435-437].

Akt has been shown to act as a mediator of survival signals that protect cells from apoptosis in multiple cell lines [Brunet A., et al. Cell (1999) 96: 857-868; Downward J, Curr Opin Cell Biol 10: 262-267, 1998). For example, phosphorylation of the pro-apoptotic Bad protein by Akt was found to decrease apoptosis by preventing Bad from binding to the anti-apoptotic protein Bcl-XL [Dudek H, et al. Science (1997) 275: 661-665; Datta S R, et al. Cell (1997) 91: 231-241]. Akt was also shown to promote cell survival by activating NF-kB [Cardone M H, et al. Science (1998) 282: 1318-1321; Khwaja A, Nature (1999) 401: 33-34] and inhibiting the activity of the cell death protease caspase-9 [Kennedy S G., et al. Mol Cell Biol (1999) 19: 5800-5810].

mTOR signaling pathway: Mechanistic Target Of Rapamycin (mTOR) is an atypical serine/threonine kinase that is present in two distinct complexes. The first, mTOR complex 1 (mTORC1), is composed of mTOR, Raptor, GβL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumor-suppressor genes mutated in the tumor syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1). [Huang, J. Manning B D, Biochem J. (2008) 412(2): 179-90]. Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GβL, Sin1, PRR5/Protor-1, and DEPTOR. mTORC2 promotes cellular survival by activating Akt, regulates cytoskeletal dynamics by activating PKCa, and controls ion transport and growth via SGK1 phosphorylation. Aberrant mTOR signaling is involved in many disease states.

PI3K also acts as a branch point in response to TGF-β, leading to activation of PAK2/c-Abl, which stimulates collagen gene expression in normal fibroblasts, and induces fibroblast proliferation, thereby increasing the number of myofibroblast precursors. [He, W and Dai, C. Curr. Pathobiol. Rep. (2015) 3: 183-92, citing Wilkes, M C and Leof, EB. J. Biol. Chem. (2006) 281: 27846-27854]. PAK2/c-Abl promotes fibrosis through its downstream mediators, including PKC6/Fli-1 and early growth response (Egr)-1, -2, and -3 [Id., citing Tsou, P S., et al. A. J. Physiol. Cell Physiol. (2014) 307: C2-C13; Bhattacharyya, S., et al. J. Pathol. (2013) 229: 286-297; Fang, F., et al. Am. J. Pathol. (2013) 183: 1197-1208].

The term “photoaging” as used herein refers to the cumulative detrimental effects (such as wrinkles or dark spots) on skin that result from long-term exposure to sunlight, e.g., ultraviolet light.

The term “polymerase chain reaction” or “PCR” is a laboratory technique used to amplify small segments of DNA sequences.

The term “polypeptide” is used herein in its broadest sense to refer to a sequence of subunit amino acids, amino acid analogs or peptidomimetics, wherein the subunits are linked by peptide bonds. Polypeptides can be chemically synthesized or recombinantly expressed. Synthetic polypeptides prepared using the techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R1-CH2-NH—R2, where Ri and R2 are amino acid residues or sequences. A reduced peptide bond may be introduced as a dipeptide subunit. Such a polypeptide would be resistant to protease activity and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids.

The term “protein” as used herein refers to a large complex molecule or polypeptide composed of amino acids.

The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

Use herein of the terms “peptide”, “peptides”, “polypeptide”, “peptidomimetic” or “protein” should be taken to include reference to “derivatives” of such compounds, unless the context requires otherwise, and to include “prodrugs.”

The term “pharmaceutical composition” is used herein to refer to a composition that is employed to prevent, reduce in intensity, cure or otherwise treat a target condition or disease.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “potency” and its various grammatical forms as used herein, refers to power or strength of a formulation to produce a defined effect.

The term “protective” as used herein is used in the broadest pharmacological sense to mean any agent that isolates the exposed surface of the skin or other membrane from harmful or annoying stimuli.

The term “purification” and its various grammatical forms as used herein refers to the process of isolating or freeing from foreign, extraneous, or objectionable elements.

The term “Reactive Oxygen Species or “ROS” as used herein, such as free radicals and peroxides, refers to a class of molecules that are derived from the metabolism of oxygen and exist inherently in all aerobic organisms. It is a collective term to include superoxide (O2·-), hydrogen peroxide (H₂O₂), hydroxyl radical (OH·), singlet oxygen (1O2), peroxyl radical (LOO·), alkoxyl radical (LO·), lipid hydroperoxide (LOOH), peroxynitrite (ONOO—), hypochlorous acid (HOCl), and ozone (O3), among others. [Li, R., et al. React. Oxyg. Species (Apex) (2016) 1 (1): 9-21] The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH—) and the superoxide ion (O2-) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO—), alkoxyl (RO—) and hydroxyl (OH—) radicals.

Many abnormal physiological and metabolic processes in organisms produce biological oxidants such as superoxide radicals (O₂·—), hydroxyl radicals (OH), and hydrogen peroxide (H₂O₂), which could endow several different biological targets with reactivity and instability, such as DNA, proteins, and membrane lipids [Zheng, M., et al. Antioxidants (2023) 12: 1675, citing Taverne, Y. J. H. J., et al. Oxidative Med. Cell Longev. (2013) 2013: 862423]. In the oxidative stress state, biological oxidants lead to an imbalance between oxidation and antioxidation in vivo, cellular oxidants activate various apoptosis inducers through certain transcription factors or directly lead to lipid peroxidation, protein and DNA damage, and enzyme expression disorders. Physiological production of superoxide radical anion and hydrogen peroxide is essential for redox signaling in cells, but their excessive generation could lead to oxidative stress and oxidative modifications of biomolecules and lipid peroxidation, which could be related to pathophysiological processes.

Aerobic organisms possess antioxidant defense systems that deal with reactive oxygen species (ROS) produced as a consequence of aerobic respiration and substrate oxidation. In the process of normal cellular metabolism, oxygen undergoes a series of univalent reductions, leading sequentially to the production of O₂·—, hydrogen peroxide (H₂O₂), and H₂O. Potential enzymatic source of ROS includes components of the mitochondrial electron transport chain, xanthine oxidase, the cytochrome p450 monooxygenases, lipoxygenase, nitric oxide synthase (NOS), and the NADPH oxidase (Fukai, T. and Ushio-Fukai, Antioxidants & Redox Signaling (2011) 15 (6): 1583-1606, citing Guzik, T J and Harrison, DG. Drug Discov. Today (2006) 11: 524-533). Superoxide anion is dismutated by superoxide dismutases (SODs) to H₂O₂ that is catalyzed to H₂O by catalase, peroxiredoxins (Prxs), or glutathione peroxidases (GPx) Low levels of either intracellular or extracellular ROS (e.g., superoxide and H2O2) are indispensable in many biochemical processes, including intracellular signaling, defense against microorganisms, and cell function (Id., citing Go, YM and Jones, DP. Free Radic. Biol. Med. (2011) 50 (4): 495-509; Lassegrue, B. and Griendling, KK. (2010) Arterioscler. Thromb. Vase. Biol. (2010) 30: 653-61; Ushio-Fukai, M. Antiox. Redoc. Signal. (2009) 11: 1289-1299). In contrast, high dose and/or inadequate removal of ROS, especially superoxide anion, results in oxidative stress, which has been implicated in the pathogenesis of many cardiovascular diseases, including hypercholesterolemia, atherosclerosis, hypertension, diabetes, and heart failure. ROS also represent a component of the innate immune system; they are not only involved in the respiratory burst of neutrophils but also signal inflammatory cell chemotaxis into sites of inflammation [Id., citing Bogdan, C., et al. Curr. Opin. Immunol. (2000) 12: 64-76].

Nitric oxide (NO), which has anti-inflammatory and anticoagulant properties as well as a vasodilator effect, can be rapidly inactivated by reaction with O2·- leading to the production of the strong oxidant peroxynitrite (ONOO—). This reaction is important in common conditions leading to endothelial and mitochondrial dysfunction, including hypercholesterolemia, hypertension, diabetes, and aging, in which vascular production of O2·- is increased (Id., citing Guzik T J. Harrison D G. Drug Discov. Today. (2006) 11:524-533; Madamanchi N R. and Runge M S. Circ Res. (2007) 100: 460-473).

The term “reads” as used in next-generation sequencing, refers to the DNA sequence from one fragment (meaning a small section of DNA). Next-generation sequencing read length refers to the number of base pairs (bp) sequenced from a DNA fragment. After sequencing, the regions of overlap between reads are used to assemble and align the reads to a reference genome, reconstructing the full DNA sequence.

The term “redox signaling” as used herein refers to a physiological process, where ROS act as second messengers to mediate responses that are required for proper function and survival of the cell. On the other hand, redox modulation (or redox regulation) refers to a process wherein ROS alter the activity or function of the redox-sensitive molecular targets, including signaling proteins and metabolic enzymes, leading to either physiological or pathophysiological responses. When pathophysiological responses occur, it is also known as oxidative stress. [Li, R., et al. React. Oxyg. Species (Apex) (2016) 1 (1): 9-21].

The term “rejuvenate” and its various grammatical forms as used herein refer to making young or youthful again. The term “resuscitate” as used herein refers to being restored to life or being revived.

The term “renewal” or “self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state.

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function.

The term “reverse” as used herein refers to turning backward or in an opposite direction.

The term “rubefacient” as used herein refers to an agent that induces hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

The term “sclerosant” as used herein refers to an agent used as a chemical irritant injected into a vein in sclerotherapy. Examples of sclerosants include, but are not limited to, morrhuate sodium, sodium tetradecyl sulfate, laureth 9 and ethanolamine oleate.

The term “semaphorins” or “Semas” as used herein refer to a large and diverse family of proteins that are divided into 8 classes based on structural features and distribution among different phyla. Class 1 and 2 Semas are found only in invertebrates, while class 3-7 are found only in vertebrates (with the one exception being Sema-5c, which is also found in invertebrates). Class V Semas are found in viruses. Class 1, 4, 5, and 6 members are transmembrane, class 2, 3 and V members are secreted, and class 7 members are glycosylphosphatidylinositol (GPI)-linked. In addition, class 4, 5 and 7 members, and possibly others, are cleaved and released extracellularly. Plexin receptors, the predominant receptors for Semas, are grouped into 4 classes (A-D) and each plexin receptor class interacts with a particular Sema class or classes to mediate signaling. A number of other membrane-associated receptors and co-receptors are also important for Sema signal transduction. These proteins directly bind Semas and initiate signaling (e.g., integrins), act as ligand binding co-receptors (e.g., Npn1,2), and/or work as part of multimeric receptor complexes (e.g., RTKs). The hallmark of the Sema protein family is the sema domain, an approximately 500 amino acid extracellular domain (2). the sema domain, is present as a single copy located at the N-terminus of Sema proteins and is essential for Sema signaling. [Alto, L T and Terman, JR, Methods Mol Biol. (2017) 1493: 1-25]. Plexin B1, the Sema 4D receptor, is a tumor suppressor protein for melanoma, in part through inhibition of the oncogenic c-Met tyrosine kinase receptor. Sema4D is a protective paracrine factor for normal human melanocyte survival in response to ultraviolet irradiation; it stimulates proliferation and regulates the activity of the c-Met receptor. C-Met receptor signaling stimulates melanocyte migration, in part through down-regulation of the cell adhesion molecule E-cadherin. [Soong, J. et al. J. Invest. Dermatol. (2012) 132 (4): 1230-1238].

The term “signature” as used herein refers to a specific and complex combination of biomarkers that reflect a biological state.

The term “skin clarity” as used herein refers to a smooth, even skin texture that's free of lumps and bumps.

The term “skin luminosity” as used herein refers to a measure of the intensity of the light reflected from the skin's surface as it bounces back into the observer's eye. The amount of light reflected from the skin is dependent on how smooth the skin is.

The term “skin radiance” as used herein refers to the skin's ability to reflect light. Radiance is about evenness. Dark spots and uneven skin tone absorb light, minimizing the healthy glow from your complexion.

The term “skin rejuvenation” as used herein refers to treatments that aim to restore skin from any damage. Skin damage can be a result of sun exposure, an underlying health condition, or a normal sign of aging.

The term “skin texture” as used herein refers to condition of the surface of the skin. Young healthy skin is well hydrated, and firm with a smooth, soft texture and luminosity. In aged skin, cell turnover slows, resulting in a buildup of dead skin cells which contributes to the appearance of dull skin and creates clogged pores.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. Among the acceptable vehicles and solvents that may be employed are water, Ringer's solution, and isotonic sodium chloride solution.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “stem cells” refers to undifferentiated cells having high proliferative potential with the ability to self-renew that can generate daughter cells that can undergo terminal differentiation into more than one distinct cell phenotype.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

The term “superoxide dismutases” or “SODs” as used herein refers to major antioxidant defense systems against superoxide anions, which consist of three isoforms of SOD in mammals: the cytoplasmic Cu/ZnSOD (SOD1), the mitochondrial MnSOD (SOD2), and the extracellular Cu/ZnSOD (SOD3), all of which require catalytic metal (Cu or Mn) for their activation. Evidence suggests that in each subcellular location, SODs catalyze the conversion of O2·-H2O2, which may participate in cell signaling. In addition, SODs play a critical role in inhibiting oxidative inactivation of nitric oxide, thereby preventing peroxynitrite formation and endothelial and mitochondrial dysfunction.

Superoxide dismutase (SOD) is widely distributed in plants, animals, and microorganisms [Zheng, M. Antioxidants (Basel) (2023) 12 (9): 1675, citing Zelko, IN., et al. Free Radic. Biol. Med. (2002) 33: 337-49]. Intracellular and extracellular O2·- is produced by plasma membrane-bound NADPH oxidase (NOX). Extracellular H₂O₂ is transported to cells through the aquaporin (AQP) channel and converted to water by the activity of catalase (CAT), peroxidase (PRX) and glutathione peroxidase (GPX) [Id., citing Wu, C. et al. Nano Today (2022) 46: 101574]. When human skin is in direct contact with oxygen, it will cause skin aging and damage [Id., citing Altobelli, G G., et al. Front. Med. (2020) 7: 183]. SOD is a powerful oxygen-free radical scavenger, which has sunscreen and radiation protection effects.

The term “suspension” as used herein refers to a dispersion (mixture) in which a finely divided species is combined with another species, with the former being so finely divided and mixed that it doesn't rapidly settle out. In everyday life, the most common suspensions are those of solids in liquid.

The term “symptom” as used herein refers to a sign or an indication of disorder or disease, especially when experienced by an individual as a change from normal function, sensation, or appearance.

The term “synthetic” as used herein refers to production of a substance artificially, e.g., by chemical synthesis, rather than by natural origin.

The term “TH1 cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. TH1 cells secrete IFN-gamma, IL-2, IL-10, and TNF-alpha/beta. IL-12 and IFN-γ make naive CD4+ T cells highly express T-bet and STAT4 and differentiate to TH1 cells. [Zhang, Y., et al. Adv. Exp. Med. Bio. (2014) 841: 15-44].

The term “TH2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STAT6 and GATA3 and differentiate to TH2 cells. [Zhang, Y. et al. Adv. Exp. Med. Bio. (2014) 841: 15-44].

The term “TH17 cells” as used herein refers to a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, LA., et al., Immunol. Rev. (2008) 223: 87-113]. The key transcription factor in TH17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated Tconv activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/fimmu.2019.01098].

The term “tissue-resident memory T cell” or “T_{RM}” as used herein refers to memory lymphocytes that do not migrate after taking up residence in barrier tissues, where they are retained long term. They appear to be specialized for rapid effector function after restimulation with antigen or cytokines at sites of pathogen entry.

The term “tolerance” as used herein refers to the failure to respond to a particular antigen. Tolerance mechanisms that operate in the thymus before the maturation and circulation of T cells are referred to as “central tolerance.” Not all antigens of which T cells need to be tolerant are expressed in the thymus, and therefore central tolerance mechanisms alone are insufficient. Additional tolerance mechanisms exist to restrain the numbers and or function of T cells that are reactive to developmental or food antigens, which are not thymically expressed. Tolerance acquired by mature circulating T cells in the peripheral tissues is called “peripheral tolerance.”

As used herein, the term “therapeutic agent” or “active agent” refers to refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

The term “topical” as used herein refers to administration of an inventive composition at, or immediately beneath, the point of application. The term “topical administration” and “topically applying” as used herein are used interchangeably to refer to delivering a composition comprising plant exosomes comprising a cargo comprising a peptide, a nucleic acid, or both, onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

Substances generally are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is the formation of a film. Film formation may be protective (e.g., sunscreen) and/or occlusive (e.g., to provide a moisturizing effect by diminishing loss of moisture from the skin surface). One example of an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into or binding with skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

Medically, “topically” means applied to the surface of the skin or some other surface—Many topical medications are epicutaneous, meaning that they are applied directly to the skin. Topical medications may also be inhalational, such as asthma medications, or applied to the surface of tissues other than the skin, such as eye drops applied to the conjunctiva, or ear drops placed in the ear, or medications applied to the surface of a tooth.

As used herein, the term “tissue” refers to a collection of similar cells and the intercellular substances surrounding them. For example, adipose tissue is a connective tissue consisting chiefly of fat cells surrounded by reticular fibers and arranged in lobular groups or along the course of smaller blood vessels. Connective tissue is the supporting or framework tissue of the body formed of fibrous and ground substance with numerous cells of various kinds. It is derived from the mesenchyme, and this in turn from the mesoderm. The varieties of connective tissue include, without limitation, areolar or loose; adipose; sense, regular or irregular, white fibrous; elastic; mucous; lymphoid tissue; cartilage and bone.

The term “transcription” as used herein refers to the synthesis of an RNA strand in a process in which ribonucleoside 5′-triphosphates (rNTPs) base-pair sequentially with nucleotides in a template strand and are polymerized in the 5′ to 3′ direction (with elimination of PPi) by an RNA polymerase.

The term “transformation” as used herein refers to the transfer of a nucleic acid molecule into a host organism or host cell by any method. A nucleic acid molecule that has been transformed into an organism/cell may be one that replicates autonomously in the organism/cell, or that integrates into the genome of the organism/cell, or that exists transiently in the cell without replicating or integrating. Non-limiting examples of nucleic acid molecules suitable for transformation are disclosed herein, such as plasmids and linear DNA molecules.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tune” and its various grammatical forms as used herein, refers to an ability to vary the amount, contents or both of a cargo associated with a population of exosomes for delivery

The term “vascular endothelial growth factor” or “VEGF” as used herein refers to the principal angiogenic growth factor that modulates angiogenesis through receptor tyrosine kinase VEGF receptors (VEGFRs). Multiple VEGFs (e.g., VEGF-A, VEGF-B, VEGF-C, and VEGF-D) interact with VEGFRs, such as VEGFR1, VEGFR2, and VEGFR3. VEGF signaling is induced by the binding of VEGF ligands to their cognate membrane-bound receptors, which results in the activation of multiple downstream pathways. The VEGF signaling cascade includes: the Ras/MAPK pathway, regulating cell proliferation and gene expression; the FAK/paxillin pathway, involved in the rearrangement of the cytoskeleton; the PI3K/AKT pathway, regulating cell survival; and the phospholipase C gamma (PLCγ) pathway, controlling vascular permeability

The term “vitamin” as used herein, refers to any of various organic substances essential in minute quantities to the nutrition of most animals. Vitamins act especially as coenzymes and precursors of coenzymes in the regulation of metabolic processes. Non-limiting examples of vitamins usable in context of the described invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

The term “wound healing” refers to the process by which the body repairs trauma to any of its tissues, especially those caused by physical means and with interruption of continuity.

A wound-healing response often is described as having three distinct phases-injury, inflammation and repair. Generally speaking, the body responds to injury with an inflammatory response, which is crucial to maintaining the health and integrity of an organism. If, however, it goes awry, it can result in tissue destruction.

Although these three phases are often presented sequentially, during chronic or repeated injury, these processes function in parallel, placing significant demands on regulatory mechanisms. (Wilson and Wynn, Mucosal Immunol., 2009, 3(2): 103-121).

### Phase I: Injury

Injury caused by factors including, but not limited to, autoimmune or allergic reactions, environmental particulates, or infection or mechanical damage, often results in the disruption of normal tissue architecture, initiating a healing response. Damaged epithelial and endothelial cells must be replaced to maintain barrier function and integrity and prevent blood loss, respectively. Acute damage to endothelial cells leads to the release of inflammatory mediators and initiation of an anti-fibrinolytic coagulation cascade, temporarily plugging the damaged vessel with a platelet and fibrin-rich clot. For example, lung homogenates, epithelial cells or bronchoalveolar lavage fluid from idiopathic pulmonary fibrosis (IPF) patients contain greater levels of the platelet-differentiating factor, X-box-binding protein-1, compared with chronic obstructive pulmonary disease (COPD) and control patients, suggesting that clot-forming responses are continuously activated. In addition, thrombin (a serine protease required to convert fibrinogen into fibrin) is also readily detected within the lung and intra-alveolar spaces of several pulmonary fibrotic conditions, further confirming the activation of the clotting pathway. Thrombin also can directly activate fibroblasts, increasing proliferation and promoting fibroblast differentiation into collagen-producing myofibroblasts. Damage to the airway epithelium, specifically alveolar pneumocytes, can evoke a similar anti-fibrinolytic cascade and lead to interstitial edema, areas of acute inflammation, and separation of the epithelium from the basement membrane.

Platelet recruitment, degranulation and clot formation rapidly progress into a phase of vasoconstriction with increased permeability, allowing the extravasation (movement of white blood cells from the capillaries to the tissues surrounding them) and direct recruitment of leukocytes to the injured site. The basement membrane, which forms the extracellular matrix underlying the epithelium and endothelium of parenchymal tissue, precludes direct access to the damaged tissue. To disrupt this physical barrier, zinc-dependent endopeptidases, also called matrix metalloproteinases (MMPs), cleave one or more extracellular matrix constituents allowing extravasation of cells into, and out of, damaged sites.

### Phase II: Inflammation

Once access to the site of tissue damage has been achieved, chemokine gradients recruit inflammatory cells. Neutrophils, eosinophils, lymphocytes, and macrophages are observed at sites of acute injury with cell debris and areas of necrosis cleared by phagocytes.

The early recruitment of eosinophils, neutrophils, lymphocytes, and macrophages providing inflammatory cytokines and chemokines can contribute to local TGF-β and IL-13 accumulation. Following the initial insult and wave of inflammatory cells, a late-stage recruitment of inflammatory cells may assist in phagocytosis, in clearing cell debris, and in controlling excessive cellular proliferation, which together may contribute to normal healing. Late-stage inflammation may serve an anti-fibrotic role and may be required for successful resolution of wound-healing responses. For example, a late-phase inflammatory profile rich in phagocytic macrophages, assisting in fibroblast clearance, in addition to IL-10-secreting regulatory T cells, suppressing local chemokine production and TGF-β, may prevent excessive fibroblast activation.

The nature of the insult or causative agent often dictates the character of the ensuing inflammatory response. For example, exogenous stimuli like pathogen-associated molecular patterns (PAMPs) are recognized by pathogen recognition receptors, such as toll-like receptors and NOD-like receptors (cytoplasmic proteins that have a variety of functions in regulation of inflammatory and apoptotic responses), and influence the response of innate cells to invading pathogens. Endogenous danger signals also can influence local innate cells and orchestrate the inflammatory cascade.

The nature of the inflammatory response dramatically influences resident tissue cells and the ensuing inflammatory cells. Inflammatory cells themselves also propagate further inflammation through the secretion of chemokines, cytokines, and growth factors. Many cytokines are involved throughout a wound-healing and fibrotic response, with specific groups of genes activated in various conditions. Fibrotic lung disease (such as idiopathic pulmonary fibrosis) patients more frequently present pro-inflammatory cytokine profiles (including, but not limited to, interleukin-1 alpha (IL-1α), interleukin-1 beta (IL-1), interleukin-6 (IL-6), tumor necrosis factor alpha (TNF-α), transforming growth factor beta (TGF-β), and platelet-derived growth factors (PDGFs)). Each of these cytokines has been shown to exhibit significant pro-fibrotic activity, acting through the recruitment, activation and proliferation of fibroblasts, macrophages, and myofibroblasts.

### Phase III: Tissue Repair and Contraction

The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization. The vast majority of studies elucidating the processes involved in this phase of wound repair have come from dermal wound studies and in vitro systems.

Myofibroblast-derived collagens and smooth muscle actin (α-SMA) form the provisional extracellular matrix, with macrophage, platelet, and fibroblast-derived fibronectin forming a fibrin scaffold. Collectively, these structures are commonly referred to as granulation tissues. Primary fibroblasts or alveolar macrophages isolated from IPF patients produce significantly more fibronectin and α-SMA than control fibroblasts, indicative of a state of heightened fibroblast activation. It has been reported that IPF patients undergoing steroid treatment had similar elevated levels of macrophage-derived fibronectin as IPF patients without treatment. Thus, similar to steroid resistant IL-13-mediated myofibroblast differentiation, macrophage-derived fibronectin release also appears to be resistant to steroid treatment, providing another reason steroid treatment may be ineffective. From animal models, fibronectin appears to be required for the development of pulmonary fibrosis, as mice with a specific deletion of an extra type III domain of fibronectin (EDA) developed significantly less fibrosis following bleomycin administration compared with their wild-type counterparts.

In addition to fibronectin, the provisional extracellular matrix consists of glycoproteins (such as PDGF), glycosaminoglycans (such as hyaluronic acid), proteoglycans and elastin. Growth factor and TGF-β-activated fibroblasts migrate along the extracellular matrix network and repair the wound. Within skin wounds, TGF-β also induces a contractile response, regulating the orientation of collagen fibers. Fibroblast to myofibroblast differentiation, as discussed above, also creates stress fibers and the neo-expression of α-SMA, both of which confer the high contractile activity within myofibroblasts. The attachment of myofibroblasts to the extracellular matrix at specialized sites called the “fibronexus” or “super mature focal adhesions” pull the wound together, reducing the size of the lesion during the contraction phase. The extent of extracellular matrix laid down and the quantity of activated myofibroblasts determines the amount of collagen deposition. To this end, the balance of matrix metalloproteinases (MMPs) to tissue inhibitor of metalloproteinases (TIMPs) and collagens to collagenases vary throughout the response, shifting from pro-synthesis and increased collagen deposition towards a controlled balance, with no net increase in collagen. For successful wound healing, this balance often occurs when fibroblasts undergo apoptosis, inflammation begins to subside, and granulation tissue recedes, leaving a collagen-rich lesion. The removal of inflammatory cells, and especially α-SMA-positive myofibroblasts, is essential to terminate collagen deposition.

### Embodiments

According to one aspect, the present disclosure provides a composition comprising plant-derived exosome-like nanoparticles derived from plant tissue. According to some embodiments, the plant-derived exosome-like nanoparticles are isolated from a tissue of a vascular plant. According to some embodiments, examples of the tissue include roots, and/or stems, and/or leaves, and/or flowers, and/or seeds, and/or fruits, and/or liquid extracts of any of the foregoing, and/or a nut milk.

Non-vascular plants are simple, low-growing, nonflowering plants (such as a moss or liverwort) that lacks specialized conducting channels for transporting water and nutrients (e.g., xylem and phloem) and in which the photosynthetic gametophyte is the dominant stage of the life cycle. Gametophytes are plants that produce gametes through mitosis to produce a zygote. Nonvascular plants lack vascular tissues and lack true leaves, roots and stems.

Vascular plants, which are plants characterized by the presence of conducting tissue, are eukaryotes. They include all the seed-containing plants, angiosperms (flowering plants), gymnosperms, and the pteridophytes (lycophytes, horsetails, and ferns). The vascular tissue of vascular plants includes xylem and phloem. Xylem is vascular tissue that transports water and dissolved minerals from roots to stems and leaves. This type of tissue consists of dead cells that lack end walls between adjacent cells. The side walls are thick and reinforced with lignin, which makes them stiff and waterproof. Phloem is vascular tissue that transports food (sugar dissolved in water) from photosynthetic cells to other parts of the plant for growth or storage. This type of tissue consists of living cells that are separated by end walls with tiny perforations, or holes. The vascular tissue allows plants to grow tall in the air without drying out and to take advantage of sunlight. Vascular plants reproduce through a process known as alternation of generations, which involves a life cycle from the haploid phase to the diploid phase and vice versa. While studying the vascular plant life cycle, the diploid phase is considered as the sporophyte and the haploid phase as the gametophyte. Sporophytes are plants that produce spores through meiosis to produce gametophytes further. The period of each phase during the life cycle depends on the type of vascular plant.

The three organs of vascular plants are roots, stems and leaves. Leaves acquire sunlight and carry out photosynthesis to fees the organism; roots explore the soil and acquire the waters and nutrients required for photosynthesis and growth; stem connect the photosynthetic part with the water and nutrient acquiring part and also serve to distribute the leaves effectively in their aerial environment. Each of these three organs possess three fundamental tissues: a ‘skin’ (dermal tissue), transport tissue (vascular tissue), and ground tissue (everything else, the tissue that fills the spaces between dermal tissue and vascular tissue). Cells of vascular plants show substantially more specialization than is found in non-vascular plants and multiple cell types have been defined, primarily on the basis of:
(1) whether the plant cell is alive or dead at maturity,
(2) Cell wall characteristics. All plant cells have what is called a primary cell wall composed of cellulose microfibrils embedded in a matrix of hemicellulose and pectins, molecules that bind cellulose microfibrils to each other and also absorb water, forming a gel. The primary cell wall is present as the cell is growing; when the cell expands the wall yields to the pressures that are present inside the cell. The cell stops growing when the cell wall stiffens and no longer yields to the pressures generated inside it. At this point some cells deposit a distinct type of cell wall material, called a secondary cell wall, inside the primary cell wall. Since the cell is not growing, the more secondary cell wall that is deposited, the smaller the space inside the cell wall becomes. When the cell dies, this space, where the cytosol (usually with a large vacuole) used to be, is termed the lumen. Like the primary cell wall, the secondary cell wall contains cellulose microfibrils, but they are embedded in a matrix of lignin. Lignin is a complex polymer composed of phenolic subunits. Unlike the primary cell wall, the secondary cell wall has substantial compressive strength and does not need a cell membrane and the pressurization of water inside the cell in order for the cell to resist compression. Plants or plant parts (e.g., spinach and many other leaves) with cells possessing only a primary cell wall are called herbaceous and are much less resistant to forces produced by gravity or the wind. Such plants/plant parts lose all structural integrity if the cell membrane is destroyed or if lost water is not replaced
(3) Cell shape. Plant cells come in a variety of shapes. Many cells are round or nearly so, or rectangular with their long dimension being two to ten times that of the short dimensions. Other cells are very elongate with their long dimension being up to 1000 times that of their diameter. Generally, the long axis of cells runs the same direction as the long axis of the plant, i.e., up and down the stem/root.

According to some embodiments, the plant-derived exosome-like nanoparticles can be isolated/sourced from entire plants of the following families, including seeds:

### Plant Families:

Asphodelaceae
Solanaceae
Curcurbitaceae
Fabaceae
Meliaceae
Oleaceae
Asteraceae
Plumbaginaceae
Juglandaceae
Droseraceae
Polygonaceae
Rosaceae
Liliaceae
Orobanchaceae
Apiaceae
Betulaceae
Passifloraceae
Papaveraeeae
Lamiaceae
Rubiaceae

### Sea Weed:

Alariaceae
Laminariaceae
Lessoniceae
Ulvaceae
Gigartinaceae
Passiflora ligularis (granadilla)
Passiflora edulis
Morinda citrifolia (Noni)
Agastache Mexicana
Laminaria Digitata
Chondrus crispus
Macrocystis pyrifera

According to some embodiments, the plant-derived exosome-like nanoparticles can be isolated/sourced from entire plants of the following plants, including seeds:
All Asphodelacae Family
Subfamily: Xanthorrhoeaceae
Subfamily: Hemerocallidoideae
Subfamily: Asphodeloideae

### General:

Genus: Aloe vera
Mascarene aloes

For example:
Aloe barbadensis Miller
Aloe ferox
Aloe arborescens
Aloe perryi Baker
Aloe succotrina Weston
Aloe maculata All.
Cape Aloe
Aloe tormentorri
Aloe purpurea
Aloe macra
Aloe lomatophylloides
Aloe macra Haw
Genus: Haworthia (150 species)
Genus: Gasteria (31 species)
Genus: Xanthorrhoea (30 species of flowering plants)

Other plants sources can include:
Plant and beans/fruit of Vicia Faba/Vicia faba L. minor
Plant and fruit of Lansium Domesticum
Plant and fruit of Momordica Charantia
Plant and flowers of Syringa Vulgaris
Plant and flowers of Genus: Artemesia

For example,
Artemisia Absinthium
Artemisia Vulgaris
Sagebrush Artemisia species
Plants flowers and fruit of Genus: Plumbago
Juglans nigra (leaves flowers and fruit)
Genus: Drosera
Drosera rotundifolia
Drosera intermedia
Drosera anglica
Drosera ramentacea
Drosera madagascariensis
Lustwort
Fo ti plant: (also known as he shou wu)
Reynoutria multiflora
Fallopia multiflora
Polygonum multiflorum
Plant, flowers and fruit from Prunus persica
Pumpkin: Plant, flowers and fruit
Granadilla or Grenadia: Passiflora ligularis
Morinda citrifolia (Noni)
Agastache Mexicana
Laminaria Digitata
Chondrus crispus
Macrocystis pyrifera

### Sea Weed Families:

Alariaceae
Laminariaceae
Lessoniceae
Ulvaceae
Gigartinaceae
Flower and plants of Curcuma longa
Plant and Fruits of Family: Solanaceae
Plant and Fruits of Family: Curcurbitaceae
Chelidonium majus
Lilium lancifolium
Lilium tigrinum
Rehmanniae radix
Centella asiatica
Betula pendula

According to some embodiments, purification of the plant-derived exosome-like nanoparticles can be carried out at 4° C. by traditional methods [Fujita, D., et al. Mol. Pharm. (2018) 15 (12): 5772-80], e.g.,
(1) homogenizing fresh plant tissue with a high-speed grinder to form a juice;
(2) filtering the juice to remove large solid impurities and residues to form a crude solution;
(3) centrifuging the juice twice at low speed;
(4) centrifuging the supernatant at a speed of 100,000-120,000×g for 90 minutes in a high speed refrigerated centrifuge;
(5) resuspending the pellet in phosphate-buffered saline for collection;
(6) layering the pellet on a sucrose gradient comprising 8%, 30%, 45%, and 60% sucrose in PBS; and
(7) extracting the plant-derived exosome-like nanovesicles at their characteristic density zone (1.13-1.19 g/ml).

According to some embodiments, a layer of highly concentrated sucrose is added to the bottom of the centrifuge tube as a cushion, a layer of lower-concentration sucrose is added over the previous layer, and after high-speed ultracentrifugation, specific vesicles should gather between the sucrose layers and maintain their representative structures and shapes [Kim. J. et al. citing Li, P, et al. Theranostics (2017) 7(3): 789-804]; Li, Z., et al. Sci. Rep. (2018) 8(1): 14644; Stanley, C., et al. Methods Mol. Biol. (2016) 1459: 259-269].

According to some embodiments, for leaf section preparation, the inner leaf pulp containing parenchymal mesophyll cells is carefully removed to avoid collecting any outer leaf skin material and then homogenized. Homogenization can be performed using a Precellys tissue homogenizer, ceramic beads and sterile PBS to disrupt the plant pulp tissue. The homogenate is transferred to a larger tube and the beads are washed four times with sterile PBS to collect all the homogenate.

According to some embodiments, homogenates are diluted in PBS at a ratio of about 16g homogenate per 50 ml PBS. The diluted homogenate mixture is put through a freeze-thaw cycle by freezing at −80° C. and then thawed at room temperature. The volume is increased to 300 ml by adding PBS. The diluted homogenate mixture is then fractionated by differential centrifugation to isolate the plant exosomes. The diluted homogenate mixture is centrifuged at 4000×g for 20 minutes at room temperature to pellet cell debris. The clarified supernatant is then centrifuged at 12,000×g for 30 minutes at room temperature to remove any intact chloroplasts. The subsequent clarified supernatant is then brought up to 360 mL with PBS and subjected to centrifugation at 100,000×g for 2 hours at 4° C. to pellet exosomes. The supernatant is aspirated and the pellet(s) suspended in a minimum volume of DPBS (300-1000 μl).

According to some embodiments, size of the purified exosome-like nanovesicles is about 50-500 nm, inclusive, i.e., 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 300 nm, 310 nm, 320 nm, 330 nm, 340 nm, 350 nm, 400 nm, 410 nm, 420 nm, 430 nm, 440 nm, 450 nm, 460 nm, 470 nm, 480 nm, 490 nm, or 500 nm.

According to some embodiments, by TEM or SEM the purified exosome-like nanovesicles have a cup-shaped morphology.

According to some embodiments, the membranes of the purified exosome-like nanovesicles comprise phosphatidic acid (PA), phosphatidylcholines (PC), digalactosyldiacylglycerol (DGDG), and monogalactosyldiacylglycerol (MGDG)

According to some embodiments, the purified exosome-like nanovesicles display a negative charge between −25 and −15 nM, inclusive.

According to some embodiments, the purified exosome-like nanovesicles are isolated from abiotically stressed plants. According to some embodiments, the abiotic stress conditions cause the plant source to modulate signaling pathways and metabolism to ensure its growth and development in a challenging environment. According to some embodiments, the purified exosome-like nanovesicles derived from an abiotically stressed plant contain a cargo. According to some embodiments, the plant(s) are subjected to combinations of conditions of abiotic stress in order to tune the cargo of the exosome-like nanovesicles. According to some embodiments, the conditions of primary stress include high/low temperature, salinity; drought, dehydration; flooding; heavy metal chemical pollutants; light stresses, and physical wounding. According to some embodiments, these primary stresses produce secondary stresses, e.g., oxidative stress and osmotic stress. For example, the plant(s) can be subjected to at least 1 stress condition. According to some embodiments, the plant(s) can be subjected to a combination of at least 2 different stress conditions, for example, high/low temperature and salinity; high/low temperature and drought; high/low temperature and dehydration; high/low temperature and dehydration; high/low temperature and flooding; high/low temperature and heavy metal chemical pollutants; high/low temperature and light stresses; high/low temperature and physical wounding.

According to some embodiments, the cargo comprises a nutraceutical, cosmetic, cosmeceutical or therapeutic signature including expression of proteins, nucleic acids, or both that is not expressed in a plant not subject to the abiotic stress conditions.

According to some embodiments, the protein cargo of the plant-derived exosomes includes heat shock protein (HSP) chaperones induced under physiological stress, heat shock transcription factors (HSFs), or both. According to some embodiments, the HSPs includes an HSP100 class, an HSP90 class, an HSP70 class, an HSP60 class, a small hsp class or a combination thereof. According to some embodiments, the hsfs induced under physiological stress include hsfA, hsfB, hsfC or a combination thereof.

According to some embodiments, the plant protein signature cargo of Aloe-derived exosomes comprises a ribulose 1,5-bisphosphate carboxylase/oxygenase subunit, a calcium dependent protein kinase, a PLAC8 family protein, and a glutathione transferase as shown in Appendix A. In plants, PLAC8 motif-containing proteins are involved in the determination of organ size and growth, response to infection, Ca²⁺ influx, Cd resistance, and Zn detoxification. In mammals, PLAC8 is a cysteine-rich protein described as a central mediator of tumor evolution. The human PLAC8 gene is involved in contact hypersensitivity response and plays a role in psoriatic skin. [Cabreira-Cagliari, C. et al. Genome (2018) 61 (12): 857-65].

According to some embodiments, the protein signature cargo of Aloe exosomes correlated to relevant human proteins obtained from existing databases comprises keratin 1, cytoskeletal 6B keratin type II, Keratin 6B, semaphorin receptor; plexin-B1; mitogen-activated protein kinase kinase kinase 2 (MEKK2), diacylglyerol kinase; chondroadherin, T cell receptor beta chain, and fez family zinc finger protein isoform 1 and isoform 2. as shown in Appendix B.

According to some embodiments, the signature cargo of plant-derived exosomes when isolated from heat-stressed Aloe vera plants through RNASeq. as described in Example 3, including only those miRNAs >100, contains: ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR43; ath-miR838; ath-miR5654-5p; ath-miR72d-5p; ath-miR5642a; ath-miR420; ath-miR83-3p; ath-miR156d-3p; ath-miR5018; ath-miR8168; ath-miR866-3p; ath-miR87-3p; ath-miR395b; ath-miR395c; ath-miR78.2; ath-miR67c-5p; ath-miR393a-3p; ath-miR395f; or a combination thereof. According to some embodiments, the functional outcome of miRNA transfer via plant-derived exosomes depends on the cell type affected and the inducing signal, here abiotic stress.

The sequence of the Arabidopsis reference mature miRNA strand is shown in Table 2 [source, miRBase.org].

TABLE 2
Signature miRNAs

| | | SEQ ID | miRBase |
|---|---|---|---|
| Signature miRNA | Mature miRNA Sequence | NO: | Accession |
| ath-miR166a-3p | UCGGACCAGGCUUCAUUCCCC | 1 | MIMAT0000189 |
| ath-miR166b-3p | UCGGACCAGGCUUCAUUCCCC | 2 | MIMAT0000190 |
| ath-miR166e-3p | UCGGACCAGGCUUCAUUCCCC | 3 | MIMAT0000193 |
| ath-miR396a-5p | UUCCACAGCUUUCUUGAACUG | 4 | MIMAT0000944 |
| ath-miR396b-5p | UUCCACAGCUUUCUUGAACUU | 5 | MIMAT0000945 |
| ath-miR156f-5p | UGACAGAAGAGAGUGAGCAC | 6 | MIMAT0000171 |
| ath-miR168b-5p | UCGCUUGGUGCAGGUCGGGAA | 7 | MIMAT0000199 |
| ath-miR156c-5p | UGACAGAAGAGAGUGAGCAC | 8 | MIMAT0000168 |
| ath-miR162a-3p | UCGAUAAACCUCUGCAUCCAG | 9 | MIMAT0000182 |
| ath-miR162b-3p | UCGAUAAACCUCUGCAUCCAG | 10 | MIMAT0000183 |
| ath-miR396a-3p | GUUCAAUAAAGCUGUGGGAAG | 11 | MIMAT0031908 |
| ath-miR168a-5p | UCGCUUGGUGCAGGUCGGGAA | 12 | MIMAT0000198 |
| ath-miR156b-5p | UGACAGAAGAGAGUGAGCAC | 13 | MIMAT0000167 |
| ath-miR156a-5p | UGACAGAAGAGAGUGAGCAC | 14 | MIMAT0000166 |
| ath-miR156d-5p | UGACAGAAGAGAGUGAGCAC | 15 | MIMAT0000169 |
| ath-miR164c-5p | UGGAGAAGCAGGGCACGUGCG | 16 | MIMAT0001017 |
| ath-miR408-3p | AUGCACUGCCUCUUCCCUGGC | 17 | MIMAT0001011 |
| ath-miR165a-3p | UCGGACCAGGCUUCAUCCCCC | 18 | MIMAT0000187 |
| ath-miR160a-5p | UGCCUGGCUCCCUGUAUGCCA | 19 | MIMAT0000178 |
| ath-miR157b-5p | UUGACAGAAGAUAGAGAGCAC | 20 | MIMAT0000173 |
| ath-miR157a-5p | UUGACAGAAGAUAGAGAGCAC | 21 | MIMAT0000172 |
| ath-miR164b-5p | UGGAGAAGCAGGGCACGUGCA | 22 | MIMAT0000186 |
| ath-miR5016 | UUCUUGUGGAUUCCUUGGAAA | 23 | MIMAT0020520 |
| ath-miR5998b | ACAGUUUGUGUUUUGUUUUGU | 24 | MIMAT0023522 |
| ath-miR5020a | UGGAAGAAGGUGAGACUUGCA | 25 | MIMAT0020524 |
| ath-miR836 | UCCUGUGUUUCCUUUGAUGCGUGG | 26 | MIMAT0004257 |
| ath-miR158a-5p | CUUUGUCUACAAUUUUGGAAA | 27 | MIMAT0031873 |
| ath-miR395e | CUGAAGUGUUUGGGGGAACUC | 28 | MIMAT0000942 |
| ath-miR402 | UUCGAGGCCUAUUAAACCUCUG | 29 | MIMAT0001003 |
| ath-miR5662 | AGAGGUGACCAUUGGAGAUG | 30 | MIMAT0022439 |
| ath-miR5630a | GCUAAGAGCGGUUCUGAUGGA | 31 | MIMAT0022389 |
| ath-miR5630b | GCUAAGAGCGGUUCUGAUGGA | 32 | MIMAT0022399 |
| ath-miR3933 | AGAAGCAAAAUGACGACUCGG | 33 | MIMAT0018348 |
| ath-miR5998a | ACAGUUUGUGUUUUGUUUUGU | 34 | MIMAT0023521 |
| ath-miR172e-3p | GGAAUCUUGAUGAUGCUGCAU | 35 | MIMAT0001019 |
| ath-miR5024-3p | CCGUAUCUUGGCCUUGUCAUU | 36 | MIMAT0021048 |
| ath-miR447a-3p | UUGGGGACGAGAUGUUUUGUUG | 37 | MIMAT0002113 |
| ath-miR414 | UCAUCUUCAUCAUCAUCGUCA | 38 | MIMAT0001322 |
| ath-miR167a-3p | GAUCAUGUUCGCAGUUUCACC | 39 | MIMAT0031883 |
| ath-miR172b-5p | GCAGCACCAUUAAGAUUCAC | 40 | MIMAT0000204 |
| ath-miR5636 | CGUAGUUGCAGAGCUUGACGG | 41 | MIMAT0022396 |
| ath-miR824-3p | CCUUCUCAUCGAUGGUCUAGA | 42 | MIMAT0032024 |
| ath-miR172e-5p | GCAGCACCAUUAAGAUUCAC | 43 | MIMAT0031918 |
| ath-miR404 | AUUAACGCUGGCGGUUGCGGCAGC | 44 | MIMAT0001005 |
| ath-miR 447b | UUGGGGACGAGAUGUUUUGUUG | 45 | MIMAT0002114 |
| ath-miR826b | UGGUUUUGGACACGUGAAAAU | 46 | MIMAT0035543 |
| ath-miR169g-5p | UGAGCCAAGGAUGACUUGCCG | 47 | MIMAT0000911 |
| ath-miR868-3p | CUUCUUAAGUGCUGAUAAUGC | 48 | MIMAT0004319 |
| ath-miR830-3p | UAACUAUUUUGAGAAGAAGUG | 49 | MIMAT0004248 |
| ath-miR169f-5p | UGAGCCAAGGAUGACUUGCCG | 50 | MIMAT0000910 |
| ath-miR828 | UCUUGCUUAAAUGAGUAUUCCA | 51 | MIMAT0004244 |
| ath-miR8182 | UUGUGUUGCGUUUCUGUUGAUU | 52 | MIMAT0032781 |
| ath-miR160a-3p | GCGUAUGAGGAGCCAUGCAUA | 53 | MIMAT0031874 |
| ath-miR5635d | UGUUAAGGAGUGUUAACGGUG | 54 | MIMAT0022405 |
| ath-miR399c-3p | UGCCAAAGGAGAGUUGCCCUG | 55 | MIMAT0000953 |
| ath-miR5635c | UGUUAAGGAGUGUUAACGGUG | 56 | MIMAT0022428 |
| ath-miR398a-3p | UGUGUUCUCAGGUCACCCCUU | 57 | MIMAT0000948 |
| ath-miR391-5p | UUCGCAGGAGAGAUAGCGCCA | 58 | MIMAT0000933 |
| ath-miR781a | UUAGAGUUUUCUGGAUACUUA | 59 | MIMAT0003940 |
| ath-miR157c-3p | GCUCUCUAUACUUCUGUCACC | 60 | MIMAT0031872 |
| ath-miR399b | UGCCAAAGGAGAGUUGCCCUG | 61 | MIMAT0000952 |
| ath-miR5014b | AUUUGUACACCUAGAUCUGUA | 62 | MIMAT0023524 |
| ath-miR5635b | UGUUAAGGAGUGUUAACGGUG | 63 | MIMAT0022418 |
| ath-miR779.2 | UGAUUGGAAAUUUCGUUGACU | 64 | MIMAT0004329 |
| ath-miR390b-5p | AAGCUCAGGAGGGAUAGCGCC | 65 | MIMAT0000932 |
| ath-miR833a-3p | UAGACCGAUGUCAACAAACAAG | 66 | MIMAT0004253 |
| ath-miR849 | UAACUAAACAUUGGUGUAGUA | 67 | MIMAT0004271 |
| ath-miR5635a | UGUUAAGGAGUGUUAACGGUG | 68 | MIMAT0022395 |
| ath-miR841b-3p | CAAUUUCUAGUGGGUCGUAUU | 69 | MIMAT0017742 |
| ath-miR390a-5p | AAGCUCAGGAGGGAUAGCGCC | 70 | MIMAT0000931 |
| ath-miR447c-3p | UUGGGGACGACAUCUUUUGUUG | 71 | MIMAT0002115 |
| ath-miR835-3p | UGGAGAAGAUACGCAAGAAAG | 72 | MIMAT0004256 |
| ath-miR5638b | ACAGUGGUCAUCUGGUGGGCU | 73 | MIMAT0022407 |
| ath-miR2112-3p | CUUUAUAUCCGCAUUUGCGCA | 74 | MIMAT0011155 |
| ath-miR5653 | UGGGUUGAGUUGAGUUGAGUUGGC | 75 | MIMAT0022425 |
| ath-miR166a-5p | GGACUGUUGUCUGGCUCGAGG | 76 | MIMAT0031880 |
| ath-miR159b-5p | GAGCUCCUUGAAGUUCAAUGG | 77 | MIMAT0031889 |
| ath-miR166b-5p | GGACUGUUGUCUGGCUCGAGG | 78 | MIMAT0031881 |
| ath-miR843 | UUUAGGUCGAGCUUCAUUGGA | 79 | MIMAT0004265 |
| ath-miR5015 | UUGGUGUUAUGUGUAGUCUUC | 80 | MIMAT0020519 |
| ath-miR781b | UUAGAGUUUUCUGGAUACUUA | 81 | MIMAT0022420 |
| ath-miR4245 | ACAAAGUUUUAUACUGACAAU | 82 | MIMAT0023523 |
| ath-miR169b-5p | CAGCCAAGGAUGACUUGCCGG | 83 | MIMAT0000906 |
| ath-miR5013 | UUUGUGACAUCUAGGUGCUUU | 84 | MIMAT0020517 |
| ath-miR864-5p | UCAGGUAUGAUUGACUUCAAA | 85 | MIMAT0004311 |
| ath-miR866-5p | UCAAGGAACGGAUUUUGUUAA | 86 | MIMAT0004315 |
| ath-miR5595a | ACAUAUGAUCUGCAUCUUUGC | 87 | MIMAT0023517 |
| ath-miR403-3p | UUAGAUUCACGCACAAACUCG | 88 | MIMAT0001004 |
| ath-miR164c-3p | CACGUGUUCUACUACUCCAAC | 89 | MIMAT0031916 |
| ath-miR835-5p | UUCUUGCAUAUGUUCUUUAUC | 90 | MIMAT0004255 |
| ath-miR165b | UCGGACCAGGCUUCAUCCCCC | 91 | MIMAT0000188 |
| ath-miR3434-5p | ACUUGGCUGAUUCUAUUAUU | 92 | MIMAT0017737 |
| ath-miR8176 | GGCCGGUGGUCGCGAGAGGGA | 93 | MIMAT0032775 |
| ath-miR5631 | UGGCAGGAAAGACAUAAUUUU | 94 | MIMAT0022390 |
| ath-miR399a | UGCCAAAGGAGAUUUGCCCUG | 95 | MIMAT0000951 |
| ath-miR 4227 | UCACUGGUACCAAUCAUUCCA | 96 | MIMAT0017943 |
| ath-miR5666 | AUGGGACAUCGAGCAUUUAAU | 97 | MIMAT0022444 |
| ath-miR778 | UGGCUUGGUUUAUGUACACCG | 98 | MIMAT0003937 |
| ath-miR851-3p | UGGGUGGCAAACAAAGACGAC | 99 | MIMAT0004274 |
| ath-miR5663-3p | UGAGAAUGCAAAUCCUUAGCU | 100 | MIMAT0032129 |
| ath-miR832-3p | UUGAUUCCCAAUCCAAGCAAG | 101 | MIMAT0004251 |
| ath-miR5646 | GUUCGAGGCACGUUGGGAGG | 102 | MIMAT0022410 |
| ath-miR856 | UAAUCCUACCAAUAACUUCAGC | 103 | MIMAT0004300 |
| ath-miR837-5p | AUCAGUUUCUUGUUCGUUUCA | 104 | MIMAT0004258 |
| ath-miR846-3p | UUGAAUUGAAGUGCUUGAAUU | 105 | MIMAT0004269 |
| ath-miR827 | UUAGAUGACCAUCAACAAACU | 106 | MIMAT0004243 |
| ath-miR5633 | UAUGAUCAUCAGAAAACAGUG | 107 | MIMAT0022393 |
| ath-miR413 | AUAGUUUCUCUUGUUCUGCAC | 108 | MIMAT0001321 |
| ath-miR838 | UUUUCUUCUACUUCUUGCACA | 109 | MIMAT0004260 |
| ath-miR5654-5p | AUAAAUCCCAACAUCUUCCA | 110 | MIMAT0022426 |
| ath-miR172d-5p | GCAACAUCUUCAAGAUUCAGA | 111 | MIMAT0031901 |
| ath-miR5642a | UCUCGCGCUUGUACGGCUUU | 112 | MIMAT0022403 |
| ath-miR420 | UAAACUAAUCACGGAAAUGCA | 113 | MIMAT0001328 |
| ath-miR831-3p | UGAUCUCUUCGUACUCUUCUUG | 114 | MIMAT0004249 |
| ath-miR156d-3p | GCUCACUCUCUUUUUGUCAUAAC | 115 | MIMAT0031868 |
| ath-miR5018 | UUAAAGCUCCACCAUGAGUCCAAU | 116 | MIMAT0020522 |
| ath-miR8168 | AGGUGCUGAGUGUGCUAGUGC | 117 | MIMAT0032766 |
| ath-miR866-3p | ACAAAAUCCGUCUUUGAAGA | 118 | MIMAT0004316 |
| ath-miR8170-3p | UUGCUUAAAGAUUUUCUAUGU | 119 | MIMAT0032769 |
| ath-miR395b | CUGAAGUGUUUGGGGGGACUC | 120 | MIMAT0000939 |
| ath-miR395c | CUGAAGUGUUUGGGGGGACUC | 121 | MIMAT0000940 |
| ath-miR780.2 | UUCUUCGUGAAUAUCUGGCAU | 122 | MIMAT0003939 |
| ath-miR167c-5p | UAAGCUGCCAGCAUGAUCUUG | 123 | MIMAT0001018 |
| ath-miR393a-3p | AUCAUGCUAUCUCUUUGGAUU | 124 | MIMAT0031905 |
| ath-miR395f | CUGAAGUGUUUGGGGGGACUC | 125 | MIMAT0000943 |

According to the miR database (mirbase.org), miR166 is highly conserved and has diverse functions across plant species. In Vaccinium corymbosum, for example, conserved Vco-miR166 family members display functional diversification but also coordinately influence plant responses to abiotic stress. [Li, Y., et al. Front. Genet. (2022) 13: 919856].

According to the miR database (mirbase.org) miR166a, like miR165, is thought to target mRNAs coding for HD-Zip transcription factors including Phabulosa (PHB) and Phavoluta (PHV) that regulate axillary meristem initiation and leaf development in Arabidopsis thaliana; [Id., citing Rhoades, M W., et al. Cell (2002) 110: 513-520]].

According to the miR database (mirbase.org), the miR396 family of mature miRNAs are predicted to target mRNAs coding for Growth Regulating Factor (GRF) transcription factors, rhodenase-like proteins, and kinesin-like protein B [Id., citing Xie, Z., et al. Plant Physiol. (2005) 138: 2145-2154].

According to the miR database (mirbase.org), ath-miR-156c, ath-miR157b-5p, and ath-miR156f mature miRNAs are thought to target 10 mRNAs coding for proteins containing the Squamosa-promoter Binding Protein (SBP) box [Id., citing Reinhart, B J et al. Genes Dev. (2002) 16: 1616-1626]. The complementary sites are downstream of this conserved domain, within a poorly conserved protein-coding context or the Y UTR [Id., citing Rhoades, M W., et al. Cell (2002) 110: 513-520].

According to the miR database (mirbase.org), mature ath-miR-168b is thought to target mRNAs coding for Argonaute (AGO1), which is required for axillary shoot meristem formation and leaf development in Arabidopsis. It has been suggested that AGO1 may also influence miRNA accumulation in plants and that miR168 may act as a negative-feedback mechanism for controlling expression of the AGO1 gene [Id., citing Rhoades, M W et al. Cell (2002) 110: 513-520].

According to the miR database (mirbase.org), mature ath-miR-164c-5p is a predicted paralogue of the previously identified miR164 family [Id., citing Rajagopalan, R. et al. Genes Dev. (2006) 20: 3407-3425]. It is predicted to target mRNAs coding for NAC domain transcription factors.

According to the miR database (mirbase.org), mature miR160a-5p is thought to target mRNAs coding for auxin response factor proteins [Id., citing Rhoades, M W., et al. Cell (2002) 110: 513-520].

According to the miR database (mirbase.org) miR395 is thought to target mRNAs coding for ATP sulphurylases in Arabidopsis thaliana [Id., citing Xie, Z, et al. Plant Physiol (2005) 138:2145-2154].

According to the miR database (mirbase.org) miR172 is thought to target mRNAs coding for APETALA2-like transcription factors in Arabidopsis thaliana [Id., citing Xie, Z, et al. Plant Physiol (2005) 138:2145-2154].

According to the miR database (mirbase.org) miR167, like miR160, is thought to target mRNAs coding for auxin response factors, DNA binding proteins that are thought to control transcription in response to the phytohormone auxin. Transcription regulation is important for many of the diverse developmental responses to auxin signals, which include cell elongation, division, and differentiation in both roots and shoots in Arabidopsis thaliana [Id., citing Rhoades, M W., et al. Cell (2002) 110:513-520].

According to the miR database (mirbase.org) miR169 is thought to target mRNAs coding for CCAAT Binding Factor (CBF) and HAP2-like transcription factors in Arabidopsis thaliana [Id., citing Rajagopalan, R, et al. Genes Dev (2006) 20:3407-3425, and Moldovan, D., et al., J Exp Bot (2010) 61:165-177].

According to the miR database (mirbase.org) miR399 is thought to target mRNAs coding for phosphatase transporters in Arabidopsis thaliana [Id., citing Xie, Z, et al. Plant Physiol (2005) 138:2145-2154].

According to the miR database (mirbase.org) miR398 is thought to target mRNAs coding for copper superoxide dismutases and cytochrome C oxidase subunit V in Arabidopsis thaliana [Id., citing Lu, C., et al. Genome Res (2006) 16:1276-1288].

According to the miR database (mirbase.org) miR157, like miR156, is thought to target mRNAs coding for proteins containing the squamosa-promoter binding protein (SBP) box in Arabidopsis thaliana [Id., citing Rhoades, M W, et al. Cell (2002) 110:513-520].

According to the miR database (mirbase.org) miR159 is thought to target mRNAs coding for MYB proteins which are known to bind to the promoter of the floral meristem identity gene LEAFY in Arabidopsis thaliana [Id., citing Lu, C., et al. Genome Res (2006) 16:1276-1288].

According to the miR database (mirbase.org) miR164 is thought to target mRNAs coding for NAC domain transcription factors in Arabidopsis thaliana [Id., citing Rajagopalan, R, et al. Genes Dev (2006) 20:3407-3425].

According to the miR database (mirbase.org) miR165 is thought to target mRNAs coding for HD-Zip transcription factors including Phabulosa (PHB) and Phavoluta (PHV) that regulate axillary meristem initiation and leaf development in Arabidopsis thaliana [Id., citing Rhoades, M W., et al. Cell (2002) 110:513-520].

According to the miR database (mirbase.org) miR393 is thought to target mRNAs coding for F-box proteins and bHLH transcription factors in Arabidopsis thaliana [Id., citing Lu, C., et al. Genome Res (2006) 16:1276-1288].

According to some embodiments, reactome analysis correlating the signature Aloe miRNA cargo to known human pathways indicates that these miRNAs can affect signaling pathways in the immune system, the nervous system, and the skin. According to some embodiments, the correlated signaling pathways include ERK/MAPK signaling; and/or leptin signaling; and/or cytokine signaling; and/or interleukin signaling, and/or type 1 insulin-like growth factor 1 receptor (IGF-1R) signaling; and/or VEGFA/VEGFR2 signaling, semaphorin signaling; and/or sirtuins signaling; and/or LRP1 signaling.

Sadagurski et al. showed that IGF-1R plays an inhibitory role in the regulation of skin development and differentiation. [Sadagurski, M., et al. Molec. & Cellular Biol. (2006) 26: (7): 2675-2687]. For example, they showed that IGF-1R deficient skin cultures show abnormal patterns of maturation and differentiation, and IGF-1R null keratinocytes exhibit accelerated differentiation and decreased proliferation; that phosphatidylinositol 3-kinase (PI3K) and extracellular signal-regulated kinase 1 and 2 are involved in the regulation of skin keratinocyte differentiation and take some part in mediating the inhibitory signal of IGF-1R on differentiation and that mammalian target or rapamycin (mTOR) has been shown to play a specific role in mediating IGF-1R impedance of action on keratinocyte differentiation. The development and progression of skin cancers involve multiple signaling pathways, including that of IGF-1R. [Wang, X., et al. Theranostics (2022) 12 (6): 2613-2630]. Lewis, et al showed that IGF-1R pays a role in suppressing UVB-induced carcinogenesis and suggested that fibroblasts play a critical role in maintaining appropriate activation of the keratinocyte IGF-1R, and that reduced expression of IGF-1 in geriatric skin could be important component in the development of aging-related non-melanoma skin cancer [Lewis, D A., et al. Oncogene (2010) 29 (10): 1475-1485].

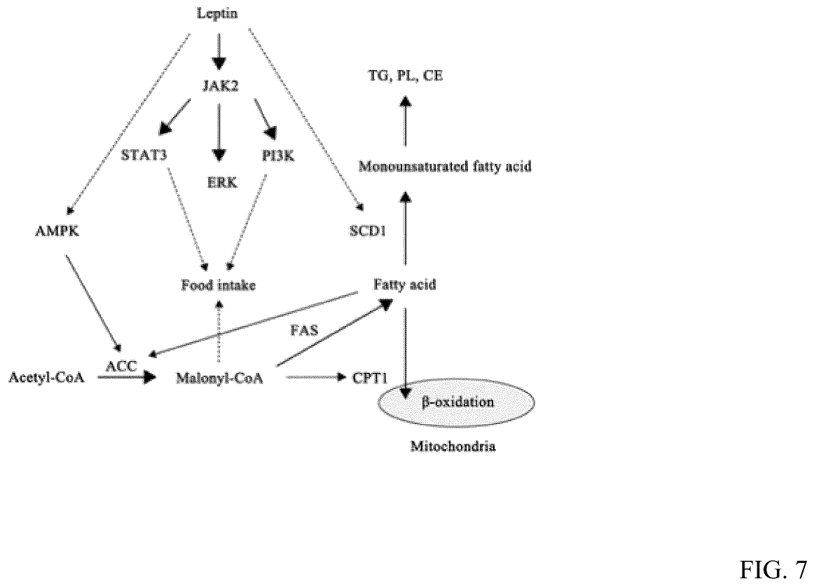

Leptin, a 16 kD protein, is synthesized and secreted by white adipose tissue. Leptin possesses pleiotropic functions, including stimulation of angiogenesis, modulation of the hormone system, and augmentation of production of pro-inflammatory cytokines. Its activity has been associated with the development and maintenance of pro-inflammatory immune responses where it acts as a pleiotropic hormone and activator of the cytokine cascade. The effects of leptin on the processes taking place within the skin and hair, as well as its role in the pathogenesis of different skin diseases, have been confirmed [see, e.g., Dopytalska, K., et al. Lipids Health Dis. (2020) 19: 215]. A schematic representation of leptin and leptin receptor signal transduction pathways is shown in FIG. 7.

Signal transduction networks initiated by VEGFA/VEGFR2, the most prominent ligand-receptor complex in the VEGF system, leads to endothelial cell proliferation, migration, survival and new vessel formation involved in angiogenesis [Abhinand, C S., et al. J. Cell Commun. Signal. (2016) 10 (4): 347-354]. VEGF modulates the innate and adaptive immune response directly or indirectly in three different ways: through its interaction with immune cells, by modulating protein expression in endothelial cells, or by modulating vascular permeability. In turn, immunosuppressive immune cells can produce proangiogenic factors and promote angiogenesis, creating a positive feedback loop [Geindreau, M. et al. Int. J. Mol. Sci. (2021) 22 (9): 4871].

According to some embodiments, the cargo of plant-derived exosomes can modulate the bioactivities of recipient cells. According to some embodiments, the recipient cells are mammalian cells. According to some embodiments, the recipient cells are human cells. Examples of human cells include, without limitation, fibroblasts, keratinocytes, melanocytes, Langerhans cells in skin; immune cells, such as B cells, T cells, dendritic cells, macrophages, etc.

According to some embodiments, a composition comprising abiotically stressed plant-derived exosome-like nanoparticles comprising a cargo is applied topically to human skin.

According to some embodiments, the loading of the plant-derived exosome-like nanovesicles can be modulated/tuned to result in plant-derived exosome-like nanoparticles comprising cargo with enhanced skin conditioning activities.

According to some embodiments, a composition comprising the exosome-like nanoparticles derived from abiotically stressed plants comprising a cargo modulates gene expression in human immune cells, keratinocytes, melanocytes, or fibroblasts in vitro.

According to some embodiments, a composition comprising the exosome-like nanoparticles derived from abiotically stressed plants comprising a cargo induces collagen production in human dermal fibroblasts in vitro.

According to some embodiments, a composition comprising the exosome-like nanoparticles derived from abiotically stressed plants comprising a cargo increases elastin production in human dermal fibroblasts in vitro.

According to some embodiments, a composition comprising the exosome-like nanoparticles derived from abiotically stressed plants comprising a cargo increases hyaluronic acid production in human dermal fibroblasts in vitro.

According to some embodiments, a composition comprising the exosome-like nanoparticles derived from abiotically stressed plants comprising a cargo regulates interferon α2 production in PBMCs exposed to P. acnes in vitro.

According to some embodiments, a composition comprising the exosome-like nanoparticles derived from abiotically stressed plants comprising a cargo increases VEGF-A production in human dermal fibroblasts in vitro. It has been reported that VEGF-A mediated signaling is both required and sufficient for rejuvenation of human skin [Keren, A., et al. Sci. Adv. (2022) 8 (25): eabm6756].

According to some embodiments, a composition comprising the exosome-like nanoparticles derived from abiotically stressed plants comprising a cargo modulates melanin production and impacts skin pigmentation in B16 melanoma cells in vitro. B16 melanoma is a murine tumor cell line used for research as a model for human skin cancers.

According to some embodiments, a composition comprising the exosome-like nanoparticles derived from abiotically stressed plants comprising a cargo when applied to the skin may modulate gene expression in immune cells, keratinocytes, melanocytes, or fibroblasts in the skin.

According to some embodiments, a composition comprising the exosome-like nanoparticles derived from abiotically stressed plants comprising a cargo when applied to human skin may improve the youthful appearance of the skin.

According to some embodiments, a composition comprising the exosome-like particles derived from abiotically stressed plants comprising a cargo may reduce the appearance of wrinkles by stimulating hyaluronic acid and collagen production.

According to some embodiments, a composition comprising the exosomes derived from abiotically stressed plants comprising a cargo when applied to skin may improve the overall appearance of the skin.

For example, according to some embodiments, a composition comprising the exosome-like nanoparticles derived from abiotically stressed plants comprising a cargo when applied topically to human skin may improve skin clarity. According to some embodiments, a composition comprising the exosome-like nanoparticles derived from abiotically stressed plants comprising a cargo when applied topically to human skin may improve skin texture. According to some embodiments, a composition comprising the exosome-like nanoparticles derived from abiotically stressed plants comprising a cargo when applied topically to human skin may improve skin luminosity. According to some embodiments, a composition comprising the exosome-like nanoparticles derived from abiotically stressed plants comprising a cargo when applied topically to human skin may improve skin radiance. According to some embodiments, a composition comprising the exosome-like nanoparticles derived from abiotically stressed plants comprising a cargo may rejuvenate the appearance of skin.

According to some embodiments, a composition comprising the exosome-like nanoparticles derived from abiotically stressed plants comprising a cargo may modulate a pathway that contributes to inflammation, immune dysfunction, or both. According to some embodiments, the pathway is a phosphoinositide 3-kinase/Akt/mTOR pathway, a mitogen-activated protein kinase pathway, an IGF-1 signaling pathway, a sirtuin pathway, an LRP1 pathway or a combination thereof. According to some embodiments, a composition comprising the exosome-like nanoparticles derived from abiotically stressed plants comprising a cargo may modulate the circadian clock of the skin and its components.

### Compositions/Formulations

Compositions of the described invention may be prepared using technology, which is known in the art, such as described in Remington's Pharmaceutical Sciences, 18th or 19th editions, published by the Mack Publishing Company of Easton, Pennsylvania, which is incorporated herein by reference.

According to some embodiments, the present disclosure provides topical compositions comprising the plant-derived exosome-like nanoparticles of the described invention and a carrier. Topical administration may also involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

Dissolvable microneedles comprising patches composed of microscopic cones and often composed of sugar and salt solutions can deliver compositions directly to the dermis and the immune cells that reside there. The composition is added to the sugar and salt solution and dried; once the microneedles are applied to the skin, the moisture in a patient's skin can dissolve the microneedles and absorb the composition components.

According to some embodiments, the present disclosure further provides a method for treating an epithelial-related condition including topically applying onto an epithelial surface of a mammal, in need thereof including a human, a composition comprising the plant-derived exosome-like nanoparticles of the described invention and a carrier.

The compositions of the described invention may be usefully employed in cosmetic, cosmeceutical and general skincare compositions as well as in pharmaceutical compositions. According to some embodiments, the composition is a cosmetic composition comprising a cosmetic amount of purified enriched plant-derived exosomes comprising a cargo comprising a cosmetic signature and a cosmetically acceptable carrier. According to some embodiments, the composition is a cosmeceutical composition comprising a cosmeceutical amount of purified enriched plant-derived exosomes comprising a cargo comprising a cosmeceutical signature and a cosmeceutical carrier. According to some embodiments, a nutraceutical composition providing health benefits in addition to its basic nutritional value and not intended to diagnose, treat, cure or prevent any disease comprises a nutraceutical amount of purified enriched plant-derived exosomes comprising a cargo comprising a dietary signature and an acceptable carrier. According to some embodiments, where the intent is to diagnose, treat, cure or prevent a disease, the composition is a pharmaceutical composition comprising a pharmaceutical amount of purified enriched plant-derived exosomes comprising a cargo comprising a therapeutic signature and a pharmaceutically acceptable carrier. Suitable cosmetically acceptable carriers are described in the CTFA International Cosmetic Ingredient Dictionary and Handbook, 8th edition, edited by Wenninger and Canterbery, (The Cosmetic. Toiletry, and Fragrance Association, Inc., Washington, D, C, 2000), which is herein incorporated by reference. Also included are the carriers described hereinabove. It will be understood that cosmetically acceptable carriers, cosmeceutically acceptable carriers and pharmaceutically acceptable carriers are similar, if not often identical, in nature.

The phrase “pharmaceutically acceptable carrier” is art recognized. It is used to mean any substantially non-toxic carrier conventionally useable for administration of pharmaceuticals in which the isolated plant-derived exosome-like nanoparticles (PDELNs) of the present invention will remain stable and bioavailable. The pharmaceutically acceptable carrier must be of sufficiently high purity and of sufficiently low toxicity to render it suitable for administration to the mammal being treated. It should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. According to some embodiments, the pharmaceutically acceptable carrier comprises a pulmonary surfactant. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the subject by employing procedures well known in the art.

As is widely recognized in the art, since the pH of human skin is 5.5, to avoid irritation, compositions for topical skin application should have a pH value of between pH 4.0 and pH 7.0, inclusive. In some embodiments, the pH of the composition is between pH 5.0 and pH 7.0, inclusive. In some embodiments, the pH is about 5.5. Hence, a pH adjusting composition is typically added to bring the pH of the composition to the desired value. The compositions of the described invention therefore can be formulated to have a pH value of about 7.0, Suitable pH adjusting agents include, for example, but are not limited to, one or more adipic acids, glycines, citric acids, calcium hydroxides, magnesium aluminometasilicates, buffers or any combinations thereof.

### Administration

The described invention relates to all routes of administration including intramuscular, subcutaneous, sublingual, intravenous, intraperitoneal, intranasal, intratracheal, intravaginal, transvaginal, topical, intradermal, intramucosal, intracavernous, intrarectal, into a sinus, gastrointestinal, intraductal, intrathecal, intraventricular, intrapulmonary, into an abscess, intraarticular, subpericardial, into an axilla, into the pleural space, intrabuccal, transmucosal, transdermal, via inhalation, via nebulizer, and via subcutaneous injection. Alternatively, the plant-derived exosome composition may be introduced by various means into cells that are removed from the individual. Such means include, for example, microprojectile bombardment, via liposomes or via other nanoparticle device.

Transmucosal drug administration refers to the absorption of drug through the mucosal epithelium into the systemic circulation. Intranasal, oral transmucosal and rectal routes are the major transmucosal routes. Mucus is a universal delivery barrier at the mucosal surface, and drugs are usually absorbed through the mucosa into the systemic circulation through either the transcellular or paracellular route. They typically have low molecular weight (<500 Da) with a high log P value (>2.0), where P is the octanol-water partition coefficient. The higher the log P, the more lipophilic the drug is. [Lam, J K W., et al. Adv. Drug Deliv. Rev. (2020) 160: 234-43]. Only a small number of drugs exhibit the desirable physicochemical properties to cross the mucosal epithelium effectively. In order to enhance drug absorption, penetration enhancers (or permeation enhancers) are commonly investigated in the development of transmucosal formulations to overcome the epithelial barrier, especially in intranasal, buccal, and to a lesser extent, rectal formulations.

The term “transcellular route” refers to drug permeation through the cells while the term “paracellular route” refers to drug permeation between adjacent cells. Both routes belong to a passive transport process driven by a local concentration gradient. Whether a drug molecule can cross the epithelium and which route it takes are dependent on its intrinsic physicochemical properties. Hydrophilicity/lipophilicity, molecular weight and degree of ionization are the three major determinants. Lipophilic molecules can diffuse freely through the phospholipid bilayers of the cell membrane and therefore prefer the transcellular route. On the other hand, hydrophilic molecules cannot diffuse across the cell membrane, hence the paracellular route becomes significant to these molecules. However, the tight junction between adjacent cells limits the efficacy of this route of transportation. The smaller the molecules, the more effectively they can permeate through the tight junction. The degree of ionization is dependent on the pKa of the drug and the pH of the environment. Only the non-ionized species of a drug can permeate (by drug partitioning) through the cell membrane effectively, and this affects mainly the transcellular route. In general, small lipophilic molecules that are non-ionized at their surrounding pH are favorable for transmucosal absorption. Apart from the three aforementioned properties, aqueous solubility and dose of drug can also influence its absorption. However, absorption also relies on the volume of fluid available at the site of administration for drug dissolution to take place, hence the exact properties of a drug required in order to achieve effective transmucosal delivery is specific to each route of administration.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

Ocular formulations are intended to be applied on the anterior surface (topical route) of the eye, delivered intraocularly (inside the eye), periocularly (subtenon or juxtascleral), or in combination with ocular devices. Ocular dosage forms may be liquid, semi-solid, solid, or mixed. Liquid dosages include drops, suspension, and emulsion. Eye drops represent more than 95% of the marketed ocular products and are used for delivering the medication into the anterior part of the eye but with short residence time. Ocular suspensions and emulsions have the ability to deliver hydrophobic drugs but may lead to blurred vision. Ocular gels and ointments (semi-solid) may significantly enhance residence time. Solid dosage forms may be used to deliver water-sensitive drugs (powder), provide zero order release, or to sustain residence time (therapeutic contact lens). Effective ocular absorption necessitates appropriate corneal penetration along with effective precorneal residence time, so as to reach and preserve an acceptable drug concentration with the minimum quantity of the active therapeutic constituent. There are many ocular barriers such as tear film, corneal, conjunctival, and blood-ocular barriers that hinder their therapeutic efficacy. Because conventional eye drops are wasted by blinking and tear flow, their bioavailability is minimized to less than 5%. The cornea is composed of epithelium, stroma, and endothelium. The corneal epithelium allows only the passage of small and lipophilic drug. However, the corneal stroma allows the passage of hydrophilic drugs. The corneal endothelium conserves the transparency of the cornea and affords selective entry for hydrophilic drugs and macromolecules into the aqueous humor. The conjunctiva provides a minor impact to drug absorption compared to the cornea, though certain macromolecular nanomedicines, peptides, and oligonucleotides penetrate to the deep layers of the eye absolutely through these tissues. [Ahmed, S., et al. AAPS PharmSciTech (2023) 24: article 66]

Intracameral injections involve injection of antibiotic directly into the anterior segment of the eyeball or in the vitreous cavity. It is done usually subsequent to cataract surgery to avoid endophthalmitis initiated by a contagion of the eye that can occur after cataract surgery. Intravitreal injection is a delivery of medicine into the vitreous that is close to the retina at the back of the eye. Juxtascleral injections are used for treatment of some posterior part complaints that cannot be handled through conventional topical route. It is used for the treatment of cystoid macula edema, trauma, and diabetic-related conditions. Retrobulbar route involves the injection of a needle through the eyelid and orbital fascia to deliver the medication behind the globe into the retrobulbar space. Subconjunctival injection is frequently used in cases of very low drug penetration into the anterior part of the eye after topical administration. Subconjunctival injection of steroids fabricated as PEGylated liposome for handling of uveitis showed sustained anti-inflammatory activity and targeting the required ocular tissue. [Ahmed, S., et al. AAPS PharmSciTech (2023) 24: article 66]

### Dosage Forms

Formulations containing the plant-derived exosomes of the described invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the described invention may be in a form suitable for oral use, for example, as tablets, troches, lozenges, aqueous or oily suspensions, sprays, dispersible powders or granules, emulsions, hard or soft capsules or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be coated for controlled release.

Compositions of the present disclosure also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the present disclosure may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the present disclosure may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the present invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents may also be present.

Compositions of the invention also may be formulated as a beverage or as an additive to a beverage, where the term “beverage” refers to any non-alcoholic flavored carbonated drink, soda water, non-alcoholic still drinks, diluted fruit or vegetable juices whether sweetened or unsweetened, seasoned or unseasoned with salt or spice, or still or carbonated mineral waters used as a drink. The term “additive” as used herein refers to any substance the intended use of which results, or may reasonably be expected to result, directly or indirectly, in its becoming a component or otherwise affecting the characteristics of any beverage. In some embodiments, the beverage is a flavored carbonated beverage. In some embodiments, the beverage is a flavored non-carbonated beverage. In some embodiments, the beverage is a natural fruit beverage. The beverage also may contain one or more coloring agents, one or more flavoring agents, one or more sweetening agents, one or more antioxidant agents, and one or more preservatives.

Compositions of the invention also may be in the form of oil-in-water emulsions. The oily phase may be a vegetable oil, for example, olive oil or arachis oil, or a mineral oil, for example a liquid paraffin, or a mixture thereof. Suitable emulsifying agents may be naturally occurring gums, for example, gum acacia, naturally occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

Compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

The compositions of the present disclosure may be in the form of a sterile injectable aqueous or oleaginous suspension. The term “parenteral” as used herein includes subcutaneous injections, intravenous, intramuscular, intra-articular, intracisternal injection, or infusion techniques. Injectable preparations, such as sterile injectable aqueous or oleaginous suspensions, solutions or emulsions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. The parenteral composition can be formulated, for example, as a solution, suspension, emulsion or lyophilized powder in association with a pharmaceutically acceptable parenteral vehicle. Among the acceptable vehicles and solvents that may be employed are water, Ringer's solution, and isotonic sodium chloride solution. In addition, sterile, fixed oils are conventionally employed as a solvent or suspending medium. For parenteral application, exemplary vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers. Formulations for injection can be presented in unit dosage form, e.g., in ampoules or in multi-dose containers, with an added preservative. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

The compositions of the present disclosure may be in the form of a dispersible dry powder for pulmonary delivery. Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the present invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. Nos. 4,227,522; 4,192,309; and 4,105,027. Suitable containers also include those used in conjunction with Glaxo's Ventolin® Rotohaler™ brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

Administration by inhalation or insufflation (either through the mouth or through the nose, respectively) may involve an inhalation delivery device or a solid particulate therapeutic aerosol generator. According to some embodiments, the solid particulate aerosol generator is an insufflator. According to some embodiments, the inhalation delivery device is a nebulizer, a metered-dose inhaler, or a dry powder inhaler (DPI). According to some embodiments, respirable particles range in size from about 1 to 10 microns, inclusive; or particles for nasal administration (insufflation), range in size from 10-500 μM, inclusive.

According to some embodiments, the dry powder may be produced by a spray drying process.

The compositions of the present invention may be in the form of suppositories for rectal administration of the composition. These compositions may be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

In addition to the formulations described previously, the compositions of the described invention also can be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising any one or plurality of the active agents disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

Additional Active Ingredients. Additional active ingredients included in the compositions of the present disclosure to treat an epithelial-related condition include, without limitation, either alone or in combination, a serine protease inhibitor agent; a protective agent, an emollient, an astringent, an irritant, a keratolytic, a sun screening agent, a sun tanning agent, an antibiotic agent, a non-imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

### Serine Protease Inhibitors.

There are three distinct types of protein serine protease inhibitors.

The largest group of protein inhibitors is canonical inhibitors (for example, Cucurbita maxima trypsin inhibitor (CMTI trypsin inhibitor). These are small proteins that block the enzyme at the distorted Michaelis complex reaction stage; the protease-binding loop of canonical inhibitors is kept in a well-ordered conformation. [Krowarsch, D., et al. Cell Mol. Life Sci. (2003) 60: 2427-2444].

Serpins (serine protease inhibits) (e.g., alpha-1-antitrypsin, are much larger, typically 350-500 amino acids in size and are distributed from viruses to mammals. [Id, citing Gettins, PG. Chem. Rev. (2002) 102: 4751-4804; Silverman, GA., et al. J. Biol. Chem. (2001) 276: 33293-33296]. Like canonical inhibitors, serpins interact with their target proteases in a substrate manner. Unlike canonical inhibitors, the binding loop of serpins is much longer, about 17 amino acids, and able to adopt different conformations. In contrast to canonical inhibitors, serpins utilize the kinetic features of a hydrolytic reaction to form a very stable acyl-enzyme intermediate. The enzyme-serpin complex is a covalent acyl-enzyme adduct and upon acylation, the protease is translocated by over 70 A from its initial recognition site [Id. citing Huntington, J A., et al. Nature (2000) 407: 923-926]. Serpins are the only family of serine protease inhibitors for which complex formation with non-serine enzymes (cysteine proteases (Id., citing Komiyama, T., et al. J. Biol. Chem. (1994) 269: 19331-19337) and aspartyl proteases (Id., citing Mathialagan, N. and Hansen, TR. Proc. Natl Acad. Sci. USA (1996) 93: 13653-13658) has been demonstrated. SerpinB1, also known as leukocyte elastase inhibitor (LEI) or monocyte/NE inhibitor (MNE1)) is another example. [Majewski, P., et al. Front. Immunol. (2016) 7: 261].

Non-canonical inhibitors (e.g., ornithodorin: thrombin) interact through their N-terminal segment, which binds to the protease active site forming a short parallel β-sheet. These inhibitors, which only occur in blood sucking organisms and inhibit proteases involved in clot formation (thrombin or factor Xa) also form extensive secondary interactions with the target protease outside the active site. The classic example is recognition of thrombin by hirudin [Id., citing Stubbs, MT., et al. FEBS Letters (1995) 375: 103-107].

Table 3 below shows representative protein serine protease inhibitor families and their enzyme complexes (taken from Table 2 in Krowarsch, D., et al. Cell Mol. Life Sci. (2003) 60: 2427-2444).

TABLE 3
Representative Protein Serine Protease Inhibitor
Families and their Enzyme Complexes

| | | Enzyme- |
|---|---|---|
| Family | Representative | inhibitor complex |
| BPTI | bovine pancreatic trypsin | BPTI: rat trypsin |
| | inhibitor (BPTI) | |
| Kazal | Silver pheasant ovomucoid | Turkey ovomucoid |
| | third domain (OMSVP3) | (OMTKY)-chymotrypsin |
| Potato 1 | Chymotrypsin inhibitor 2 | Eglin c: subtilisin |
| | (CI-2) | |
| Squash | Cucurbita maxima trypsin | (Cucurbita pepo trypsin |
| | inhibitor I (CMTI 1) | inhibitor II |
| | | (CPTIII):- trypsin |
| Ecotin | Ecotin | Ecotin: crab |
| STI | soybean trypsin inhibitor | STI: porcine trypsin |
| | (STI) | |
| BBI | Barley Bowman-Birk | Mung bean Bowman-Birk |
| | inhibitor (BBBI) | inhibitor (MbBBI): Ns3- |
| | | protease |
| BBI (SFTI) | Sunflower trypsin inhibitor | SFTI-1: trypsin |
| | (SFTI-1) | |
| Antistasin | Hirustasin | Bdellastasin: |
| | | porcine trypsin |
| Ascaris | Apis mellifera chymotrypsin | C/E-1 inhibitor: porcine |
| | inhibitor (AMCI) | elastase |
| Grasshopper | Pars intercerebralis major | PMP-C_chymotrypsin |
| | peptide (PMP-C) | |
| SSI | Streptomyces subtilisn | SSI: subtilisin |
| | inhibitor (SSI) | |
| Potato 2 | Trypsin inhibitor from | PCI 1: SGPB |
| | Nicotiana alata (T1) | |
| Cereal | Corn Hageman factor | |
| | inhibitor (CHFI) | |
| Chelonianin | R-elafin | Elafin: porcine elastase |
| Rapeseed | Arabidopsis thaliana trypsin | |
| | inhibitor (ATTₚ) | |
| Arrowhead | | |

Mechanisms. Protein protease inhibitors can interact with proteases in different ways, although there are two mechanisms of interaction widely distributed in nature [Clemente, M. et al. Int. J. Mol. Sci. (2019) 20(6): 13453, citing Rawlings, N D et al. Biochem. J. (2004) 378: 705-716]. One of them is the irreversible trapping reaction; the best-characterized families of protease inhibitors that show this mechanism correspond to the families of serpins (I4), a2 macroglobulins (I39) and baculovirus protein p35 inhibitors (I50) [Id., citing Rawlings, N D et al., Biochem. J. (2004) 378: 705-16; Rawlings, ND. Biochimie (2010) 92: 1463-1483]. In this type of inhibition mechanism, the protease-inhibitor interaction induces the cleavage of an internal peptide bond in the inhibitor structure, triggering a conformational change. This reaction is not reversible, and the inhibitor never recovers its initial structure. For this reason, the inhibitors that participate in trapping reactions are also known as suicide inhibitors.

The other mechanism generally observed of protease-inhibitor interaction is known as a tight-binding reaction. This mechanism is also called a standard mechanism [Id., citing Lashowski, M. and Qasim, MA. Biochim. Biphys. Acta (2000) 1477: 324-337; Farady, C J and Craik, CS (ChemBioChem. (2010) 11: 2341-2346). All inhibitors that operate by this mechanism are canonical and it was demonstrated for serine protease inhibitors [Id., citing Rawlings, ND., et al. Biochem. J. (2004) 378: 705-716]. The majority of plant serine protease inhibitors (SPIs) adopt the standard mechanism of inhibition [Id., citing Bateman, KS and James, MN. Curr. Protein Pept. Sci. (2011) 12: 340-347]. In tight-binding reactions, the inhibitors interact with the protease active site (P1) in a similar way to the enzyme-substrate interaction. The protease-inhibitor complex co-exists in a stable equilibrium among the intact form of the inhibitor and the modified forms of the inhibitor where the peptide bond of the reactive site is cleaved. Therefore, the inhibitor in the complex is dissociated to its intact or its modified form. The canonical inhibitors can also inhibit serine proteinases with differing P1 specificities.

The Bowman Birk, Potato II and Kunitz families are able to target more than one proteinase at a time, often with different specificities [Id., citing Joshi, R S., et al. Biochim. Biophys. Acta (2013) 1830: 5087-5094]. Plant SPIs that apply this strategy allow plants to prepare themselves against unwanted proteolytic activity, whether to control development or to defend against pest attack.

Chemical serine protease inhibitors. Phenyl-methyl-sulfonyl fluoride (PMSF) is an exemplary chemical protease inhibitor that reacts with serine residues to inhibit trypsin, chymotrypsin, thrombin and papain.

Protectives. Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a desiccant. The absorption of skin moisture decreases friction and also discourages certain bacterial growth. Some of the materials used as protective adsorbents include bentonite, insoluble salts of bismuth, boric acid, calcium carbonate, (precipitated), cellulose, corn starch, magnesium stearate, talc, titanium dioxide, zinc oxide, and zinc stearate.

Protectives can also be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

According to some embodiments, protectives included in the composition of the invention are demulcents. According to some embodiments, the irritant is a rubefacient.

Sun screening agents. Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpniyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnaraoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyqumoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Sunless tanning agents. Representative examples of sunless tanning agents usable in the described invention include, without limitation, dihydroxyacetone, glyceraldehyde, indoles and their derivatives. The sunless tanning agents can be used in combination with the sunscreen agents.

Vitaminized peptides. According to some embodiments, the composition further comprises vitaminized peptides. In some embodiments, the composition further comprises vitaminized proteins.

Cleansing agents. Exemplary cleansing agents which may be use in the described invention include surfactant based cleansing agents, examples of which have been listed hereinabove. Other non-surfactant-based cleansing agents known to those of skill in the art may also be employed.

The topical compositions of the described invention can be applied locally to the skin and may be in any form including solutions, oils, creams, ointments, gels, lotions, shampoos, milks, cleansers, moisturizers, sprays, skin patches and the like.

Additional compositions of the described invention can be readily prepared using technology which is known in the art such as described in Remington's Pharmaceutical Sciences, 18th or 19th editions, published by the Mack Publishing Company of Easton, Pennsylvania.

According to the foregoing embodiments, the pharmaceutical composition may be administered once, for a limited period of time or as a maintenance therapy over an extended period of time, for example until the condition is ameliorated, cured or for the life of the subject. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the pharmaceutical composition may be administered for more than one year, for about 2 years, for about 3 years, for about 4 years, or longer.

According to the foregoing embodiments, the composition or pharmaceutical composition may be administered once daily, twice daily, three times daily, four times daily or more.

### Methods

According to some embodiments, a method for improving appearance of human skin comprises
exposing a vascular plant to combinations of abiotic stress conditions that cause the plant to modulate its signaling pathways and metabolism to ensure its survival in a challenging environment;
purifying from tissue of the vascular plant exposed to the combinations of abiotic conditions a population of plant-derived exosome-like nanoparticles (plant-derived exosomes), wherein size of the plant-derived exosomes is about 50 nm-500 nm inclusive;
preparing a composition comprising about 1×10E8 to about 1×10E12, inclusive [i.e., 1×10E8, 2×10E8, 3×10E8, 4×10E8, 5×10E8, 6×10E8, 7×10E8, 8×10E8, 9×10E8; 1×10E9, 2×10E9, 3×10E9, 4×10E9, 5×10E9, 6×10E9, 7×10E9, 8×10E9, 9×10E9, 1×10E10, 1×10E10, 2×10E10, 3×10E10, 4×10E10, 5×10E10, 6×10E10, 7×10E10, 8×10E10, 9×10E10; 1×10E11, 2×10E11, 3×10E11, 4×10E11, 5×10E11, 6×10E11, 7×10E11, 8×10E11, 9×10E11, or 1×10E12], abiotically stressed plant-derived exosome-like nanoparticles containing the tuned cargo and a cosmetically acceptable carrier; and
applying the composition topically to human skin;
wherein the plant-derived exosomes comprise a tuned cargo comprising:
a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and
a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase or a combination thereof.

According to some embodiments, the plant tissue includes roots, stems, leaves, flowers, seeds, fruits, a liquid extract of the plant tissue, a nut milk or a combination thereof.

According to some embodiments, the HSP chaperone induced under the abiotic stress conditions comprise HSP100, HSP90, HSP70, HSP60, a small HSP or a combination thereof; and/or the Hsf induced under the abiotic stress conditions comprises HsfA, HsfB, HsfC or a combination thereof.

According to some embodiments, primary abiotic stress conditions including high/low temperature; salinity; drought; dehydration; flooding; heavy metal chemical pollutants; light stresses or physical wounding produce secondary stresses comprising oxidative stress and osmotic stress.

According to some embodiments, the tuned protein cargo of the plant-derived exosome-like nanoparticles correlates to a protein signature including a keratin; semaphorin receptor plexin-B1 mitogen-activated protein kinase kinase 2 (MEKK2), diacylglycerol kinase; T cell receptor beta chain; a fez family zinc finger protein or a combination thereof.

According to some embodiments, the tuned cargo of the plant-derived exosomes can modulate bioactivities of mammalian cells directly or indirectly. According to some embodiments, the bioactivities comprise a correlated signaling pathway in the human cells. According to some embodiments, the correlated human signaling pathway includes PI3K signaling, ERK/MAPK signaling; insulin growth factor 1 receptor (IGF1R) signaling, VEGFA/VEGFR2 signaling; leptin signaling; cytokine signaling; interleukin signaling, semaphorin signaling; sirtuin signaling; LRP1 signaling, or a combination thereof.

According to some embodiments the composition comprising the tuned cargo of the exosome-like plant nanoparticles when applied to the skin may modulate gene expression in immune cells, keratinocytes, melanocytes or fibroblasts in the skin; modulate a signaling pathway that contributes to inflammation, immune dysfunction or both in the skin; modulate circadian rhythms of the skin and its components; and/or rejuvenate appearance of the skin by: improving youthful appearance of skin and/or reducing appearance of wrinkles, e.g., by stimulating hyaluronic acid and collagen production; and/or improving skin clarity; and/or improving skin texture; and/or improving skin luminosity; and/or improving skin radiance, or a combination thereof.

According to some embodiments of the method, the pathway is a PI3K/AKT/mTOR pathway; and/or an MAPK pathway; and/or an IGF-1R pathway; and/or a sirtuins pathway; and/or LRP1 signaling or a combination thereof.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Modulating Heat Shock Proteins/Stress Response

Aloe vera plants were grown and sampled under the following conditions. Initially, the plants were maintained at room temperature, approximately 25° C. with ample water and light. Leaf samples from plants grown at room temperature were taken by removing a 3 inch leaf section for further preparation. The plant was then heat shocked by warming it to 35° C. in an incubator for 2 hours. After the two hour incubation, 3 inch leaf sections from the heated plant were dissected for further preparation.

For leaf preparation, the inner leaf pulp containing parenchymal mesophyll cells was carefully removed to avoid collecting any outer leaf skin material and then homogenized. Homogenization was performed using a Precellys tissue homogenizer, ceramic beads and sterile PBS to disrupt the plant pulp tissue. The homogenate was transferred to a large tube and the beads were washed 4 times with sterile PBS to collect all of the homogenate, which was also transferred to the larger tube for storage at −80° C. until used for exosome isolation.

In separate preparations, the same procedure was followed except that the temperature used in the heat shock step was about 42° C. in some preparations and about 45° C. in other preparations, and the time period of heat shock ranged from 1 hour to about 3 hours.

Exosomes produced by the plants were isolated from the inner leaf pulp homogenate according to the following procedure. The homogenate was diluted in PBS at a ratio of about 16 g of homogenate per 50 mL PBS. The diluted homogenate mixture was put through a freeze-thaw cycle by freezing at −80° C. and then allowing the mixture to thaw at room temperature, whereupon the volume was increased to 300 mL by adding PBS.

The diluted homogenate mixture was then fractioned by differential centrifugation to isolate the exosomes produced by the plants. In particular, the diluted homogenate mixture was subjected to centrifugation at 4,000×g for 20 min at room temperature to pellet cell debris. The clarified supernatant was collected and subjected to centrifugation at 120,000×g (Avg. RCF) for 30 minutes at room temperature to remove any intact chloroplasts. The subsequent clarified supernatant was brought up to 360 mL with PBS and subjected to centrifugation at 100,000×g (Avg. RCF) for 2 hrs at 4° C. to pellet exosomes. The supernatant was aspirated and the pellet(s) suspended in minimum volume of d-PBS (3000-10000 μL).

The manufacturer's instructions were followed to estimate protein and RNA concentration using a NANODROP™ (ThermoFisher®) spectrophotometer. Particle diameter and concentration were assessed by tunable resistive pulse sensing (TRPS; (qNano, Izon Science Ltd) using a NP150 nanopore membrane at a 47 mm stretch. The concentration of particles was standardized using multi-pressure calibration with 110 nm carboxylated polystyrene beads at a concentration of 1.2×10E13 particles/mL. The isolated exosomes were aliquoted into appropriate volumes into 1.5 mL screw cap tubes.

It was determined that the isolated exosomes described above could be stored at −80° C. and then thawed at a later date for use without a detectable decrease in activity.

Table 4 below shows processing yields from this process.

| | | | | | 2 × 10¹⁰ Aloe gel | |
|---|---|---|---|---|---|---|
| | | | | | exosome RNA | |
| | | Raw | Aloe gel exosomes | | | Total |
| | | material | Particulates | Protein | RNA | RNA |
| | | gel (g) | per mL | (mg/ml) | (ng/ul) | (:ng) |
| Prep 1 | Pre | 1.42 | 2.2E+11 | 0.09 | 51.24 | 768.6 |
| Prep 2 | 2 h 35° C. | 1.42 | 6E+11 | 0.08 | 35.03 | 526.45 |
| Prep 3 | 3 h 45° C. | 1.21 | 3.6E+11 | 0.21 | 20.77 | 311.55 |
| Prep 4 | 6 h post | 1.13 | 2E+11 | 0.36 | 25.95 | 389.25 |
| Prep 5 | 24 h post | 2 | 2.1E+11 | 0.95 | 43.18 | 647.7 |
| | Whole | 2 | | | 238.42 | 3576.3 |
| | plant | | | | | |

### Example 2. Heat Shock Induces Stress Response Molecules in Plant-Derived Exosome Like Nanoparticles (“Plant Exosomes”)

Isolated exosomes produced according to the method described in Example 1 were characterized using the following procedures.

To determine and compare changes in heat shock stress response molecules in the isolated plant exosomes, total RNA was isolated from exosomes derived from the leaf pulp of Aloe vera plants exposed to no heat shock, 35° C. heat shock for 2 hours, 45° C. heat shock for 3 hours, and 24 hour room temperature recovery after 45° C. heat shock for 3 hours. Specifically, 2×10¹⁰ exosomes from each treatment were used to isolate total RNA using a micro RNeasy kit from Qiagen and the manufacturer's protocol. Total RNA was quantified spectrophometrically using manufacturer's instructions and a NANODROP™ (ThermoFisher®) spectrophotometer. cDNA was prepared using 300 ng total RNA per reaction in 20 microliter final volume and qPCR performed using 10 ng of cDNA per triplicate, primers for HSP70 and 18s genes, and SYBR Green master mix in a final volume of 15 microliters. Relative gene expression was determined by the AAC method using 18s gene expression as the internal housekeeping gene and comparisons made to the no heat shock control exosomes. HSP70 is a heat stress response molecule. In this testing, HSP70 was used as a marker to show that heat stress responses to the plant were transferred to the plant exosomes. One of skill in the art will understand that other stress response molecules can be transferred to the plant exosomes.

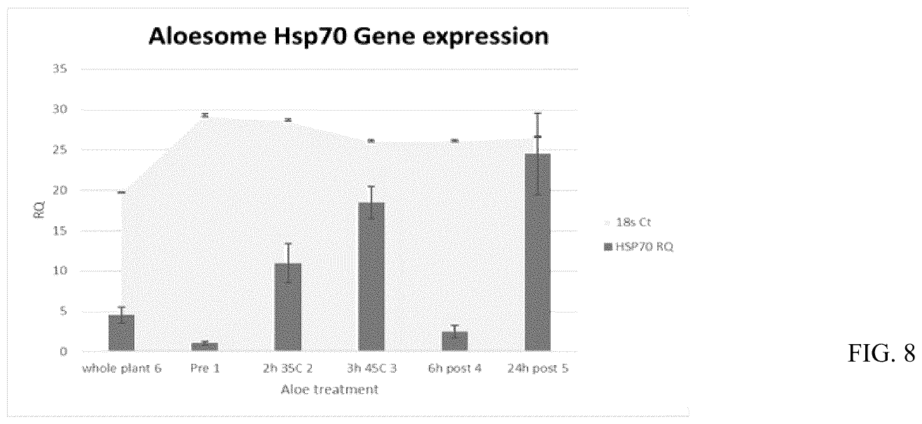

FIG. 8 shows the relative changes in HSP70 mRNA transcript levels in exosomes purified from Aloe vera plants for each of the treatments set forth above. Values shown are Relative Quantification (RQ) values determined by the AACt method (Fold Change) relative to the non-heat shocked exosome values. Statistical significance compared to the non-heat shocked sample was determined using student's t-test and a p-value <0.05, significance is denoted by a As can be seen in FIG. 8, induction of a heat shock stress at both 35° C. and 45° C. increased the transcript level of the stress response molecule, HSP70, relative to that for non-heat treated plants. The transcript level at a heat shock temperature of 35° C. was less than the transcript level at a heat shock temperature of 45° C. The data shows a 10× increase in transcript level of the stress response molecule HSP70 after a heat shock of 35° C. in comparison to the same for non-heat shocked exosomes. The transcript level of HSP70 after a heat shock of 45° C. was about 17× that for non-heat shocked exosomes, and transcript level of HSP70 after heat shock of 45° C. and 24 hour waiting period was about 22× that for non-heat shocked exosomes. The increase in stress response molecules was maintained for at least 24 hours after removing the heat shock stress. The data indicate that stress response molecules are significantly increased in isolated exosomes by subjecting the Aloe vera plant to a heat shock for 2-3 hours, and that these effects persist for at least 24 hours after the heat shock. Temperatures higher than 45° C. were not tested because of concerns that higher temperatures would detrimentally affect the health of the plant.

### Example 3. Determination of Signature Cargo of Heat-Stressed Aloe vera Plants of Example 2 by RNA-Seq

RNA-seq. is a process of creating short sequencing reads from RNA molecules. The steps consist of first converting the RNA into cDNA; then (optionally) amplifying the cDNA by PCR; and finally fragmenting the cDNA into short pieces/fragments. After the sequencing library is prepared, the fragments are used as input for next-generation sequencing. The resulting sequence reads contained in FASTQ files are then aligned to a known reference sequence. [Deshpande, D., et al. Frontiers Genetics (2023) 14: 997383].

The signature cargo of exosomes when isolated from heat-stressed Aloe vera plants through RNA-seq., including only those miRNAs >100, contains: ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof.

### Example 4. Human Reactome Analysis

A reactome analysis was obtained by synching these signature Aloe miRNAs to their human homologs. The pathways and genes these miRNAs would affect were identified through AI databases. The reactome analysis revealed the following pathways and genes:
Sema4D mediated inhibition of cell attachment and migration;
Sema4D in semaphorin signaling;
Ras activation upon Ca2+ inflex through NMDA receptor;
PTK6 Regulates RHO GTPases, RAS GTPase, and MAP kinases;
CREB phosphorylation through the activation of Ras;
Semaphorin interactions;
Rho GTPase cycle;
Post NMDA receptor activation events;
Activation of NMDA receptor upon glutamate binding and postsynaptic events;
RAF/MAP kinase cascade;
SOS-mediated signaling;
Axon guidance;
GRB2 events in EGFR signaling;
Frs2-mediated activation;
MAPK1/MAPK3 signaling;
Interleukin-2 signaling;
VEGFR2 mediated cell proliferation;
ARMS-mediated activation;
RET signaling;
Prolonged ERK activation events;
Signaling by leptin;
NCAM signaling for neurite outgrowth;
Signaling to ERKs;
FCERI mediated MAPK activation;
Signaling by PTK6;
Interleukin-3, 5 and GM-CSF signaling;
IRS-mediated signaling;
IRS-related events triggered by IGF1R;
IGF1R signaling cascade;
Signaling by Type 1 Insulin-like growth factor 1 receptor (IGF1R);
Insulin receptor signaling cascade;
Neurotransmitter receptor binding and downstream transmission in the postsynaptic cell;
Signaling by insulin receptor;
MAPK family signaling cascades;
Signaling by SCF-KIT;
Gastrin-CREB signaling pathway via PKC and MAPK;
DAP12 signaling;
DAP12 interactions;
VEGFA-VEGFR2 pathway;
Downstream signal transduction;
Signaling by Rho GTPases;
Signaling by VEGF;
Signaling by PDGF;
Transmission across chemical synapses;
Developmental biology;
Fc epsilon receptor (FCEFI) signaling;
NGF signaling via TAKA from the plasma membrane;
Signaling by EGFR;
Neuronal system;
Signaling by Interleukins;
Signaling by NGF;
Cytokine signaling in immune system;
Signaling by GPCR;
Innate immune system;
Signal transduction;
Immune system.

### Example 5. In Vitro Functional Analyses of Aloe-Derived Exosomes

The studies presented in Example 5 below show that Aloe derived exosomes influence the biological activity of recipient cells via their exosomal cargo to:
Induce collagen production;
Increase elastin production;
Increase hyaluronic acid production;
Regulate interferon production, which is important in aging skin;
Increase VEGF-A production, which is essential for skin function; VEGF-A mediated signaling is both required and sufficient for rejuvenation of human skin; and
Modulate melanin production and impact skin pigmentation.

### 5.1 Collagen Production: In Vitro Human Dermal Fibroblast Cell Model

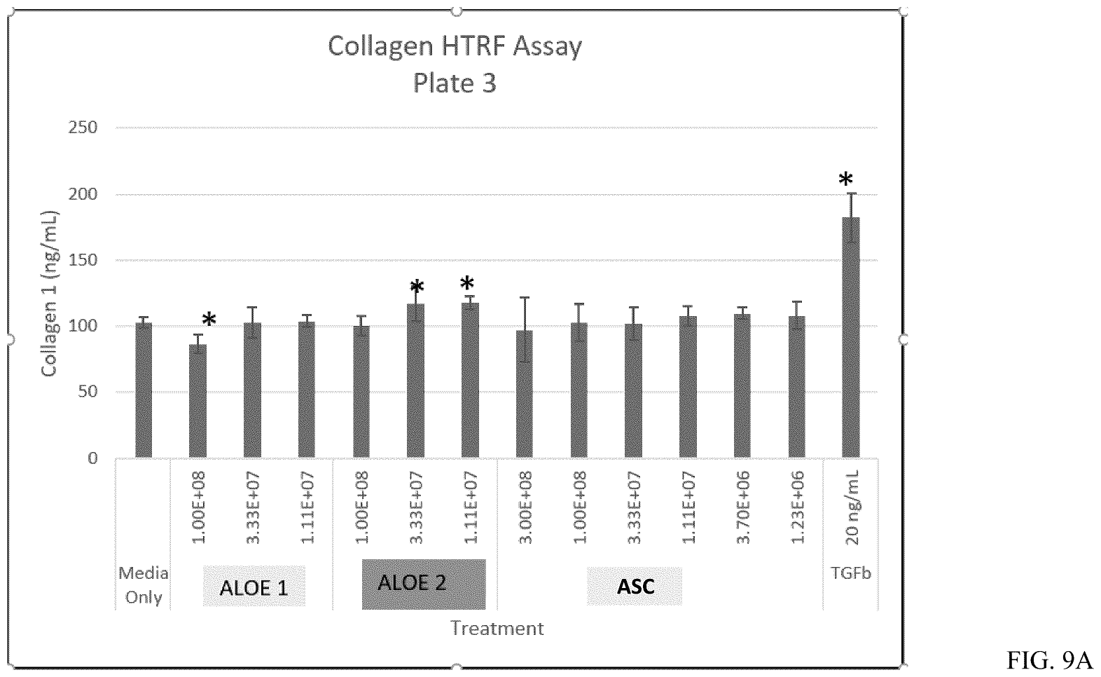

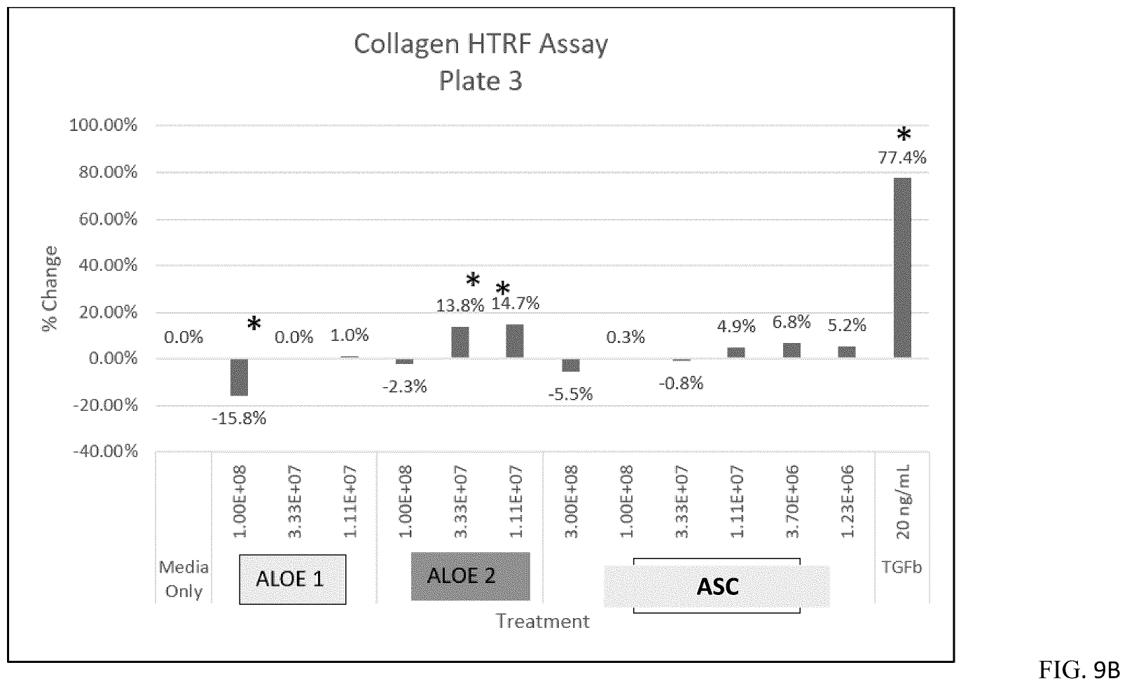

To determine if Aloe exosomes could induce collagen I synthesis in human dermal fibroblasts, the dermal fibroblasts were plated (10,000/well) and allowed to attach overnight. The cells were treated with adipose stromal stem cell exosomes and Aloe exosomes (heat-shocked and non-heat shocked) and incubated for 48 hours. Exosome samples from heat-shocked plants were obtained at the following times: 1) after heat shock of 35° C. for 2 hours, 2) after 3 hours of 45° C. heat shock, and 3) after 24 hours post 45° C. heat shock. The media was collected, clarified by centrifugation, and analyzed for Procollagen Type I production via Homogenous Time Resolved Fluorescence (HTRF). TGFb was included as a positive control. The results are shown in FIG. 9A and FIG. 9B. FIG. 9A is a bar graph showing that Aloe exosomes can induce collagen I synthesis in human dermal fibroblasts. Values shown in FIG. 9A are ng/ml of collagen. FIG. 9B is a bar graph showing percent change of collagen in human dermal fibroblasts. As shown in FIG. 9B, Collagen I production was increased by 13.8% and 14.7% respectively for the heat-shocked Aloe vera plant exosome types relative to media alone from Aloe 2. Exosomes from Aloe vera plants not receiving a heat shock stress did not increase collagen I production.

### 5.2 Vascular Endothelial Growth Factor A (VEGF-A) Production: In Vitro Dermal Fibroblast Model

To determine the effect of the Aloe-derived exosomes on VEGF-A production, dermal fibroblasts were cultured with standard growth media until 70% confluent. Dermal fibroblasts then were serum-starved for 18 hours with 0.5% FBS. Once serum-starved dermal fibroblasts were treated for 48 hours with IL-8, different dosages of Aloe EVs or 0.5% FBS (vehicle control). DMEM was the base media for all treatment conditions. After 48 hours, the conditioned media was collected and VEGF-A was measured using a Quantikine ELISA (R & E Systems).

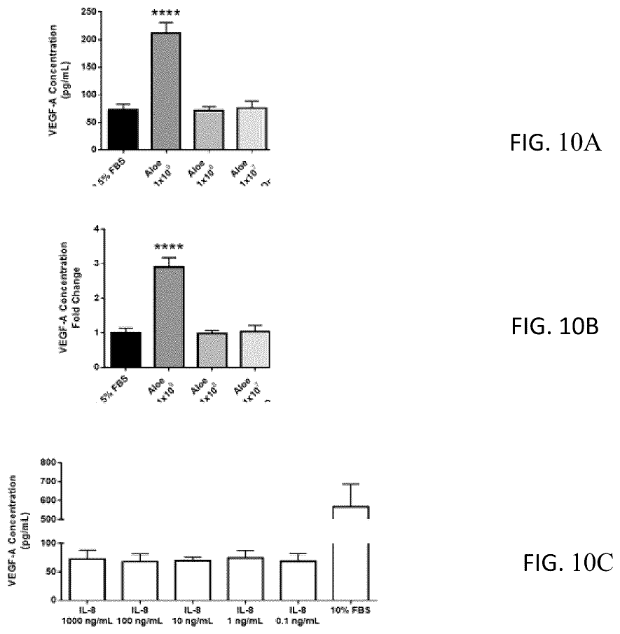

VEGF-A concentrations in human dermal fibroblasts were statistically compared to the vehicle control to determine concentration change due to the treatment. Results are expressed in mean±SD (n=3 biological replicates). Two-way ANOVA Dunnett post hoc test. **** p<0.0001, **pp<0.01. Results are shown in the bar graphs of FIG. 10. FIG. 10A is a bar graph of VEGF-A concentration (ng/mL) vs. a positive 5% FBS control, Aloe exosomes (1×10E9) Aloe Exosomes (1×10E8), and Aloe exosomes (1×10E7). FIG. 10B shows VEGFA concentration fold change; vs. 5% FBS positive control, Aloe exosomes (1×10E9) Aloe Exosomes (1×10E8), and Aloe exosomes (1×10E7). FIG. 10C shows VEGFA concentration (ng/mL) versus 11-8 (1000 ng/mL). IL-8 (100 mg/mL), IL-8 (10 ng/mL), IL-8 (1 ng/mL), IL8 (0.1 ng/mL), and 10% FBS positive control

### 5.3 Hyaluronic Acid and Elastin Production: In Vitro Human Dermal Fibroblast Model.

To determine the effect of the Aloe-derived exosomes on the production of hyaluronic acid (HA) and elastin in human skin cells, dermal fibroblasts were plated (10,000/well) and allowed to attach overnight. The cells were then treated with aloe exosomes and incubated for 48 hours. The media was collected and analyzed for hyaluronic acid (HA) and Elastin (ELN) production with ELISA assay.

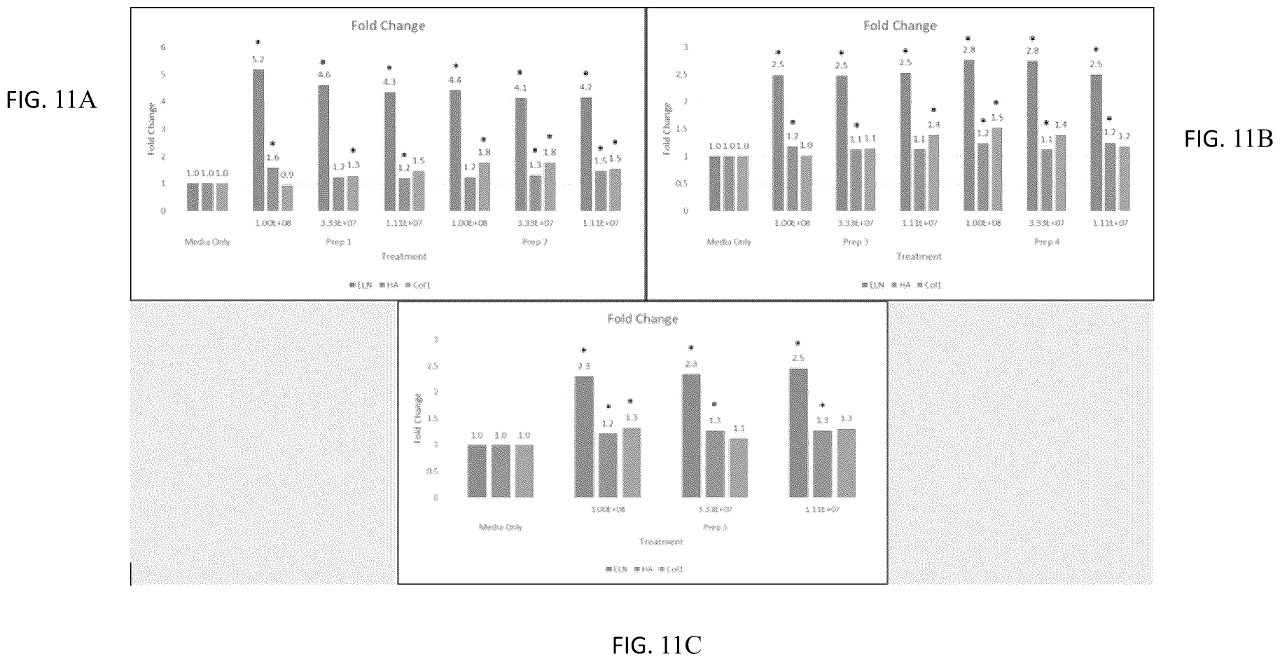

Results are shown in the bar graphs of FIG. 11A, FIG. 11B and FIG. 11C showing fold change in production of elastin (blue), hyaluronic acid (orange) and collagen 1 (gray) in human skin dermal fibroblasts treated with Aloe exosomes FIG. 11A Prep 1 [1×10E8, 3.33×10E7, 1.11×10E7], and Prep 2 (1.00×10E8, 3.33E7, 1.11×10E7); FIG. 11B Prep 3 [1×10E8, 3.33×10E7, 1.11×10E7], and Prep 4 (1.00×10E8, 3.33×10E7, 1.11×10E7), and FIG. 11C Prep 5 [1×10E8, 3.33×10E7, 1.11×10E7] compared to a media control. The Aloe-derived exosomes increased both hyaluronic acid and elastin production in human dermal fibroblasts compared to the control.

### 5.4 Inflammation/Inflammatory Cytokines Production: In Vitro PBMC Model-Bacterial Exposure

Experimental. To determine the effect of Aloe derived exosomes on inflammatory cytokine production, adult keratinocytes (10,000/well) were plated and allowed to attach overnight. Peripheral blood mononuclear cells (PBMCs) were seeded into 96-well plates and cultured overnight in growth media. The following day, media were gently removed and cells were treated with exosomes *ASC and Aloe); (P. acnes and/or LPS stimulated) treatments at varied concentrations in reduced serum media. Treated cells were incubated for 24 hours. After 24 hours of treatment, media were removed. Any leftover cell debris was removed by centrifugation at 300×g for 10 minutes. The clarified culture media was removed to a new plate and stored at −80 C until the start of the ELISA assay. The manufacturer's instructions were followed to measure cytokine production using culture media. In brief, the media was analyzed for interferon a2 production via MagPix. Viability was measured via Cell Titer Blue.

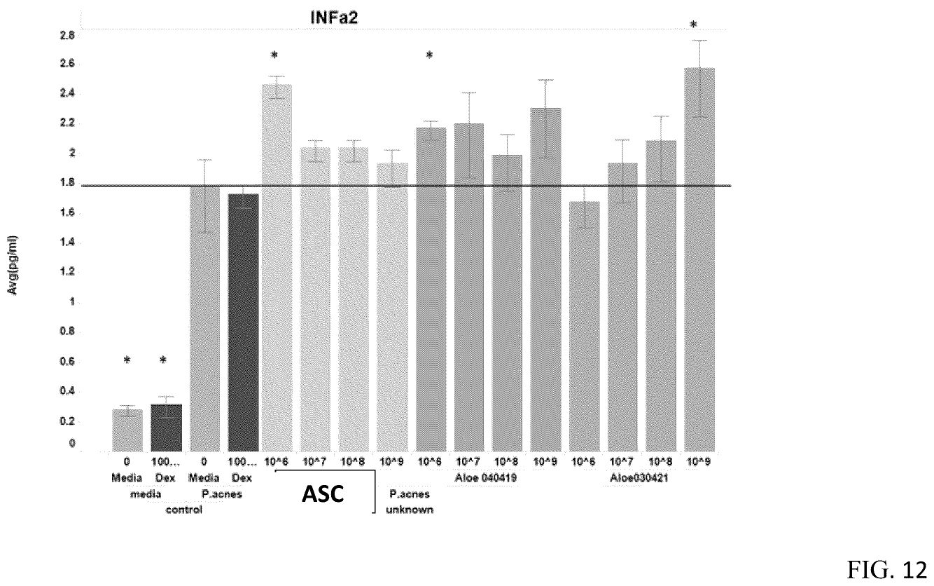

The results expressed as the average cytokine production (pg/mL interferon a2 (IFNa2) in PBMCs in vitro in response to bacterial exposure are shown in FIG. 12. Treatment groups from left to right are: media controls (dark green=media only, black=media plus dextran); media+P. acnes; adipose stromal stem cell exosomes (yellow); Aloe exosomes (light green=prep 1; and aloe exosomes light blue=prep 2). The results show that Aloe-derived exosomes modulate the inflammatory response by increasing interferon alpha 2a cytokine production.

### Example 5.5 Pigmentation: In Vitro B16 Cell Model

To determine the effects on melanin production, B16 cells were plated (3,500/well) and allowed to attach overnight. The cells were treated with Aloe derived exosomes in clear media containing 10 nM aMSH and incubated for 72 hours. Melanin content was read and MTT assay (a colorimetric assay for assessing cell viability) was performed.

Aloe-derived exosomes demonstrated one significant decrease in pigmentation (Skin Prep 6, 3.33×10E+7 exosomes/mL (data not shown).

### Example 6 RNA Labeling and Exosome Cargo Delivery

### A. Cell Culture

The purpose of this study was to demonstrate that Aloe-derived exosomes bind to skin cells and can deliver a labeled cargo (RNA) into the cell cytoplasm.

### Procedure:

Both Heat-Shocked (HS) Aloe and Heat-shocked adipose MSC exosomes were labeled with a fluorescent RNA dye (ExoGlow RNA EV Labeling Kit; SBI #EXOGR800A-1) as per manufacturer's instructions.

Unincorporated dye was removed by ultracentrifugation of labeled exosomes at 100,000×g, 2 hr, at 4° C.

Labeled exosomes (approximately 10E09 calculated from concentration of original labeling volume) were incubated with human skin fibroblasts (seeded onto 24-well plates overnight, 100,000 cells/well) in serum-free media (250 uL/well) at 37° C. for 2 hr to facilitate attachment and cargo delivery.

Negative controls, treated as above, were incubated at 4° C. (exosomes will attach poorly and will not be able to deliver RNA cargo properly).

Cells were washed 3× with PBS to remove unincorporated labeled exosomes immediately before analysis using fluorescent microscopy.

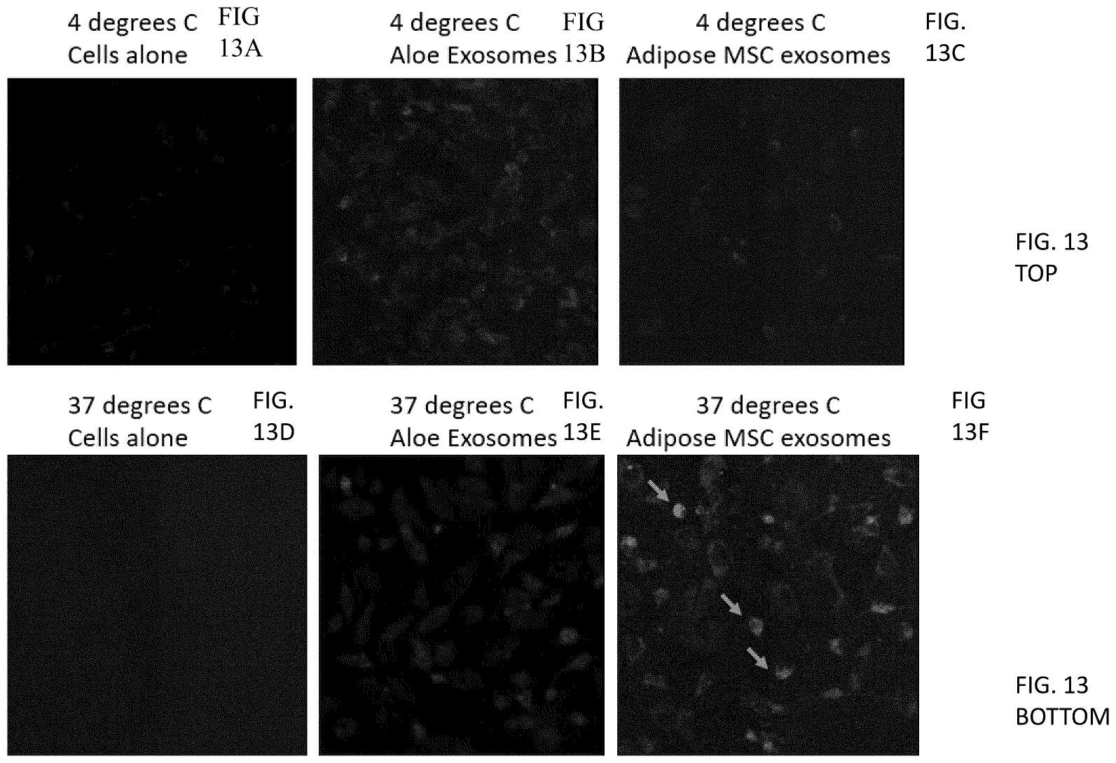

Results are shown in FIGS. 13A, 13B, 13C, 13D, 13E, and 13F. Top row: negative controls at 4° C.; FIG. 13A (cells alone (no addition of labeled exosomes) incubated at 4° C.), FIG. 13B (addition of Aloe exosomes at 4° C.), FIG. 13C (addition of adipose MSC exosomes at 4° C.). Bottom row, incubation at 37° C.: FIG. 13D (cells alone incubated, at 37° C., no addition of labeled exosomes), FIG. 13E (addition of aloe exosomes at 37° C.) and FIG. 13F (addition of adipose MSC exosomes at 37° C.).

At 4° C., minimal Aloe and MSC exosome fluorescence were expected since 4° C. impedes exosome binding and internalization.

Bottom row: Aloe exosome fluorescence is similar to negative control at 4° C.). Evidence of labeled RNA cargo delivery is not conclusive since background fluorescence at 4° C. is similar. This is perhaps due to non-specific or passive binding, which appears to be much greater compared to that of the MSC exosomes. At 37° C., MSC exosome fluorescence is readily visible (blue arrows, compare to negative control at 4° C.), indicating successful labeled RNA cargo delivery.

Based on similar amounts of fluorescence labeling observed at both 4° C. and 37° C. (little to no labeling was expected at 4° C.), this adherent cell culture approach did not convincingly demonstrate Aloe-derived exosomes binding to and delivering their labeled RNA to the fibroblast cell cytoplasm.

B. As a second approach to demonstrate labeled exosome attachment to cells, a suspension culture and FACS was used.

### Procedure

Remaining HS Aloe and HS Adipose MSC exosomes with fluorescence-labeled RNA from the previous cell culture experiment (approximately 1×10E9 labeled particles) were mixed with 1 million human dermal fibroblast cells in suspension in serum-free media (500 uL) and incubated at 37° C. for 2 hr in a water bath (gently mixing cell suspension by inversion every 20 min) to facilitate attachment and cargo delivery. Cells were washed 3× with 1 mL PBS to remove unincorporated labeled exosomes immediately prior to analysis using FACS.

The negative control was cells alone with no addition of labeled exosomes. The cells were incubated at 37° C. for 2 hr in a water bath with gentle mixing.

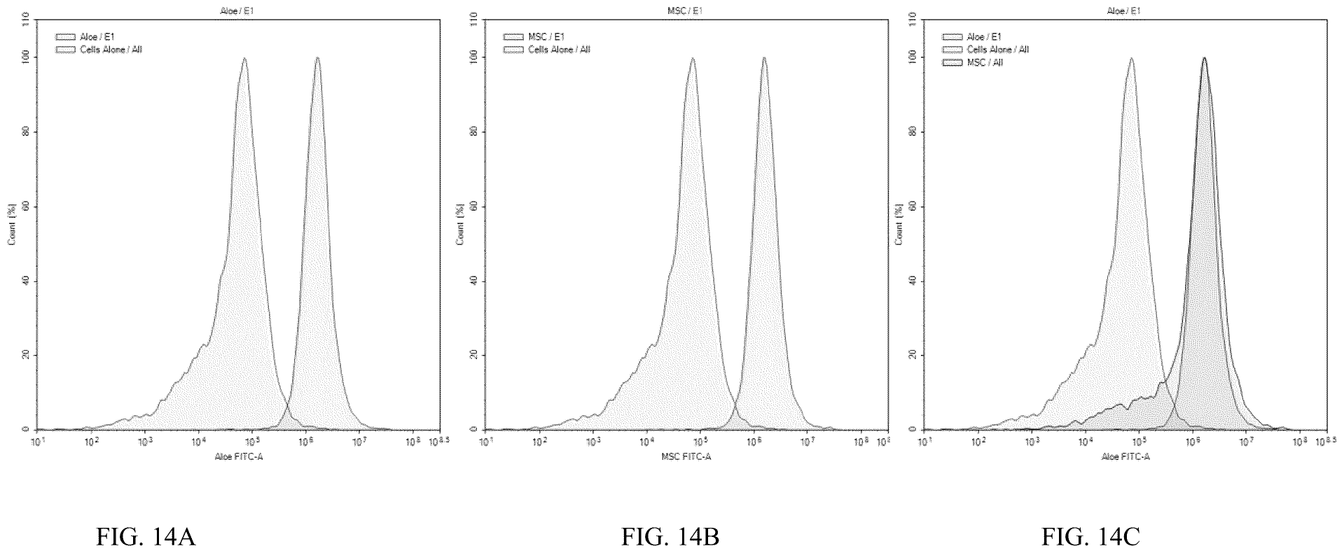

Results are shown in FIG. 14A, FIG. 14B and FIG. 14C. FIG. 14A: cells alone, no fluorescence (green curve); cells incubated with labeled HS Aloe exosomes saw a wavelength shift of cell peak to the right (red curve), indicative of labeled exosome uptake. FIG. 14B: same as FIG. 14A, using labeled HS MSC exosomes; saw an identical wavelength shift of the cell peak to the right (red curve), indicative of labeled exosome uptake. FIG. 14C is a merging of left and right panels, showing overlay of labeled HS Aloe and HS MSC peaks.

Conclusion: both HS Aloe and HS Adipose MSC RNA labeled exosomes bind to human dermal fibroblast cells.

### Example 7. Dermal Papillae Study on Cell Proliferation

Test system: primary cells

Each experiment was performed two times. 3 wells per sample PP6T

TABLE 5
Experiment 1. Cell Proliferation

| Sample | Number of cells × 10E3 | Counts |
|---|---|---|
| Control media | 91 | 11, 6, 8 |
| Test media, no exosomes | 29 | 3, 4, 8 |
| Test media + heat-shocked | 99 | 7, 10, 10 |
| human exosomes | | |
| Test media + Heat-shocked | 88 | 7, 8, 9 |
| aloe exosomes | | |

TABLE 6
Experiment 2: Cell Proliferation

| Sample | Number of Cells × 10E3 | Counts |
|---|---|---|
| Control media | 55 | 4, 5, 6 |
| Test media, no exosomes | 22 | 2, 3, 1 |
| Test media + HS human | 77 | 5, 4, 3 |
| exosomes | | |
| Test media + HS Aloe | 44 | 6, 9, 6 |
| exosomes | | |
| Test media + human + aloe | 63 | 5, 4, 8 |
| exosomes | | |

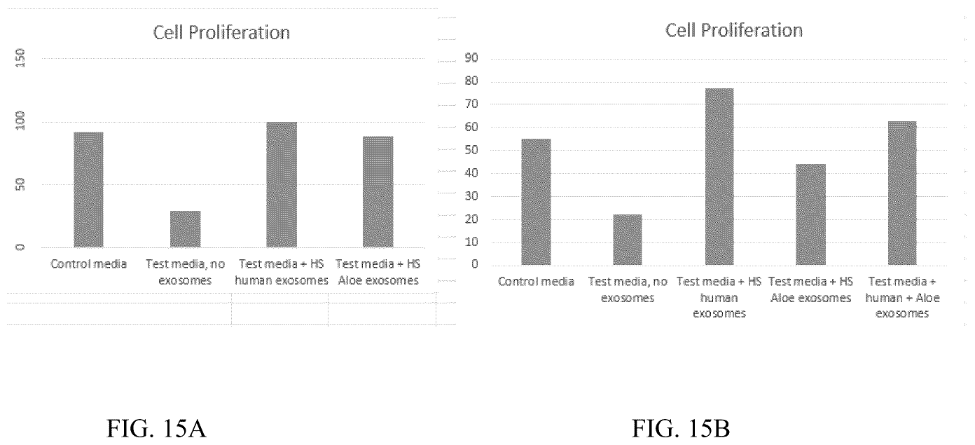

Results were averaged and multiplied by 1.1×10000. Results are shown in the bar graphs of FIG. 15A and FIG. 15B.

Cell proliferation in response to test media containing heat shock (HS) human adipose stromal stem cell (ASC) exosomes and test media containing HS Aloe exosomes is shown in FIG. 15A. Control media is manufacturer's recommended growth media, where increased cell number (proliferation) is anticipated. Proliferation in test media, which is a 10% dilution of control media is not anticipated. Cell proliferation was greater than in test media with no exosomes, with proliferation in response to test media HS human exosomes greater than for test media+HS aloe exosomes. Proliferation was observed in the Control media.

Cell proliferation in response to HS human exosomes, HS aloe exosomes, and human+aloe exosomes in test media is shown in FIG. 15B. Proliferation in test media+HS human exosomes was greater than in test media+human+aloe exosomes, which was greater than in test media+HS aloe exosomes, which was greater than the test media control. Proliferation was observed in the Control media.

### Experiment 3. Cell Proliferation—Aloe, Adipose MSCs (AdMSCs) and Amniotic Fluid (AF) Exosomes

95,000 cells/well were seeded onto 3 wells for each sample and media+/−exosomes was added to the wells and incubated. Exosomes were added to each of 3 wells in 1.5 mL of test media/well. Cells were removed from each well after 48 hr incubation and counted. An average of the three counts/sample (3 counts/well) were recorded and graphed.

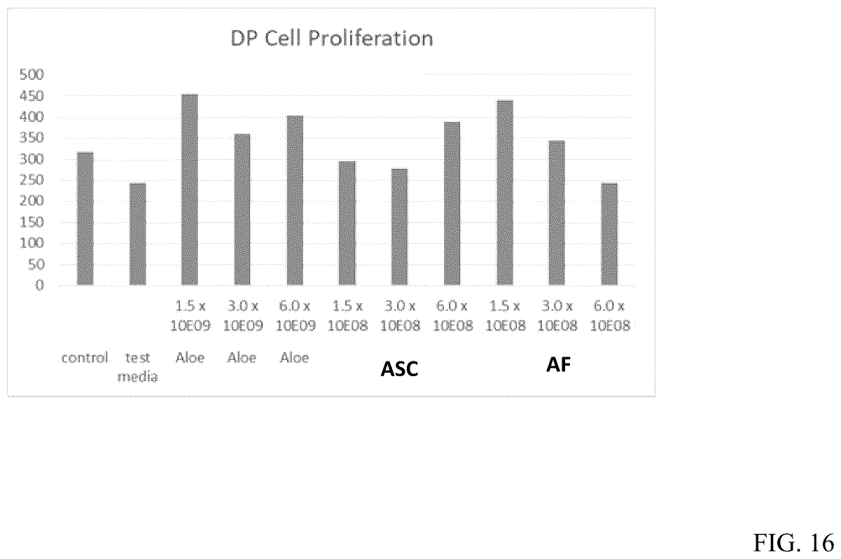

The results are shown in FIG. 16. FIG. 16 is a bar graph showing cell counts (×1000) vs. exosome preparation (left to right, control media; test media; Aloe-exosomes (1.5×10E9; 3.0×10E9; 6.0×10E9); adipose stromal stem cells (ASC) (1.5×10E8; 3.0×10E8; 6.0×10E8); amniotic fluid (AF) (1.5×10E8; 3.0×10E8; 6.0×10E8). Overall, all test concentrations of exosomes showed greater cell proliferation than the Test Media alone. All but 1.5 and 3.0 for AdMSC and 6.0 for AF showed equal to or greater cell proliferation than the Control growth media.

The Aloe exosome samples peaked at 1.5×10E9, 3.0×10E9 and 6.0×10E9 were comparable, i.e., in the same range, as 1.5×10E9.

The AdMSC sample peaked at 6.0×10E8, with 1.5×10E8 and 3.0×10E8 comparable, meaning in the same range as 6.0×E108.

AF peaked at 1.5×10E8 and then declined for 3.0×10E8 and 6.0×10E8. There is a large difference between 1.5×10E8 and 6.0×10E8.

### Example 8. In Vitro Antioxidant Activity of Aloe and Adipose EVS by ABTS and ORAC Assays

ABTS and ORAC assays were performed on 1E10, 1E9 and 1E8 particles of different EV samples to determine innate antioxidant activity. Antioxidant assay values represent the antioxidant capacity compared to Trolox, or a water soluble Vitamin E analog. ABTS assay values represent inhibition of oxidation of ABTS by electron transfer radical scavenging. ORAC assay values represent inhibition of oxidation or fluorescein by hydrogen atom transfer.

### (A) 2,2′-azino-bis(3-ethylbenzothiazoline-6-sulfonic acid) diammonium salt (ABTS) Assay

The ABTS antioxidant assay measures ABTS+radical cation formation induced by metmyoglobin and hydrogen peroxide. Trolox (6-hydroxy-2,5,7,8-tetramethylchroman-2-carboxylic acid), a water soluble vitamin E analog, serves as a positive control inhibiting the formation of the radical cation in a dose-dependent manner. The antioxidant activity in biological fluids, cells, tissues and natural extracts can be normalized to equivalent Trolox units to quantify the composite antioxidant activity present. This assay measures radical scavenging by electron donation and can provide analysis of a test sample's antioxidant activity.

### Principle of the Assay

A ferryl myoglobin radical is formed from metmyoglobin and hydrogen peroxide. The ferryl myoglobin radical can oxidize ABTS to generate a radical cation, ABTS+, that is green in color and can be measured by absorbance at 405 nm. Antioxidants suppress this reaction by electron donation radical scavenging and inhibit the formation of the colored ABTS radical. The concentration of antioxidant in the test sample is inversely proportional to the ABTS radical formation and 405 nm absorbance.

### Materials and Reagents:

Trolox: Sigma 238813
Myoglobin from equine heart: Sigma M1882
ABTS: ThermoFisher Cat. #37615
Molecular Grade H₂O
Stop Solution
PBS
96 well assay plate (cleaer)
Fluorescent Plate Reader: SpectraMax iD3

### Procedure:

1. Trolox standards were prepared for final concentrations of 300, 150, 75, 37.5, and 18.75 μM. PBS was designated as blank standard.

2. Myoglobin working solution was prepared in Molecular Grade H₂O.

3. 10 μl of samples or Trolox standards were added to each individual well in 96 well plate according to the ABTS template plate map. 10 μl of PBS were added to individual wells as a negative control (Plate Blank).

4. 20 μl of myoglobin working solution was added to each individual well.

5. The assay was initiated by adding 100 μl of the ABTS solution to each of the wells containing standards and placed on a plate shaker at room temperature for 5 minutes. Reaction was stopped by adding 50 μl of Stop Solution per well.

6. Absorbance (O.D.) was measured using a plate reader at a wavelength of 405 nm.

### (B) Oxygen Radical Absorbance Assay (ORAC)

The ORAC assay measures the loss of fluorescein fluorescence over time due to peroxyl-radical formation by the breakdown of AAPH [2,2′-azobis(2-amidinopropane) dihydrochloride]. Trolox [6-hydroxy-2,5,7,8-tetramethylchroman-2-carboxylic acid], a water soluble vitamin E analog, serves as a positive control inhibiting fluorescein decay in a dose dependent manner. The ORAC assay is a kinetic assay measuring fluorescein decay and antioxidant protection over time. The antioxidant activity in biological fluids, cells, tissues, and natural extracts can be normalized to equivalent Trolox units to quantify the composite antioxidant activity present.

### Principle of Assay

A peroxyl radical (ROO—) is formed from the breakdown of AAPH at 37° C. The peroxyl radical can oxidize fluorescein (3′,6′-dihydroxy-spiro[isobenzofuran-1[3H]. 9′[9H]-xanthen]-3-one) to generate a product without fluorescence. Antioxidants suppress this reaction by a hydrogen atom transfer mechanism, inhibiting the oxidative degradation of the luoresein signal. The fluorescein signal is measured over 30 minutes by excitation at 485 nm, emission at 538 nm, and cutoff=530 nm. The concentration of antioxidant in the test sample is proportional to the fluorescence intensity through the course of the assay and is assessed by comparing the net area under the curve to that of a known antioxidant, Trolox.

### Materials and Reagents

AAPH: Cayman Chemical #82235
Trolox: Sigma 238813
Fluorescein: Acros 17324-1000
PBS
96 well assay plate (black, clear bottom): Costar 3904
Fluorescent Plate Reader—SpectraMax iD3

### Procedures:

1. Fluorescein working solution was prepared from stock solution and protected from light.

2. Trolox standards were prepared for final concentrations of 100, 50, 25, 12.5, and 6.25 uM. PBS was designated as blank standard.

3. 150 μL of the working fluorescein solution was added to each of the inner 60 wells of the black clear bottom assay plate.

4. 25 μL of samples or Trolox standards was added to each individual assay well according to the ORAC template plate map. 25 μL of PBS was added to individual wells as a negative control (Plate Blank). Plate was placed at 37° C. for at least 10 minutes in the plate reader chamber of incubator.

5. While the assay plate equilibrated to 37° C., AAPH working solution was prepared and placed on ice until needed.

6. The assay was initiated by adding 25 μL of the AAPH working solution to each of the wells containing standards and samples.

7. A kinetic read was performed on the plate for 30 minutes with 1 minute intervals at excitation=485 nm and emission=528 nm.

8. Upon completion of the assay area under the curve (AUC), et AUC and uM Trolox values were calculated for each unknown.

### Results

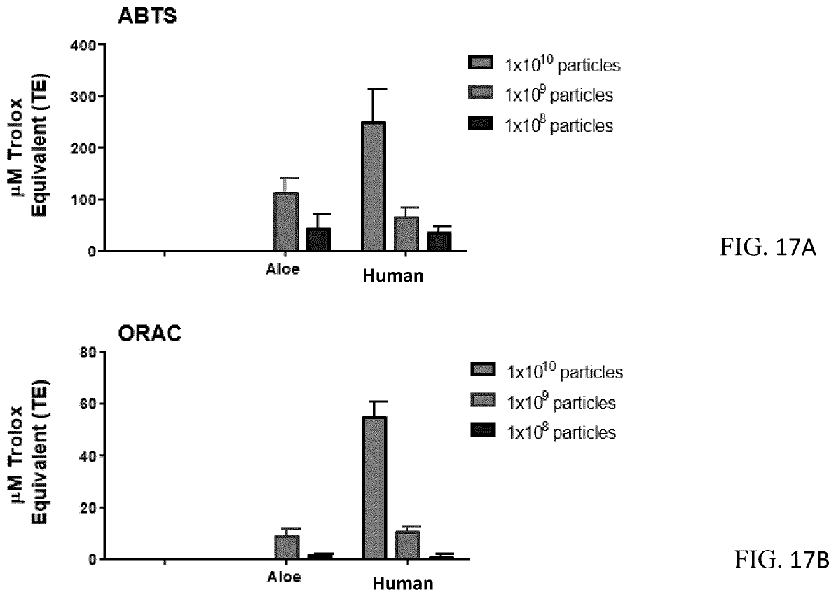

Results are shown in the bar graphs of FIG. 17A (ABTS assay) and FIG. 17B (ORAC assay). They suggest that the aloe-derived EVs and adipose-derived exosomes have some antioxidant capacity. Further testing is needed.

### Example 9. Proposed Preclinical Studies

### 8.1 Flavonoid Analysis of Aloe Exosomes and Supernatants

Rationale. Different plants use different approaches to overcome water shortage for their survival, for instance, drought avoidance and drought tolerance. Plant species opt for a strategy depending on two parameters, intensity and exposure time to drought, and it also includes the ability of that plant to accomplish molecular, biochemical, and physiological variations [Kubra, G. et al. Front. Plant Sci. (2021) 12: 680368; citing Xoconostle-Cazares, B. et al., Am. J. Plant Physiol. (2010) 5: 241-256]. Flavonoids are low molecular weight polyphenolic metabolites that are widely distributed and have distinct biological activities in plants. Flavonoids contribute to plant responses to severe abiotic stresses and have a major role in cell differentiation, growth, and defense signaling [Id., citing Ma, D. et al. Plant Physiol. Biochem. (2014) 80: 60-66; Khalid, M. et al., J. Integr. Agric. (2019) 18: 211-230].

Isolated aloe exosomes will be analyzed for flavonoids using an OxiSelect™ Flavonoid Assay Kit (Cell Biolabs #XAN-5077) according to manufacturers' instructions.

Serial dilutions of 1.5×10E9 exosomes will be tested in triplicate.

Serial dilutions of Aloe vera gel will also be tested in triplicate.

Serial dilutions of supernatants taken prior to exosome isolation will be tested in triplicate.

### 8.2 Evaluate Sirtuins Expression in Human Skin Cells Post Treatment Aloe Exosomes, as Well as Non-Heat-Shocked Aloe Exosomes

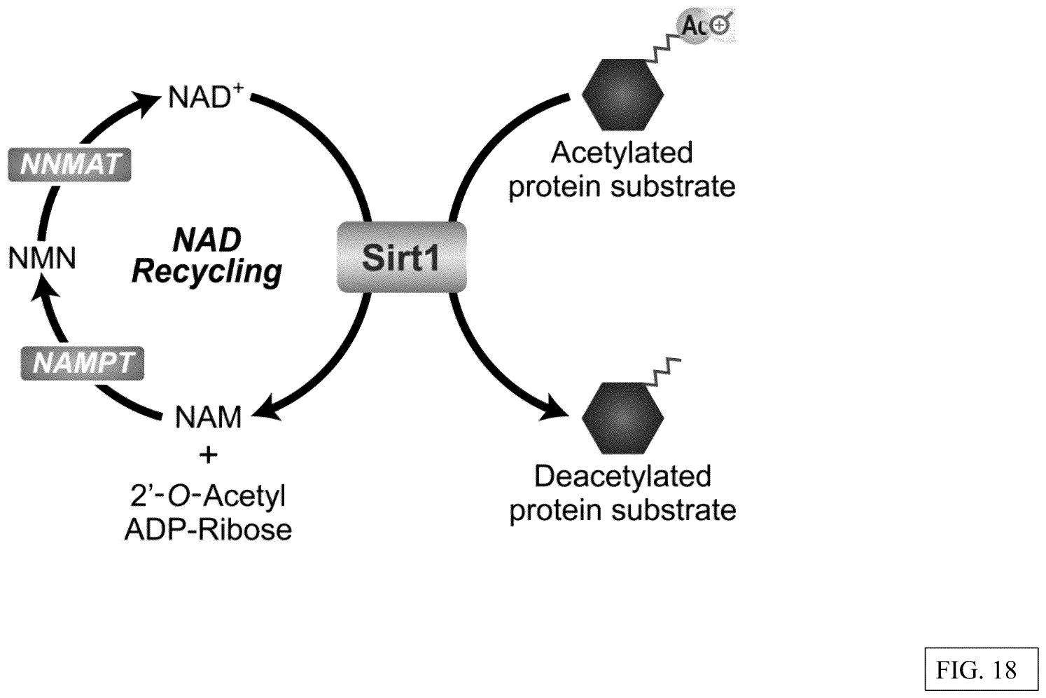

Rationale. Sirtuins are a family of seven proteins in humans (SIRT1-SIRT7) that are involved in multiple cellular processes relevant to dermatology. SIRTs share a nicotine adenine dinucleotide+(NAD)+-binding catalytic domain and may act specifically on different substrates depending on the biological processes in which they are involved [Wu, Q-J et al. Signal Transduction and Targeted Therapy (2002) 7: 402, citing Min, J. et al. Cell (2001) 105: 269-279]. FIG. 18 schematically depicts the enzymatic reaction catalyzed by Sirt1, Sirt1 catalyzes the deacetylation of several proteins by consuming nicotinamide adenine dinucleotide (NAD+), generating nicotinamide (NAM) and 2′-O-Acetyl-ADP-Ribose. NAM is recycled back into NAD+ by the enzymes nicotinamide phosphorybosyltransferase (NAMPT), nicotinamide mononucleotide adenylyltransferase (NMNAT), and the nicotinamide mononucleotide (NMN) intermediate. [Revollo, JR and Li, X. Trends Biochem. Sci. (2013) 38 (3): 160-167].

Sirtuins function in the cell via histone deacetylase and/or adenosine diphosphate ribosyltransferase enzymatic activity that target histone and non-histone substrates, including transcription regulators, tumor suppressors, structural proteins, DNA repair proteins, cell signaling proteins, transport proteins and enzymes. They are involved in cellular pathways related to skin structure and function, including aging, ultraviolet-induced photoaging, inflammation, epigenetics, cancer and a variety of cellular functions including cell cycle, DNA repair and proliferation.

The regulatory role of SIRTs in inflammation. The SIRT family may regulate the activation or differentiation of inflammatory cells, such as DCs and macrophages in the immune system. [Wu, Q-J., et al. Signal Transduction and Targeted Therapy (2002) 7: 402]. For example, SIRTs, especially SIRT1 and SIRT6, can affect the secretion of inflammatory mediators and play a central role in regulating the differentiation of dendritic cells (DCs) and the activation of macrophages. [Id., citing Liu, G., et al. Proc. Natl Acad. Sci. USA (2015) 112: E957-65; Woo, S J., et al. EBioMedicine (2018) 38: 228-237]. SIRT1 participates in mediating inflammatory signaling in DCs, consequentially modulating the balance of proinflammatory T helper type 1 cells and anti-inflammatory Foxp3(+) regulatory T cells. SIRT1 knockout (KO) in DCs restrained the generation of regulatory T cells while driving T helper 1 cell development, resulting in enhanced T-cell-mediated inflammation against microbial responses. [Id., citing Liu, G., et al. Proc. Natl Acad. Sci. USA (2015) 112: E957-E965]. Moreover, SIRT6 deficiency in macrophages resulted in inflammation with increases in acetylation and greater stability of the forkhead box protein O1 (FoxO1). Conversely, the ectopic overexpression of SIRT6 in KO cells reduced the inflammatory response. [Id., citing Woo, S J., et al. EBioMedicine (2018) 38: 228-237]. Moreover, results from in vivo experiments demonstrated that SIRT3 overexpression in transfused macrophages not only induced M2 macrophage polarization but also alleviated inflammation. [Id., citing Xi, J., et al. J. Cell Phys. (2019) 234: 11463-11473].

The effect of SIRTs on inflammatory mediators. Inflammatory mediators are closely regulated by the SIRT protein family. For example, overexpressed or activated SIRTs, mainly SIRT1-3, can reduce the inflammatory response through anti-inflammatory effects, such as tumor necrosis factor-alpha (TNF-α), a multifunctional pro-inflammatory cytokine, which is produced by macrophages/monocytes during acute inflammation, and plays a critical role with orchestrating the cytokine cascade in various inflammatory diseases. [Id., citing Wu, L., et al. Biochem. Biohys. Res. Commun. (2020) 521: 98-105] For instance, increased SIRT1 protein expression can reduce acetylation of the nuclear factor kappa-B (NF-κB) p65 subunit, which results in the suppression of TNF-α-induced NF-κB transcriptional activation and reduction of TNF-α secretion in a SIRT1-dependent manner.5 [Id., citing Yang, H., et al. PLoS One (2012) 7: e46364; Jung, Y J., et al. Biochem. Biophys. Res. Commun. (2012) 419: 206-210]. In addition, SIRT1 knockdown increased, while SIRT1 activator treatment decreased TNF-α secretion from macrophages. Id., citing Jung, Y J., et al. Biochem. Biophys. Res. Commun. (2012) 419: 206-210] One study verified that SIRT6 suppressed inflammatory responses and downregulated the expression of inflammatory factors interleukin (IL)-6 and TNF-α via the NF-κB pathway. [Id., citing Jiang, X., et al. Oxid. Med. Cell Longev. (2022) 1619651] Both SIRT1 and SIRT6 inhibited TNF-α-induced inflammation of vascular adventitial fibroblasts through reactive oxygen species (ROS) and the protein kinase B (Akt) signaling pathway. [Id., citing He, Y., et al. Exp. Cell Res. (2017) 357: 88-97]. SIRT1 exerted anti-inflammatory effects against IL-1β-mediated pro-inflammatory stress through the Toll-like receptor 2 (TLR2)/SIRT1/NF-κB pathway. [Id., citing Shen, J., et al. Pain Phys. (2016) 19: E215-E226]. SIRT1 deficiency increased microvascular inflammation in obese septic mice, while resveratrol treatment decreased leukocyte/platelet adhesion and E-selectin/intercellular adhesion molecule (ICAM-1) expression accompanied by increased SIRT1 expression and improved survival. {Id., citing Wang, X., et al. Obes (Silver Spring) (2015) 23: 1209-12171 In addition, SIRT1 and SIRT6 inhibited inflammation by decreasing pro-inflammatory cytokines such as IL-6, IL-β, cytochrome oxidase subunit 2 and ICAM-1. [Id., citing Lappas, M. Mediators Inflamm. (2012) 2012: 597514]. Moreover, SIRT1 exerted anti-inflammatory effects against IL-1β-mediated pro-inflammatory stress through the TLR2/SIRT1/NF-κB pathway. [Id., citing Shen, J. et al. Pain Phys. (2016) 19: E215-E226]. SIRT1 deficiency increased microvascular inflammation in obese septic mice, while resveratrol treatment decreased leukocyte/platelet adhesion and E-selectin/ICAM-1 expression accompanied by increased SIRT1 expression and improved survival. [Id., citing Wang, X., et al. Obes. (Silver Spring) (2015) 23: 1209-1217]. SIRT2 as modulators have been shown to be effective in inhibiting lipopolysaccharide-stimulated production of TNF-α to suppress neuroinflammation. [Id., citing Zhang, Y., et al. J. Neural Transm. (Vienna) (2021) 128: 631-644; Wang, B., et al. Neurochem. Res. (2016) 41: 2490-2500]. Kurundkar, et al. reported that SIRT3 deficiency altered the proinflammatory responses of macrophages to lipopolysaccharides, with a greater increase in TNF-α production. [Id., citing Kurundkar, D., et al. JCI Insight (2019): 4: 20722] Several studies have reported that SIRT3 downregulates IL-1β and IL-18, inhibits inflammasomes and attenuates oxidative stress. [Id., citing Zhao, WY., et al. Sci. Rep. (2016) 6: 33201; Palomer, X. et al. Signal Transduct. Target Ther. (2020) 5: 14]. SIRT3 KO mice have significantly increased inflammatory cell infiltration. [Id., citing Kim, D., et al. Mol. Med Rep. (2018) 18: 3665-3672].

The effect of SIRTs on inflammatory pathway components There are many studies on the mechanisms by which the SIRT family participates in inflammation, especially pathways involving NF-κB, TNF-α, and the NOD-, LRR- and pyrin domain-containing protein 3 (NLRP3) inflammasome. Growing evidence suggests the significant role of SIRTs in the regulation of inflammation. SIRT1 has anti-inflammatory effects mediated by the deacetylation and inactivation of the p65 subunit of NF-κB.78 SIRT1 inhibits the transcriptional activity of NF-κB via deacetylation of the p65 (RelA) subunit at Ac-Lys310. [Id., citing Yeung, F., et al. EMBO J. (2004) 23: 2369-2380]. The finding that lower SIRT1 activity levels may increase the expression of NF-κB, thus driving inflammation [Id., citing Li, G., et al. Biosci Rep. (2018) 38: BSR20180541] also highlights the important role of SIRT1 during inflammation. Repression of NF-κB activity is responsible for the anti-inflammatory effect of SIRT6. [Id., citing Kawahara, T L., et al. Cell (2009) 136: 62-74]. For instance, SIRT6 attenuated NF-κB expression by deacetylating histone H3K9 in the promoters of NF-κB target genes, hence decreasing inflammation. Additionally, SIRT6 overexpression suppressed NF-κB-mediated inflammatory responses in osteoarthritis (OA) development. [Id., citing Wu, Y., et al. Sci. Rep. (2015) 5: 17602].

The effect of SIRTs targeting noncoding RNAs on the inflammatory pathway. MicroRNAs (miRNAs) can negatively regulate inflammation by repressing SIRT1. For example, downregulation of miRNAs such as miR-217 and miR-543 mitigated the inflammatory response by regulating the SIRT1/AMPK/NF-κB signaling pathway. [Id., citing Xia, K., et al. Int. J. Mol. Med. (2020) 45: 634-646]. miR-378 reduced SIRT1 activity and facilitated the inflammatory pathway involving NF-κB-TNFα by targeting 5′-AMPK subunit gamma-2. [Id., citing Zhang, T, et al. J. Hepatol. (2019) 70: 87-96]. The RNase monocyte chemoattractant protein-induced protein 1 (MCPIP1) alleviated inflammatory responses by promoting the expression of SIRT1 mediated via miR-9. [Id., citing Hans, S., et al. J. Cell Physiol. (2019) 234: 22450-22462]. SIRT1 also attenuated cerebral ischemia/reperfusion injury by targeting the p53/miR-22 axis to suppress inflammation, cyclooxygenase (COX)-2 and inducible nitric oxide synthase (iNOS) expression. [Id., citing Lu, H. and Wang, B. Int. J. Mol. Med. (2017) 39: 208-216].

The role of SIRTs in metabolism. The SIRT proteins play roles in maintaining metabolic homeostasis by participating in the regulation of glucose, glutamine, and lipid metabolism. The roles of SIRTs in glucose metabolism have been established. For example, SIRT1 is a key positive regulator of systemic insulin sensitivity and regulates pancreatic insulin secretion, thus contributing to increased systemic insulin sensitivity, which triggers glucose uptake and utilization. [Id., citing Peng, Y., et al. J. Gastrointest. Srg. (2010) 14: 221-8; Zhang, Y., et al. Int. J. Mol. Med. (2020) 46: 2225-2234; Gouranton, E., et al. Adipocyte (2014) 3: 180-189]. Mechanistically, SIRT1 participates in the regulation of glucose metabolism by upregulating AMPK, and activation of AMPK can ameliorate the glucose metabolic imbalance. [Id., citing Peng, Y., et al. J. Gastrointest. Surg. (2010) 14: 221-228; Silvestre, M F., et al. Life Sci. (2014) 100: 55-60]. SIRT1 increases insulin sensitivity and lowers blood sugar by downregulating protein tyrosine phosphatase 1B, a key negative regulatory protein in the insulin signal transduction pathway. [Id., citing Gouranton, E., et al. Adipocyte (2014) 3: 180-189].

SIRT3 and SIRT 6 also participate in glucose metabolism. SIRT3 negatively regulates aerobic glycolysis by inhibiting hypoxia-inducible factor 1α (HIF-1α). [Id., citing Chang, H., et al. Hepatol. (2021) 36: 2978-2988]. SIRT6 takes part in the maintenance of glucose metabolic homeostasis in the whole body and in local tissues such as liver and skeletal muscle. [Id., citing Khan, D., et al. J. Cell Physiol. (2018) 233: 5478-5489; Cui, X., et al. Am. J. Physiol. Endocrinol. Metab. (2017) 313: E493-E505]; for example, SIRT6 in pancreatic R cells deacetylated FoxO1 and subsequently increased the expression of glucose-dependent transporter 2 to maintain the glucose-sensing ability of pancreatic β cells and systemic glucose tolerance. [Id., citing Song, M Y., et al. Sci. Repts (2016) 6: 30321]. Improvement in SIRT6-mediated insulin signaling transduction has been reported in the liver of obese rats after exercise. [Id., citing Zhang, Y., et al. Can. J. Physiol. Pharmacol. (2021) 99: 506-511]. Enhancement of insulin sensitivity in skeletal muscle and liver by physiological overexpression of SIRT6 has been described, [Id., citing Anderson, J G., et al. Mol. Metab. (2015) 4: 846-856].

Lipid metabolism. Qiang, et al. found that SIRT1-dependent cAMP Response Element Binding protein (Creb) deacetylation regulates lipid metabolism. [Id., citing Qiang, L., et al. Cell Metab. (2011) 14: 758-767]. Mechanistically, Lys136 is a substrate for SIRT1-dependent deacetylation that affects Creb activity by preventing cyclic adenosine monophosphate (cAMP)-dependent phosphorylation, leading to the promotion of hepatic lipid accumulation and secretion. Moreover, SIRT1 activates AMPK, which leads to lipid-lowering effects in vitro and in vivo. Id., citing Chen, W L, et al. Diabetologia (2012) 55: 1824-1835]. SIRT2 prevents liver steatosis and lipid metabolic disorders by deacetylation of hepatocyte nuclear factor 4a. [Id., citing Ren, H., et al. Hepatology (2021) 74: 723-740]. SIRT3 acts as a bridge in the lipid metabolism pathway. For example, pancreatic SIRT3 deficiency promoted hepatic steatosis by enhancing 5-hydroxytryptamine synthesis in mice with diet-induced obesity. [Id., citing Ming, X., et al. Diabetes (2021) 70: 119-131]. Roles in lipid metabolism for SIRT5 and SIRT6 also were identified. [Id., citing Hong, J., et al. Genomics (2020) 112: 1065-1076; Zhu, C., et al. Biochim. Biophys. Acta. Mol. Basis Dis. (2021) 1867: 166249; Masri, S., et al. Cell (2014) 158: 6599-6672; Xiong, X., et al. J. Endocrinol. (2017) 233: 307-314]. Thus, SIRT5 inhibited preadipocyte differentiation and lipid deposition by activating AMPK and repressing mitogen-activated protein kinase (MAPK) signaling pathways, which has been verified in obese mice. [Id., citing Hong, J., et al. Genomics (2020) 112: 1065-1076]. Compared with control wild-type mice, SIRT6-KO mice had a significant increase in both body weight and fat mass and exhibited glucose intolerance and insulin resistance. [Id., citing Xiong, X., et al. J. Endocrinol. (2017) 233: 307-314]; mechanistically, SIRT6-KO decreased expression of the adiponectin gene and Akt in white adipose tissue, while expression of the thermogenic gene UCP1 was diminished in brown adipose tissue.

The role of SIRTs in oxidative stress. SIRT proteins contribute to cellular tolerance to oxidative stress by regulating many genes and their related signaling pathways.

SIRT1, SIRT3 and SIRT6 act to counter oxidative stress by directly or indirectly interacting with AMPK. SIRT1 participates in regulating AMP-activated protein kinase (AMPK) and its related pathways. For example, AMPK can be activated by liver kinase B1 (LKB1), the upstream regulator of AMPK, while activated AMPK reduces oxidative stress injury by promoting insulin sensitivity, fatty acid oxidation and mitochondrial biosynthesis to generate ATP. [Id., citing Galdieri, L, et al. J. Biol. Chem. (2016) 291: 25154-25166]. SIRT1 overexpression leads to the deacetylation of LKB1, the translocation of LKB1 from the nucleus to the cytoplasm, and the activation of AMPK to alleviate oxidative stress. [Id., citing Vancura, A., et al. Int. J. Mol. Sci. (2018) 19: 3314]. Additionally, SIRT1 lowers LKB1 activation in the liver, which subsequently abrogates Thr172-AMPKα phosphorylation, thereby increasing oxidative stress in severe acute hypoxia. [Id., citing Morales-Alamo, D., et al. J. Appl. Physiol. (1985) 113: 917-928]. Thus, SIRT1 may activate AMPK by regulating LKB1, thereby resisting oxidative stress damage and promoting cell survival.

SIRT3 and SIRT6 can also interact with AMPK to exert an anti-oxidative effect. Deficiency of AMPKα resulted in elevated expression of SIRT3, which modulated oxidative stress in heart tissue both in vitro and in vivo. [Id., citing Guo, Z., et al. Int. J. Biol. Sci. (2022) 18: 826-840]. It has also been shown that AMPK activated SIRT3, limited oxidative stress and improved mitochondrial DNA integrity and function. [Id., citing Chen, L Y., et al. Osteoarthr. Cartil. (2018) 26: 1539-1550]. In addition, SIRT3 reduced ROS and lipid peroxidation by improving mitochondrial function via deacetylation of LKB1 and activation of AMPK. [Id., citing Li, M., et al. J. Cell Mol. Med. (2020) 24: 5109-5131] SIRT6 also promoted AMPK expression, thus upregulating antioxidant-encoding gene expression of manganese superoxide dismutase (MnSOD) and catalase (CAT), thereby suppressing oxidative stress. [Id., citing Wang, X X., et al. Basic Res. Cardiol. (2016) 111: 13].

The effect of SIRT1, SIRT 2, and SIRT6 on Nuclear erythroid 2-related factor 2 (Nrf2) Nrf2 is a leucine transcription factor that plays important roles in antioxidant response element (ARE)-dependent transcriptional regulation of defense genes. When stimulated, Nrf2 dissociates from suppressor protein Keap1, an adaptor protein of Cullin3-based E3 ligase, in the nucleus and interacts with AREs to regulate the expression of antioxidant genes, suggesting a close association between Nrf2 and oxidative stress. [Id., citing Tang, W, et al. World J. Gastroenterol. (2014) 20: 13079-13087].

SIRTs including SIRT1, SIRT2 and SIRT6 can activate Nrf2, regulate antioxidant gene expression, and thus fight oxidative stress damage. For example, SIRT1 activated Nrf2 by changing the structure of Keap1, leading to Nrf2 nuclear transfer and promoting the expression of antioxidant genes, such as glutathione S transferase and glucuronyl transferase. [Id., citing Zhang, Y K., et al. PLoS One (2013) 8: e59122; Chen, Z, et al. Biochem. Pharm. (2020) 177: 113951]. It was reported that SIRT2 was downregulated in the spinal cord of rats after peripheral nerve injury, which subsequently inhibited Nrf2 activity, leading to increased oxidative stress. [Id., citing Zhao, M., et al. Front. Pharm. (2021) 12: 646477]. The overexpression of SIRT6 in the brain through in vivo gene transfer enhanced Nrf2 signaling and reduced oxidative stress. [Id., citing Zhang, W., et al. Neuroscience (2017) 366: 95-104; Ka, SO., et al. FASEB J. (2017) 31: 3999-4010]. SIRT6 protected human lens epithelial cells from oxidative damage via activation of Nrf2 signaling. [Id., citing Sun, G L., et al. Biochem. Biophys. Res. Commun. (2019) 514: 777-784]. SIRT6 protects cells against hydrogen peroxide-induced oxidative stress by promoting Nrf2/ARE signaling. [Id., citing Yu, J., et al. Chem. Biol. Interact. (2019) 300: 151-158].

The effect of SIRT1 and SIRT3 on FoxOs. A family of SIRT targets are class 0 mammalian forkhead transcription factors (FoxO1, FoxO3, FoxO4 and FoxO6), which participate in regulating oxidative stress. FoxO1 can scavenge excessive reactive oxygen species (ROS) through the regulation of downstream target genes such as manganese superoxide dismutase (MnSOD) and catalase (CAT) genes and thus reduce cellular oxidative stress damage. SIRT1 alleviates oxidative stress by controlling nuclear shuttling and transcriptional activity of FoxO1 and FoxO3a. For instance, SIRT1 induced the transfer of FoxO1 to the nucleus and increased the level of FoxO1 protein in adipocytes, reducing the production of ROS and oxidative stress. [Id., citing Subauste, AR and Burant, CF. Am. J. Physiol. Endocrinol. Metabol. (2007) 293: E159-E164] Moreover, SIRT1 promoted early-onset age-related hearing loss by suppressing FoxO3a-mediated oxidative stress resistance in vivo. [Id., citing Han, C., et al. Neurobiol. Aging (92016) 43: 58-71]. Apart from SIRT1, SIRT3 has also been shown to participate in the regulation of oxidative stress via FoxO3. [Id., citing Rangarajan, P. et al. Neuroscience (2015) 311: 398-414]; Sundaresan, N R et al. J. Clin. Invest. (2009) 119: 2758-2771]. Mechanistically, SIRT3 activated FoxO3 gene expression, which increased transcription of MnSOD and CAT, enabling the elimination of ROS. [Id., citing Kwon, D N et al. Aging (Albany, NY) (2015) 7: 579-594; Yang, Y. et al. Toxicol. Vitr. (2016) 34: 128-37]. The aforementioned studies show that SIRT1 and SIRT3 can interact with FoxOs to counteract oxidative stress.

The effect of SIRT1 and SIRT3 on PGC-1α. PGC-1α is a coactivator (meaning an activity that activates or increases the transcription of specific gene sets by binding to a DNA-bound DNA-binding transcription factor, either on its own or as part of a complex) of peroxisome proliferator-activated receptor-7, which can act to block oxidative stress damage by scavenging excess ROS, inducing antioxidant enzyme expression and maintaining mitochondrial function. [Id., citing Rius-Perez, S. et al. Oxid. Med. Cell Longev. (2020) 2020: 1452696]. SIRT1 can activate PGC-1α through deacetylation, scavenge ROS caused by oxidative stress, and alleviate oxidative stress injury. Activation of the SIRT1-PGC-1α axis implies activation of antioxidant defense mechanisms, alleviating mitochondrial oxidative stress. [Id., citing Waldman, M., et al. Exp. Cell Res. (2018) 373: 112-118; Zhu, H., et al. Diabetes Metab. Syndr. Obes. (2021) 14: 355-366; Wang, S J., et al. Mol. Med. Resp. (2015) 11: 521-526]. Both SIRT1 and SIRT3 may interact with PGC-1α in order to resist oxidative stress damage. PGC-1α and SIRT3 can interact directly; PGC-1α increased respiratory capacity and reduced oxidative stress through SIRT3-mediated reduction of mitochondrial ROS. [Id., citing Rato, L., et al. Biochim. Biophys. Acta (2014) 1837: 335-344; Zhang, K., et al. Med. Sci. Monit. (2020) 26: e923688]. Loss of SIRT3 resulted in the expression of PGC-1α, which produced a decrease in mitochondrial respiration. Inhibition of SIRT3 reduced PGC-1α expression and mitochondrial function, thereby lowering oxidative stress resistance. [Id., citing Chen, J., et al. J. Periodontal Res. (2021) 56: 1163-1173; Paku, M. et al. Ann. Surg. Oncol. (2021) 28: 4720-4732].

The effect of SIRT1 and SIRT6 on p53. p53 is a stress response transcription factor and was the earliest discovered physiological substrate of SIRT1. It can promote oxidative stress injury by regulating different target proteins and further induce cellular responses. [Id., citing Yuan, Y., et al. Cell Physiol. Biochem. (2015) 37: 1240-1256]. p53 exerted pro-oxidant activity and promoted oxidative damage by regulating its transcriptional targets, including p53-inducible gene 3, glutathione/NADH, p-FoxO3a and B-cell lymphoma-2-associated-X-protein (Bax). [Id., citing Liu, X., et al. Oxid. Med. Cell Longev. (2020) 2020: 6039769]. In contrast, p53 can act as an antioxidant factor to suppress oxidative stress by regulating several redox-related proteins, such as MnSOD, glutathione peroxidase 1, and Jun N-terminal kinase (JNK). [Id., citing Liu, X., et al. OxId. Med. Cell Longev. (2020) 2020: 6039769]. When cells are under oxidative stress, multiple sites in the N-terminal of p53 are phosphorylated and multiple lysine sites in the C-terminal are acetylated. [Id., citing Gu, W. and Roeder, RG. Cell (1997) 90: 595-606]. SIRT1 has been reported to have a negative regulatory effect on p53; for example, depletion of SIRT1 abolished the increase in oxidative stress induced by p53 acetylation in THP-1 cells. [Id., citing de Kreutzenberg, S V., et al. Diabetes (2010) 59: 1006-1015]. SIRT1 activation also reversed p53 expression and accumulation brought on by H₂O₂-induced oxidative stress. Id., citing Kim, H J., et al. Biochim. Biophys. Acta (2015) 1852: 1550-1559]. The small molecule activator SRT2104 was reported to enhance renal SIRT1 expression and activity and deacetylated p53, resulting in activation of antioxidant signaling. [Id., citing Ma, F., et al. Biochim. Biophys. Acta Mol. Cell Res. (2019) 1866: 1272-1281] As for the role of SIRT6 in oxidative stress, SIRT6 protected cardiomyocytes by inhibiting p53/Fas-dependent cell death and augmenting endogenous antioxidant defense mechanisms. Id., citing Wu, S., et al. Cell Biol. Toxicol. (2023) 39 (1): 237-258].

The effect of SIRT1, SIRT3, and SIRT6 on NF-κB. NF-κB is a nuclear transcription factor. Activated NF-κB factors promote the production of ROS that damage tissues and organs. [Id.]. When oxidative stress occurs, enhanced ROS activity can stimulate the activation of NF-κB and induce the expression of ICAM-1 and monocyte chemotactic factor 1, which further activate NF-κB and lead to oxidative stress. [Id., citing Sies, H., et al. Annu. Rev. Biochem. (2017) 86: 715-748]. SIRTs inhibited transcription by deacetylating the NF-κB subunit Rel/p65, reducing the production of oxygen radicals. [Id., citing Li, G., et al. Biosci. Rep. (2018) 38: BSR20180541]. It has been reported that SIRT1, SIRT3, and SIRT6 can block oxidative stress damage by inhibiting NF-κB activity through deacetylation. For example, downregulation of SIRT1 protein levels by NF-κB led to oxidative stress. [Id., citing Rada, P., et al. Antioxid. Redox Signal. (2018) 28: 1187-1208] In addition, SIRT3 regulated ROS generation, causing suppression of NF-κB activation and oxygen radicals. [Id., citing Chen, I C., et al. Toxicol. Sci. (2016) 152: 113-127] Moreover, loss of SIRT6 in cutaneous wounds aggravated the proinflammatory response by increasing NF-κB activation and promoting oxidative stress. [Id., citing Thandavarayan, R A., et al. Exp. Dermatol. (2015) 24: 773-778].

The effect of SIRT1 on FOXO-dependent apoptosis. SIRT1 can regulate the activity of FoxO, thereby modulating the balance between anti-apoptotic and apoptotic genes. [Id., citing Frampton, G., et al. Am. J. Physiol. Gastrointest. Liver Physiol. (2012) 303: G1202-G1211]. SIRT1 is a key regulator of cell defenses and survival in response to stress, which deacetylates and represses FoxO-dependent apoptosis. [Id., citing Marfe, G., et al. PLoS One (2011) 6: e27313; Motta, M C., et al. Cell (2004) 116: 551-563] SIRT1 mediates cell apoptosis through the deacetylation of FoxO proteins including FoxO1 [Id., citing Yu, SL., et al. Reprod. Biol. (2022) 22: 100672] and upregulation of SIRT1 can inhibit apoptosis via the FoxO1/β-catenin pathway. [Id., citing Yao, H., et al. Mol. Med. Rep. (2018) 17: 6681-6690] It has been suggested that SIRT1, FoxO1, and sterol regulatory element binding protein-1 (SREBP-1) may act as a pathway and play crucial roles in apoptosis; thus, at both the protein and mRNA levels, SIRT1 and SREBP-1 were upregulated in progestin-resistant cells, while FoxO1 was downregulated. [Id., citing Wang, Y., et al. Arch. Gynecol. Obstet. (2018) 298: 961-969]. SIRT1 also may be a potential target for cross-regulation of post-transcriptional modifications. For example, acetylation was required for FoxO3-induced apoptosis through phosphorylated-FoxO3 (p-FoxO3) formation, while expression or activation of SIRT1 blocked p-FoxO3 formation and apoptosis. [Id., citing Li, Z., et al. Oncogene (2017) 36: 1887-1898]. Deacetylation of FoxO3 by SIRT1 resulted in S-phase kinase-associated protein 2-mediated FoxO3 ubiquitination and degradation. [Id., citing Wang, F, et al. Oncogene (2012) 31: 1546-1557]. These fine-tuning mechanisms of FoxO3 regulation modulated by PTMs may be a method to regulate apoptosis in a coordinated manner.

miRNAs play important roles in the regulation of SIRT1. SIRT1 has been revealed to be targeted by miRNAs such as miR-34a, miR-181, miR-128, miR-449 and miR-30a-5p. For example, Yamakuchi et al. demonstrated a negative correlation between the expression of miR-34a and SIRT1, suggesting SIRT1 was a target of miR-34a. [Id., citing Yamakuchi, M., et al. Proc. Natl Acad. Sci. USA (2008) 105: 13421-13426]. In addition, SIRT1 is a key player in the protection provided by miR-34a-5p inhibition during apoptosis. [Id., citing Wang, G., et al. Antioxid. Redox Signal. (2016) 24: 961-973] The overexpression of miR-181d-5p inhibited cell apoptosis and renal fibrosis in a mouse model by downregulating the SIRT1/p53 pathway. [Id., citing Fan, X., et al. Ann. Transl. Med. (2021) 9: 1571]. Furthermore, miR-181a increased FoxO1 acetylation and promoted granulosa cell apoptosis via SIRT1 downregulation. [Id., citing Zhang, M., et al. Cell Death Dis. (2017) 8: e3088]. The previous study also suggested that miR-128 promoted apoptosis in human cancers via the p53/Bak axis. [Id., citing Adlakha, Y K and Saini, N. Cell Death Dis. (2013) 4: e542]. Upregulation of miR-128 promoted apoptosis in an epilepsy model in vivo and in vitro through the SIRT1/p53/Bax/cytochrome c/caspase signaling pathway. [Id., citing Chen, D Z., et al. Int. J. Mol. Med. (2019) 44: 694-704] Other miRNAs, such as miR-449, have been investigated in a model of acute kidney injury model by detecting expression of its target SIRT1 and downstream factors p53/Bax.234 Inhibition of miR-449 elevated SIRT1 expression and inhibited acetylated p53 and Bax protein levels. [Id., citing Qin, W., et al. Med. Sci. Monit. (2016) 22: 818-823]. miR-30a-5p was reported to target SIRT1 to activate the NF-κB/NLRP3 signaling pathway, resulting in cardiomyocyte apoptosis. [Id., citing Wu, Y X., et al. Cardiovasc. Drugs. Ther. (2023) 37 (6): 1065-1076].

Effect of SIRT1 on cell proliferation. SIRT1 is involved in regulating cell proliferation by regulating both protein expression and acetylation. [Id., citing Huang, S. et al. Cell Biol. Int. (2021) 45: 1050-105 9; Wang, X., et al. Oncol. Rep. (2021) 45: 1009]. Opposite effects of SIRT1 on cell proliferation have been observed among different cell types or the regulation of different downstream molecules.

For example, SIRT1 promotes cell proliferation by regulating LC3 and retinoblastoma (Rb) acetylation. At the molecular level, SIRT1 promotes the proliferation of endometrial cancer (EC) cells by reducing acetylation of LC3.272 SIRT1 deacetylates Rb protein in the Rb/E2F transcription factor 1 (E2F1) complex, leading to dissociation of E2F1 and enhanced oligodendrocyte progenitor cell proliferation. [Id., citing Jablonska, B., et al. Nt. Commun. (2016) 7: 13866] SIRT1 directly regulates expression of transcription factor proteins, such as E2F1 and p53, subsequently promoting macrophage and HCC cell proliferation, respectively. [Id., citing Imperatore, F., et al. EMBO J. (2017) 36: 2353-2372; Lin, X L., et al. Phytomedicine (2020) 66: 153122].

However, SIRT1 can have an antiproliferative role by regulating expression of key proteins related to cell proliferation, such as AMPK and signal transducer and activator of transcription 3 (STAT3). For example, SIRT1 exerts antiproliferative effects via the AMPK/mTOR pathway in the context of mutant p53 in HCC cells. [Id., citing Zhang, Z Y., et al. J. Hepatol. (2015) 62: 121-130] SIRT1 overexpression inhibits the proliferation of renal cancer cells, while inhibition of SIRT1 expression has the opposite effect. [Id., citing Wang, X., et al. Oncol. Rep. (2021) 45: 109]. SIRT1 has been reported to inhibit gastric cancer cell proliferation via the STAT3/matrix metalloproteinase (MMP)-13 signaling pathway. [Id., citing Zhang, S., et al. J. Cell Physiol. (2019) 234: 15395-15406].

Effect of SIRT1 on cell migration and invasion. SIRT1, as a deacetylase, influences the biological functions of proteins by regulating protein deacetylation, such as deacetylation of Beclin-1. In melanoma cells, SIRT1 deacetylates Beclin-1 and then accelerates autophagic degradation of the epithelial marker E-cadherin, finally promoting endothelial to mesenchymal transition (EMT). [Id., citing Sun, T., et al. Cell Death Dis. (2018) 9: 136]. It has been suggested that SIRT1 could regulate the expression levels of several proteins that participate in cell migration and invasion, resulting in promotion of EMT. For example, both in vivo and in vitro studies have shown that expression of SIRT1 in chondrosarcoma cells could effectively take part in the metastatic plasticity of the cells by inducing EMT by enhancing expression of Twist protein, which is a critical transcriptional factor of EMT. [Id., citing Feng, H., et al. Sci. Rep. (2017) 7: 41203]. Zinc finger E-box binding homeobox 1 is an E-cadherin-related transcription factor; Yu et al. have reported that there is positive feedback between SIRT1 and Zinc finger E-box binding homeobox 1, which enhances EMT of osteosarcoma. [Id., citing Yu, XJ., et al. J. Cell Biochem. (2019) 120: 3727-3735] Epidermal SIRT1 plays a role in wound repair; SIRT1 knockdown inhibits EMT, cell migration, and TGF-β signaling in keratinocytes. [Id., cting Qiang, L., et al. Sci. Rep. (2017) 7: 14110].

The activity of Sirt1 itself can be fine-tuned in response to biological stimuli by mechanisms that alter Sirt1 expression levels. For example,

Regulation by transcription factor. Sirt1 activity is not only controlled by the accessibility of its substrates but also by its expression levels. [Revollo, and Li, X. Trends Biochem. Sci. (2013) 38 (3): 160-167] The expression of Sirt1 expression is dependent on transcription factors like cyclic AMP response-element binding protein (CREB), carbohydrate response-element binding protein (ChREBP), forkhead box 01 (FOXO1), FOXO3, several peroxisome proliferator-activated receptors (PPARs), and hypermethylated in cancer protein 1 (HIC1). In addition, Sirt1 expression can also be modulated by several miRNAs that affect the stability of its mRNA, and by ubiquitylation, which shortens the half-life of the Sirt1 protein.

A long and growing list of transcription factors can mediate changes in the expression of Sirt1. In response to fasting, for instance, cyclic AMP response-element-binding protein (CREB), a transcription factor whose activation is mediated by protein kinase A (PKA) in response to low nutrient availability, induces the expression of Sirt1 [Id., citing Noriega, L G et al. EMBO Reports (2011) 12 (10): 1069-1076; Fusco, S., et al. Proc. Natl Acad. Sci. USA (2012) 109 (2): 621-626]. Conversely, the carbohydrate response-element-binding protein (ChREBP) responds to re-feeding and represses Sirt1 [Id., citing Noriega, L G., et al. EMBO Reports (2011) 12 (10): 1069-1076]. Unlike CREB, which acts through several CREB-binding sites distributed throughout the Sirt1 gene, ChREBP works through a single ChREBP-response element located in the Sirt1 promoter [Id., citing Noriega, L G et al. EMBO Reports (2011) 12 (10): 1069-1076].

Members of the FOXO family of transcription factors also regulate the expression of Sirt1. FOXO1 induces Sirt1 expression by binding to FOXO1-response elements in the Sirt1 promoter [Id., citing Nemoto, S., et al. Science (2004) 306 (5704): 2105-2108; Xiong, S., et al. J. Biol. Chem. (2011) 286 (7): 5289-5299]. Sirt1 can also deacetylate FOXO1 and increase its transcriptional activity, suggesting a positive feedback loop between Sirt1 and FOXO1 [Id., citing Xiong, S., et al. J. Biol. Chem. (2011) 286 (7): 5289-5299]. FOXO3A, in contrast, translocates into the nucleus under low-energy conditions, where it interacts with p53 at p53 response-elements in the Sirt1 promoter to activate Sirt1 [Id., citing Nemoto, S., et al. Science (2004) 306 (5704): 2105-2108]. FOXO3A is also a target of the Sirt1 deacetylase activity, but unlike FOXO1, Sirt1 can either activate or inhibit the transcriptional activity of FOXO3A, depending on environmental stimuli [Id., citing Brunet, A., et al. Science (2004) 303 (5666): 2011-15; Motta, M C., et al. Cell (2004) 116 (4): 551-563].

Peroxisome proliferator-activated receptors (PPARs) are nuclear receptors that function as transcription factors and are also able to regulate the expression levels of Sirt1. PPARα can increase Sirt1 expression levels during a 24-hour fast in mice, presumably through a PPAR-responsive element (PPRE) present in the Sirt1 promoter [Id., citing Hayashida, S. et al. Molec. & Cellular Biochem. (2010) 339 (1-2): 285-292]. Unlike FOXOs, there is no evidence that PPARα is a deacetylation target of Sirt1, but Sirt1 can enhance the activity of PPARα through its co-activators, suggesting a positive feedback loop [Id., citing Purushotham, A. et al. Cell Metabolism (2009) 9 (4): 327-38]. PPARβ/α is yet another transcription factor capable of increasing the expression of Sirt1 [Id., citing Okazaki, M. et al. Endocrine J. (2010) 57 (5): 403-413]. Its actions seem to be mediated by Sp1, another positive regulator of Sirt1 expression [Id., citing Okazaki, M. et al. Endocrine J. (2010) 57 (5): 403-413]. PPARγ, in contrast, represses the Sirt1 gene, presumably by directly interacting with the Sirt1 promoter [Id., citing Han, L. et al. Nucleic Acids Res. (2010) 38 (21): 7458-7471]. In addition, because PPARγ is a deacetylation target of Sirt1, its negative effects on Sirt1 suggest the existence of a negative PPARγ-Sirt1 feedback loop [Id., citing Han, L., et al. Nucleic Acids Res. (2010) 38 (21): 7458-7471].

Finally, the hypermethylated in cancer protein 1 (HIC1) can negatively regulate the expression of Sirt1 [Id., citing Chen, WY., et al. Cell (2005) 123 (3): 437-448]. Like PPARγ, Sirt1 is able to interact with and deacetylate HIC1, thereby reducing its inhibitory actions and suggesting the existence of yet another negative feedback loop [Id., citing Dehennaut, V., et al. Biochem. Biophys. Res. Commun. (2012) 421 (2): 384-388].

Regulation by RNA stability. The abundance of Sirt1 is not only controlled at the transcriptional level, but also by post-transcriptional events, such as RNA stability. In this regard, the Hu antigen R (HuR) plays a major role in stabilizing the Sirt1 mRNA transcript. In the presence of HuR, Sirt1 mRNA exhibits a half-life more than 8 hours, but in its absence, the Sirt1 mRNA half-life declines to only about 1 hour; such a decline inevitably leads to lower Sirt1 expression and activity [Id., citing Abdelmohsen, K., et al. Molecular Cell. (2007) 25 (4): 543-557]. HuR stabilizes the Sirt1 mRNA transcript by binding with high affinity to three HuR RNA-recognition motifs located at the 3′-untranslated region of the Sirt1 mRNA [Id., citing Abdelmohsen, K., et al. Molecular Cell. (2007) 25 (4): 543-557]. This stabilizing interaction, however, is regulated by certain physiological conditions. During oxidative stress, for instance, the checkpoint kinase 2 (Chk2) phosphorylates HuR at multiple residues and promotes its dissociation from the Sirt1 mRNA, leading to a reduction in the expression levels and activity of Sirt1 [Id., citing Abdelmohsen, K., et al. Molecular Cell. (2007) 25 (4): 543-557].

### SIRT-1 Expression

Human skin fibroblast cells will be seeded onto 6-well plates (50,000 cells/well) and incubated overnight in complete cell growth media containing serum and growth factors.

The next day, cells will be washed 3× with PBS and then incubated for 24 hr in:
Complete cell growth media (positive control) (3 wells);
10% complete cell growth media (complete media diluted 1:10 with serum-free, growth factor-free basal media; negative control (3 wells);
10% complete cell growth media containing 1., 3.0, or 6.0×10E9 aloe exosomes (9 wells);
10% complete cell growth media containing 1.5, 3.0 or 6.0×10E8 adipose MSC exosomes (9 wells);

Cells will be collected and stained with antibody to SIRT-1 (abeam ab110304 or 19A7AB4).

The population of positively stained cells (those expressing SIRT-1) will be quantitated by FACS.

### Example 10. Clinical Case Studies, EXO FORTIFY® Revitalizing Hair and Scalp Treatment

The following clinical case studies demonstrated the safety and effectiveness of the EXO FORTIFY® composition (Table 7).

TABLE 7
EXO FORTIFY ® Composition components:

| Ingredient | Range of Concentrations/Dosage (v/v) |
|---|---|
| Aloe³ | 1 × 10E8-25 × 10E9 |
| Niacinamide | 1%-10% |
| AnaGain ™ | 1%-5% |
| HAIRGENYL ® | 0.05%-0.5% |
| Copper tripeptide (GHK-Cu) | 0.05-3% |
| Biotin | 0.000001-0.1% |
| Antifungal agent | 0.5-0.2% |
| Saw palmetto | 2-20% |
| Panthenol (humectant) | 0.5-5% |

The term “volume/volume percentage” (“v/v %”) refers to a measure of the concentration of a substance in a solution. It is expressed as the ratio of the volume of the solute to the total volume of the solution multiplied by 100.

Composition components include:

“Aloe³ Exosomes” refers to exosomes isolated from heat-shocked Aloe plants Extracellular vesicles are isolated from Aloe vera barbadensis. The isolated exosomes are obtained from the plant's leaf flesh conditioned by growing the plant under conditions that include a heat shock of the plant at a temperature of from about 33° C. to about 45° C. for about 1 hour to about 3 hours. Between 1.0×10E8-25×10E9 of exosomes are used for the active formulation.

Niacinamide (AnMar), a type of vitamin B3 (formula C₆H₆N₂O)

AnaGain™ (Mibelle AG Biochemistry), is a hair growth promoting natural plant extract derived from organic pea shoots (Pisum sativum).

HAIRGENYL® (Silab), is a hair growth extract derived from the yeast Pichia minuta and isolated from the flowers of azalea, which supports the biological activity of the dermal papilla and stimulates growth of the hair follicle.

Copper tripeptide (GHK-Cu, Paramount Peptides) comprises complexes comprising glycyl-L-histidyl-1-lysine tripeptides (GHK) and divalent copper (see Liu, T. et al. Bioact. Mater. (2023) 32: 502-513, citing Shou, M. et al. Chem. Eng. J. (2021) 422 doi: 10.1016/j.cej.2021.130147; Li, Y. et al. Chem. Eng. J. (2023) 455 doi: 10.1016/j.cej.2022.140848; Pickart L. et al. Biomed Res. Int. (2014) 2014 doi: 10.1155/2014/151479].

Biotin C₁₀H₁₆N₂O₃S, CID 171548 (https://pubchem.ncbi.nlm.nih.gov/compound/Biotin) (AnMar) (vitamin B7), is a water-soluble vitamin

The terms “anti-fungal agent” or “antifungal agent” are used interchangeably to refer to any of a group of chemical substances having the capacity to inhibit the growth of or to destroy fungi. Anti-fungal agents including but not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate. Extracts, constituents or phytochemicals from the following plants contain natural antifungal compounds: Echinacea purpurea; Daikon radish; Vicia faba; Artemisia annua; Melaleuca alternifolia; Syzygium aromaticum; Salvia rosmarinus; Calendula officinalis; Azadirachta indica; Curcuma longa; Lavandula angustifolia; Hypericum perforatum; Pistacia lentiscus; Psidium guajava; Punica granatum; Rubia tinctorum; Prunus yedoensis; and Cassia tora.

The term “panthenol”, also known as provitamin B5, as used herein refers to a water-soluble alcohol analog of pantothenic acid (vitamin B5) hygroscopic substance that promotes moisture retention.

The term “saw palmetto” as used herein refers to an herbal extract of the fruit of the saw palmetto dwarf palm.

Procedure: Subjects topically applied the composition to the scalp in areas of hair loss, twice daily. No adverse effects were reported. Subjects reported that their scalp condition was improved. 100% of subjects were satisfied with the treatment and want to continue.

Rapid hair regrowth is shown in each case.

Subject 1: Female, with hair loss from salon coloring treatments every two weeks.

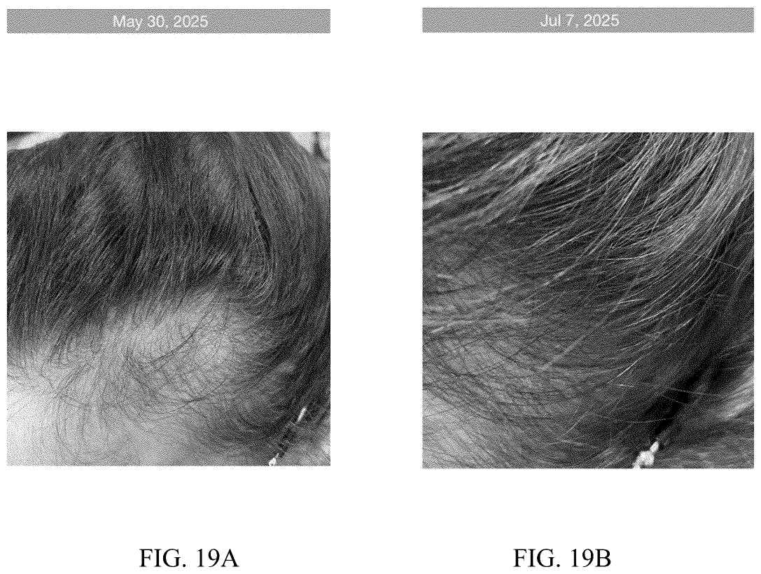

FIG. 19A is a photograph of the scalp of Subject 1 showing hair loss before treatment, FIG. 19B is a photograph of the same area of the scalp after 5 weeks of treatment.

Subject 2: Female, with hair loss due to androgenetic alopecia

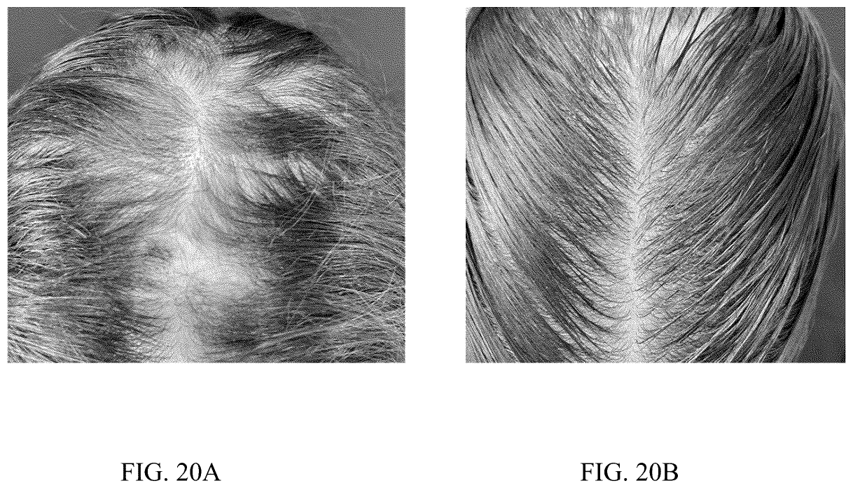

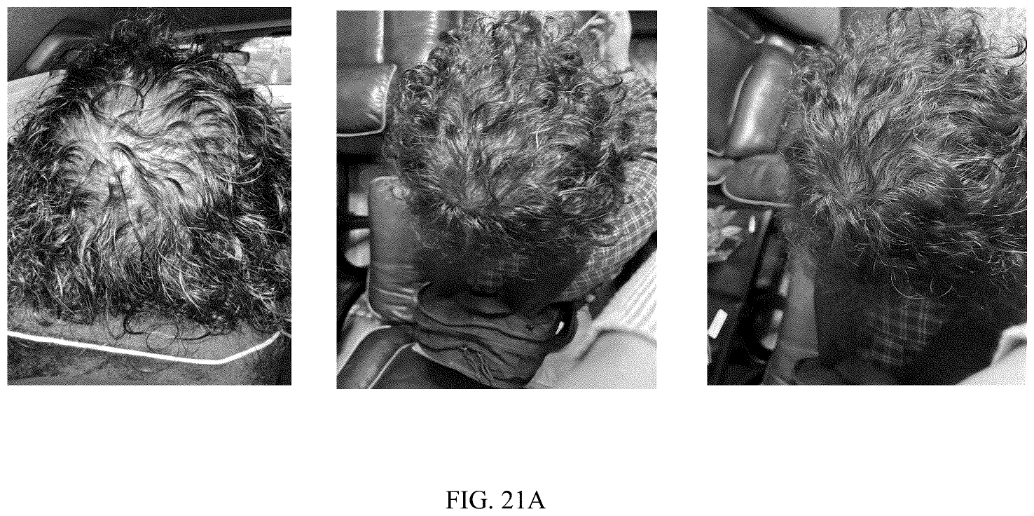

FIG. 20A is a photograph of the scalp of Subject 2 showing hair loss before treatment, FIG. 20B is a photograph of the same area of the scalp after 8 weeks of treatment.

Subject 3: Male, with hair loss post COVID-19

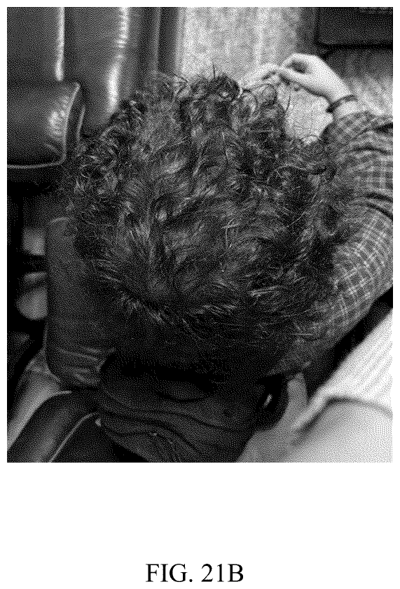

FIG. 21A is a photograph of the scalp of Subject 3 showing hair loss before treatment, FIG. 21B is a photograph of the same area of the scalp after 15 weeks of treatment.

Subject 4: Male, hair loss of unknown pathology

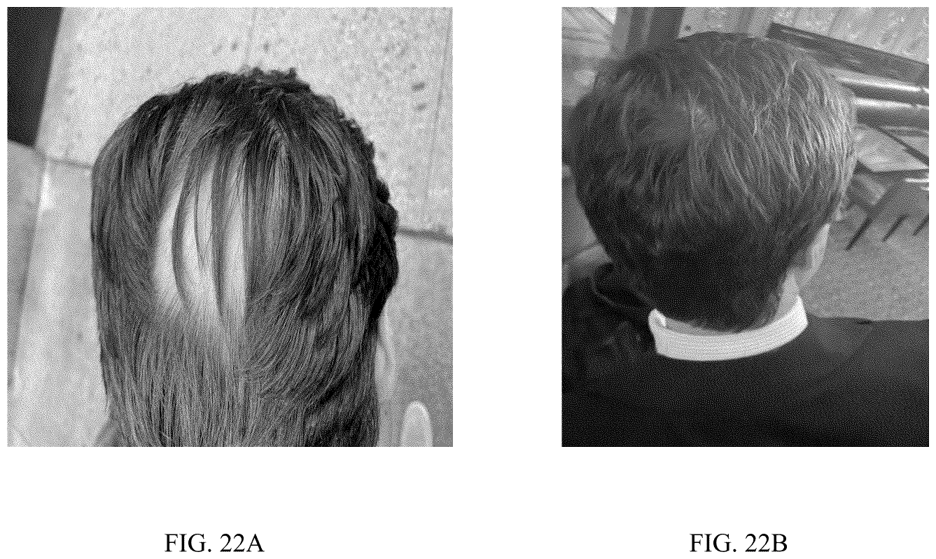

FIG. 22A is a photograph of the scalp of Subject 4 showing hair loss before treatment, FIG. 22B is a photograph of the same area of the scalp after 20 weeks of treatment.

Subject 5: male with alopecia areata

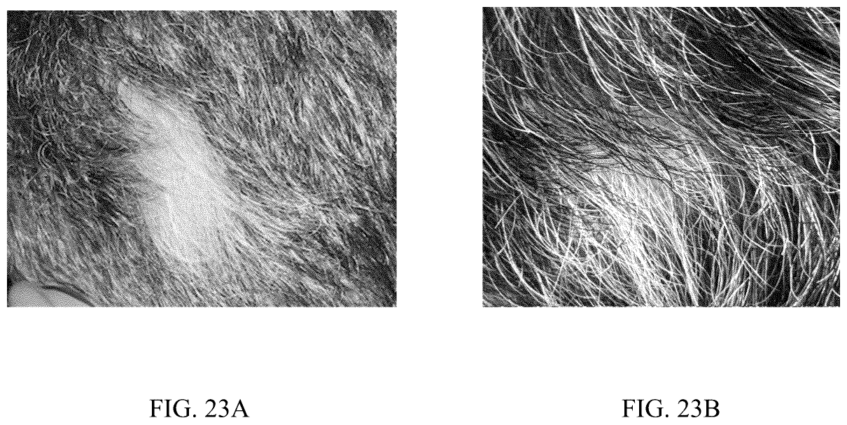

FIG. 23A is a photograph of the scalp of Subject 5 showing hair loss before treatment, FIG. 23B is a photograph of the same area of the scalp after 12 weeks of treatment.

Subject 6: 58 year old male with androgenetic alopecia and stress

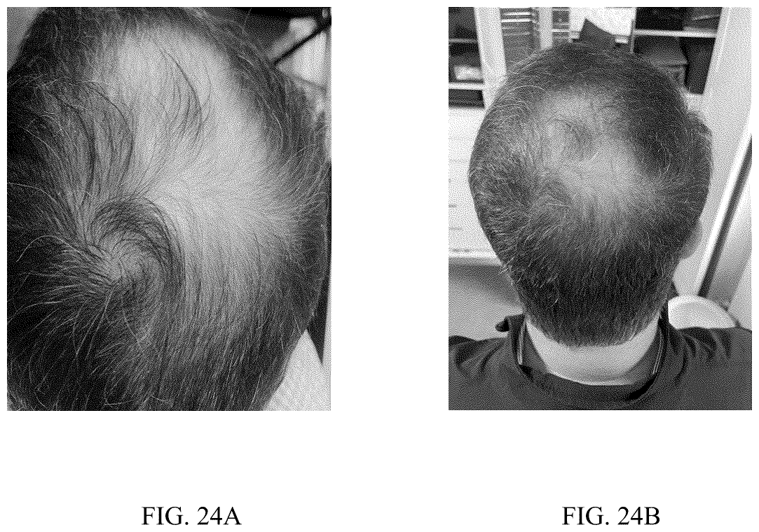

FIG. 24A is a photograph of the scalp of Subject 6 showing hair loss before treatment, FIG. 24B is a photograph of the same area of the scalp after 20 weeks of treatment.

### Example 11. Determination of the Signature Cargo of Exosomes Derived from Heat-Stressed Aloe vera Plants Vs. Native Aloe vera Plants

The signature cargo of exosomes were isolated from heat stressed Aloe vera plants and native Aloe vera plants, as described in Example 1. Total RNA was isolated from the isolated exosomes, as described in Example 2.

RNA sequencing (RNA-Seq) was performed on the total RNA isolated from the exosomes that were isolated from heat stressed Aloe vera plants and native Aloe vera plants and differential miRNA expression was determined.

Results: Inducing stress causes differential expression of miRNA in Aloe vera-derived exosomes isolated from heat stressed Aloe vera plants compared to native Aloe vera plants, as shown in Table 8.

TABLE 8
Differential Expression of miRNA in Exosomes Derived from Heat-
Stressed Aloe vera Plants vs. Native Aloe vera Plants

| | | | | Exosomes | |
|---|---|---|---|---|---|
| | | | | Derived | Exosomes |
| | | | | from | Derived |
| | MiRBase | | | Heat- | from |
| | Accession | | SEQ ID | Stressed | Native |
| miRNA | Number | Mature miRNA Sequence | NO: | Aloe vera | Aloe vera |
| ath- | MIMAT0020520 | UUCUUGUGGAUUCCUUG | 23 | + | − |
| miR5016 | | GAAA | | | |
| ath- | MIMAT0023522 | ACAGUUUGUGUUUUGUU | 24 | + | − |
| miR5998b | | UUGU | | | |
| ath- | MIMAT0020524 | UGGAAGAAGGUGAGACU | 25 | + | − |
| miR5020a | | UGCA | | | |
| ath- | MIMAT0004257 | UCCUGUGUUUCCUUUGA | 26 | + | − |
| miR836 | | UGCGUGG | | | |
| ath- | MIMAT0031873 | CUUUGUCUACAAUUUUG | 27 | + | − |
| miR158a- | | GAAA | | | |
| 5p | | | | | |
| ath- | MIMAT0000942 | CUGAAGUGUUUGGGGGA | 28 | + | − |
| miR395e | | ACUC | | | |
| ath- | MIMAT0001003 | UUCGAGGCCUAUUAAAC | 29 | + | − |
| miR402 | | CUCUG | | | |
| ath- | MIMAT0022439 | AGAGGUGACCAUUGGAG | 30 | + | − |
| miR5662 | | AUG | | | |
| ath- | MIMAT0022389 | GCUAAGAGCGGUUCUGA | 31 | + | − |
| miR5630a | | UGGA | | | |
| ath- | MIMAT0022399 | GCUAAGAGCGGUUCUGA | 32 | + | − |
| miR5630b | | UGGA | | | |
| ath- | MIMAT0018348 | AGAAGCAAAAUGACGAC | 33 | + | − |
| miR3933 | | UCGG | | | |
| ath- | MIMAT0023521 | ACAGUUUGUGUUUUGUU | 34 | + | − |
| miR5998 | | UUGU | | | |
| ath- | MIMAT0001019 | GGAAUCUUGAUGAUGCU | 35 | + | − |
| miR172e- | | GCAU | | | |
| 3 | | | | | |
| ath- | MIMAT0021048 | CCGUAUCUUGGCCUUGU | 36 | + | − |
| miR5024- | | CAUU | | | |
| 3p | | | | | |
| ath- | MIMAT0002113 | UUGGGGACGAGAUGUUU | 37 | + | − |
| miR447a- | | UGUUG | | | |
| 3p | | | | | |
| ath- | MIMAT0001322 | UCAUCUUCAUCAUCAUC | 38 | + | − |
| miR414 | | GUCA | | | |
| ath- | MIMAT0031883 | GAUCAUGUUCGCAGUUU | 39 | + | − |
| miR167a- | | CACC | | | |
| 3p | | | | | |
| ath- | MIMAT0000204 | GCAGCACCAUUAAGAUU | 40 | + | − |
| miR172b- | | CAC | | | |
| 5p | | | | | |
| ath- | MIMAT0022396 | CGUAGUUGCAGAGCUUG | 41 | + | − |
| miR5636 | | ACGG | | | |
| ath- | MIMAT0032024 | CCUUCUCAUCGAUGGUC | 42 | + | − |
| miR824- | | UAGA | | | |
| 3p | | | | | |
| ath- | MIMAT0031918 | GCAGCACCAUUAAGAUU | 43 | + | − |
| miR172e- | | CAC | | | |
| 5p | | | | | |
| ath- | MIMAT0001005 | AUUAACGCUGGCGGUUG | 44 | + | − |
| miR404 | | CGGCAGC | | | |
| ath- | MIMAT0002114 | UUGGGGACGAGAUGUUU | 45 | + | − |
| miR447b | | UGUUG | | | |
| ath- | MIMAT0035543 | UGGUUUUGGACACGUGA | 46 | + | − |
| miR826b | | AAAU | | | |
| ath- | MIMAT0000911 | UGAGCCAAGGAUGACUU | 47 | + | − |
| miR169g- | | GCCG | | | |
| 5p | | | | | |
| ath- | MIMAT0004319 | CUUCUUAAGUGCUGAUA | 48 | + | − |
| miR868- | | AUGC | | | |
| 3 | | | | | |
| ath- | MIMAT0004248 | UAACUAUUUUGAGAAGA | 49 | + | − |
| miR830- | | AGUG | | | |
| 3p | | | | | |
| ath- | MIMAT0000910 | UGAGCCAAGGAUGACUU | 50 | + | − |
| miR169f- | | GCCG | | | |
| 5p | | | | | |
| ath- | MIMAT0004244 | UCUUGCUUAAAUGAGUA | 51 | + | − |
| miR828 | | UUCCA | | | |
| ath- | MIMAT0032781 | UUGUGUUGCGUUUCUGU | 52 | + | − |
| miR8182 | | UGAUU | | | |
| ath- | MIMAT0031874 | GCGUAUGAGGAGCCAUG | 53 | + | − |
| miR160a- | | CAUA | | | |
| 3p | | | | | |
| ath- | MIMAT0022405 | UGUUAAGGAGUGUUAAC | 54 | + | − |
| miR5635d | | GGUG | | | |
| ath- | MIMAT0000953 | UGCCAAAGGAGAGUUGC | 55 | + | − |
| miR399c- | | CCUG | | | |
| 3p | | | | | |
| ath- | MIMAT0022428 | UGUUAAGGAGUGUUAAC | 56 | + | − |
| miR5635c | | GGUG | | | |
| ath- | MIMAT0000948 | UGUGUUCUCAGGUCACC | 57 | + | − |
| miR398a- | | CCUU | | | |
| 3p | | | | | |
| ath- | MIMAT0000933 | UUCGCAGGAGAGAUAGC | 58 | + | − |
| miR391- | | GCCA | | | |
| 5p | | | | | |
| ath- | MIMAT0003940 | UUAGAGUUUUCUGGAUA | 59 | + | − |
| miR781a | | CUUA | | | |
| ath- | MIMAT0031872 | GCUCUCUAUACUUCUGU | 60 | + | − |
| miR157c- | | CACC | | | |
| 3p | | | | | |
| ath- | MIMAT0000952 | UGCCAAAGGAGAGUUGC | 61 | + | − |
| miR399b | | CCUG | | | |
| ath- | MIMAT0023524 | AUUUGUACACCUAGAUC | 62 | + | − |
| miR5014b | | UGUA | | | |
| ath- | MIMAT0022418 | UGUUAAGGAGUGUUAAC | 63 | + | − |
| miR5635b | | GGUG | | | |
| ath- | MIMAT0004329 | UGAUUGGAAAUUUCGUU | 64 | + | − |
| miR779.2 | | GACU | | | |
| ath- | MIMAT0000932 | AAGCUCAGGAGGGAUAG | 65 | + | − |
| miR390b- | | CGCC | | | |
| 5 | | | | | |
| ath- | MIMAT0004253 | UAGACCGAUGUCAACAA | 66 | + | − |
| miR833a- | | ACAAG | | | |
| 3p | | | | | |
| ath- | MIMAT0004271 | UAACUAAACAUUGGUGU | 67 | + | − |
| miR849 | | AGUA | | | |
| ath- | MIMAT0022395 | UGUUAAGGAGUGUUAAC | 68 | + | − |
| miR5635a | | GGUG | | | |
| ath- | MIMAT0017742 | CAAUUUCUAGUGGGUCG | 69 | + | − |
| miR841b- | | UAUU | | | |
| 3 | | | | | |
| ath- | MIMAT0000931 | AAGCUCAGGAGGGAUAG | 70 | + | − |
| miR390a- | | CGCC | | | |
| 5p | | | | | |
| ath- | MIMAT0002115 | UUGGGGACGACAUCUUU | 71 | + | − |
| miR447c- | | UGUUG | | | |
| 3p | | | | | |
| ath- | MIMAT0004256 | UGGAGAAGAUACGCAAG | 72 | + | − |
| miR835- | | AAAG | | | |
| 3p | | | | | |
| ath- | MIMAT0022407 | ACAGUGGUCAUCUGGUG | 73 | + | − |
| miR5638b | | GGCU | | | |
| ath- | MIMAT0011155 | CUUUAUAUCCGCAUUUG | 74 | + | − |
| miR2112- | | CGCA | | | |
| 3p | | | | | |
| ath- | MIMAT0022425 | UGGGUUGAGUUGAGUUG | 75 | + | − |
| miR5653 | | AGUUGGC | | | |
| ath- | MIMAT0031880 | GGACUGUUGUCUGGCUC | 76 | + | − |
| miR166a- | | GAGG | | | |
| 5p | | | | | |
| ath- | MIMAT0031889 | GAGCUCCUUGAAGUUCA | 77 | + | − |
| miR159b- | | AUGG | | | |
| 5p | | | | | |
| ath- | MIMAT0031881 | GGACUGUUGUCUGGCUC | 78 | + | − |
| miR166b- | | GAGG | | | |
| 5p | | | | | |
| ath- | MIMAT0004265 | UUUAGGUCGAGCUUCAU | 79 | + | − |
| miR843 | | UGGA | | | |
| ath- | MIMAT0020519 | UUGGUGUUAUGUGUAGU | 80 | + | − |
| miR5015 | | CUUC | | | |
| ath- | MIMAT0022420 | UUAGAGUUUUCUGGAUA | 81 | + | − |
| miR781b | | CUUA | | | |
| ath- | MIMAT0023523 | ACAAAGUUUUAUACUGA | 82 | + | − |
| miR4245 | | CAAU | | | |
| ath- | MIMAT0000906 | CAGCCAAGGAUGACUUG | 83 | + | − |
| miR169b- | | CCGG | | | |
| 5p | | | | | |
| ath- | MIMAT0020517 | UUUGUGACAUCUAGGUG | 84 | + | − |
| miR5013 | | CUUU | | | |
| ath- | MIMAT0004311 | UCAGGUAUGAUUGACUU | 85 | + | − |
| miR864- | | CAAA | | | |
| 5p | | | | | |
| ath- | MIMAT0004315 | UCAAGGAACGGAUUUUG | 86 | + | − |
| miR866- | | UUAA | | | |
| 5p | | | | | |
| ath- | MIMAT0023517 | ACAUAUGAUCUGCAUCU | 87 | + | − |
| miR5595a | | UUGC | | | |
| ath- | MIMAT0001004 | UUAGAUUCACGCACAAA | 88 | + | − |
| miR403- | | CUCG | | | |
| 3p | | | | | |
| ath- | MIMAT0031916 | CACGUGUUCUACUACUC | 89 | + | − |
| miR164c- | | CAAC | | | |
| 3p | | | | | |
| ath- | MIMAT0004255 | UUCUUGCAUAUGUUCUU | 90 | + | − |
| miR835- | | UAUC | | | |
| 5p | | | | | |
| ath- | MIMAT0000188 | UCGGACCAGGCUUCAUC | 91 | + | − |
| miR165b | | CCCC | | | |
| ath- | MIMAT0017737 | ACUUGGCUGAUUCUAUU | 92 | + | − |
| miR3434- | | AUU | | | |
| 5p | | | | | |
| ath- | MIMAT0032775 | GGCCGGUGGUCGCGAGA | 93 | − | + |
| miR8176 | | GGGA | | | |
| ath- | MIMAT0022390 | UGGCAGGAAAGACAUAA | 94 | − | + |
| miR5631 | | UUUU | | | |
| ath- | MIMAT0000951 | UGCCAAAGGAGAUUUGC | 95 | − | + |
| miR399a | | CCUG | | | |
| ath- | MIMAT0017943 | UCACUGGUACCAAUCAU | 96 | − | + |
| miR4227 | | UCCA | | | |
| ath- | MIMAT0022444 | AUGGGACAUCGAGCAUU | 97 | − | + |
| miR5666 | | UAAU | | | |
| ath- | MIMAT0003937 | UGGCUUGGUUUAUGUAC | 98 | − | + |
| miR778 | | ACCG | | | |
| ath- | MIMAT0004274 | UGGGUGGCAAACAAAGA | 99 | − | + |
| miR851- | | CGAC | | | |
| 3p | | | | | |
| ath- | MIMAT0032129 | UGAGAAUGCAAAUCCUU | 100 | − | + |
| miR5663- | | AGCU | | | |
| 3p | | | | | |
| ath- | MIMAT0004251 | UUGAUUCCCAAUCCAAG | 101 | − | + |
| miR832- | | CAAG | | | |
| 3p | | | | | |
| ath- | MIMAT0022410 | GUUCGAGGCACGUUGGG | 102 | − | + |
| miR5646 | | AGG | | | |
| ath- | MIMAT0004300 | UAAUCCUACCAAUAACU | 103 | − | + |
| miR856 | | UCAGC | | | |
| ath- | MIMAT0004258 | AUCAGUUUCUUGUUCGU | 104 | − | + |
| miR837- | | UUCA | | | |
| 5p | | | | | |
| ath- | MIMAT0004269 | UUGAAUUGAAGUGCUUG | 105 | − | + |
| miR846- | | AAUU | | | |
| 3p | | | | | |
| ath- | MIMAT0004243 | UUAGAUGACCAUCAACA | 106 | − | + |
| miR827 | | AACU | | | |
| ath- | MIMAT0022393 | UAUGAUCAUCAGAAAAC | 107 | − | + |
| miR5633 | | AGUG | | | |
| ath- | MIMAT0001321 | AUAGUUUCUCUUGUUCU | 108 | − | + |
| miR413 | | GCAC | | | |
| ath- | MIMAT0004260 | UUUUCUUCUACUUCUUG | 109 | − | + |
| miR838 | | CACA | | | |
| ath | MIMAT0022426 | AUAAAUCCCAACAUCUU | 110 | − | + |
| miR5654- | | CCA | | | |
| 5p | | | | | |
| ath- | MIMAT0031901 | GCAACAUCUUCAAGAUU | 111 | − | + |
| miR172d- | | CAGA | | | |
| 5p | | | | | |
| ath- | MIMAT0022403 | UCUCGCGCUUGUACGGC | 112 | − | + |
| miR5642a | | UUU | | | |
| ath- | MIMAT0001328 | UAAACUAAUCACGGAAA | 113 | − | + |
| miR420 | | UGCA | | | |
| ath- | MIMAT0004249 | UGAUCUCUUCGUACUCU | 114 | − | + |
| miR831- | | UCUUG | | | |
| 3p | | | | | |
| ath- | MIMAT0031868 | GCUCACUCUCUUUUUGU | 115 | − | + |
| miR156d- | | CAUAAC | | | |
| 3p | | | | | |
| ath- | MIMAT0020522 | UUAAAGCUCCACCAUGA | 116 | − | + |
| miR5018 | | GUCCAAU | | | |
| ath- | MIMAT0032766 | AGGUGCUGAGUGUGCUA | 117 | − | + |
| miR8168 | | GUGC | | | |
| ath- | MIMAT0004316 | ACAAAAUCCGUCUUUGA | 118 | − | + |
| miR866- | | AGA | | | |
| 3p | | | | | |
| ath- | MIMAT0032769 | UUGCUUAAAGAUUUUCU | 119 | − | + |
| miR8170- | | AUGU | | | |
| 3p | | | | | |
| ath- | MIMAT0000939 | CUGAAGUGUUUGGGGGG | 120 | − | + |
| miR395b | | ACUC | | | |
| ath- | MIMAT0000940 | CUGAAGUGUUUGGGGGG | 121 | − | + |
| miR395c | | ACUC | | | |
| ath- | MIMAT0003939 | UUCUUCGUGAAUAUCUG | 122 | − | + |
| miR780.2 | | GCAU | | | |
| ath- | MIMAT0001018 | UAAGCUGCCAGCAUGAU | 123 | − | + |
| miR167c- | | CUUG | | | |
| 5p | | | | | |
| ath- | MIMAT0031905 | AUCAUGCUAUCUCUUUG | 124 | − | + |
| miR393a- | | GAUU | | | |
| 3p | | | | | |
| ath- | MIMAT0000943 | CUGAAGUGUUUGGGGGG | 125 | − | + |
| miR395f | | ACUC | | | |

### Example 12. Gene Expression Analysis of In Vitro Dermal Papillae Cells 24 Hours after Treatment with Test Materials

### 12.1. Project Summary

This study was conducted in order to understand how different test materials (TMs) influence gene expression in human follicle dermal papilla cells (HFDPCs).

HFDPCs are a group of mesenchymal-derived cells at the base of the hair follicle that regulates hair follicle growth through the expression and secretion of specific biomarkers.

Cells from a Caucasian female donor (age: 50) were used for this study.

Gene expression was assessed using the Genemarkers® Dermal Papilla Cell Panel (see, Table 9), a qPCR-based gene expression panel which contains 52 target genes and 4 endogenous control genes.

Gene expression was assessed 24 hours following the application of the TMs.

The following treatment groups were including in the study (n=4):
1. Untreated Group (negative control);
2. Media+Serum (positive control);
3. PBS treated for 1 hour (vehicle control);
4. TM1—1.5 billion native (not heat shocked) aloe exosomes;
5. TM2—1.5 billion engineered (heat shocked) aloe-derived exosomes (low dose);
6. TM3—5 billion engineered (heat shocked) aloe-derived exosomes (high dose); and
7. TM4—5 billion engineered (heat shocked) human adipose stromal stem cell (ASC)-derived exosomes.

TABLE 9
Dermal Papilla Cell Panel Gene Information and Potential
Expression Change to Promote Hair Growth

| | | | Potential |
|---|---|---|---|
| | | | Expression |
| | | Associatedin | Change to |
| | | Function(s) | Promote Hair |
| Gene ID | Gene Name | the Skin | Growth |
| AR | Androgen Receptor | Hormone Regulation | Decrease |
| ASIP | Agouti Signaling Protein | Pigmentation | Increase |
| AXIN2 | Axin 2 | Wnt/BCT Pathway | Increase |
| BAX | BCL2 Associated X, Apoptosis | Proliferation, Cell Cycle, | Unknown |
| | Regulator | and Apoptosis | |
| BCL2 | BCL2 Apoptosis Regulator | Proliferation, Cell Cycle, | Increase |
| | | and Apoptosis | |
| BMP2 | Bone Morphogenic Protein 2 | BMP Pathway | Decrease |
| BMP4 | Bone Morphogenic Protein 4 | BMP Pathway | Decrease |
| BMP6 | Bone Morphogenic Protein 6 | BMP Pathway | Decrease |
| CCND1 | Cyclin D1 | Proliferation, Cell Cycle, | Increase |
| | | and Apoptosis | |
| CDKN2A | Cyclin Dependent Kinase | Proliferation, Cell Cycle, | Decrease |
| | Inhibitor 2A | and Apoptosis | |
| CORIN | Corin, Serine Peptidase | Anagen Marker/Inducer | Unknown |
| CTNNB1 | Catenin Beta 1 | Wnt/BCT Pathway | Unknown |
| DCN | Decorin | Anagen Marker/Inducer | Increase |
| DKK1 | Dickkopf WNT Signaling | Wnt/BCT Pathway | Decrease |
| | Pathway Inhibitor 1 | | |
| ESR1 | Estrogen Receptor 1 | Hormone Regulation | Increase |
| ESR2 | Estrogen Receptor 2 | Hormone Regulation | Increase |
| FGF10 | Fibroblast Growth Factor 10 | Anagen Marker/Inducer | Increase |
| FGF5 | Fibroblast Growth Factor 5 | Catagen Marker/Inducer | Unknown |
| FGF7 | Fibroblast Growth Factor 7 | Anagen Marker/Inducer | Increase |
| GLI1 | GLI Family Zinc Finger 1 | Hair Follicle Regrowth | Increase |
| GSK3B | Glycogen Synthase Kinase 3 | Wnt/BCT Pathway | Unknown |
| | Beta | | |
| HES1 | Hes Family BHLH | Notch Signaling Pathway | Increase |
| | Transcription Factor 1 | | |
| HEY1 | Hes Related Family BHLH | Anagen Marker/Inducer | Increase |
| | Transcription Factor with | | |
| | YRPW Motif 1 | | |
| HGF | Hepatocyte Growth Factor | Proliferation, Cell Cycle, | Increase |
| | | and Apoptosis | |
| IGF1 | Insulin Like Growth Factor 1 | Proliferation, Cell Cycle, | Increase |
| | | and Apoptosis | |
| IL1B | Interleukin 1 Beta | Catagen Marker/Inducer | Decrease |
| IL6 | Interleukin 6 | Catagen Marker/Inducer | Decrease |
| KITLG | KIT Ligand | Pigmentation | Increase |
| LEF1 | Lymphoid Enhancer Binding | Wnt/BCT Pathway | Unknown |
| | Factor 1 | | |
| LEP | Leptin | Anagen Marker/Inducer | Increase |
| MC1R | Melanocortin 1 Receptor | Pigmentation | Unknown |
| MKI67 | Marker of Proliferation Ki-67 | Proliferation, Cell Cycle, | Increase |
| | | and Apoptosis | |
| NOG | Noggin | BMP Pathway | Increase |
| NOTCH1 | Notch Receptor 1 | Notch Signaling Pathway | Increase |
| PDGFA | Platelet Derived Growth Factor | Proliferation, Cell Cycle, | Unknown |
| | Subunit A | and Apoptosis | |
| PROM1 | Prominin 1 | Anagen Marker/Inducer | Unknown |
| PTCH1 | Patched 1 | Hair Follicle Regrowth | Increase |
| SFRP1 | Secreted Frizzled Related | Wnt/BCT Pathway | Decrease |
| | Protein 1 | | |
| SOX2 | SRY-Box Transcription Factor 2 | BMP Pathway | Increase |
| SRD5A2 | Steroid 5 Alpha-Reductase 2 | Hormone Regulation | Decrease |
| STAT3 | Signal Transducer and Activator | JAK-STAT Pathway | Unknown |
| | of Transcription 3 | | |
| STAT5A | Signal Transducer and Activator | JAK-STAT Pathway | Increase |
| | of Transcription 5A | | |
| STAT6 | Signal Transducer and Activator | JAK-STAT Pathway | Decrease |
| | of Transcription 6 | | |
| TCF4 | Transcription Factor 4 | Wnt/BCT Pathway | Increase |
| TGFB1 | Transforming Growth Factor | Wnt/BCT Pathway | Decrease |
| | Beta 1 | | |
| TGFB2 | Transforming Growth Factor | Catagen Marker/Inducer | Unknown |
| | Beta 2 | | |
| VCAN | Versican | Anagen Marker/Inducer | Increase |
| VEGFA | Vascular Endothelial Growth | Proliferation, Cell Cycle, | Increase |
| | Factor A | and Apoptosis | |
| WIF1 | WNT Inhibitory Factor 1 | Wnt/BCT Pathway | Unknown |
| WNT10B | Wnt Family Member 10B | Wnt/BCT Pathway | Unknown |
| WNT3A | Wnt Family Member 3A | Wnt/BCT Pathway | Unknown |
| WNT5A | Wnt Family Member 5A | Wnt/BCT Pathway | Increase |

### Gene Annotations for Table 9:

AR (Hormone Regulation). The Androgen Receptor (AR) gene is involved in the regulation of androgen hormones. As people age, androgens tend to stimulate hair growth in some areas of the body (such as facial hair) and inhibit hair growth in other areas (such as the scalp). AR expression can also downregulate BMP2 and BMP4.^{1, 2, 3}

Agouti/ASIP (Pigmentation). Agouti Signaling Protein (ASIP/ASP) is a gene that changes pigmentation through its interactions with MC1R and MSH. Proteins produced by ASIP can bind to MC1R, inhibiting MSH binding and the eumelanogenic effects that go along with it. This can cause a switch from eumelanogenesis (brown to black hair color) to pheomelanogenesis (red and yellow color), leading to red hair in humans and yellow hair in mice. A SNP of ASIP has been found to be significantly associated with red hair in humans. This gene is highly expressed in the early growth phase of dermal papilla cells.^{4, 5}

AXIN2 (Hair growth/Hair homeostasis). Axin2 is a well-established Wnt/β-catenin target gene that is expressed in hair follicle stem cells and in the outer bulge throughout telogen and anagen. Upregulated expression of axis inhibition protein 2 (Axin2) prevents the onset of catagen, with the result being significantly longer and thicker hair.^{6,7,8,9}

BCL2 (Hair loss/Apoptosis). The BCL family is important for regulation of apoptosis, with BCL2 Apoptosis Regulator (BCL2) inhibiting and BCL2-associated X (BAX) promoting. Maintaining a good ratio of gene expression for these genes is essential for cell health. Expression of BAX is increased during the catagen phase (when hair stops growing and becomes detached from the follicle) and the ratio of BCL2/BAX is decreased. DPCs tend to show high levels of BCL2 and are generally resistant to apoptosis.^{10, 11, 12}

BMP2, BMP4, BMP6 (Growth Factor/BMP Pathway). Bone morphogenetic proteins (BMPs) are a group of peptide growth factors that belong to the TGF-b superfamily. They regulate gene expression via regulating the recruitment of transcription factors. Traditionally, the inhibition of BMPs has been shown to promote anagen in hair follicles, where the downregulation of BMPs is associated with increased activation of Wnt signaling. Accumulation of BMP-inhibitory signals can initiate fresh hair growth. Downregulation of BMP2 was seen in cultured human hairs with prolonged anagen phases and in the accelerated regrowth of hair in mice. BMP4 is expressed in the hair shaft precursors (precortical matrix) and in the dermal papilla. BMP4 regulates hair follicle cycling and is important for hair cycle morphogenesis. BMP2 and BMP4 are expressed in arrested hair follicles. Downregulation of BMP4 leads to anagen development in hair follicles. Upregulation of BMP6 in dermal papilla cells leads to decreased cell migration. BMP4 and BMP6 also arrest the hair follicle during the telogen phase (resting stage). BMP6 and Wnt10b competitively regulate the telogen-anagen transition. When BMP6 is upregulated and Wnt10b is downregulated, cells do not process to anagen and remain in telogen; when BMP6 is downregulated and Wnt10b is upregulated, cells transition to anagen normally and hair growth occurs.^{13, 14, 15, 16, 17, 18, 19, 20, 21, 22, 23}

DCN (Anagen Marker/Inducer). Decorin (DCN) is a small, leucine-rich proteoglycan found in the extracellular matrix of the dermal cell layer. Proteoglycans have been suggested to play an important role in hair biology. Studies have shown that DCN is highly expressed in hair follicle epithelial cells and dermal papilla cells during the anagen phase. Other studies were able to confirm that the addition of exogenous DCN accelerated anagen and delayed catagen. In decorin knockdown mice, the telogen-to-anagen transition was delayed in hair follicles while the anagen-to-catagen transition was accelerated.^{24, 25, 26}

ESR1/alpha. Oestrogen receptor alpha (ESR1; ERalpha) is a ligand-activated Transcription factor; expressed in the human HF (dermal papilla) mediates 17-β. Oestradiol-dependent effects on HF cycling, observed to stimulate hair shaft prolongation of male frontotemporal HFs in vitro. Estrogens can modulate hair growth. When ERalpha is stimulated, the telogen-to-anagen transition in hair follicles is inhibited. ERalpha is an activator in the presence of 17 β-estradiol while ERbeta is an inhibitor in the presence of 17 β-estradiol. ERalpha and ERbeta are seen in the dermal papilla during anagen. In early catagen, ERalpha is downregulated in the dermal papilla while ERbeta is seen during early and late catagen. During the anagen-to-catagen transition and telogen, ERalpha is upregulated in telogen hair follicles; ER [symbol] is also seen in telogen hair follicles. In summary, ERbeta is expressed during the entirety of the hair cycle while ERalpha peaks during telogen and is at its lowest in early catagen. In ERalpha-knockout mice, the development of catagen is accelerated.^{27, 28, 29}

ESR2/beta. Oestrogen receptor beta (ESR2; ERP) is the principal ER in the hair follicle. Cultured mesenchymal hair follicle cells (dermal papilla and dermal), as well as interfollicular dermal fibroblasts, predominantly express ESR2. Positive expression in hair follicle structures including the inner and outer root sheaths, bulge region and dermal papilla. See ESR1/alpha for more information.^{30, 31, 32}

FGF7/KGF: FGF10 (Anagen Marker/Proliferation). Fibroblast Growth Factors (FGFs) help proliferate and differentiate progenitor hair follicles. They are involved in the promotion of proliferation of epithelial cells and wound healing. Expression is shown to be upregulated in telogen as they initiate the transition from telogen to anagen. It is thought that FGF7 and FGF10 signaling instruct hair follicles to proliferate and initiate a new hair cycle. FGF7 is downregulated at the end of anagen and not detected in DPCs during telogen or catagen; this downregulation may initiate catagen. FGF7 is also upregulated in graying/white hair; aging hair is an indication of hair growth. The addition of FGF10 is shown to increase the proliferation and migration potential of dermal papilla cells and also upregulates B-catenin. In mice lacking the FGF10 gene, PCNA expression (a marker for cell proliferation) was lowered and the number of proliferating cells was significantly decreased in the basal layer of the skin.^{33, 34, 35, 36, 37, 38}

HES1. Hes Family BHLH Transcription Factor 1 (HES1) regulates initiation of anagen and hair follicle regeneration. During telogen, HES1 is expressed at low levels compared to its increased expression in growing hair follicles (HES1 is upregulated during anagen). In mice lacking Hes1, hair follicles were found to be shorter than control mice; the transition from telogen to anagen in the knockout mice was also delayed.^{39, 40, 41}

HEY1. Hes Related Family BHLH Transcription Factor with YRPW Motif 1 (HEY1) is expressed during anagen (in anagen hair bulbs).^{42, 43}

HGF (Proliferation). Hepatocyte Growth Factor (HGF) regulates cell growth and motility in many cell types. It can regulate the interaction between epithelial keratinocytes and dermal papilla cells. The addition of exogenous HGF has been shown to increase hair growth in mice. HGF may also have an angiogenic effect on different types of cells involved in the formation of hair follicles. Addition of FGF2 also leads to the secretion of HGF, which promotes angiogenesis. HGF is shown to activate Wnt/B-catenin activity and induce human hair growth.^{44, 45, 46, 47}

IL1B (Inflammation/Immune Response). Interleukin 1, beta (IL1B) is a cytokine upregulated during inflammatory response. In wounds, IL1b may be activated by macrophages to induce hair regrowth in hair stem cells. It was previously thought that IL1b cytokines inhibit hair follicle regeneration and lead to hair loss. However, more recent studies have suggested that increased expression of IL1b may promote hair growth by regulating dermal papilla cells, especially in patients with alopecia.^{48, 49, 50}

IL6 (Inflammation/Immune Response). Interleukin 6 [IL6] is a cytokine that is increased in skin as a response to UV irradiation but may be regulated by the skin's microbiome. Interestingly, IL6 has shown both pro and anti-inflammatory activity. IL6 also increases expression with age and, when unregulated, can be involved in autoimmune diseases and cancers. IL6 is also upregulated in balding DPCs. IL6 inhibits elongation of the hair shaft and in mice, IL6 accelerates the transition from anagen to catagen. In IL6-knockout mice, wound closure is impaired and healing takes much longer than wild-type mice.^{51, 52, 53, 54, 55, 56}

KITLG/SCF (Growth Factor/Pigmentation). Kit ligand (KITLG), also known as stem cell factor (SCF), binds to the c-Kit receptor. KITLG regulates hair pigmentation and the differentiation, survival, and migration of mast cells. KITLG is expressed in keratinocytes, fibroblasts and immune cells of the skin. It is induced by UV; it acts as a paracrine factor that regulates the expression of MITF to promote melanin synthesis. KITLG plays an essential role in inducing follicular melanogenesis (hair pigmentation). SCF downregulation causes a dramatic reduction in melanoblast numbers as well as severe defects in hair pigmentation.^{57, 58, 59, 60, 61, 62}

LEP (Wound Healing). Leptin [LEP] is a hormone which exhibits various physiological properties; shown to promote wound healing but also increases inflammation in conditions such as psoriasis. Defining its place in hair follicle cycling, LEP immunoreactivity has been detected during catagen, telogen, and early anagen, in dermal papilla cells. It is suggested that LEP activates anagen to promote hair growth and stimulates anagen conversion in hair follicles at rest.^{63, 64, 65, 66, 67}

MKI67. Marker of Proliferation Ki-67 (MKI67) is a known marker of hair follicle proliferation and morphogenesis. MKI67 is only expressed in hDPCs during active growth phases of the cell cycle (G1, S, G2, and mitosis). Ki-67 (protein encoded by MKI67) is downregulated via transcriptional control by tumor suppressor gene p53; the cell cycle is blocked through retinoblastoma protein (RB) and the p53-p21-DREAM pathway.^{68, 69, 70}

NOG. Noggin (NOG) is an antagonistic inhibitor of BMP proteins, and its expression is upregulated BMP proteins in the DP cells. Negative feedback loop between BMPs and the regulator NOG in DPCs, indicating BMP signaling is tightly regulated by its negative regulator. Acts as a short-range BMP inhibitory signal that promotes placode boundary formation when overexpressed placode formation lasts longer and is disproportional in size. Serves as a marker for hair follicle-inductive features in DP cells. The Noggin-Shh signaling loop facilitates hair follicle regeneration. In noggin-knockout mice, hair follicle morphogenesis is arrested.^{71, 72, 73, 74}

NOTCH1. Notch Receptor 1 (NOTCH1) is involved in the late stages of hair follicle development and its reduction causes the sustained activation of Gli1 and enhances β-catenin activity. Important for hair homeostasis and differentiation of the hair follicle matrix cells into the hair shaft cells. Notch1 deletion inhibits the maturation of hair follicles from the late embryonic stage. Inhibition of Notch signaling suppresses epidermal growth factor-induced DP cell proliferation; Notch1 is important for hair follicle anagen. Notch pathway is required for expression of follicle regulatory genes Survivin and Msx2 in DPC. In Notch1-deficient mice, premature catagen occurs as well as the gradual loss of hair follicles.^{75, 76, 77, 78}

PTCH1. Protein Patched Homolog 1 (PTCH1) is a 12-transmembrane protein and an important regulator of the Sonic Hedgehog signaling pathway. Hedgehog signaling pathways are involved in many biological processes including hair follicle development. Binding of the glycoprotein sonic hedgehog (SHH) to its receptor Patched inactivates Patched and prevents its inhibition of smoothened (SMO). Since PTCH1 acts as an inhibitor of SMO (Smoothened), downstream GLI target genes (e.g., GLI1) are suppressed. When SMO is no longer inhibited, GLI target genes downstream genes are activated. GLI1 (also known as glioma-associated oncogene homolog 1) functions as a transcriptional activator. When Gli is activated, it translocates to the nucleus, where it activates target genes. Its upregulation results in DPC proliferation. PTCH1 functions as a sort of tumor suppressor by regulating cell cycle progression.^{79, 80}

SFRP1. Secreted Frizzled Related Protein 1 (SFRP1) expression is significantly increased in hair follicles and DPCs of androgenetic alopecia (AGA) subjects compared to normal hair subjects. Exogenous SFRP1 treatment of DPCs causes G1 cell cycle arrest. Conversely, SFRP1 inhibition leads to enhanced hair shaft production and inhibited spontaneous catagen, promoting hair growth.^{81, 82, 83}

SOX2. Sex Determining Region Y-Box 2 (SOX2), is a transcription factor important in progenitor cells and stem cells. SOX2 has been identified to have high expression in dermal papilla cells while not being expressed in hair follicle stem cells. Hair growth could be affected due to the transcriptional control of SOX2 on dermal papilla cells. SOX2 does act as a direct transcriptional regulator in dermal papilla cells in the early growth and development of the hair follicles. SOX2 also has an effect on BMP signal regulation in the HF. Specifically, when SOX2 is downregulated, both BMP6 and WIF1 are upregulated; these proteins inhibit hair growth.^{84, 85, 86, 87}

SRD5A2. 5a-reductase type a2 (SRD5A2) converts Testosterone into dihydrotestosterone (DHT). Inhibition of SRD5A2 inhibits the conversion of testosterone to DHT, inducing hair growth and slowing the progression of hair loss. Inhibition or down-regulation of SRD5A2 in DPC is effective in the prevention of AGA (androgenetic alopecia) and plays a major role in AGA development. Expression may influence hair growth. SRD5A2 variant is associated with decreased risk of baldness. Higher expression leads to an increase in DHT production (from testosterone) and can result in the inhibition of β-catenin activity, which is important for HF integrity, regeneration, and general physiology.^{88, 89, 90, 91}

STAT (Signal transduction/Transcription activation). Members of the signal transducer and activator of transcription (STAT) protein family are intracellular transcription factors that mediate many aspects of cellular immunity, proliferation, apoptosis and differentiation. They are primarily activated by membrane receptor-associated Janus kinases (JAK). The topical application of JAK inhibitors has been observed to trigger hair telogen-to-anagen transition, suggesting the importance of JAK/STAT signaling in the development of HF stem cells (HFSCs).^{92, 93, 94, 95}

STAT5A. Signal transducer and activator of transcription 5A (STAT5A) and 5B (STAT5B) are upregulated in DPCs. STAT5 activation is crucial for the transition from telogen to anagen in the hair growth cycle. Deletion of STAT5 in DPCs delays the hair follicle's entry into the anagen phase.⁹⁶

Stat5 was identified as a prolactin-induced mammary gland transcription factor. It was shown that two Stat5 genes encode proteins that are approximately 95% identical in amino acid sequence and that these two genes colocalized to murine chromosome 11, tightly linked to Stat3. Premature hair follicle stem cell (HFSC) activation can be spurred by genetic deletion of STAT5. STAT5 phosphorylation in DP cells can be increased by CXCR4 activation by CXCL12.^{97, 98}

STAT6. Signal Transducer and Activator of Transcription 6 (STAT6) plays an important role in IL4-mediated signaling. In a study investigating the effects of 3-Deoxysappanchalcone (3-DSC) on hair growth, it was found that 3-DSC promoted DPC proliferation via inhibition of STAT6. Normally, STAT6 is activated in quiescent hair follicles (hair follicles undergoing telogen). Other studies have found that plant extracts increase hair growth and promote DPC proliferation.^{99, 100}

TCF4. Transcription Factor 4 (TCF4) in conjunction with β-catenin form a complex within the nucleus resulting in transcriptional activation of downstream genes of the Wnt/β-catenin signaling pathway, a pathway significant in hair physiology. TCF4 is known to be a positive regulator in the maintenance of biological features of DPCs as well as upregulated in DPCs. In addition to its purpose in the biological functions of DPCs, evidence supports a close relation to DPC proliferation as well. When TCF4 is downregulated, hair follicles are phenotypically normal but exist in a prolonged telogen state.^{101, 102, 103, 104, 105}

TGFB1 (Cell Renewal/Regeneration/Wound Healing). Transforming growth factor beta 1 (TGFB1) is a multifunctional cytokine involved in tissue repair and remodeling, as well as cell cycle and differentiation. TGFB1 expression is able to regulate extracellular matrix homeostasis, partially through affecting the balance between MMPs and their inhibitors, the TIMPs. TGFB1 is involved in each stage of wound repair, including inflammation, proliferation, and extracellular matrix remodeling. Once secreted, TGF-β can remain in a latent state for some time, allowing for stock build-up in the extracellular matrix (ECM). In the skin, TGF-β isoforms are differentially expressed by nearly all classes of its constituent cells. For the case of healthy human epidermis, some TGF-β1 but mostly TGF-β3 expression has been described at the basal cellular layer. Increased TGF-β signaling can result in diseases like psoriasis, and on the contrary, reduced TGF-β levels, is believed to be a cause for chronic wounds. In hair, TGFB1 upregulation prevents the hair follicle from transitioning from telogen into anagen and leads to hair loss seen in androgenetic alopecia (AGA). When TGFB1 is downregulated in mice skin, keratinocytes are able to proliferate and results in the prolongation of anagen (hair growth).^{106, 107, 108, 109, 110, 111, 112, 113, 114, 115, 116}

TGFB2. Transforming Growth Factor Beta 2 (TGFB2), synthesis is prompted by dihydrotestosterone (DHT) in DPC at initiation. TGFB2 is suspected to suppress hair elongation; it induces premature catagen of hair follicles in adult hair cycling. It is produced mainly in the dermal papilla region and stimulates the proliferation of hair follicle stem cells (HFSCs). TGFB2 mRNA and protein expression is downregulated in senescent (aging) hDPCs. TGFB2 is involved in the aggregative ability and anti-aging properties of hDPCs through the regulation of collagen genes COL13A1 and COL15A1 expression. Upregulation of these collagen genes increases the spheroidal formation of DPCs and overall hair inductivity. TGFB2 is downregulated in senescent DPCs (aging cells).^{117, 118, 119}

VCAN (ECM Integrity). Versican (VCAN) is an aggregating proteoglycan, an extracellular matrix component involved with the elastic fiber system of the skin. VCAN interacts with hyaluronan, tenascin and fibulin molecules. Versican is expressed in fibroblasts, and in keratinocytes only in the zone of proliferation; VCAN levels decrease with confluence in cultured keratinocytes. High levels of Versican are seen as modulators of cell proliferation and migration: VCAN binds to cell adhesion molecules, lessening adhesion and promoting migration. VCAN is a hyalectan, which can interact with Hyaluronic Acid and other matrix components and cell surface receptors creating large complexes that retain large amounts of water—this creates a viscous extracellular matrix. Cells lacking Versican have been found to be unable to induce the formation of hair follicles; low expression of Versican is seen in androgenetic alopecia.^{120, 121, 122, 123, 124, 125, 126, 127}

### Wnt—Development and Regeneration Control

Wnt genes encode short-range secreted signaling molecules that regulate cell fate, adhesion, shape, proliferation, differentiation and movement, and are required for the development of multiple organ systems. WNT proteins can be grouped into two functional classes. Class I WNTs act through a ‘canonical’ signaling pathway that requires Disheveled (DVL) protein and causes stabilization of cytoplasmic β-catenin and its translocation to the nucleus, where it forms transcriptional complexes with members of the lymphoid enhancer factor/T cell factor (LEF/TCF) family of DNA binding factors to control the expression of WNT target genes. Class II WNTs operate via less well-characterized pathways that mediate proliferation, cell polarity and cell movements in gastrulation.

Broad dermal Wnt signaling is required for patterned induction of hair follicle placodes and subsequent Wnt signaling in placode stem cells is essential for induction of dermal condensates, cell clusters of precursors for the hair follicle dermal papilla (DP) WNT/β-catenin is essential for proliferation and differentiation of hair shaft, and WNT/β-catenin signaling by DP cells is required to maintain hair inductivity. formation of new DP can be induced in adult skin by activating the Wnt pathway in the epidermis.

Activation of the canonical Wnt/β-catenin signaling in DPC induces hair growth.

Wnt5a Differentiation/Aging. Mesenchymal Wnt5a mediates epithelial Notch/CSL signaling to control keratinocyte differentiation in the hair follicle. Wnt5a is largely associated with maintenance of stem cell fate and inhibits hair follicle differentiation (inhibits the transition from telogen to anagen). The expression of Wnt5a fades in the dermal papilla of postnatal follicles during anagen and intensifies in subsets of cells of the outer root sheath and outer layers of the inner root sheath. Aged HFSCs (Human Fibroblast Stem Cells) present with a decrease in canonical Wnt signaling and a shift towards non-canonical Wnt5a driven signaling which antagonizes canonical Wnt signaling. Aged HFSCs show elevated levels of expression of Wnt5a.

When Wnt5a is downregulated, no phenotypic change is observed in DPCs and this is likely due to redundant functions to other Wnt ligands.

### 12.2 Experimental Procedure

Test Materials: The following TMs were included in the study:
1. TM1—1.5 billion native (not heat shocked) aloe-derived exosomes;
2. TM2—1.5 billion engineered (heat shocked) aloe-derived exosomes (low dose);
3. TM3—5 billion engineered (heat shocked) aloe-derived exosomes (high dose); and
4. TM4—5 billion engineered (heat shocked) human adipose stromal stem cell (ASC)-derived exosomes.

All TMs were stored at −80° C. and protected from light until use.

Test Material and Media Preparation: Each TM was provided in a ready to use PBS solution. The maintenance media was prepared using BenchStable™ RPMI 1640 culture medium (Gibco®), supplemented with L-glutamine (Gibco®), and Penicillin-Streptomycin (Gibco®). Once prepared, the maintenance media was stored at 4° C. and protected from light until use.

Cell Culture: The human follicle dermal papilla cells (HFDPCs) used in this study were sourced from the temple skin of a 50 year old Caucasian female (PromoCell™, Cat #C-12071). A cryopreserved cell culture inventory grown from a primary cell stock was maintained. Working cell stocks (WCS) were prepared and frozen after 4 passages. All experiments in this study were performed using cells that had undergone ≤1 additional passage form the thaw of the WCS vial.

Culture Seeding: Four days before treatment, HFDPCs were seeded into 6-well plates at 7,000 cells per cm². After a cell attachment time of 72 hours, the media was replaced with fresh HFDPC growth media and the cells were returned to the incubator at 37° C. with 5% CO₂ and about 95% relative humidity for 24 hours.

Cell Culture Treatment: Following overnight incubation, cells were observed under a microscope to visually confirm confluency. The media was then removed, and each well was rinsed twice with PBS before proceeding with the test groups (n=4), as outlined below:
Test Materials—1 mL of each TM was added to the corresponding test group well;
Vehicle Control (VEH)—1 mL of PBS was added to the Vehicle Control wells;
Positive Control—2.5 mLs of maintenance media supplemented with 4% FBS (Gibco®) was added to the corresponding positive control wells; and
Untreated Group—2.5 mLs of maintenance media was added to the corresponding untreated control wells.

The plates were returned to the incubator at 37° C. with 5% CO₂ and about 95% relative humidity. During the first hour of incubation, the TM and VEH plates were gently rocked every 15 minutes.

After 1 hour of exposure to the TMs, 1.5 mL of fresh maintenance media was added to each of the test group and vehicle control wells and returned to the incubator at 37° C. with 5% CO₂ and about 95% relative humidity for 24 hours.

After 24 hours, cells were observed under a microscope and collected in RNAlater™. The media was collected for the LDH assay. For the LDH positive control group, two wells were treated with 1% Triton X-100™ (100 L per 6-well) added to each culture well.

LDH Cytotoxicity Assay: Each sample of used culture media was diluted 1:10 with sterile phosphate-buffered saline (PBS). A background control (diluted culture media that was not used for cell culture), a “low control” (diluted treatment media collected from the untreated culture wells), and a “high control” (diluted culture media collected from the 1% Triton-X 100™ treated culture wells) were included in the assay. Each diluted sample was added to an optically clear, flat-bottom 96-well plate in duplicate.

The LDH reaction mixture (Roche®) was prepared and added to each aliquot of diluted media (1:1). The reaction plate was incubated for about 20 minutes at room temperature, protected from light. Stopping solution (1.0N HCl) was the added to each well and absorbance was measured at 492 nm with a reference filter at 620 nm. Each sample absorbance value was calculated as the mean OD492-OD620 value for the duplicate reaction wells, with the blank absorbance value subtracted. The % cytotoxicity was then calculated relative to the Untreated Control (negative control, set to 0% cytotoxicity) and the Triton X-100™ treated control (positive control, set to 100% cytotoxicity) absorbance values, according to kit instructions:

% Cytotoxicity=[(Test Media Value−Low Control)/(High Control−Low Control)]*100.

RNA Isolation: Total RNA isolation was performed using a Qiagen® RNeasy® Kit following manufacturer's instructions for cultured mammalian cells. RNA concentration and purity were determined using a Nanodrop™ 2000 spectrophotometer (Table 10).

A260/280 readings indicate sample purity with ideal measurements that range from 1.8-2.1. The A260/230 ratio is an additional measure of sample purity. All samples showed high-quality A260/280 metrics. Four samples showed 260/230 metrics below 1.0, which did not affect downstream qPCR processing (see, Table 10).

TABLE 10
RNA Sample Quantity and Quality

| | Treatment | RNA Isolation | | |
|---|---|---|---|---|
| Sample (n) | Group | ng/μL RNA | 260/280 | 260/230 |
| 1 | Untreated | 102.0 | 2.07 | 2.11 |
| 2 | | 111.8 | 2.07 | 2.04 |
| 3 | | 121.1 | 2.05 | 2.05 |
| 4 | | 129.4 | 2.05 | 2.15 |
| 1 | Positive Control | 180.7 | 2.07 | 2.12 |
| 2 | (4% FBS Media) | 184.5 | 2.07 | 2.11 |
| 3 | | 193.1 | 2.07 | 1.63 |
| 4 | | 217.2 | 2.07 | 2.04 |
| 1 | Vehicle Control | 104.2 | 2.07 | 2.11 |
| 2 | (PBS) | 107.2 | 2.06 | 20.6 |
| 3 | | 96.6 | 2.08 | 1.76 |
| 4 | | 126.4 | 2.06 | 2.10 |
| 1 | TM1 | 91.3 | 2.06 | 1.95 |
| 2 | | 106.9 | 2.06 | 2.10 |
| 3 | | 103.7 | 2.06 | 1.85 |
| 4 | | 122.9 | 2.06 | 2.07 |
| 1 | TM2 | 43.6 | 2.05 | 1.16 |
| 2 | | 83.0 | 2.09 | 0.70 |
| 3 | | 89.3 | 2.06 | 2.00 |
| 4 | | 105.6 | 2.07 | 2.08 |
| 1 | TM3 | 81.5 | 2.06 | 0.93 |
| 2 | | 98.2 | 2.06 | 2.07 |
| 3 | | 121.5 | 2.06 | 2.16 |
| 4 | | 102.0 | 2.05 | 1.98 |
| 1 | TM4 | 78.6 | 2.05 | 1.81 |
| 2 | | 92.6 | 2.07 | 0.72 |
| 3 | | 94.2 | 2.05 | 2.05 |
| 4 | | 77.4 | 2.07 | 0.73 |

cDNA Synthesis: cDNA was generated from 400 ng of RNA using a Superscript™ Vilo RT Kit and a custom primer pool according to the manufacturer's instructions (ThermoFisher®). cDNA samples were pre-amplified for 12 cycles and diluted 1:20 with 1× Tris-EDTA (TE) Buffer for qPCR processing.

qPCR Processing: qPCR reactions were run in an OpenArray™ format using validated Taqman® gene expression assays in a Life Technologies® QuantStudio™ 12K Flex instrument. Each gene was assayed in duplicate.

Data Analysis: qPCR data quality and statistical analysis were assessed and performed on the raw data files using ThermoFisher® Connect Software (Life Technologies®). Statistical analysis was performed using the relative quantitation (RQ) method. In the first step of an RQ analysis, the Cq value of the target gene is normalized to the Cq value of an endogenous control gene to generate the delta Cq (dCq). dCq values are calculated in order to normalize for variability between the samples that may occur during the experimental procedures.

Statistical Data Analysis Using ThermoFisher® Connect Software: Unpaired t-tests were carried out using ThermoFisher® Connect Software. The statistical comparison generated delta delta Cq (ddCq) values (the mean dCq of the treated group−the mean Cq of the control group). The statistical software converts the ddCq values into log and linear RQ values for export [RQ=2^{-ddCq}]. The linear RQ values were converted to linear fold-change values to simplify data interpretation; linear fold-change data was calculated from exported linear RQ values using Microsoft Excel®:
For RQ values≥1.0: Linear fold-change value=RQ value
For RQ values<1.0: Linear fold-change value=−1/RQ value.

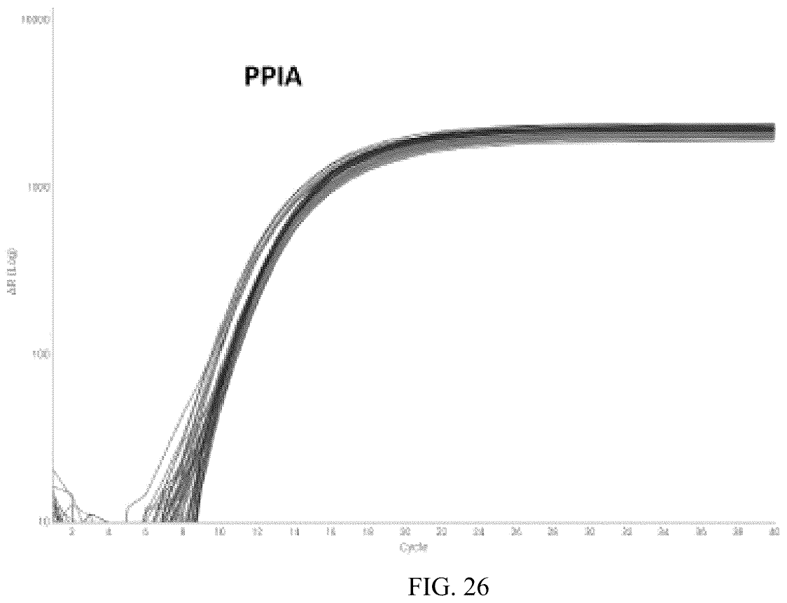

Endogenous Control Gene Selection: It is important to select an endogenous control gene that is consistently expressed in all of the samples of a comparison. Four candidate control genes (GUSB, HPRT1, PPIA, and UBC) were analyzed. The most consistent endogenous control gene was chosen based on the stability score and ranges generated by the ThermoFisher® Data Connect RQ software. Lower stability scores for each endogenous control gene are shown in Table 11. Based on the stability and rage scores, PPIA was selected as the endogenous control gene. Statistics (unpaired t-tests) were carried out for each comparison using dCq values normalized to PPIA. qPCR amplification curves for PPIA are shown in FIG. 26.

TABLE 11
Stability Scores for 4 Endogenous Control Genes

| | | Stability Score |
|---|---|---|
| Gene ID | Gene Name | (OpenArray) |
| GUSB | Glucuronidase Beta | 0.362 |
| HPRT1 | Hypoxanthine | 0.399 |
| | Phopshoribosyltransferase 1 | |
| PPIA | Peptidylprolyl Isomerase A | 0.303 |
| UBC | Ubiquitin C | 0.310 |

qPCR Data Quality and Statistical Data Analysis: qPCR data quality was assessed using a combination of factors, including visual analysis of the shape of the qPCR curve and the Cq value. Cq values are an indication of the total amount of transcript present in the sample and can impact the quality of the qPCR data. qPCR amplification takes place over a total of 40 cycles and typically occurs before cycle 28. The relative amount of the gene transcript is associated with the Cq value of the PCR reaction. Cq values typically correspond with the following:
Cq values less than 28: associated with high transcript levels and robust, high quality PCR data;
Cq values greater than 28: lower level transcripts, less robust qPCR data; data to be reviewed cautiously.

### 12.3 Cytotoxicity (LDH Activity) Assay Results

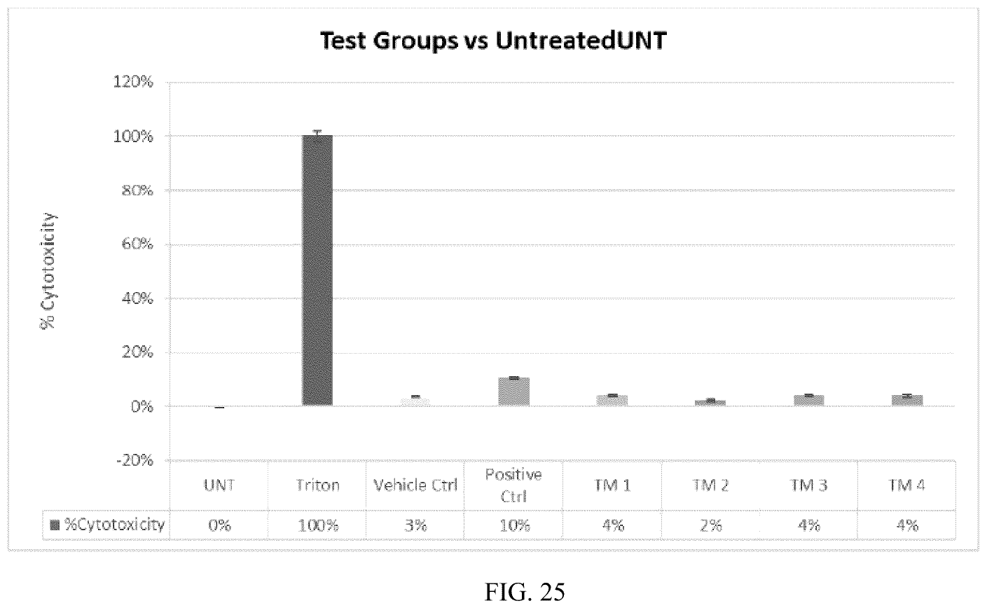

FIG. 25 shows a lactate dehydrogenase (LDH) cytotoxicity assessment that was performed using media collected from the wells of the cells analyzed for differential gene expression. When cells are damaged, their membranes become compromised, which allows LDH, which is normally contained within the cell, to leak out into the surrounding media. Increased LDH activity is an indicator of damaged or dead cells.

The following treatment groups were included in the study: HFDPCs treated with 1.5 billion native, not heat shocked, aloe-derived exosomes (TM1), 1.5 billion engineered, heat shocked, aloe-derived exosomes (TM2), 5 billion engineered, heat shocked, aloe-derived exosomes (TM3), and 5 billion engineered, heat shocked) human adipose stromal stem cell (ASC)-derived exosomes (TM4) compared to an untreated negative control (UNT), a media+serum positive control (Positive Ctrl), a Triton X-100® treated positive control (Triton), and a PBS treated for 1 hour vehicle control (Vehicle Ctrl).

HFDPCs treated with Triton X-100® was included in the analysis for reference. Triton X-100® is a nonionic detergent that permeates cells by disrupting the lipid bilayer of the cell membrane. This is achieved by the insertion of detergent molecules into the lipid membrane, creating pores and damaging the membrane's integrity. This allows molecules to pass through the cell membrane that normally could not pass through the lipid bilayer.

Results: low levels of cytotoxicity were observed for the treatment groups compared to the untreated control group and the Triton X-100@control group at 24 hours.

### 12.4 Gene Expression Results

Quantitative PCR (qPCR) is used to quantify gene expression by measuring the amount of a specific RNA transcript (mRNA) in a sample. RNA is isolated from a sample and reverse transcribed into cDNA. The cDNA serves as a template for the qPCR reaction. Fluorescent dyes or probes are added to the reaction mixture and upon amplification, the fluorescent signal increases proportionally to the amount of DNA produced. The fluorescence is measured in real time at each cycle of the PCR reaction and plotted in an amplification curve.

qPCR amplification curves typically have a sigmoidal shape with three distinct phases. Phase 1, usually representing the first 10-20 cycles of the PCR reaction, is characterized by a slow upward trend in the curve. Phase 2, usually representing cycles 10-35 of the PCR reaction, is characterized by a strong upward swing in the curve. Phase 3, usually representing cycles 25+ of the PCR reaction, is characterized by a plateau where the amplification signal tapers off and ceases to grow.

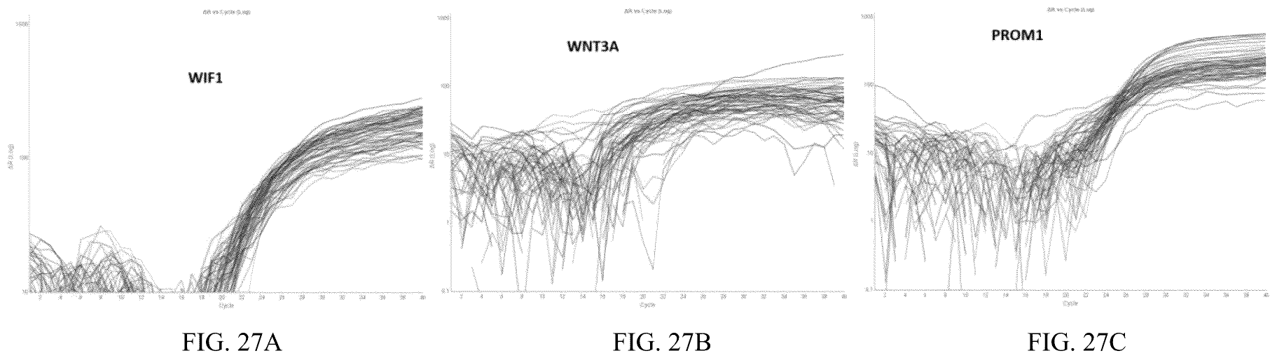

Low/Poor Quality qPCR Amplification: Three of the genes (WIF1, WNT3A, and PROM1) on the gene panel showed poor quality qPCR amplification. These genes were removed from the dataset prior to statistical analysis. The poor quality amplification curves are shown in FIG. 27A, FIG. 27B and FIG. 27C. These curves are characterized by their poor sigmoidal shape and inconsistent fluorescent signal, indicating poor amplification.

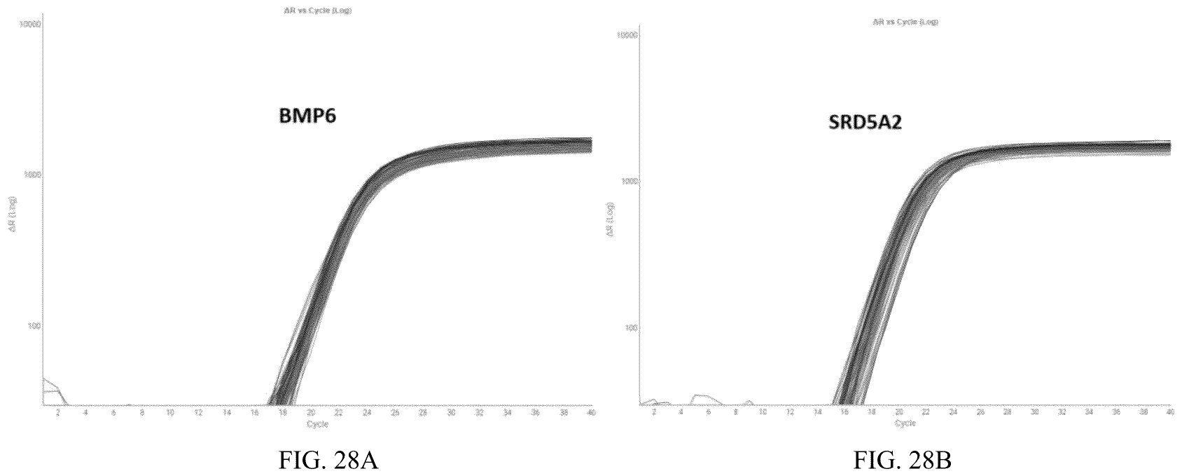

Early Cycle, High Quality Amplification: All of the other genes in the study showed high quality qPCR amplification curves. Exemplary high quality amplification curves are shown in FIG. 28A and FIG. 28B. These curves are characterized by their strong sigmoidal shape and consistent fluorescent signal, indicating strong amplification.

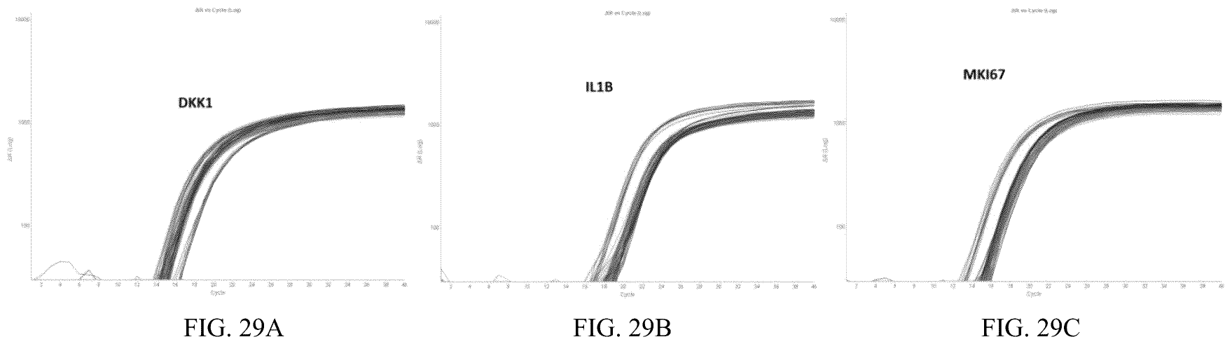

High Fold-Change Amplification: Some of the genes in the study showed high fold-change differences in amplification when compared to the Vehicle or Untreated Groups. Exemplary high fold-change amplification curves are shown in FIG. 29A, FIG. 29B and FIG. 29C. These curves are characterized by their strong sigmoidal shape and consistent fluorescent signal, indicating strong amplification, but they are further characterized by the differential fluorescent signal between samples, while maintaining the strong sigmoidal shape and strong amplification.

Statistics (unpaired t-test, p<0.05, n=4) were performed to compare the treatment groups to the Vehicle Control group at 24 hours following exposure to the TMs (Table 12), as well as comparing the Vehicle Control group to the Untreated Control group (Table 13).

The genes with linear fold change values (>1.5) are included in the tables, with fold change values >2.0 listed in bold text. Statistically insignificant results are listed as “n.s.”.

Genes are sorted alphabetically by Gene ID and percent change is provided as an additional way to view the data. Linear fold-change values of 1.5 or greater are considered biologically relevant.

TABLE 13
Fold-Change Values for Statistically Significant
Genes: Treatment Groups vs. Vehicle Control Group

| | | | | | | Desired |
|---|---|---|---|---|---|---|
| | | | | | | Expression |
| | Treatment Groups vs. Vehicle | | | | | Change to |
| | | | | | Positive | Promote |
| | TM1 | TM2 | TM3 | TM4 | Control | Hair |
| Gene ID | Linear FC | Linear FC | Linear FC | Linear FC | Linear FC | Growth |
| AR | n.s. | n.s. | n.s. | n.s. | −2.26 | Decrease |
| ASIP | n.s. | n.s. | n.s. | n.s. | −3.32 | Increase |
| AXIN2 | n.s. | n.s. | n.s. | n.s. | −1.95 | Increase |
| BCL2 | n.s. | n.s. | n.s. | n.s. | −2.87 | Increase |
| BMP2 | n.s. | n.s. | n.s. | n.s. | −6.06 | Decrease |
| BMP4 | n.s. | n.s. | n.s. | n.s. | −3.47 | Decrease |
| BMP6 | n.s. | −1.53 | n.s. | n.s. | −2.00 | Decrease |
| DCN | n.s. | n.s. | n.s. | n.s. | −2.36 | Increase |
| ESR1 | n.s. | n.s. | n.s. | n.s. | −1.80 | Increase |
| ESR2 | n.s. | n.s. | n.s. | n.s. | −2.31 | Increase |
| FGF10 | n.s. | n.s. | n.s. | n.s. | −2.23 | Increase |
| FGF7 | n.s. | n.s. | n.s | n.s. | −1.90 | Increase |
| HES1 | n.s. | n.s. | n.s. | n.s. | −2.14 | Increase |
| HEY1 | n.s. | n.s. | n.s. | −1.64 | n.s. | Increase |
| HGF | n.s. | n.s. | n.s. | n.s. | −3.25 | Increase |
| IL1B | n.s. | n.s. | n.s. | −1.52 | n.s. | Decrease |
| IL6 | 1.53 | n.s. | n.s. | n.s. | n.s. | Decrease |
| KITLG | n.s. | n.s. | n.s. | n.s. | −2.88 | Increase |
| LEP | n.s. | n.s. | n.s. | n.s. | 1.72 | Increase |
| MKI67 | n.s. | n.s. | n.s. | n.s. | 2.30 | Increase |
| NOG | n.s. | n.s. | n.s. | n.s. | 2.23 | Increase |
| NOTCH1 | n.s. | n.s. | n.s. | n.s. | −1.78 | Increase |
| PTCH1 | n.s. | n.s. | n.s. | n.s. | −1.68 | Increase |
| SRFP1 | n.s. | n.s. | n.s. | n.s. | −2.08 | Decrease |
| SOX2 | n.s. | n.s. | n.s. | n.s. | −2.59 | Increase |
| SRD5A2 | n.s. | n.s. | n.s. | n.s. | −1.83 | Decrease |
| STAT5A | n.s. | n.s. | n.s. | n.s. | −2.60 | Increase |
| STAT6 | n.s. | n.s. | n.s. | n.s. | −1.76 | Decrease |
| TCF4 | n.s. | n.s. | n.s. | n.s. | −2.55 | Increase |
| TGFB1 | n.s. | n.s. | n.s. | n.s. | −1.92 | Decrease |
| TGFB2 | n.s. | n.s. | n.s. | n.s. | −3.31 | Decrease |
| VCAN | n.s. | n.s. | n.s. | n.s. | −2.13 | Increase |
| WNT5A | n.s. | n.s. | n.s. | n.s. | −1.95 | Increase |

TABLE 13
Fold-Change Values for Statistically Significant Genes:
Vehicle Control vs. Untreated Control Group

| | Vehicle vs. | Desired Expression |
|---|---|---|
| | Untreated | Change to Promote |
| Gene ID | Linear FC | Hair Growth |
| ASIP | −1.77 | Increase |
| BMP2 | −1.34 | Decrease |
| BMP6 | 1.32 | Decrease |
| CCND1 | 1.30 | Increase |
| CDKN2A | −1.17 | Decrease |
| DCN | −1.51 | Increase |
| DKK1 | 2.55 | Decrease |
| ESR1 | −1.51 | Increase |
| FGF7 | 1.48 | Increase |
| GLI1 | −1.36 | Increase |
| HGF | −1.4 | Increase |
| IGF1 | −1.88 | Increase |
| IL6 | 7.60 | Decrease |
| LEP | −1.10 | Increase |
| NOG | 1.38 | Increase |
| NOTCH1 | −1.20 | Increase |
| PTCH1 | −1.23 | Increase |
| SRD5A2 | 1.90 | Decrease |
| VCAN | −1.30 | Increase |
| VEGFA | 1.13 | Increase |

### 12.5 Conclusions

Treatment groups TM1-TM4 showed little to no change in gene expression at 24 hours.

The positive control regulated most of the genes on the panel with high fold change values. The direction of the changes were mixed, with changes in a direction that were beneficial for hair growth and others that were not. Those that showed fold changes in a direction not beneficial for hair growth were likely due to the timepoint.

When comparing the vehicle control with the untreated control, there were several genes that showed statistically significant changes, including the increased expression of IL-6.

Gene expression is a dynamic process that can be influenced by the duration of exposure to the test material. Depending on the gene, changes in expression may be observed in as early as two hours or as late as several days.

### Example 13. Gene Expression Analysis of In Vitro Dermal Papillae Cells 72 Hours after Treatment with Test Materials

### 13.1. Project Summary

This study was conducted as a repeat analysis of the study described in Example 12 in order assess the impact of the test materials at a different time point (72 hours) and at a higher concentration.

HFDPCs are a group of mesenchymal-derived cells at the base of the hair follicle that regulates hair follicle growth through the expression and secretion of specific biomarkers.

Cells from a Caucasian female donor (age: 50) were used for this study.

Gene expression was assessed using the Genemarkers® Dermal Papilla Cell Panel (see, Table 8), a qPCR-based gene expression panel which contains 52 target genes and 4 endogenous control genes.

Gene expression was assessed 72 hours following the application of the TMs.

The following treatment groups were including in the study (n=4):
1. Untreated Control: normal maintenance media;
2. Positive Control: normal maintenance media supplemented with 4% fetal bovine serum (FBS);
3. Negative Control: 1:10 dilution of normal maintenance media into PBS;
4. Vehicle Control: PBS;
5. EXO1—5 billion engineered (heat shocked) aloe-derived exosomes;
6. EXO2—5 billion native (not heat shocked) aloe-derived exosomes;
7. EXO3—5 billion engineered (heat shocked) human adipose stromal stem cell (ASC)-derived exosomes; and
8. EXO4—5 billion native (not heat shocked) human ASC-derived exosomes.

### 13.2 Experimental Procedure

Test Materials: The following TMs were included in the study:
1. EXO1—5 billion engineered (heat shocked) aloe exosomes;
2. EXO2—5 billion native (not heat shocked) aloe-derived exosomes;
3. EXO3—5 billion engineered (heat shocked) human adipose stromal stem cell (ASC)-derived exosomes; and
4. EXO4—5 billion native (not heat shocked) human ASC-derived exosomes.

All TMs were stored at −80° C. and protected from light until use.

Test Material and Media Preparation: Each TM was provided in a ready to use PBS solution. The maintenance media was prepared using BenchStable™ RPMI 1640 culture medium (Gibco®), supplemented with L-glutamine (Gibco®), and Penicillin-Streptomycin (Gibco®). Once prepared, the maintenance media was stored at 4° C. and protected from light until use.

Cell Culture: The human follicle dermal papilla cells (HFDPCs) used in this study were sourced from the temple skin of a 50 year old Caucasian female (PromoCell®, Cat #C-12071). A cryopreserved cell culture inventory grown from a primary cell stock was maintained. Working cell stocks (WCS) were prepared and frozen after 4 passages. All experiments in this study were performed using cells that had undergone ≤1 additional passage form the thaw of the WCS vial.

Culture Seeding: Four days before treatment, HFDPCs were seeded into 12-well plates at 8,100 cells per cm². After a cell attachment time of 24 hours, the media was replaced with fresh HFDPC growth media and the cells were returned to the incubator at 37° C. with 5% CO₂ and about 95% relative humidity for 96 hours with the media replaced every other day.

Cell Culture Treatment: During cell culture, cells were observed daily under a microscope to visually confirm confluency until full confluency was reached. The media was then removed, and each well was rinsed twice with HBSS (PromoCell®) before proceeding with the test groups (n=4), as outlined below:
Test Materials—0.5 mL of each TM was added to the corresponding test group well;
Vehicle Control (VEH)—0.5 mL of PBS was added to the Vehicle Control wells;
Positive Control—0.5 mL of maintenance media supplemented with 4% FBS (Gibco®) was added to the corresponding positive control wells;
Negative Control—0.5 mL of maintenance media diluted 1:10 with PBS was added to the corresponding negative control wells; and
Untreated Group—0.5 mL of maintenance media was added to the corresponding untreated control wells.

The plates were returned to the incubator at 37° C. with 5% CO₂ and about 95% relative humidity and rocked on a plate shaker at 40 rpm/min for 1 hour.

After 1 hour of exposure to the TMs, 1 mL of fresh maintenance media was added to each of the test group, positive/negative control, and vehicle control wells and returned to the incubator at 37° C. with 5% CO₂ and about 95% relative humidity for 72 hours.

After 72 hours, cells were observed under a microscope and collected in RLT lysis buffer (Qiagen®). The media was collected for the LDH assay. For the LDH positive control group, two wells were treated with 1% Triton X-100™ (15 μL per 12-well) added to each culture well.

LDH Cytotoxicity Assay: Each sample of used culture media was diluted 1:10 with sterile phosphate-buffered saline (PBS). A background control (diluted culture media that was not used for cell culture), a “low control” (diluted treatment media collected from the untreated culture wells), and a “high control” (diluted culture media collected from the 1% Triton-X 100™ treated culture wells) were included in the assay. Each diluted sample was added to an optically clear, flat-bottom 96-well plate in duplicate.

The LDH reaction mixture (Roche®) was prepared and added to each aliquot of diluted media (1:1). The reaction plate was incubated for about 20 minutes at room temperature, protected from light. Stopping solution (1.0N HCl) was the added to each well and absorbance was measured at 492 nm with a reference filter at 620 nm. Each sample absorbance value was calculated as the mean OD492-OD620 value for the duplicate reaction wells, with the blank absorbance value subtracted. The % cytotoxicity was then calculated relative to the Untreated Control (negative control, set to 0% cytotoxicity) and the Triton X-100™ treated Control (positive control, set to 100% cytotoxicity) absorbance values, according to kit instructions:

% Cytotoxicity=[(Test Media Value−Low Control)/(High Control−Low Control)]*100.

RNA Isolation: Total RNA isolation was performed using a Qiagen® RNeasy® Kit following manufacturer's instructions for cultured mammalian cells. RNA concentration and purity were determined using a Nanodrop™ 2000 spectrophotometer (Table 14).

A260/280 readings indicate sample purity with ideal measurements that range from 1.8-2.1. The A260/230 ratio is an additional measure of sample purity. All samples showed high-quality A260/280 metrics. Two samples showed 260/230 metrics below 1.0, which did not affect downstream qPCR processing (see, Table 14).

TABLE 14
RNA Sample Quantity and Quality

| | Treatment | RNA Isolation | | |
|---|---|---|---|---|
| Sample (n) | Group | ng/μL RNA | 260/280 | 260/230 |
| 1 | Untreated | 22.9 | 2.01 | 1.51 |
| 2 | | 23.9 | 1.99 | 1.18 |
| 3 | | 26.7 | 2.03 | 1.56 |
| 4 | | 27.7 | 2.05 | 1.63 |
| 1 | Negative | 23.3 | 1.97 | 1.47 |
| 2 | Control (1:10 | 21.4 | 2.08 | 1.26 |
| 3 | dilution into | 26.0 | 2.04 | 1.33 |
| 4 | PBS) | 28.4 | 2.10 | 1.52 |
| 1 | Positive Control | 95.5 | 2.07 | 2.03 |
| 2 | (4% FBS Media) | 86.6 | 2.07 | 1.82 |
| 3 | | 74.6 | 2.06 | 1.56 |
| 4 | | 67.0 | 2.06 | 1.95 |
| 1 | Vehicle Control | 19.0 | 2.11 | 0.64 |
| 2 | (PBS) | 21.1 | 2.00 | 1.23 |
| 3 | | 26.1 | 2.04 | 1.09 |
| 4 | | 15.7 | 1.97 | 1.43 |
| 1 | EXO1 | 24.5 | 2.03 | 1.44 |
| 2 | | 28.6 | 2.04 | 1.31 |
| 3 | | 31.8 | 2.08 | 1.51 |
| 4 | | 25.6 | 2.15 | 1.69 |
| 1 | EXO2 | 28.4 | 2.03 | 1.81 |
| 2 | | 26.1 | 2.00 | 1.54 |
| 3 | | 28.2 | 2.04 | 1.17 |
| 4 | | 28.3 | 2.06 | 1.80 |
| 1 | EXO3 | 21.3 | 2.00 | 1.40 |
| 2 | | 23.8 | 2.02 | 0.62 |
| 3 | | 23.8 | 2.01 | 1.69 |
| 4 | | 17.1 | 2.05 | 1.34 |
| 1 | EXO4 | 19.8 | 2.01 | 1.68 |
| 2 | | 27.8 | 2.05 | 1.51 |
| 3 | | 23.8 | 2.04 | 1.58 |
| 4 | | 15.6 | 2.07 | 1.00 |

cDNA Synthesis: cDNA was generated from 150 ng of RNA using a Superscript™ Vilo RT Kit and a custom primer pool according to the manufacturer's instructions (ThermoFisher®). cDNA samples were pre-amplified for 12 cycles and diluted 1:20 with 1× Tris-EDTA (TE) Buffer for qPCR processing.

qPCR Processing: qPCR reactions were run in an OpenArray™ format using validated Taqman® gene expression assays in a Life Technologies® QuantStudio™ 12K Flex instrument. Each gene was assayed in duplicate.

Data Analysis: qPCR data quality and statistical analysis was assessed and performed on the raw data files using ThermoFisher® Connect Software (Life Technologies®). Statistical analysis was performed using the relative quantitation (RQ) method. In the first step of an RQ analysis, the Cq value of the target gene is normalized to the Cq value of an endogenous control gene to generate the delta Cq (dCq). dCq values are calculated in order to normalize for variability between the samples that may occur during the experimental procedures.

Statistical Data Analysis Using ThermoFisher® Connect Software: Unpaired t-tests were carried out using ThermoFisher® Connect Software. The statistical comparison generated delta delta Cq (ddCq) values (the mean dCq of the treated group−the mean Cq of the control group). The statistical software converts the ddCq values into log and linear RQ values for export [RQ=2^{-ddCq}]. The linear RQ values were converted to linear fold-change values to simplify data interpretation; linear fold-change data was calculated from exported linear RQ values using Microsoft Excel®:
For RQ values≥1.0: Linear fold-change value=RQ value
For RQ values<1.0: Linear fold-change value=−1/RQ value.

Endogenous Control Gene Selection: It is important to select an endogenous control gene that is consistently expressed in all of the samples of a comparison. Four candidate control genes (GUSB, HPRT1, PPIA, and UBC) were analyzed. The most consistent endogenous control gene was chosen based on the stability score and ranges generated by the ThermoFisher® Data Connect RQ software. Lower stability scores for each endogenous control gene are shown in Table 15. Based on the stability and rage scores, PPIA was selected as the endogenous control gene. Statistics (unpaired t-tests) were carried out for each comparison using dCq values normalized to PPIA. qPCR amplification curves for PPIA are shown in FIG. 31.

TABLE 15
Stability Scores for 4 Endogenous Control Genes

| | | Stability Score |
|---|---|---|
| Gene ID | Gene Name | (OpenArray) |
| GUSB | Glucuronidase Beta | 0.355 |
| HPRT1 | Hypoxanthine Phosphoribosyltransferase 1 | 0.246 |
| PPIA | Peptidylprolyl Isomerase A | 0.304 |
| UBC | Ubiquitin C | 0.251 |

qPCR Data Quality and Statistical Data Analysis: qPCR data quality was assessed using a combination of factors, including visual analysis of the shape of the qPCR curve and the Cq value. Cq values are an indication of the total amount of transcript present in the sample and can impact the quality of the qPCR data. qPCR amplification takes place over a total of 40 cycles, and typically occurs before cycle 28. The relative amount of the gene transcript is associated with the Cq value of the PCR reaction. Cq values typically correspond with the following:
Cq values less than 28: associated with high transcript levels and robust, high quality PCR data;
Cq values greater than 28: lower level transcripts, less robust qPCR data; data to be reviewed cautiously.

### 13.3 Cytotoxicity (LDH Activity) Assay Results

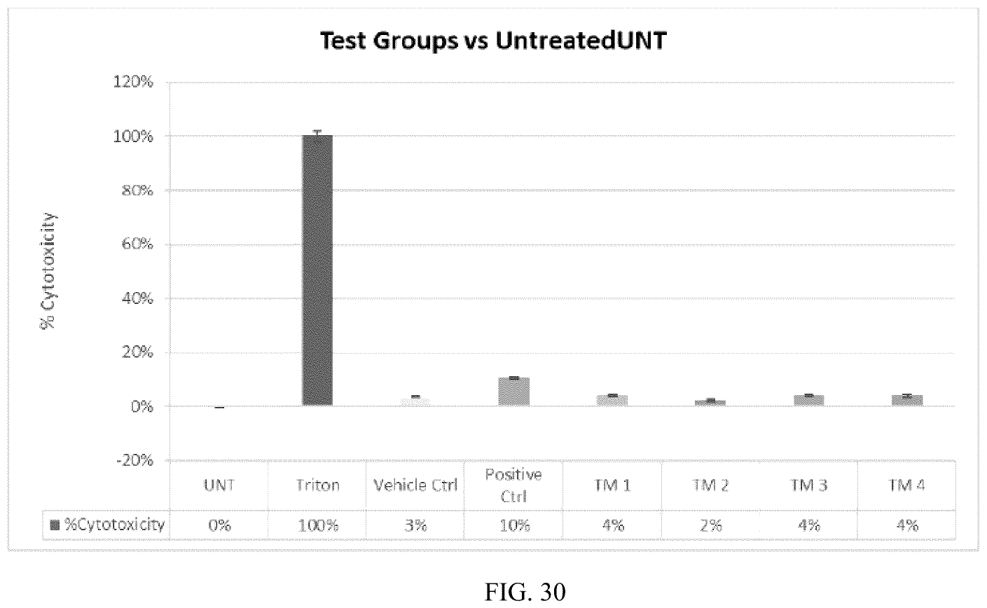

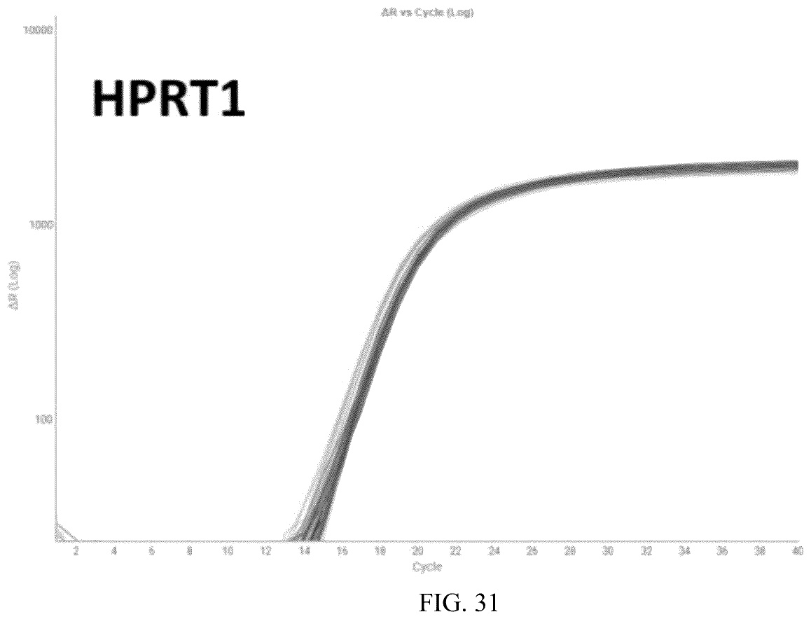

FIG. 30 shows a lactate dehydrogenase (LDH) cytotoxicity assessment that was performed using media collected from the wells of the cells analyzed for differential gene expression. When cells are damaged, their membranes become compromised, which allows LDH, which is normally contained within the cell, to leak out into the surrounding media. Increased LDH activity is an indicator of damaged or dead cells.

The following treatment groups were included in the study: HFDPCs treated with 5 billion engineered (heat shocked) aloe exosomes (TM 1), 5 billion native (not heat shocked) aloe-derived exosomes (TM 2), 5 billion engineered (heat shocked) human adipose stromal stem cell (ASC)-derived exosomes (TM 3), and 5 billion native (not heat shocked) human ASC-derived exosomes (TM 4), compared to an untreated control (UNT), a media+serum positive control (Positive Ctrl), a Triton X-100® treated positive control (Triton), and a PBS treated vehicle control (Vehicle Ctrl).

HFDPCs treated with Triton X-100® was included in the analysis for reference. Triton X-100® is a nonionic detergent that permeates cells by disrupting the lipid bilayer of the cell membrane. This is achieved by the insertion of detergent molecules into the lipid membrane, creating pores and damaging the membrane's integrity. This allows molecules to pass through the cell membrane that normally could not pass through the lipid bilayer.

Results: low levels of cytotoxicity were observed for the treatment groups compared to the untreated control group and the Triton X-100@control group at 72 hours.

### 13.4 Gene Expression Results

Quantitative PCR (qPCR) is used to quantify gene expression by measuring the amount of a specific RNA transcript (mRNA) in a sample. RNA is isolated from a sample and reverse transcribed into cDNA. The cDNA serves as a template for the qPCR reaction. Fluorescent dyes or probes are added to the reaction mixture and upon amplification, the fluorescent signal increases proportionally to the amount of DNA produced. The fluorescence is measured in real time at each cycle of the PCR reaction and plotted in an amplification curve.

qPCR amplification curves typically have a sigmoidal shape with three distinct phases. Phase 1, usually representing the first 10-20 cycles of the PCR reaction, is characterized by a slow upward trend in the curve. Phase 2, usually representing cycles 10-35 of the PCR reaction, is characterized by a strong upward swing in the curve. Phase 3, usually representing cycles 25+ of the PCR reaction, is characterized by a plateau where the amplification signal tapers off and ceases to grow.

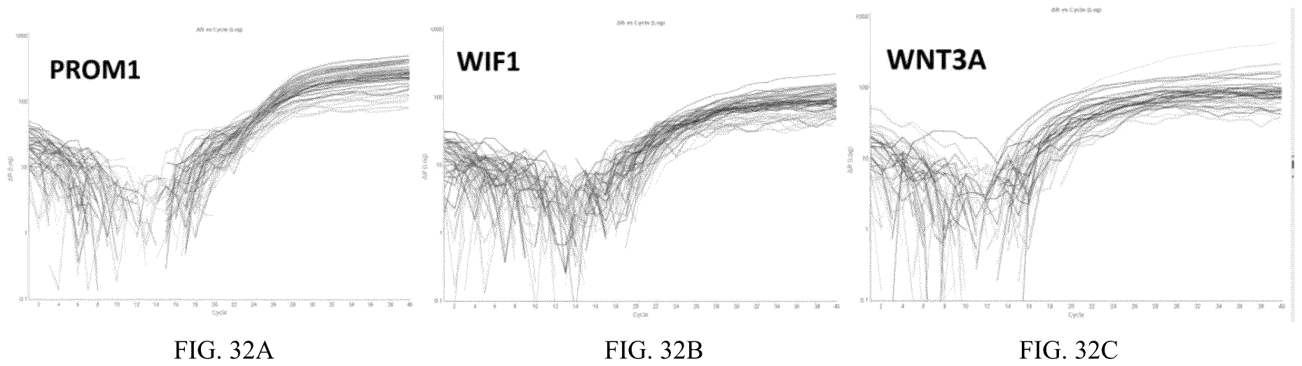

Low/Poor Quality qPCR Amplification: Three of the genes (WIF1, WNT3A, and PROM1) on the gene panel showed poor quality qPCR amplification. These genes were removed from the dataset prior to statistical analysis. The poor quality amplification curves are shown in FIGS. 32A-32C. These curves are characterized by their poor sigmoidal shape and inconsistent fluorescent signal, indicating poor amplification.

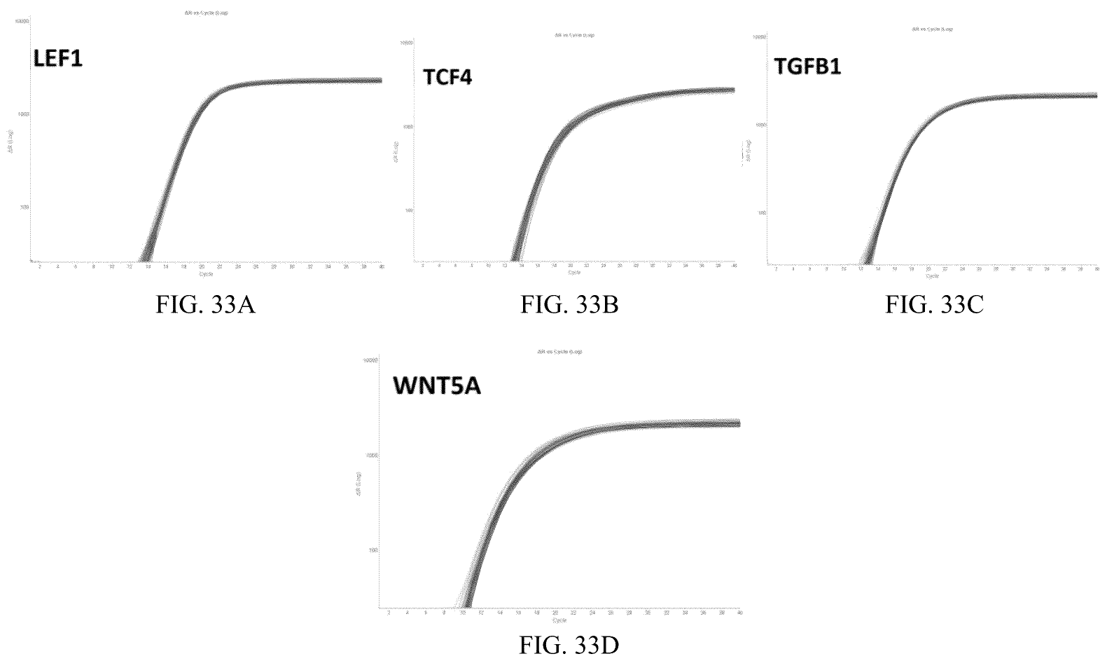

Early Cycle, High Quality Amplification: All of the other genes in the study showed high quality qPCR amplification curves. Exemplary high quality amplification curves are shown in FIGS. 33A-33D. These curves are characterized by their strong sigmoidal shape and consistent fluorescent signal, indicating strong amplification.

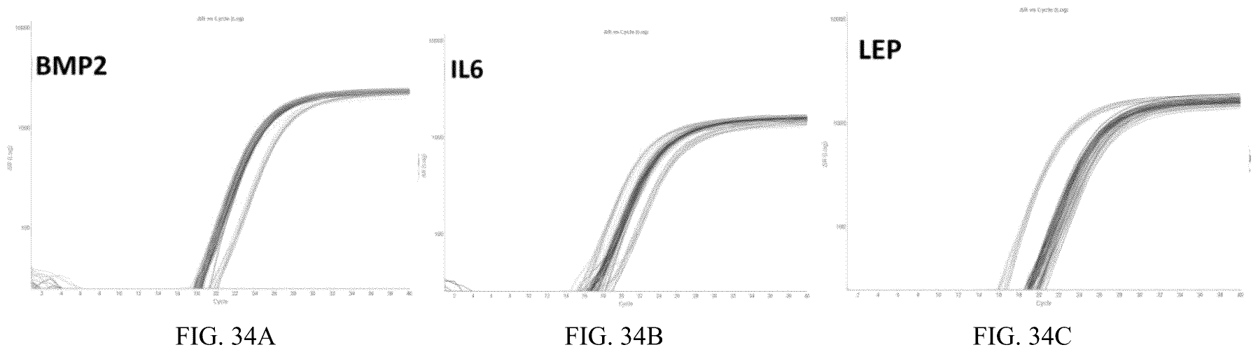

High Fold-Change Amplification: Some of the genes in the study showed high fold-change differences in amplification when compared to the Vehicle or Untreated Groups. Exemplary high fold-change amplification curves are shown in FIG. 34A, FIG. 34B, and FIG. 34C. These curves are characterized by their strong sigmoidal shape and consistent fluorescent signal, indicating strong amplification, but they are further characterized by the differential fluorescent signal between samples, while maintaining the strong sigmoidal shape and strong amplification.

Statistics (unpaired t-test, p<0.05, n=4) were performed to compare the treatment groups to the Vehicle Control group at 24 hours following exposure to the TMs (Table 16), as well as comparing the Vehicle Control group to the Untreated Control group (Table 17).

The genes with linear fold change values (>1.5) are included in the tables, with fold change values >2.0 listed in bold text. Statistically insignificant results are listed as “n.s.”.

Genes are sorted alphabetically by Gene ID and percent change is provided as an additional way to view the data. Linear fold-change values of 1.5 or greater are considered biologically relevant.

TABLE 16
Fold-Change Values for Statistically Significant
Genes: Treatment Groups vs. Vehicle Control Group

| | | | | | | | Desired |
|---|---|---|---|---|---|---|---|
| | Treatment Groups vs. Vehicle | | | | | | Expression |
| | Negative | | | | | Positive | Change to |
| | Control | EXO1 | EXO2 | EXO3 | EXO4 | Control | Promote |
| | Linear | Linear | Linear | Linear | Linear | Linear | Hair |
| Gene ID | FC | FC | FC | FC | FC | FC | Growth |
| AR | n.s. | n.s. | n.s. | n.s. | n.s. | −2.73 | Decrease |
| ASIP | n.s. | n.s. | n.s. | n.s. | n.s. | −2.45 | Increase |
| AXIN2 | n.s. | n.s. | n.s. | n.s. | n.s. | −2.95 | Increase |
| BAX | n.s. | n.s. | n.s. | n.s. | n.s. | −1.89 | Decrease |
| BCL2 | n.s. | n.s. | n.s. | n.s. | n.s. | −3.7 | Increase |
| BMP2 | n.s. | n.s. | n.s. | n.s. | n.s. | −7.63 | Decrease |
| BMP4 | n.s. | n.s. | n.s. | n.s. | −1.5 | −8.26 | Decrease |
| BMP6 | n.s. | n.s. | n.s. | n.s. | n.s. | −1.79 | Decrease |
| CDKN2A | n.s. | n.s. | n.s. | n.s. | n.s. | −1.57 | Decrease |
| CORIN | n.s. | 1.85 | 1.74 | n.s. | n.s. | 4.19 | Increase |
| DCN | n.s. | n.s. | n.s. | n.s. | n.s. | −2.56 | Increase |
| ESR1 | n.s. | n.s. | n.s. | n.s. | n.s. | −2.72 | Increase |
| ESR2 | n.s. | n.s. | n.s. | n.s. | n.s. | −2.6 | Increase |
| FGF10 | n.s. | n.s. | n.s. | n.s. | n.s. | −2.51 | Increase |
| FGF5 | n.s. | n.s. | n.s. | n.s. | n.s. | −1.63 | Increase |
| HEY1 | n.s. | n.s. | n.s. | n.s. | −2.57 | n.s. | Increase |
| HGF | n.s. | n.s. | n.s. | n.s. | n.s. | −1.94 | Increase |
| IGF1 | n.s. | n.s. | n.s. | −2.07 | −2.02 | n.s. | Increase |
| IL1B | n.s. | 1.51 | n.s. | n.s. | n.s. | 1.70 | Decrease |
| IL6 | n.s. | 1.87 | n.s. | n.s. | n.s. | 2.24 | Decrease |
| KITLG | n.s. | n.s. | n.s. | n.s. | n.s. | −3.04 | Increase |
| LEP | n.s. | 1.82 | 1.82 | n.s. | n.s. | 8.41 | Increase |
| MC1R | n.s. | n.s. | n.s. | n.s. | n.s. | −1.72 | Increase |
| MKI67 | n.s. | n.s. | n.s. | n.s. | n.s. | 2.8 | Increase |
| NOG | n.s. | −1.81 | −1.62 | n.s. | n.s. | n.s. | Increase |
| NOTCH1 | n.s. | n.s. | n.s. | n.s. | n.s. | −2.09 | Increase |
| PTCH1 | n.s. | n.s. | n.s. | n.s. | n.s. | −2.22 | Increase |
| SRFP1 | n.s. | n.s. | n.s. | n.s. | n.s. | −3.8 | Decrease |
| SOX2 | n.s. | n.s. | n.s. | n.s. | n.s. | −3.64 | Increase |
| SRD5A2 | −1.64 | −1.5 | −1.57 | n.s. | n.s. | −3.92 | Decrease |
| STAT5A | n.s. | n.s. | n.s. | n.s. | n.s. | −2.81 | Increase |
| STAT6 | n.s. | n.s. | n.s. | n.s. | n.s. | −1.88 | Decrease |
| TCF4 | n.s. | n.s. | n.s. | n.s. | n.s. | −2.26 | Increase |
| TGFB2 | n.s. | n.s. | n.s. | n.s. | −1.54 | −2.61 | Decrease |
| WNT10B | n.s. | n.s. | n.s. | n.s. | n.s. | 1.61 | Increase |

TABLE 17
Fold-Change Values for Statistically Significant Genes:
Vehicle Control vs. Untreated Control Group

| | | Desired |
|---|---|---|
| | Vehicle vs. | Expression Change |
| | Untreated | to Promote |
| Gene ID | Linear FC | Hair Growth |
| DKK1 | 1.86 | Decrease |
| IL6 | 2.83 | Decrease |
| NOG | 1.68 | Increase |
| SRD5A2 | 2.37 | Decrease |
| VCAN | −1.54 | Increase |

### 13.5 Conclusions

Compared to the initial study (as described in Example 12), in this study, the TMs produced several changes in gene expression.

EXO1 and EXO2 test groups increased expression of CORIN and LEP, which are associated with an increase in hair growth.

EXO1 test group increased expression of both IL1B and IL6, indicating a potential inflammatory response.

EXO1 and EXO2 test groups decreased expression of SRD5A2, which is associated with hormone regulation. Decreases in SRD5A2 are associated with increased hair growth.

EXO4 test group decreased both expression of BMP4 and TGFB1, which are associated with an increase in hair growth.

EXO3 and EXO4 test groups both decreased IGF1, while EXO4 also decreased HEY1. Decreases in both of these genes are associated with decreased or inhibited hair growth.

Treatment groups TM1-TM4 showed little to no change in gene expression at 24 hours.

Gene expression is a dynamic process that can be influenced by the duration of exposure to the test material. Depending on the gene, changes in expression may be observed in as early as two hours or as late as several days.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A composition comprising a purified population of plant-derived exosome-like nanoparticles isolated from tissue of a vascular plant, wherein:
size of the exosome-like nanoparticles is about 50 nm-500 nm inclusive
the exosome-like nanoparticles comprise a tuned cargo comprising,
a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and
a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase, or a combination thereof;
wherein the tuned cargo of the plant-derived exosome-like nanoparticles is produced by exposure of the plant to combinations of abiotic stress conditions that cause the plant to modulate its signaling pathways and metabolism to ensure its survival in a challenging environment.

2. The composition of claim 1, wherein
a. the plant is from Family Asphodelaceae or is an Aloe vera plant, or
b. the plant is from Family Papaveraceae and is a Celandine plant; or
c. the plant is from Family Passifloraceae and is a Passiflora ligularis or a Passiflora edulis plant; or
d. the plant is from Family Rubiaceae and is a Morinda citrifolia plant.

3. The composition of claim 1, wherein the plant tissue includes roots, stems, leaves, flowers, seeds, fruits, a liquid extract of the plant tissue, a nut milk, or a combination thereof.

4. The composition of claim 1, wherein
a. the HSP chaperones induced under the abiotic stress conditions include HSP100, HSP90, HSP70, HSP60, a small HSP or a combination thereof; and
b. the Hsfs induced under the abiotic stress conditions include HsfA, HsfB, HsfC or a combination thereof.

5. The composition of claim 1, wherein primary abiotic stress conditions, which include high/low temperature; salinity; drought; dehydration; flooding; heavy metal chemical pollutants; light stresses or physical wounding, produce secondary stresses comprising oxidative stress and osmotic stress.

6. The composition of claim 5, wherein the tuned cargo of the plant-derived exosomes is a result of exposure of the plant to two high temperature abiotic stress conditions.

7. The composition of claim 1, wherein the tuned protein cargo of the plant-derived exosomes correlates to a protein signature comprising human proteins including a keratin; semaphorin receptor plexin-B1 mitogen-activated protein kinase kinase 2 (MEKK2), diacylglycerol kinase; T cell receptor beta chain; a fez family zinc finger protein or a combination thereof.

8. The composition of claim 1, wherein the tuned cargo of the plant-derived exosome-like nanoparticles can modulate bioactivities of mammalian cells directly or indirectly.

9. The composition of claim 8, wherein
a. the mammalian cells are human cells; and
b. the bioactivities comprise a correlated signaling pathway in the human cells.

10. The composition of claim 9, wherein the human mammalian cells are cells of human skin.

11. The composition of claim 9, wherein the correlated human signaling pathways includes PI3K signaling, ERK/MAPK signaling; insulin growth factor 1 receptor (IGF1R) signaling, VEGFA/VEGFR2 signaling; leptin signaling; cytokine signaling; interleukin signaling, semaphorin signaling; sirtuin signaling; LRP1 signaling, or a combination thereof.

12. The composition of claim 6, wherein administration of the composition comprising the exosome-like nanoparticles comprising the tuned cargo modulates:
collagen production in human dermal fibroblasts in vitro; or
elastin production in human dermal fibroblasts in vitro; or
hyaluronic acid production in human dermal fibroblasts in vitro; or
interferon a2 production in mammalian PBMCs exposed to a microbial agent in vitro; or
VEGFA production in human dermal fibroblasts in vitro; or
a combination thereof.

13. The composition of claim 1, wherein the composition:
is a nutraceutical composition comprising a dietary amount of the purified plant-derived exosomes comprising the tuned cargo;
is a cosmetic composition comprising a cosmetic amount of the purified plant-derived exosomes and a cosmetically acceptable carrier; or
is a cosmeceutical composition comprising a cosmeceutical amount of the purified plant-derived exosomes comprising the tuned cargo and a cosmeceutically acceptable carrier; or
is a therapeutic composition; comprising a therapeutic amount of the purified plant-derived exosomes comprising the tuned cargo and a pharmaceutically acceptable carrier.

14. A method for improving appearance of human skin comprising:
exposing a vascular plant to combinations of abiotic stress conditions that cause the plant to modulate its signaling pathways and metabolism to ensure its survival in a challenging environment;
purifying from tissue of the vascular plant exposed to the combinations of abiotic conditions a population of plant-derived exosome-like nanoparticles (plant-derived exosomes) comprising a tuned cargo, wherein size of the plant-derived exosomes is about 50 nm-500 nm inclusive;
preparing a composition comprising about 1×10E8 to about 1×10E12, inclusive, abiotically stressed plant-derived exosome-like nanoparticles containing the tuned cargo and a cosmetically acceptable carrier; and
applying the composition topically to human skin;
wherein the tuned cargo of the plant-derived exosomes comprises:
a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and
a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase; or a combination thereof.

15. The method of claim 14, wherein
a. the plant is from Family Asphodelaceae or is an Aloe vera plant, or
b. the plant is from Family Papaveraceae and is a Celandine plant; or
c. the plant is from Family Passifloraceae and is a Passiflora ligularis or a Passiflora edulis plant; or
d. the plant is from Family Rubiaceae and is a Morinda citrifolia plant.

16. The method of claim 14, wherein the plant tissue includes roots, stems, leaves, flowers, seeds, fruits, a liquid extract of the plant tissue, a nut milk or a combination thereof.

17. The method of claim 14, wherein
a. the HSP chaperone induced under the abiotic stress conditions comprises HSP100, HSP90, HSP70, HSP60, a small HSP, or a combination thereof; and
b. the Hsf induced under the abiotic stress conditions comprises HsfA, HsfB, HsfC or a combination thereof.

18. The method of claim 14, wherein primary abiotic stress conditions including high/low temperature; salinity; drought; dehydration; flooding; heavy metal chemical pollutants; light stresses or physical wounding produce secondary stresses comprising oxidative stress and osmotic stress.

19. The method of claim 14, wherein the tuned protein cargo of the plant-derived exosome-like nanoparticles correlates to a protein signature comprising human proteins including a keratin; semaphorin receptor plexin-B1 mitogen-activated protein kinase kinase 2 (MEKK2), diacylglycerol kinase; T cell receptor beta chain; a fez family zinc finger protein or a combination thereof.

20. The method of claim 14, wherein the tuned cargo of the plant-derived exosomes can modulate bioactivities of mammalian cells directly or indirectly.

21. The method of claim 20, wherein the bioactivities comprise a correlated signaling pathway in the human cells.

22. The method of claim 20, wherein the correlated signaling pathways in the human cells include PI3K signaling, ERK/MAPK signaling; insulin growth factor 1 receptor (IGF1R) signaling, VEGFA/VEGFR2 signaling; leptin signaling; cytokine signaling; interleukin signaling, semaphorin signaling; sirtuin signaling; LRP1 signaling, or a combination thereof.

23. The method of claim 14, wherein the composition comprising the tuned cargo of the exosome-like plant nanoparticles when applied to the skin may
a. modulate gene expression in immune cells, keratinocytes, melanocytes or fibroblasts in the skin;
b. modulate a signaling pathway that contributes to inflammation, immune dysfunction or both in the skin;
c. modulate circadian rhythms of the skin and its components;
d. rejuvenate appearance of the skin by:
i. improving youthful appearance of skin;
ii. reducing appearance of wrinkles by stimulating hyaluronic acid and collagen production;
iii. improving skin clarity;
iv. improving skin texture;
v. improving skin luminosity;
vi. improving skin radiance;
vii. or a combination thereof.

24. The method of claim 14, wherein the pathway is a PI3K/AKT/mTOR pathway, an MAPK pathway, an IGF-1R pathway, a sirtuin pathway, an LRP1 pathway, or a combination thereof.

25. A method for promoting hair health comprising:
exposing a vascular plant to combinations of abiotic stress conditions that cause the plant to modulate its signaling pathways and metabolism to ensure its survival in a challenging environment;
purifying from tissue of the vascular plant exposed to the combinations of abiotic conditions a population of plant-derived exosome-like nanoparticles (plant-derived exosomes), wherein size of the plant-derived exosomes is about 50 nm-500 nm inclusive;
preparing a composition comprising about 1×10E8 to about 1×10E12, inclusive, abiotically stressed plant-derived exosome-like nanoparticles containing the tuned cargo and a cosmetically acceptable carrier; and
applying the composition topically to a subject in need thereof;
wherein the tuned cargo comprises:
a signature of miRNAs selected from ath-miR166a-3p; ath-miR166b-3p; ath-miR166e-3p; ath-miR396a-5p; ath-miR396b-5p; ath-miR396b-5p; ath-miR156ff-5p; ath-miR168b-5p; ath-miR156c-5p; ath-miR162a-3p; ath-miR162b-3p; ath-miR396a-3p; ath-miR168a-5p; ath-miR156b-5p; ath-mi156a-5p; ath-miR156d-5p; ath-miR164c-5p; ath-miR408-3p; ath-miR165a-3p; ath-miR160a-5p; ath-miR157b-5p; ath-miR157a-5p; ath-miR164b-5p; ath-miR5016; ath-miR5998b; ath-miR5020a; ath-miR836; ath-miR158a-5p; ath-miR395e; ath-miR402; ath-miR5662; ath-miR5630a; ath-miR5630b; ath-miR3933; ath-miR5998a; ath-miR172e-3p; ath-miR5024-3p; ath-miR447a-3p; ath-miR414; ath-miR167a-3p; ath-miR172b-5p; ath-miR5636; ath-miR824-3p; ath-miR172e-5p; ath-miR404; ath-miR447b; ath-miR826b; ath-miR169g-5p; ath-miR868-3p; ath-miR830-3p; ath-miR169f-5p; ath-miR828; ath-miR8182; ath-miR160a-3p; ath-miR5635d; ath-miR399c-3p; ath-miR5635c; ath-miR398a-3p; ath-miR391-5p; ath-miR781a; ath-miR157c-3p; ath-miR399b; ath-miR5014b; ath-miR5635b; ath-miR779.2; ath-miR390b-5p; ath-miR833a-3p; ath-miR849; ath-miR5635a; ath-miR841b-3p; ath-miR390a-5p; ath-miR447c-3p; ath-miR835-3p; ath-miR5638b; ath-miR2112-3p; ath-miR5653; ath-miR166a-5p; ath-miR159b-5p; ath-miR166b-5p; ath-miR843; ath-miR5015; ath-miR781b; ath-miR4245; ath-miR169b-5p; ath-miR5013; ath-miR864-5p; ath-miR866-5p; ath-miR5595a; ath-miR403-3p; ath-miR164c-3p; ath-miR835-5p; ath-miR165b; ath-miR3434-5p; ath-miR8176; ath-miR5631; ath-miR399a; ath-miR4227; ath-miR5666; ath-miR778; ath-miR851-3p; ath-miR5663-3p; ath-miR832-3p; ath-miR5646; ath-miR856; ath-miR837-5p; ath-miR846-3p; ath-miR827; ath-miR5633; ath-miR413; ath-miR838; ath-miR5654-5p; ath-miR172d-5p; ath-miR5642a; ath-miR420; ath-miR831-3p; ath-miR156d-3p; ath-miR5018; ath-miR8168; ath-miR866-3p; ath-miR8170-3p; ath-miR395b; ath-miR395c; ath-miR780.2; ath-miR167c-5p; ath-miR393a-3p; ath-miR395f; or a combination thereof; and
a signature of proteins selected from the group consisting of heat shock protein (HSP) chaperones; heat shock transcription factors (Hsfs); a ribulose 1,5-bisphosphate carboxylase/oxygenase subunit; a calcium dependent protein kinase, a PLA8 family protein; a glutathione transferase, or a combination thereof;
wherein the composition increases proliferation of dermal papillae cells and hair follicle stem cells and increases hair growth.

26. The method of claim 25, wherein applying topically includes applying to scalp, eyebrows, eyelashes or a combination thereof of the subject.

27. The method of claim 26, wherein the method
decreases hair loss;
increases hair density;
increases appearance of hair thickness;
improves scalp health;
improves hair shine;
improves hair volume or body; or
a combination thereof.

28. The method of claim 25, wherein the composition comprising the tuned cargo of the exosome-like plant nanoparticles, when applied topically, modulates gene expression in the dermal papillae cells, hair follicle stem cells, or a combination thereof.

29. The method of claim 28, wherein the composition comprising the tuned cargo of the exosome-like plant nanoparticles, when applied topically, modulates gene expression in human follicle dermal papilla cells (HFDPCs).

30. The method of claim 29, wherein the composition comprising the tuned cargo of the exosome-like plant nanoparticles, when applied topically, modulates gene expression of IL-6 in HFDPCs 24 hours after application.

31. The method of claim 28, wherein the composition comprising the tuned cargo of the exosome-like plant nanoparticles, when applied topically, modulates gene expression of CORIN, LEP, IL1B, IL-6, SRD5A2, BMP4, TGFB1, IGF1, HEY1, or a combination thereof in HFDPCs 72 hours after application.
